SEQUENCE LISTING (381 sequences)

SEQ ID NO: 1 - protein, 227 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 220 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 6 aa, synthetic construct
SDYAWN

SEQ ID NO: 4 - protein, 6 aa, synthetic construct
SDYAWN

SEQ ID NO: 5 - protein, 6 aa, synthetic construct
SDYAMN

SEQ ID NO: 6 - protein, 6 aa, synthetic construct
SDYAMN

SEQ ID NO: 7 - protein, 6 aa, synthetic construct
SDYAMN

SEQ ID NO: 8 - protein, 6 aa, synthetic construct
SDYAMN

SEQ ID NO: 9 - protein, 16 aa, synthetic construct
YISNSGSTSYNPSLKS

SEQ ID NO: 10 - protein, 16 aa, synthetic construct
YISNSGSISYNPSLKS

SEQ ID NO: 11 - protein, 16 aa, synthetic construct
YISNSGSTSYNPSVKS

SEQ ID NO: 12 - protein, 16 aa, synthetic construct
YISNSGSISYNPSVKG

SEQ ID NO: 13 - protein, 16 aa, synthetic construct
YISNSGSTSYNPSVKS

SEQ ID NO: 14 - protein, 16 aa, synthetic construct
YISNSGSTSYNPSVKS

SEQ ID NO: 15 - protein, 15 aa, synthetic construct
ERNYDYDDYYYAMDY

SEQ ID NO: 16 - protein, 15 aa, synthetic construct
ERNYDYDDYYYAMDY

SEQ ID NO: 17 - protein, 15 aa, synthetic construct
ERNYDYDDYYYAMDY

SEQ ID NO: 18 - protein, 15 aa, synthetic construct
ERNYDYDDYYYAMDY

SEQ ID NO: 19 - protein, 15 aa, synthetic construct
DRNYDYDDYYYAFDY

SEQ ID NO: 20 - protein, 15 aa, synthetic construct
DRNYDYDDYYYAFDY

SEQ ID NO: 21 - protein, 17 aa, synthetic construct
KSSQSLLYRSNQKNYLA

SEQ ID NO: 22 - protein, 17 aa, synthetic construct
KSSQSLLYRSNQKNYLA

SEQ ID NO: 23 - protein, 17 aa, synthetic construct
KSSQSLLYRSNNKNYLA

SEQ ID NO: 24 - protein, 17 aa, synthetic construct
KSSQSLLYRSNNKNYLA

SEQ ID NO: 25 - protein, 17 aa, synthetic construct
KSSQSLLYRSNNKNYLA

SEQ ID NO: 26 - protein, 17 aa, synthetic construct
KSSQSLLYRSNNKNYLA

SEQ ID NO: 27 - protein, 7 aa, synthetic construct
WASTRES

SEQ ID NO: 28 - protein, 7 aa, synthetic construct
WASTRES

SEQ ID NO: 29 - protein, 7 aa, synthetic construct
WASTRES

SEQ ID NO: 30 - protein, 7 aa, synthetic construct
WASTRES

SEQ ID NO: 31 - protein, 7 aa, synthetic construct
WASTRES

SEQ ID NO: 32 - protein, 7 aa, synthetic construct
WASTRES

SEQ ID NO: 33 - protein, 9 aa, synthetic construct
QQYYNYPRT

SEQ ID NO: 34 - protein, 9 aa, synthetic construct
QQYYNYPRT

SEQ ID NO: 35 - protein, 9 aa, synthetic construct
QQYYNYPRT

SEQ ID NO: 36 - protein, 9 aa, synthetic construct
QQYYNYPRT

SEQ ID NO: 37 - protein, 9 aa, synthetic construct
QQYYNYPRT

SEQ ID NO: 38 - protein, 9 aa, synthetic construct
QQYYNYPRT

SEQ ID NO: 39 - protein, 227 aa, synthetic construct
EVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 40 - protein, 220 aa, synthetic construct
DIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 227 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 42 - protein, 220 aa, synthetic construct
DIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 227 aa, synthetic construct
EVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 44 - protein, 220 aa, synthetic construct
DIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 227 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 220 aa, synthetic construct; identical to SEQ ID NO: 42

SEQ ID NO: 47 - protein, 227 aa, synthetic construct
EVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 48 - protein, 220 aa, synthetic construct; identical to SEQ ID NO: 44

SEQ ID NO: 49 - protein, 454 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 220 aa, synthetic construct; identical to SEQ ID NO: 2

SEQ ID NO: 51 - protein, 454 aa, synthetic construct
EVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 220 aa, synthetic construct; identical to SEQ ID NO: 40

SEQ ID NO: 53 - protein, 454 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 220 aa, synthetic construct; identical to SEQ ID NO: 42

SEQ ID NO: 55 - protein, 454 aa, synthetic construct
EVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 220 aa, synthetic construct; identical to SEQ ID NO: 44

SEQ ID NO: 57 - protein, 454 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 220 aa, synthetic construct; identical to SEQ ID NO: 42

SEQ ID NO: 59 - protein, 454 aa, synthetic construct
EVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 220 aa, synthetic construct; identical to SEQ ID NO: 44

SEQ ID NO: 61 - protein, 8 aa, synthetic construct
GFTFNKYA

SEQ ID NO: 62 - protein, 10 aa, synthetic construct
IRSKYNNYAT

SEQ ID NO: 63 - protein, 16 aa, synthetic construct
VRHGNFGNSYISYWAY

SEQ ID NO: 64 - protein, 8 aa, synthetic construct
GFTFQKYA

SEQ ID NO: 65 - protein, 16 aa, synthetic construct
ARHGNFGNSYISYWAY

SEQ ID NO: 66 - protein, 16 aa, synthetic construct
VAHGNFGNSYISYWAY

SEQ ID NO: 67 - protein, 16 aa, synthetic construct
VRAGNFGNSYISYWAY

SEQ ID NO: 68 - protein, 16 aa, synthetic construct
VRHANFGNSYISYWAY

SEQ ID NO: 69 - protein, 16 aa, synthetic construct
VRHGAFGNSYISYWAY

SEQ ID NO: 70 - protein, 16 aa, synthetic construct
VRHGNAGNSYISYWAY

SEQ ID NO: 71 - protein, 16 aa, synthetic construct
VRHGNFANSYISYWAY

SEQ ID NO: 72 - protein, 16 aa, synthetic construct
VRHGNFGASYISYWAY

SEQ ID NO: 73 - protein, 16 aa, synthetic construct
VRHGNFGNAYISYWAY

SEQ ID NO: 74 - protein, 16 aa, synthetic construct
VRHGNFGNSAISYWAY

SEQ ID NO: 75 - protein, 16 aa, synthetic construct
VRHGNFGNSYASYWAY

SEQ ID NO: 76 - protein, 16 aa, synthetic construct
VRHGNFGNSYIAYWAY

SEQ ID NO: 77 - protein, 16 aa, synthetic construct
VRHGNFGNSYISAWAY

SEQ ID NO: 78 - protein, 16 aa, synthetic construct
VRHGNFGNSYISYAAY

SEQ ID NO: 79 - protein, 16 aa, synthetic construct
VRHGNFGNSYISYWAA

SEQ ID NO: 80 - protein, 9 aa, synthetic construct
TGAVTSGNY

SEQ ID NO: 81 - skipped

SEQ ID NO: 82 - protein, 9 aa, synthetic construct
VLWYSNRWV

SEQ ID NO: 83 - protein, 9 aa, synthetic construct
TGAVTSANY

SEQ ID NO: 84 - protein, 9 aa, synthetic construct
ALWYSNRWV

SEQ ID NO: 85 - protein, 9 aa, synthetic construct
VAWYSNRWV

SEQ ID NO: 86 - protein, 9 aa, synthetic construct
VLAYSNRWV

SEQ ID NO: 87 - protein, 9 aa, synthetic construct
VLWASNRWV

SEQ ID NO: 88 - protein, 9 aa, synthetic construct
VLWYANRWV

SEQ ID NO: 89 - protein, 9 aa, synthetic construct
VLWYSARWV

SEQ ID NO: 90 - protein, 9 aa, synthetic construct
VLWYSNAWV

SEQ ID NO: 91 - protein, 9 aa, synthetic construct
VLWYSNRAV

SEQ ID NO: 92 - protein, 9 aa, synthetic construct
VLWYSNRWA

SEQ ID NO: 93 - protein, 10 aa, synthetic construct
VLWYSNRWVA

SEQ ID NO: 94 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 95 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 96 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 97 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCARHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 98 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVAHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 99 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRAGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 100 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 101 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGAFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 102 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNAGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 103 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFANSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 104 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGASYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 105 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNAYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 106 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSAISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 107 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYASYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 108 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYIAYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 109 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISAWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 110 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYAAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 111 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAAWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 112 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCALWYSNRWVFGGGTKLTVL

SEQ ID NO: 113 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVAWYSNRWVFGGGTKLTVL

SEQ ID NO: 114 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLAYSNRWVFGGGTKLTVL

SEQ ID NO: 115 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWASNRWVFGGGTKLTVL

SEQ ID NO: 116 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYANRWVFGGGTKLTVL

SEQ ID NO: 117 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSARWVFGGGTKLTVL

SEQ ID NO: 118 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNAWVFGGGTKLTVL

SEQ ID NO: 119 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRAVFGGGTKLTVL

SEQ ID NO: 120 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWAFGGGTKLTVL

SEQ ID NO: 121 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVAGGGTKLTVL

SEQ ID NO: 122 - protein, 249 aa, synthetic construct
EVQLVESGGGLVQPGGSLKLSCAASGFTFNKYAMNWVRQASGKGLEWVGRIRSKYNNYATYYADSVKDRFTISRDDSKNTAYLQMNSLKTEDTAVYYCVRHGNFGNSYISYWAYWGQGTLVTVSSGGGGSGGGGSGGGGSQTVVTQEPSFSVSPGGTVTLTCRSSTGAVTSGNYANWVQQTPGQAPRGLIGGTKFLAPGVPDRFSGSILGNKAALTITGAQADDESDYYCVLWYSNRWVFGGGTKLTVL

SEQ ID NO: 123 - protein, 249 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVL

SEQ ID NO: 124 - protein, 16 aa, synthetic construct
VRHGNFGNSYVSYWAY

SEQ ID NO: 125 - skipped

SEQ ID NO: 126 - protein, 220 aa, synthetic construct; identical to SEQ ID NO: 2

SEQ ID NO: 127 - protein, 474 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 220 aa, synthetic construct; identical to SEQ ID NO: 40

SEQ ID NO: 129 - protein, 474 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 220 aa, synthetic construct; identical to SEQ ID NO: 42

SEQ ID NO: 131 - protein, 474 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 220 aa, synthetic construct; identical to SEQ ID NO: 44

SEQ ID NO: 133 - protein, 474 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 220 aa, synthetic construct; identical to SEQ ID NO: 42

SEQ ID NO: 135 - protein, 474 aa, synthetic construct; identical to SEQ ID NO: 131

SEQ ID NO: 136 - protein, 220 aa, synthetic construct; identical to SEQ ID NO: 44

SEQ ID NO: 137 - protein, 474 aa, synthetic construct; identical to SEQ ID NO: 133

SEQ ID NO: 138 - protein, 220 aa, synthetic construct; identical to SEQ ID NO: 2

SEQ ID NO: 139 - protein, 474 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 220 aa, synthetic construct; identical to SEQ ID NO: 40

SEQ ID NO: 141 - protein, 474 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 220 aa, synthetic construct; identical to SEQ ID NO: 42

SEQ ID NO: 143 - protein, 474 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 220 aa, synthetic construct; identical to SEQ ID NO: 44

SEQ ID NO: 145 - protein, 474 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 220 aa, synthetic construct; identical to SEQ ID NO: 42

SEQ ID NO: 147 - protein, 474 aa, synthetic construct; identical to SEQ ID NO: 143

SEQ ID NO: 148 - protein, 220 aa, synthetic construct; identical to SEQ ID NO: 44

SEQ ID NO: 149 - protein, 474 aa, synthetic construct; identical to SEQ ID NO: 145

SEQ ID NO: 150 - protein, 220 aa, synthetic construct; identical to SEQ ID NO: 2

SEQ ID NO: 151 - protein, 704 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 220 aa, synthetic construct; identical to SEQ ID NO: 40

SEQ ID NO: 153 - protein, 704 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 220 aa, synthetic construct; identical to SEQ ID NO: 42

SEQ ID NO: 155 - protein, 704 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 220 aa, synthetic construct; identical to SEQ ID NO: 44

SEQ ID NO: 157 - protein, 704 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 220 aa, synthetic construct; identical to SEQ ID NO: 42

SEQ ID NO: 159 - protein, 704 aa, synthetic construct; identical to SEQ ID NO: 155

SEQ ID NO: 160 - protein, 220 aa, synthetic construct; identical to SEQ ID NO: 44

SEQ ID NO: 161 - protein, 704 aa, synthetic construct; identical to SEQ ID NO: 157

SEQ ID NO: 162 - protein, 220 aa, synthetic construct; identical to SEQ ID NO: 2

SEQ ID NO: 163 - protein, 704 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 220 aa, synthetic construct; identical to SEQ ID NO: 40

SEQ ID NO: 165 - protein, 704 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 220 aa, synthetic construct; identical to SEQ ID NO: 42

SEQ ID NO: 167 - protein, 704 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 220 aa, synthetic construct; identical to SEQ ID NO: 44

SEQ ID NO: 169 - protein, 704 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSDIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 220 aa, synthetic construct; identical to SEQ ID NO: 42

SEQ ID NO: 171 - protein, 704 aa, synthetic construct; identical to SEQ ID NO: 167

SEQ ID NO: 172 - protein, 220 aa, synthetic construct; identical to SEQ ID NO: 44

SEQ ID NO: 173 - protein, 704 aa, synthetic construct; identical to SEQ ID NO: 169

SEQ ID NO: 174 - protein, 481 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 175 - protein, 227 aa, synthetic construct; identical to SEQ ID NO: 1

SEQ ID NO: 176 - protein, 481 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 177 - protein, 227 aa, synthetic construct; identical to SEQ ID NO: 39

SEQ ID NO: 178 - protein, 481 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 179 - protein, 227 aa, synthetic construct; identical to SEQ ID NO: 41

SEQ ID NO: 180 - protein, 481 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 181 - protein, 227 aa, synthetic construct; identical to SEQ ID NO: 43

SEQ ID NO: 182 - protein, 481 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 183 - protein, 227 aa, synthetic construct; identical to SEQ ID NO: 45

SEQ ID NO: 184 - protein, 481 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 185 - protein, 227 aa, synthetic construct; identical to SEQ ID NO: 47

SEQ ID NO: 186 - protein, 481 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 187 - protein, 227 aa, synthetic construct; identical to SEQ ID NO: 1

SEQ ID NO: 188 - protein, 481 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 189 - protein, 227 aa, synthetic construct; identical to SEQ ID NO: 39

SEQ ID NO: 190 - protein, 481 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 191 - protein, 227 aa, synthetic construct; identical to SEQ ID NO: 41

SEQ ID NO: 192 - protein, 481 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 193 - protein, 227 aa, synthetic construct; identical to SEQ ID NO: 43

SEQ ID NO: 194 - protein, 481 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 195 - protein, 227 aa, synthetic construct; identical to SEQ ID NO: 45

SEQ ID NO: 196 - protein, 481 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 197 - protein, 227 aa, synthetic construct; identical to SEQ ID NO: 47

SEQ ID NO: 198 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 199 - protein, 227 aa, synthetic construct; identical to SEQ ID NO: 1

SEQ ID NO: 200 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 201 - protein, 227 aa, synthetic construct; identical to SEQ ID NO: 39

SEQ ID NO: 202 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 203 - protein, 227 aa, synthetic construct; identical to SEQ ID NO: 41

SEQ ID NO: 204 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 205 - protein, 227 aa, synthetic construct; identical to SEQ ID NO: 43

SEQ ID NO: 206 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 207 - protein, 227 aa, synthetic construct; identical to SEQ ID NO: 45

SEQ ID NO: 208 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 209 - protein, 227 aa, synthetic construct; identical to SEQ ID NO: 47

SEQ ID NO: 210 - protein, 719 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 211 - protein, 227 aa, synthetic construct; identical to SEQ ID NO: 1

SEQ ID NO: 212 - protein, 719 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 213 - protein, 227 aa, synthetic construct; identical to SEQ ID NO: 39

SEQ ID NO: 214 - protein, 719 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 215 - protein, 227 aa, synthetic construct; identical to SEQ ID NO: 41

SEQ ID NO: 216 - protein, 719 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 217 - protein, 227 aa, synthetic construct; identical to SEQ ID NO: 43

SEQ ID NO: 218 - protein, 719 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 219 - protein, 227 aa, synthetic construct; identical to SEQ ID NO: 45

SEQ ID NO: 220 - protein, 719 aa, synthetic construct
EVQLVESGGGLVQPGGSLKLSCAASGFTFQKYAMNWVRQAPGKGLEWVARIRSKYNNYATYYADSVKDRFTISRDDSKNTAYLQMNNLKTEDTAVYYCVRHGNFGNSYVSYWAYWGQGTLVTVSSGGGGSGGGGSGGGGSQTVVTQEPSLTVSPGGTVTLTCGSSTGAVTSANYPNWVQQKPGQAPRGLIGGTKFLAPGTPARFSGSLLGGKAALTLSGVQPEDEAEYYCALWYSNRWVFGGGTKLTVLGGGGSEVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 221 - protein, 227 aa, synthetic construct; identical to SEQ ID NO: 47

SEQ ID NO: 222 - protein, 15 aa, synthetic construct
GGGGSGGGGSGGGGS

SEQ ID NO: 223 - protein, 5 aa, synthetic construct
GGGGS

SEQ ID NO: 224 - protein, 9 aa, synthetic construct
GGGGSGGGS

SEQ ID NO: 225 - protein, 124 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 226 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 227 - protein, 5 aa, synthetic construct
TYWIE

SEQ ID NO: 228 - protein, 17 aa, synthetic construct
EILPGSGQTDFNEKFQG

SEQ ID NO: 229 - protein, 12 aa, synthetic construct
WGYYGTRGYFNV

SEQ ID NO: 230 - protein, 10 aa, synthetic construct
RASSSVSYMH

SEQ ID NO: 231 - protein, 7 aa, synthetic construct
STSNLAS

SEQ ID NO: 232 - protein, 9 aa, synthetic construct
QQRRSFPYT

SEQ ID NO: 233 - protein, 124 aa, synthetic construct
EVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSS

SEQ ID NO: 234 - protein, 113 aa, synthetic construct
DIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIK

SEQ ID NO: 235 - protein, 124 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSS

SEQ ID NO: 236 - protein, 113 aa, synthetic construct
DIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIK

SEQ ID NO: 237 - protein, 124 aa, synthetic construct
EVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSS

SEQ ID NO: 238 - protein, 113 aa, synthetic construct
DIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIK

SEQ ID NO: 239 - protein, 124 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSS

SEQ ID NO: 240 - protein, 113 aa, synthetic construct; identical to SEQ ID NO: 236

SEQ ID NO: 241 - protein, 124 aa, synthetic construct
EVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSS

SEQ ID NO: 242 - protein, 113 aa, synthetic construct; identical to SEQ ID NO: 238

SEQ ID NO: 243 - protein, 124 aa, synthetic construct; identical to SEQ ID NO: 225

SEQ ID NO: 244 - protein, 113 aa, synthetic construct; identical to SEQ ID NO: 226

SEQ ID NO: 245 - protein, 121 aa, synthetic construct [sequence omitted]

SEQ ID NO: 246 - protein, 106 aa, synthetic construct [sequence omitted]

SEQ ID NO: 247 - protein, 124 aa, synthetic construct; identical to SEQ ID NO: 225

SEQ ID NO: 248 - protein, 113 aa, synthetic construct; identical to SEQ ID NO: 226

SEQ ID NO: 249 - protein, 124 aa, synthetic construct; identical to SEQ ID NO: 225

SEQ ID NO: 250 - protein, 113 aa, synthetic construct; identical to SEQ ID NO: 226

SEQ ID NO: 251 - protein, 124 aa, synthetic construct
EVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSS

SEQ ID NO: 252 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIK

SEQ ID NO: 253 - protein, 124 aa, synthetic construct; identical to SEQ ID NO: 251

SEQ ID NO: 254 - protein, 113 aa, synthetic construct; identical to SEQ ID NO: 252

SEQ ID NO: 255 - protein, 227 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNGKVDEKVEPKSC

SEQ ID NO: 256 - protein, 220 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 224 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 258 - protein, 213 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 252 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 260 - protein, 252 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 261 - protein, 252 aa, synthetic construct
EVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIK

SEQ ID NO: 262 - protein, 252 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSS

SEQ ID NO: 263 - protein, 242 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIK

SEQ ID NO: 264 - protein, 242 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKGGGGSGGGGSGGGGSQVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSS

SEQ ID NO: 265 - protein, 254 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 266 - protein, 228 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 267 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 268 - protein, 214 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDGKVEPKSC

SEQ ID NO: 269 - protein, 232 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 10 aa, synthetic construct
GGGGSGGGGS

SEQ ID NO: 271 - protein, 20 aa, synthetic construct
GGGGSGGGGSGGGGSGGGGS

SEQ ID NO: 272 - protein, 25 aa, synthetic construct
GGGGSGGGGSGGGGSGGGGSGGGGS

SEQ ID NO: 273 - protein, 215 aa, synthetic construct; identical to SEQ ID NO: 267

SEQ ID NO: 274 - protein, 477 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 275 - protein, 215 aa, synthetic construct; identical to SEQ ID NO: 267

SEQ ID NO: 276 - protein, 477 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 277 - protein, 220 aa, synthetic construct; identical to SEQ ID NO: 2

SEQ ID NO: 278 - protein, 220 aa, synthetic construct; identical to SEQ ID NO: 2

SEQ ID NO: 279 - protein, 479 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 484 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 220 aa, synthetic construct; identical to SEQ ID NO: 2

SEQ ID NO: 282 - protein, 479 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 283 - protein, 215 aa, synthetic construct; identical to SEQ ID NO: 267

SEQ ID NO: 284 - protein, 477 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 285 - protein, 215 aa, synthetic construct; identical to SEQ ID NO: 267

SEQ ID NO: 286 - protein, 472 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 287 - protein, 215 aa, synthetic construct; identical to SEQ ID NO: 267

SEQ ID NO: 288 - protein, 472 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 289 - protein, 215 aa, synthetic construct; identical to SEQ ID NO: 267

SEQ ID NO: 290 - protein, 482 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 291 - protein, 215 aa, synthetic construct; identical to SEQ ID NO: 267

SEQ ID NO: 292 - protein, 482 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 293 - protein, 215 aa, synthetic construct; identical to SEQ ID NO: 267

SEQ ID NO: 294 - protein, 467 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 295 - protein, 215 aa, synthetic construct; identical to SEQ ID NO: 267

SEQ ID NO: 296 - protein, 467 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKGGGGSGGGGSGGGGSQVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 297 - protein, 213 aa, synthetic construct; identical to SEQ ID NO: 258

SEQ ID NO: 298 - protein, 472 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 299 - protein, 490 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 300 - protein, 228 aa, synthetic construct; identical to SEQ ID NO: 266

SEQ ID NO: 301 - protein, 490 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 302 - protein, 228 aa, synthetic construct; identical to SEQ ID NO: 266

SEQ ID NO: 303 - protein, 491 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 304 - protein, 227 aa, synthetic construct; identical to SEQ ID NO: 1

SEQ ID NO: 305 - protein, 227 aa, synthetic construct; identical to SEQ ID NO: 1

SEQ ID NO: 306 - protein, 486 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 307 - protein, 227 aa, synthetic construct; identical to SEQ ID NO: 1

SEQ ID NO: 308 - protein, 490 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 309 - protein, 228 aa, synthetic construct; identical to SEQ ID NO: 266

SEQ ID NO: 310 - protein, 485 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 311 - protein, 228 aa, synthetic construct; identical to SEQ ID NO: 266

SEQ ID NO: 312 - protein, 485 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 313 - protein, 228 aa, synthetic construct; identical to SEQ ID NO: 266

SEQ ID NO: 314 - protein, 495 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 315 - protein, 228 aa, synthetic construct; identical to SEQ ID NO: 266

SEQ ID NO: 316 - protein, 495 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 317 - protein, 228 aa, synthetic construct; identical to SEQ ID NO: 266

SEQ ID NO: 318 - protein, 480 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 319 - protein, 228 aa, synthetic construct; identical to SEQ ID NO: 266

SEQ ID NO: 320 - protein, 480 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKGGGGSGGGGSGGGGSQVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 321 - protein, 228 aa, synthetic construct; identical to SEQ ID NO: 266

SEQ ID NO: 322 - protein, 483 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 323 - protein, 224 aa, synthetic construct; identical to SEQ ID NO: 257

SEQ ID NO: 324 - protein, 220 aa, synthetic construct; identical to SEQ ID NO: 256

SEQ ID NO: 325 - protein, 232 aa, synthetic construct; identical to SEQ ID NO: 269

SEQ ID NO: 326 - protein, 452 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNGKVDEKVEPKSCDGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDGKVEPKSC

SEQ ID NO: 327 - protein, 220 aa, synthetic construct; identical to SEQ ID NO: 2

SEQ ID NO: 328 - protein, 214 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 329 - protein, 470 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 330 - protein, 220 aa, synthetic construct; identical to SEQ ID NO: 2

SEQ ID NO: 331 - protein, 704 aa, synthetic construct; identical to SEQ ID NO: 151

SEQ ID NO: 332 - protein, 709 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 714 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 709 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 336 - protein, 714 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 337 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 724 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 339 - protein, 714 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 724 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 729 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 719 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSEVQLVESGGGLVQPGGSLSLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLSLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 344 - protein, 227 aa, synthetic construct
EVQLVESGGGLVQPGGSLSLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 345 - protein, 227 aa, synthetic construct; identical to SEQ ID NO: 1

SEQ ID NO: 346 - protein, 227 aa, synthetic construct; identical to SEQ ID NO: 1

SEQ ID NO: 347 - protein, 227 aa, synthetic construct; identical to SEQ ID NO: 1

SEQ ID NO: 348 - protein, 227 aa, synthetic construct; identical to SEQ ID NO: 1

SEQ ID NO: 349 - protein, 227 aa, synthetic construct; identical to SEQ ID NO: 1

SEQ ID NO: 350 - protein, 227 aa, synthetic construct; identical to SEQ ID NO: 1

SEQ ID NO: 351 - protein, 227 aa, synthetic construct; identical to SEQ ID NO: 1

SEQ ID NO: 352 - protein, 227 aa, synthetic construct; identical to SEQ ID NO: 1

SEQ ID NO: 353 - protein, 227 aa, synthetic construct; identical to SEQ ID NO: 1

SEQ ID NO: 354 - protein, 227 aa, synthetic construct; identical to SEQ ID NO: 1

SEQ ID NO: 355 - protein, 227 aa, synthetic construct; identical to SEQ ID NO: 1

SEQ ID NO: 356 - protein, 220 aa, synthetic construct; identical to SEQ ID NO: 2

SEQ ID NO: 357 - protein, 244 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGG

SEQ ID NO: 358 - protein, 232 aa, synthetic construct; identical to SEQ ID NO: 269

SEQ ID NO: 359 - protein, 469 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGG

SEQ ID NO: 360 - protein, 220 aa, synthetic construct; identical to SEQ ID NO: 2

SEQ ID NO: 361 - protein, 244 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPELLGG

SEQ ID NO: 362 - protein, 214 aa, synthetic construct; identical to SEQ ID NO: 328

SEQ ID NO: 363 - protein, 487 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRKTHTCPPCPAPELLGG

SEQ ID NO: 364 - protein, 220 aa, synthetic construct; identical to SEQ ID NO: 256

SEQ ID NO: 365 - protein, 253 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCEKTHTCPPCPAPELLGGGDYKDDDDK

SEQ ID NO: 366 - protein, 232 aa, synthetic construct; identical to SEQ ID NO: 269

SEQ ID NO: 367 - protein, 469 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNGKVDEKVEPKSCDGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNGKVDKKVEPKSCRKTHTCPPCPAPELLGG

SEQ ID NO: 368 - protein, 213 aa, synthetic construct; identical to SEQ ID NO: 258

SEQ ID NO: 369 - protein, 450 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPCEEEYGSTYRCVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 370 - protein, 723 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSKTHTCPPCPAPPVAGPSVFLFPPKPKDTLMISRTPEVTCVVVDVKHEDPEVKFNWYVDGVEVHNAKTKPCEEQYGSTYRCVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 371 - protein, 11 aa, synthetic construct
DGGGGSGGGGS

SEQ ID NO: 372 - protein, 486 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 373-376 - skipped (4 entries)

SEQ ID NO: 377 - protein, 17 aa, synthetic construct
DKTHTCPPCPAPELLGG

SEQ ID NO: 378 - protein, 17 aa, synthetic construct
EKTHTCPPCPAPELLGG

SEQ ID NO: 379 - protein, 17 aa, synthetic construct
RKTHTCPPCPAPELLGG

SEQ ID NO: 380 - protein, 21 aa, synthetic construct
GGGGSGGGGSKTHTCPPCPAP

SEQ ID NO: 381 - protein, 214 aa, synthetic construct
PVAGPSVFLFPPKPKDTLMISRTPEVTCVVVDVKHEDPEVKFNWYVDGVEVHNAKTKPCEEQYGSTYRCVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0217864 A1
(43) Pub. Date: Jul. 30, 2026

(54) ANTIBODIES TARGETING STEAP-1 AND CD3 AND USES THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US); John LAUDENSLAGER, San Diego, CA (US)

(21) Appl. No.: 19/639,601

(22) Filed: Apr. 6, 2026

Related U.S. Application Data
(63) Continuation of application No. PCT/US2024/050265, filed on Oct. 7, 2024.
(60) Provisional application No. 63/588,826, filed on Oct. 9, 2023.

(51) Int. Cl. C07K 16/40; A61P 35/00; C07K 16/28

(52) U.S. Cl. CPC C07K 16/40; A61P 35/00; C07K 16/2809; C07K 16/2818; C07K 2317/31

(57) ABSTRACT

Disclosed herein are polypeptides or polypeptide complexes comprising an antibody or antigen binding fragment thereof that binds to six-transmembrane epithelial antigen of the prostate-1 (STEAP-1) and a second antibody or antigen binding fragment thereof that binds to CD3.

### CROSS-REFERENCE

The present application is a continuation of International Patent Application No. PCT/US2024/050265, filed Oct. 7, 2024, which claims the benefit of U.S. Provisional Application No. 63/588,826 filed on Oct. 9, 2023 which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 2, 2024, is named 52426-755_301SL.xml and is 422,352 bytes in size.

### SUMMARY

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding fragment thereof that binds to CD3.

In some embodiments, the at least one amino acid mutation is selected from V24A, Y27F, W35M, G45C, T58I, L64V, S66G, T74N, E99D, and M111F wherein the amino acid position corresponds to that of SEQ ID NO: 225, and Q3V, S9D, S12A, A13V, V15L, D17E, V19A, T22N, Q35N, K48Q, A49P, S66D, P86A, F89V, T91V, Q35N, and Q106C wherein the amino acid position corresponds to that of SEQ ID NO: 226. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M11IF wherein the amino acid position corresponds to that of SEQ ID NO: 225, and Q3V, S9D, S12A, A13V, V15L, D17E, V19A, T22N, Q35N, K48Q, A49P, S66D, P86A, F89V, T91V, Q35N, and Q106C wherein the amino acid position corresponds to that of SEQ ID NO: 226. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225, and Q3V, S9D, S12A, A13V, V15L, D17E, V19A, T22N, Q35N, K48Q, A49P, S66D, P86A, F89V, T91V, Q35N, and Q106C wherein the amino acid position corresponds to that of SEQ ID NO: 226, and the immunoglobulin heavy chain comprises an amino acid sequence with at least 90% sequence identity to SEQ ID NO: 225, and the immunoglobulin light chain comprises an amino acid sequence with at least 90% sequence identity to SEQ ID NO: 226. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225, and Q3V, S9D, S12A, A13V, V15L, D17E, V19A, T22N, Q35N, K48Q, A49P, S66D, P86A, F89V, T91V, Q35N, and Q106C wherein the amino acid position corresponds to that of SEQ ID NO: 226, and the immunoglobulin heavy chain comprises an amino acid sequence with at least 95% sequence identity to SEQ ID NO: 225, and the immunoglobulin light chain comprises an amino acid sequence with at least 95% sequence identity to SEQ ID NO: 226.

In some embodiments, the immunoglobulin heavy chain comprises mutations V24A, Y27F, T58I, and T74N, and at least 90% sequence identity to SEQ ID NO: 225. In some embodiments, the immunoglobulin heavy chain comprises mutations W35M and L64V and at least 90% sequence identity to SEQ ID NO: 225. In some embodiments, the immunoglobulin heavy chain comprises mutations V24A, Y27F, W35M, T58I, L64V, S66G, and T74N and at least 90% sequence identity to SEQ ID NO: 225. In some embodiments, the immunoglobulin heavy chain comprises mutations W35M, L64V, E99D, and M111F and at least 90% sequence identity to SEQ ID NO: 225. In some embodiments, the immunoglobulin heavy chain comprises mutations V24A, Y27F, W35M, L64V, T74N, E99D, and M111F and at least 90% sequence identity to SEQ ID NO: 225. In some embodiments, the immunoglobulin heavy chain comprises the G45C mutation and at least 90% sequence identity to SEQ ID NO: 225. In some embodiments, the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226. In some embodiments, the immunoglobulin light chain comprises the Q35N mutation and at least 90% sequence identity to SEQ ID NO: 226. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin light chain comprises the Q106C mutation and at least 90% sequence identity to SEQ ID NO: 226.

In some embodiments, the immunoglobulin heavy chain comprises mutations V24A, Y27F, T58I, and T74N, and at least 90% sequence identity to SEQ ID NO: 225, and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226. In some embodiments, the immunoglobulin heavy chain comprises mutations W35M and L64V and at least 90% sequence identity to SEQ ID NO: 225, and the immunoglobulin light chain comprises the Q35N mutation and at least 90% sequence identity to SEQ ID NO: 226. In some embodiments, the immunoglobulin heavy chain comprises mutations V24A, Y27F, W35M, T58I, L64V, S66G, and T74N and at least 90% sequence identity to SEQ ID NO: 225, and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin heavy chain comprises mutations W35M, L64V, E99D, and M111F and at least 90% sequence identity to SEQ ID NO: 225, and the immunoglobulin light chain comprises the Q35N mutation and at least 90% sequence identity to SEQ ID NO: 226. In some embodiments, the immunoglobulin heavy chain comprises mutations V24A, Y27F, W35M, L64V, T74N, E99D, and M111F and at least 90% sequence identity to SEQ ID NO: 225, and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin heavy chain comprises the G45C mutation and at least 90% sequence identity to SEQ ID NO: 225 and the immunoglobulin light chain comprises the Q106C mutation and at least 90% sequence identity to SEQ ID NO: 226. In some embodiments, the antibody or antigen binding fragment thereof comprises an engineered disulfide bond between the immunoglobulin heavy chain and the immunoglobulin light chain. In some embodiments, the antibody or antigen binding fragment thereof comprises an engineered disulfide bond between the cysteine at the position of the G45C mutation and the cysteine at the position of the Q106C mutation.

In some embodiments, the immunoglobulin light chain comprises complementarity determining regions (CDRs) CDR1-L, CDR2-L, and CDR3-L, and the immunoglobulin heavy chain comprises CDRs: CDR1-H, CDR2-H, and CDR3-H, wherein at least one of the CDRs comprises the amino acid sequence selected from SEQ ID NOs: 3-38. In some embodiments, CDR1-H comprises the amino acid sequence selected from SEQ ID NOs: 3-8, CDR2-H comprises the amino acid sequence selected from SEQ ID NOs: 9-14, and CDR3-H comprises the amino acid sequence selected from SEQ ID NOs: 15-20. In some embodiments, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38. In some embodiments, CDR1-H comprises the amino acid sequence selected from SEQ ID NOs: 3-8, CDR2-H comprises the amino acid sequence selected from SEQ ID NOs: 9-14, CDR3-H comprises the amino acid sequence selected from SEQ ID NOs: 15-20, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38.

In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 261. In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 262.

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the polypeptide or polypeptide complex comprises a second antibody or antigen binding fragment thereof that binds to CD3.

In some embodiments, the immunoglobulin heavy chain and the immunoglobulin light chain comprise a set of six CDR sequences selected from the group consisting of: CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the immunoglobulin light chain comprises a variable domain of an immunoglobulin kappa (IgK) or immunoglobulin lambda (IgL) light chain. In some embodiments, the immunoglobulin heavy chain comprises a constant domain derived from an IgG1, IgG2, IgG3, or IgG4 heavy chain.

In some embodiments, the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; and the immunoglobulin heavy chain comprises an amino acid sequence of at least 95% sequence identity to SEQ ID NO: 233 and the immunoglobulin light chain comprises an amino acid sequence of at least 95% sequence identity to SEQ ID NO: 234. In some embodiments, the immunoglobulin heavy chain comprises an amino acid sequence of at least 99% sequence identity to SEQ ID NO: 233 and the immunoglobulin light chain comprises an amino acid sequence of at least 99% sequence identity to SEQ ID NO: 234. In some embodiments, the immunoglobulin heavy chain comprises the amino acid sequence of SEQ ID NO: 233 and the immunoglobulin light chain comprises the amino acid sequence of SEQ ID NO: 234. In some embodiments, the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34, and the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40. In some embodiments, the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40. In some embodiments, the immunoglobulin heavy chain comprises the amino acid sequence of SEQ ID NO: 39 and the immunoglobulin light chain comprises the amino acid sequence of SEQ ID NO: 40.

In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; and the immunoglobulin heavy chain comprises an amino acid sequence of at least 95% sequence identity to SEQ ID NO: 235 and the immunoglobulin light chain comprises an amino acid sequence of at least 95% sequence identity to SEQ ID NO: 236. In some embodiments, the immunoglobulin heavy chain comprises an amino acid sequence of at least 99% sequence identity to SEQ ID NO: 235 and the immunoglobulin light chain comprises an amino acid sequence of at least 99% sequence identity to SEQ ID NO: 236. In some embodiments, the immunoglobulin heavy chain comprises the amino acid sequence of SEQ ID NO: 235 and the immunoglobulin light chain comprises the amino acid sequence of SEQ ID NO: 236. In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35, and the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42. In some embodiments, the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42. In some embodiments, the immunoglobulin heavy chain comprises the amino acid sequence of SEQ ID NO: 41 and the immunoglobulin light chain comprises the amino acid sequence of SEQ ID NO: 42.

In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; and the immunoglobulin heavy chain comprises an amino acid sequence of at least 95% sequence identity to SEQ ID NO: 237 and the immunoglobulin light chain comprises an amino acid sequence of at least 95% sequence identity to SEQ ID NO: 238. In some embodiments, the immunoglobulin heavy chain comprises an amino acid sequence of at least 99% sequence identity to SEQ ID NO: 237 and the immunoglobulin light chain comprises an amino acid sequence of at least 99% sequence identity to SEQ ID NO: 238. In some embodiments, the immunoglobulin heavy chain comprises the amino acid sequence of SEQ ID NO: 237 and the immunoglobulin light chain comprises the amino acid sequence of SEQ ID NO: 238. In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36, and the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44. In some embodiments, the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44. In some embodiments, the immunoglobulin heavy chain comprises the amino acid sequence of SEQ ID NO: 43 and the immunoglobulin light chain comprises the amino acid sequence of SEQ ID NO: 44.

In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and the immunoglobulin heavy chain comprises an amino acid sequence of at least 95% sequence identity to SEQ ID NO: 239 and the immunoglobulin light chain comprises an amino acid sequence of at least 95% sequence identity to SEQ ID NO: 240. In some embodiments, the immunoglobulin heavy chain comprises an amino acid sequence of at least 99% sequence identity to SEQ ID NO: 239 and the immunoglobulin light chain comprises an amino acid sequence of at least 99% sequence identity to SEQ ID NO: 240. In some embodiments, the immunoglobulin heavy chain comprises the amino acid sequence of SEQ ID NO: 239 and the immunoglobulin light chain comprises the amino acid sequence of SEQ ID NO: 240. In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37, and the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46. In some embodiments, the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46. In some embodiments, the immunoglobulin heavy chain comprises the amino acid sequence of SEQ ID NO: 45 and the immunoglobulin light chain comprises the amino acid sequence of SEQ ID NO: 46.

In some embodiments, the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and the immunoglobulin heavy chain comprises an amino acid sequence of at least 95% sequence identity to SEQ ID NO: 241 and the immunoglobulin light chain comprises an amino acid sequence of at least 95% sequence identity to SEQ ID NO: 242. In some embodiments, the immunoglobulin heavy chain comprises an amino acid sequence of at least 99% sequence identity to SEQ ID NO: 241 and the immunoglobulin light chain comprises an amino acid sequence of at least 99% sequence identity to SEQ ID NO: 242. In some embodiments, the immunoglobulin heavy chain comprises the amino acid sequence of SEQ ID NO: 241 and the immunoglobulin light chain comprises the amino acid sequence of SEQ ID NO: 242. In some embodiments, the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38, and the immunoglobulin heavy chain comprises an amino acid sequence of at least 95% sequence identity to SEQ ID NO: 47 and the immunoglobulin light chain comprises an amino acid sequence of at least 95% sequence identity to SEQ ID NO: 48. In some embodiments, the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48. In some embodiments, the immunoglobulin heavy chain comprises the amino acid sequence of SEQ ID NO: 47 and the immunoglobulin light chain comprises the amino acid sequence of SEQ ID NO: 48.

In some embodiments, the immunoglobulin heavy chain comprises the amino acid sequence of SEQ ID NO: 251 and the immunoglobulin light chain comprises the amino acid sequence of SEQ ID NO: 252.

In some embodiments, the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 255 and the immunoglobulin light chain comprises an amino acid sequence of at least 90% sequence identity to SEQ ID NO: 256. In some embodiments, the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 95% sequence identity to SEQ ID NO: 255 and the immunoglobulin light chain comprises an amino acid sequence of at least 95% sequence identity to SEQ ID NO: 256. In some embodiments, the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 99% sequence identity to SEQ ID NO: 255 and the immunoglobulin light chain comprises an amino acid sequence of at least 99% sequence identity to SEQ ID NO: 256. In some embodiments, the immunoglobulin heavy chain comprises the amino acid sequence of SEQ ID NO: 255 and the immunoglobulin light chain comprises the amino acid sequence of SEQ ID NO: 256.

In some embodiments, the immunoglobulin heavy chain comprises an amino acid sequence of at least 99% sequence identity to SEQ ID NO: 49 and the immunoglobulin light chain comprises an amino acid sequence of at least 99% sequence identity to SEQ ID NO: 50. In some embodiments, the immunoglobulin heavy chain comprises an amino acid sequence of at least 99% sequence identity to SEQ ID NO: 51 and the immunoglobulin light chain comprises an amino acid sequence of at least 99% sequence identity to SEQ ID NO: 52. In some embodiments, the immunoglobulin heavy chain comprises the amino acid sequence of SEQ ID NO: 51 and the immunoglobulin light chain comprises the amino acid sequence of SEQ ID NO: 52. In some embodiments, the immunoglobulin heavy chain comprises an amino acid sequence of at least 99% sequence identity to SEQ ID NO: 53 and the immunoglobulin light chain comprises an amino acid sequence of at least 99% sequence identity to SEQ ID NO: 54. In some embodiments, the immunoglobulin heavy chain comprises the amino acid sequence of SEQ ID NO: 53 and the immunoglobulin light chain comprises the amino acid sequence of SEQ ID NO: 54. In some embodiments, the immunoglobulin heavy chain comprises an amino acid sequence of at least 99% sequence identity to SEQ ID NO: 55 and the immunoglobulin light chain comprises an amino acid sequence of at least 99% sequence identity to SEQ ID NO: 56. In some embodiments, the immunoglobulin heavy chain comprises the amino acid sequence of SEQ ID NO: 55 and the immunoglobulin light chain comprises the amino acid sequence of SEQ ID NO: 56. In some embodiments, the immunoglobulin heavy chain comprises an amino acid sequence of at least 99% sequence identity to SEQ ID NO: 57 and the immunoglobulin light chain comprises an amino acid sequence of at least 99% sequence identity to SEQ ID NO: 58. In some embodiments, the immunoglobulin heavy chain comprises the amino acid sequence of SEQ ID NO: 57 and the immunoglobulin light chain comprises the amino acid sequence of SEQ ID NO: 58. In some embodiments, the immunoglobulin heavy chain comprises an amino acid sequence of at least 99% sequence identity to SEQ ID NO: 59 and the immunoglobulin light chain comprises an amino acid sequence of at least 99% sequence identity to SEQ ID NO: 60. In some embodiments, the immunoglobulin heavy chain comprises the amino acid sequence of SEQ ID NO: 59 and the immunoglobulin light chain comprises the amino acid sequence of SEQ ID NO: 60.

In some embodiments, the polypeptide or polypeptide complex that comprises the antibody or antigen binding fragment thereof that binds to STEAP-1 is a monoclonal antibody. In some embodiments, the polypeptide or polypeptide complex that comprises the antibody or antigen binding fragment thereof that binds to STEAP-1 is a monospecific antibody, bispecific antibody, or trispecific antibody. In some embodiments, the antibody or antigen binding fragment thereof that binds to STEAP-1 comprises a Fab, Fab′, (Fab′)₂ or a single chain variable fragment (scFv).

In some embodiments, the polypeptide or polypeptide complex comprises more than one antibody or antigen binding fragment thereof that binds to STEAP-1. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁). In some embodiments, L₁ connects an immunoglobulin heavy chain of the Fab or Fab′ that binds STEAP-1 with another immunoglobulin heavy chain of the second Fab or Fab′ that binds STEAP-1. In some embodiments, L₁ connects an immunoglobulin light chain of the Fab or Fab′ that binds STEAP-1 with another immunoglobulin light chain of the second Fab or Fab′ that binds STEAP-1. In some embodiments, L₁ connects an immunoglobulin light chain of the Fab or Fab′ that binds STEAP-1 with an immunoglobulin heavy chain of the second Fab or Fab′ that binds STEAP-1.

In some embodiments, the second antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 61 or SEQ ID NO: 64, CDR2-H comprises the amino acid sequence of SEQ ID NO: 62, CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A. In some embodiments, Z₁ is V, G, L, I, or A; Z₂ is L, V, I, or A; Z₃ is Y, W, F, or A; Z₄ is S, G, T, or A; Z₅ is N, Q, D, E, or A; Z₆ is R, K, or A; Z₇ is V, G, L, I, or A; Z₈ is F, Y, W, or A; Z₉ is V, G, L, I, or A; Z₁₀ is R, K, or A; Z₁₂ is G, S, T, or A; Z₁₃ is F, Y, W, or A; Z₁₄ is G, S, T, or A; Z₁₅ is N, Q, D, E, or A; Z₁₆ is S, G, T, or A; Z₁₇ is Y, W, F, or A; Z₁₈ is I, V, L, or A; Z₁₉ is S, G, T, or A; Z₂₀ is W, Y, F, or A; and Z₂₁ is Y, W, F, or A. In some embodiments, Z₈ is F. In some embodiments, Z₁₀ is R; Zn is H; Z₁₃ is F; Z₁₈ is I; Z₁₉ is S; and Z₂₀ is W.

In some embodiments, CDR1-H of the second antibody or antigen binding fragment thereof that binds to CD3 comprises the amino acid sequence of SEQ ID NO: 61 or SEQ ID NO: 64, CDR2-H of the second antibody or antigen binding fragment thereof that binds to CD3 comprises the amino acid sequence of SEQ ID NO: 62, CDR3-H of the second antibody or antigen binding fragment thereof that binds to CD3 comprises the amino acid sequence selected from SEQ ID NOs: 63, 65-79, and 124, CDR1-L of the second antibody or antigen binding fragment thereof that binds to CD3 comprises the amino acid sequence of SEQ ID NO: 80 or SEQ ID NO: 83, CDR2-L of the second antibody or antigen binding fragment thereof that binds to CD3 comprises the amino acid sequence of GTK, and CDR3-L of the second antibody or antigen binding fragment thereof that binds to CD3 comprises the amino acid sequence selected from SEQ ID NOs: 82 and 84-93.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: (GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 65, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 66, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 67, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 68, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 69, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 70, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 71, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 72, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 73, CDR1-L: SEQ ID NO: 80, CDR2-L: GTK, and CDR3-L: SEQ ID NO: 82.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 74, CDR1-L: SEQ ID NO: 80, CDR2-L: GTK, and CDR3-L: SEQ ID NO: 82. In some embodiments, second the antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 75, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 76, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 77, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 78, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 79, CDR1-L: SEQ ID NO: 80, CDR2-L: GTK, and CDR3-L: SEQ ID NO: 82.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 84. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 85. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 86. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 87. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 88.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 89. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 90. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 91. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 92. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 93. In some embodiments, the second antibody or antigen binding fragment thereof that binds to CD3 comprises the following set of six CDRs: CDR1-H: SEQ ID NO: 64, CDR2-H: SEQ ID NO: 62, CDR3-H: SEQ ID NO: 124, CDR1-L: SEQ ID NO: 83, CDR2-L: GTK, and CDR3-L: SEQ ID NO: 84.

In some embodiments, the second antibody or antigen binding fragment thereof that binds to CD3 comprises a variable domain of an immunoglobulin kappa (IgK) or immunoglobulin lambda (IgL) light chain. In some embodiments, the second antibody or antigen binding fragment thereof that binds to CD3 comprises a constant domain of an IgG1, IgG2, IgG3, or IgG4 heavy chain.

In some embodiments, the second antibody or antigen binding fragment thereof that binds to CD3 comprises amino acid sequences with at least 90%, 95%, 96%, 97%, 98%, 99%, or 100% sequence identity to SEQ ID NO: 94 and SEQ ID NO: 95. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 266 and SEQ ID NO: 267. In some embodiments, the second antibody or antigen binding fragment thereof that binds to CD3 comprises an amino acid sequence with at least 90%, 95%, 96%, 97%, 98%, or 100% sequence identity to SEQ ID NO: 268 and SEQ ID NO: 269.

In some embodiments, the second antibody or antigen binding fragment thereof that binds to CD3 comprises a Fab, Fab′, (Fab′)₂ or a single chain variable fragment (scFv). In some embodiments, the second antibody or antigen binding fragment thereof that binds to CD3 comprises the scFv. In some embodiments, the second antibody or antigen binding fragment thereof that binds to CD3 comprises the Fab. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 96. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 97. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 98. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 99. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 100. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 101. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 102. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 103. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 104. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 105. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 106. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 107. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 108. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 109. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 110.

In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 111. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 112. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 113. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 114. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 115. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 116. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 117. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 118. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 119. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 120. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 121. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 122. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 123.

In some embodiments, the antibody or antigen binding fragment thereof that binds to STEAP-1 is a Fab or Fab′, and the second antibody or antigen binding fragment thereof that binds to CD3 is an scFv and the Fab or Fab′ is connected to the scFv by a linker (L₂). In some embodiments, L2 comprises an amino acid sequence selected from any one of SEQ ID NOs: 222-224, 270-272, and 371.

In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin light chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin light chain.

In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to STEAP-1 is connected to the Fab or Fab′ that binds to CD3 by a linker (L₂). In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin light chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin light chain of the Fab or Fab′ that binds to STEAP-1.

In some embodiments, the antibody or antigen binding fragment thereof that binds to STEAP-1 is an scFv, and the second antibody or antigen binding fragment thereof that binds to CD3 is a Fab or Fab′ and the scFv that binds to STEAP-1 is connected to the Fab or Fab′ that binds to CD3 by a linker (L₂). In some embodiments, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin light chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin light chain of the scFv that binds to STEAP-1.

In some embodiments, the antibody or antigen binding fragment thereof that binds to STEAP-1 is an scFv, and wherein a C-terminus of the scFv that binds to STEAP-1 is connected to an N-terminus of the second antibody or antigen binding fragment thereof that binds to CD3 via a linker (L₂). In some embodiments, the antibody or antigen binding fragment thereof that binds to STEAP-1 is an scFv, and wherein an N-terminus of the scFv that binds to STEAP-1 is connected to a C-terminus of the second antibody or antigen binding fragment thereof that binds to CD3 via a linker (L₂). In some embodiments, a C-terminus of the immunoglobulin light chain of the scFv that binds to STEAP-1 is connected to an N-terminus of the immunoglobulin heavy chain of the scFv that binds to STEAP-1 via a linker. In some embodiments, a C-terminus of the immunoglobulin heavy chain of the scFv that binds to STEAP-1 is connected to an N-terminus of the immunoglobulin light chain that binds to STEAP-1 via a linker.

In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to an N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to the C terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the C terminus of one of the two Fab or Fab′ that are connected by L₁.

In some embodiments, L₁ connects an immunoglobulin heavy chain of the Fab or Fab′ that binds to STEAP-1 with an immunoglobulin light chain of the second antibody or antigen binding fragment that binds to CD3, and L₂ connects an immunoglobulin heavy chain of the second antibody or antigen binding fragment that binds to CD3 with another immunoglobulin heavy chain of the second Fab or Fab′ that binds to STEAP-1.

In some embodiments, L₁ or L₂ comprises an amino acid sequence according to any one of SEQ ID NOs: 222-224, 270-272, and 371.

In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 128-137, 139-149, 283-284, 287-288, and 291-292, and an amino acid sequence, that comprises the immunoglobulin heavy chain that binds to STEAP-1, with at least 95%, 98%, 99%, or 100% sequence identity to any one of SEQ ID NOs: 176-185, 187-197, 308-309, 312-313, and 316-317. In some embodiments, the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 126-127, 138, 273-282, 285-286, and 289-290, and an amino acid sequence, that comprises the immunoglobulin heavy chain that binds to STEAP-1, with at least 95%, 98%, or 99% sequence identity to any one of SEQ ID NOs: 174-175, 186, 299-307, 310-311, and 314-315.

In some embodiments, the polypeptide or polypeptide complex comprises an amino acid sequence, that comprises the immunoglobulin light chain that binds to STEAP-1, according to SEQ ID NO: 283 and an amino acid sequence, that comprises the immunoglobulin heavy chain that binds to STEAP-1, according to SEQ ID NO: 308.

In some embodiments, the polypeptide or polypeptide complex comprises an amino acid sequence that comprises the immunoglobulin light chain that binds to STEAP-1 according to SEQ ID NO: 284 and an amino acid sequence that comprises the immunoglobulin heavy chain that binds to STEAP-1 according to SEQ ID NO: 309.

In some embodiments, the polypeptide or polypeptide complex comprises an amino acid sequence that comprises the immunoglobulin light chain that binds to STEAP-1 according to SEQ ID NO: 287 and an amino acid sequence that comprises the immunoglobulin heavy chain that binds to STEAP-1 according to SEQ ID NO: 312.

In some embodiments, the polypeptide or polypeptide complex comprises an amino acid sequence that comprises the immunoglobulin light chain that binds to STEAP-1 according to SEQ ID NO: 288 and an amino acid sequence that comprises the immunoglobulin heavy chain that binds to STEAP-1 according to SEQ ID NO: 313.

In some embodiments, the polypeptide or polypeptide complex comprises an amino acid sequence that comprises the immunoglobulin light chain that binds to STEAP-1 according to SEQ ID NO: 291 and an amino acid sequence that comprises the immunoglobulin heavy chain that binds to STEAP-1 according to SEQ ID NO: 316.

In some embodiments, the polypeptide or polypeptide complex comprises an amino acid sequence that comprises the immunoglobulin light chain that binds to STEAP-1 according to SEQ ID NO: 292 and an amino acid sequence that comprises the immunoglobulin heavy chain that binds to STEAP-1 according to SEQ ID NO: 317.

In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 152-161 and 164-173, and an amino acid sequence, that comprises the immunoglobulin heavy chain that binds to STEAP-1, with at least 95%, 98%, 99%, or 100% sequence identity to any one of SEQ ID NOs: 200-209 and 212-221.

In some embodiments, the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 150-151, 162-163, and 330-342 and an amino acid sequence, that comprises the immunoglobulin heavy chain that binds to STEAP-1, with at least 95%, 98%, 99%, or 100% sequence identity to any one of SEQ ID NOs: 198-199, 210-211, and 343-355.

In some embodiments, the polypeptide or polypeptide complex comprises amino acid sequences with at least 95%, 98%, or 99% sequence identity to SEQ ID NOs: 324, 325, and 326.

In some embodiments, the polypeptide or polypeptide complex comprises amino acid sequences with at least 95%, 98%, or 99% sequence identity to SEQ ID NOs: 327, 328, and 329.

In some embodiments, the polypeptide or polypeptide complex comprises amino acid sequences with at least 95%, 98%, or 99% sequence identity to SEQ ID NOs: 356, 357, 358, and 359.

In some embodiments, the polypeptide or polypeptide complex comprises amino acid sequences with at least 95%, 98%, or 99% sequence identity to SEQ ID NOs: 360, 361, 362, and 363.

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33, wherein the polypeptide or polypeptide complex further comprises a second antibody or antigen binding fragment thereof that binds to CD3, wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 61 or SEQ ID NO: 64, CDR2-H comprises the amino acid sequence of SEQ ID NO: 62, CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

In some embodiments, the antibody or antigen binding fragment thereof that binds to STEAP-1 comprises an amino acid sequence of at least 95% sequence identity to SEQ ID NO: 225 and an amino acid sequence of at least 95% sequence identity to SEQ ID NO: 226. In some embodiments, the antibody or antigen binding fragment thereof that binds to STEAP-1 comprises an amino acid sequence of at least 99% sequence identity to SEQ ID NO: 225 and an amino acid sequence of at least 99% sequence identity to SEQ ID NO: 226. In some embodiments, the antibody or antigen binding fragment thereof that binds to STEAP-1 comprises the amino acid sequence of SEQ ID NO: 225 and the amino acid sequence of SEQ ID NO: 226.

In some embodiments, the antibody or antigen binding fragment thereof that binds to STEAP-1 comprises an amino acid sequence of at least 95% sequence identity to SEQ ID NO: 1 and an amino acid sequence of at least 95% sequence identity to SEQ ID NO: 2. In some embodiments, the antibody or antigen binding fragment thereof that binds to STEAP-1 comprises an amino acid sequence of at least 99% sequence identity to SEQ ID NO: 1 and an amino acid sequence of at least 99% sequence identity to SEQ ID NO: 2. In some embodiments, the antibody or antigen binding fragment thereof that binds to STEAP-1 comprises the amino acid sequence of SEQ ID NO: 1 and the amino acid sequence of SEQ ID NO: 2. In some embodiments, the antibody or antigen binding fragment thereof that binds to STEAP-1 comprises a Fab, Fab′, (Fab′)₂ or a single chain variable fragment (scFv).

In some embodiments, the polypeptide or polypeptide complex comprises more than one antibody or antigen binding fragment thereof that binds to STEAP-1. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁). In some embodiments, L connects an immunoglobulin heavy chain of the Fab or Fab′ that binds STEAP-1 with another immunoglobulin heavy chain of the second Fab or Fab′ that binds STEAP-1. In some embodiments, L₁ connects an immunoglobulin light chain of the Fab or Fab′ that binds STEAP-1 with another immunoglobulin light chain of the second Fab or Fab′ that binds STEAP-1. In some embodiments, L₁ connects an immunoglobulin light chain of the Fab or Fab′ that binds STEAP-1 with an immunoglobulin heavy chain of the second Fab or Fab′ that binds STEAP-1.

In some embodiments, CDR1-H of the second antibody or antigen binding fragment thereof that binds to CD3 comprises the amino acid sequence of SEQ ID NO: 61 or SEQ ID NO: 64, CDR2-H of the second antibody or antigen binding fragment thereof that binds to CD3 comprises the amino acid sequence of SEQ ID NO: 62, CDR3-H of the second antibody or antigen binding fragment thereof that binds to CD3 comprises the amino acid sequence selected from SEQ ID NOs: 63, 65-79, and 124, CDR1-L of the second antibody or antigen binding fragment thereof that binds to CD3 comprises the amino acid sequence of SEQ ID NO: 80 or SEQ ID NO: 83, CDR2-L of the second antibody or antigen binding fragment thereof that binds to CD3 comprises the amino acid sequence of GTK, and CDR3-L of the second antibody or antigen binding fragment thereof that binds to CD3 comprises the amino acid sequence selected from SEQ ID NOs: 82 and 84-93.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 65, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 66, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 67, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 68, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 69, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 70, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 71, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 72, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 73, CDR1-L: SEQ ID NO: 80, CDR2-L: GTK, and CDR3-L: SEQ ID NO: 82.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 74, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 75, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 76, CDR1-L: SEQ ID NO: 80, CDR2-L: GTK, and CDR3-L: SEQ ID NO: 82. In some embodiments, the antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 77, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 78, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 79, CDR1-L: SEQ ID NO: 80, CDR2-L: GTK, and CDR3-L: SEQ ID NO: 82.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 84. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 85. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 86. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 87. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 88.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 89. In some embodiments, the second antibody or antigen binding fragment thereof that binds to CD3 comprises the 63, CDR1-L: SEQ ID NO: 80, CDR2-L: GTK, and CDR3-L: SEQ ID NO: 90. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 91. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK and CDR3-L: SEQ ID NO: 92. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK and CDR3-L: SEQ ID NO: 93. In some embodiments, the second antibody or antigen binding fragment thereof that binds to CD3 comprises the following set of six CDRs: CDR1-H: SEQ ID NO: 64, CDR2-H: SEQ ID NO: 62, CDR3-H: SEQ ID NO: 124, CDR1-L: SEQ ID NO: 83, CDR2-L: GTK and CDR3-L: SEQ ID NO: 84.

In some embodiments, the second antibody or antigen binding fragment thereof that binds to CD3 comprises a variable domain of an immunoglobulin kappa (IgK) or immunoglobulin lambda (IgL) light chain. In some embodiments, the second antibody or antigen binding fragment thereof that binds to CD3 comprises a constant domain of an IgG1, IgG2, IgG3, or IgG4 heavy chain. In some embodiments, the second antibody or antigen binding fragment thereof that binds to CD3 comprises amino acid sequences with at least 90%, 95%, 96%, 97%, 98%, 99%, or 100% sequence identity to SEQ ID NO: 94 and SEQ ID NO: 95. In some embodiments, the second antibody or antigen binding fragment thereof that binds to CD3 comprises amino acid sequences with at least 90%, 95%, 96%, 97%, 98%, 99%, or 100% sequence identity to SEQ ID NO: 266 and SEQ ID NO: 267. In some embodiments, the second antibody or antigen binding fragment thereof that binds to CD3 comprises amino acid sequences with at least 90%, 95%, 96%, 97%, 98%, or 100% sequence identity to SEQ ID NO: 268 and SEQ ID NO: 269.

In some embodiments, the second antibody or antigen binding fragment thereof that binds to CD3 comprises a Fab, Fab′, (Fab′)₂ or a single chain variable fragment (scFv). In some embodiments, the second antibody or antigen binding fragment thereof that binds to CD3 comprises the scFv. In some embodiments, the second antibody or antigen binding fragment thereof that binds to CD3 comprises the Fab.

In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 96. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 97. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 98. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 99. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 100. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 101. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 102. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 103. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 104. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 105. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 106. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 107. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 108. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 109. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 110. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 111. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 112.

In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 113. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 114. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 115. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 116. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 117. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 118. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 119. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 120. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 121. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 122. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 123.

In some embodiments, the antibody or antigen binding fragment thereof that binds to STEAP-1 is a Fab or Fab′, and the second antibody or antigen binding fragment thereof that binds to CD3 is an scFv and the Fab or Fab′ is connected to the scFv by a linker (L₂). In some embodiments, L₂ comprises the amino acid sequence selected from SEQ ID NOs: 222-224, 270-272, and 371.

In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin light chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin light chain.

In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to STEAP-1 is connected to the Fab or Fab′ that binds to CD3 by a linker (L₂). In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin light chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin light chain of the Fab or Fab′ that binds to STEAP-1.

In some embodiments, the antibody or antigen binding fragment thereof that binds to STEAP-1 is an scFv, and the second antibody or antigen binding fragment thereof that binds to CD3 is a Fab or Fab′ and the scFv that binds to STEAP-1 is connected to the Fab or Fab′ that binds to CD3 by a linker (L₂). In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin light chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin light chain of the scFv that binds to STEAP-1.

In some embodiments, the antibody or antigen binding fragment thereof that binds to STEAP-1 is an scFv, and wherein a C-terminus of the scFv that binds to STEAP-1 is connected to an N-terminus of the second antibody or antigen binding fragment thereof that binds to CD3 via a linker (L₂). In some embodiments, the antibody or antigen binding fragment thereof that binds to STEAP-1 is an scFv, and wherein an N-terminus of the scFv that binds to STEAP-1 is connected to a C-terminus of the second antibody or antigen binding fragment thereof that binds to CD3 via a linker (L₂). In some embodiments, a C-terminus of the immunoglobulin light chain of the scFv that binds to STEAP-1 is connected to an N-terminus of the immunoglobulin heavy chain of the scFv that binds to STEAP-1 via a linker. In some embodiments, a C-terminus of the immunoglobulin heavy chain of the scFv that binds to STEAP-1 is connected to an N-terminus of the immunoglobulin light chain that binds to STEAP-1 via a linker.

In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to an N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to the C terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the C terminus of one of the two Fab or Fab′ that are connected by L₁.

In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by the second antibody or antigen binding fragment thereof that binds to CD3, wherein the second antibody or antigen binding fragment thereof that binds to CD3 comprises a CrossFab configuration, and wherein the Fab or Fab′ that binds to STEAP-1 is connected to the second antibody or antigen binding fragment thereof that binds to CD3 via a linker (L₁) and the second Fab or Fab′ that binds to STEAP-1 is connected to the second antibody or antigen binding fragment thereof that binds to CD3 via a linker (L₂). In some embodiments, L₁ connects an immunoglobulin heavy chain of the Fab or Fab′ that binds to STEAP-1 with an immunoglobulin light chain of the second antibody or antigen binding fragment that binds to CD3, and L₂ connects an immunoglobulin heavy chain of the second antibody or antigen binding fragment that binds to CD3 with another immunoglobulin heavy chain of the second Fab or Fab′ that binds to STEAP-1.

In some embodiments, L₁ or L₂ comprises an amino acid sequence according to any one of SEQ ID NOs: 222-224, 270-272, and 371.

In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 126-127, 138-139, 273-282, 285-286, and 289-290, and an amino acid sequence, that comprises the immunoglobulin heavy chain that binds to STEAP-1, with at least 95%, 98%, 99%, or 100% sequence identity to any one of SEQ ID NOs: 174-175, 186-187, 299-302, 372, 303-307, 310-311, and 314-315. In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 150-151, 162-163, and 330-342, and an amino acid sequence, that comprises the immunoglobulin heavy chain that binds to STEAP-1, with at least 95%, 98%, 99%, or 100% sequence identity to any one of SEQ ID NOs: 198-199, 210-211, and 343-355.

In some embodiments, the polypeptide or polypeptide complex comprises amino acid sequences with at least 95%, 98%, 99%, or 100% sequence identity to SEQ ID NOs: 327, 328, and 329. In some embodiments, the polypeptide or polypeptide complex comprises amino acid sequences with at least 95%, 98%, 99%, or 100% sequence identity to SEQ ID NOs: 366, 357, 358, and 359. In some embodiments, the polypeptide or polypeptide complex comprises amino acid sequences with at least 95%, 98%, 99%, or 100% sequence identity to SEQ ID NOs: 360, 361, 362, and 363.

Disclosed herein, in some embodiments, are pharmaceutical compositions comprising: (i) the polypeptide or polypeptide complex of any one of the embodiments disclosed herein; and (ii) a pharmaceutically acceptable excipient.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the polypeptide or polypeptide complex comprises a second antibody or antigen binding fragment thereof that binds to CD3.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; wherein the polypeptide or polypeptide complex further comprises a second antibody or antigen binding fragment thereof that binds to CD3 wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; and wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

Disclosed herein, in some embodiments, are isolated recombinant nucleic acid molecules encoding the polypeptide or polypeptide complex of any one of the embodiments disclosed herein.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the polypeptide or polypeptide complex comprises a second antibody or antigen binding fragment thereof that binds to CD3.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; wherein the polypeptide or polypeptide complex further comprises an antibody or antigen binding fragment thereof that binds to CD3 wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; and wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Zu is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

Disclosed herein, in some embodiments, are vectors comprising an isolated recombinant nucleic acid molecule encoding the polypeptide or polypeptide complex of any one of the embodiments disclosed herein.

Disclosed herein, in some embodiments, are host cells comprising the polypeptide or polypeptide complex of any one of the embodiments disclosed herein.

Disclosed herein are methods of treating cancer in a subject in need thereof comprising administering to the subject the polypeptide or polypeptide complex of any one of the embodiments disclosed herein. In some embodiments, the cancer is sarcoma or a cancer of the prostate, breast, pancreas, bladder, gastrointestinal tract, testis, ovary, or cervix. In some embodiments, the cancer is prostate cancer or Ewing sarcoma. In some embodiments, the method further comprises administering to the subject an immune checkpoint inhibitor. In some embodiments, the immune checkpoint inhibitor comprises an antibody or antigen binding fragment thereof that binds to PD-1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B illustrate schematics of exemplary 1+1 formats of T cell engagers (TCEs) of the present disclosure. FIG. 1A illustrates an exemplary configuration in which a single chain variable fragment (scFv) that binds to CD3 (CD3 scFv) is connected to a Fab that binds to STEAP-1 (STEAP-1 Fab) in a 1+1 format (one STEAP-1 Fab and one CD3 scFv) and in which the CD3 scFv is connected to an N-terminus of the STEAP-1 Fab immunoglobulin heavy chain. FIG. 1B illustrates another exemplary 1+1 format in which a CD3 scFv is connected to an N-terminus of a STEAP-1 Fab immunoglobulin light chain.

FIGS. 2A-2B illustrate schematics of exemplary 2+1 formats of TCEs of the present disclosure. FIG. 2A illustrates an exemplary configuration in which a CD3 scFv is connected to an N-terminus of a STEAP-1 Fab immunoglobulin heavy chain in a 2+1 format (two STEAP-1 Fabs and one CD3 scFv) and in which a linker connects a C-terminus of the immunoglobulin heavy chain of one STEAP-1 Fab with an N-terminus of the immunoglobulin heavy chain of a second STEAP-1 Fab. FIG. 2B illustrates another exemplary 2+1 format in which a CD3 scFv is connected to an N-terminus of a STEAP-1 Fab immunoglobulin light chain and in which a linker connects a C-terminus of the immunoglobulin light chain of one STEAP-1 Fab with an N-terminus of the immunoglobulin light chain of a second STEAP-1 Fab.

FIG. 3 illustrates STEAP-1 TCE mediated killing of STEAP-1 positive LNCaP tumor cells in the presence of CD8+ T-cells.

FIG. 4 illustrates STEAP-1 TCE mediated killing of STEAP-1 positive VCaP tumor cells in the presence of CD8+ T-cells.

FIG. 5 illustrates STEAP-1 TCE mediated killing of STEAP-1 positive 22Rv1 tumor cells in the presence of CD8+ T-cells.

FIGS. 6A-6B illustrate schematics of exemplary 1+1 TCE formats of the present disclosure in which an antibody or antigen binding fragment that binds to CD3 in a CrossFab configuration (CD3 CrossFab) is connected to the C-terminus of a STEAP1-1 Fab. In FIG. 6A, the immunoglobulin variable light chain of a CD3 CrossFab is connected to an immunoglobulin heavy chain (CH1 chain) of a STEAP-1 Fab via Linker 1. In FIG. 6B, the immunoglobulin variable light chain of a CD3 CrossFab is connected to an immunoglobulin heavy chain (CH1 chain) of a STEAP-1 Fab via Linker 1 and the STEAP-1 Fab includes charge steering mutations (EE/RK) to enrich correct chain pairing.

FIGS. 7A-7B illustrate schematics of exemplary 1+1 TCE formats of the present disclosure in which a CD3 scFv is connected to the C-terminus of a STEAP-1 Fab. In FIG. 7A, an immunoglobulin variable heavy chain of a CD3 scFv is connected to an immunoglobulin light chain (constant kappa light chain) of a STEAP-1 Fab via Linker 1. In FIG. 7B, an immunoglobulin heavy chain of a CD3 scFv is connected to an immunoglobulin heavy chain (CH1 chain) of a STEAP-1 Fab via Linker 1.

FIGS. 8A-8B illustrate schematics of exemplary 1+1 TCE formats of the present disclosure in which an scFv that binds to STEAP-1 (STEAP-1 scFv) is connected to the N-terminus of a Fab that binds CD3 (CD3 Fab). In FIG. 8A, an immunoglobulin variable heavy chain of the STEAP-1 scFv is connected to an immunoglobulin variable heavy chain of a CD3 Fab via Linker 1. In FIG. 8B, an immunoglobulin variable light chain of aSTEAP-1 scFv is connected to an immunoglobulin variable heavy chain of the CD3 Fab via Linker 1.

FIGS. 9A-9B illustrate schematics of exemplary 1+1 TCE formats of the present disclosure in which a CD3 scFv is connected to the N-terminus of a STEAP-1 Fab. In FIG. 9A, an immunoglobulin variable light chain of a CD3 scFv is connected to an immunoglobulin variable heavy chain of a STEAP-1 Fab via Linker 1. In FIG. 9B, an immunoglobulin variable light chain of a CD3 scFv is connected to an immunoglobulin variable light chain of a STEAP-1 Fab via Linker 1.

FIGS. 10A-10B illustrate schematics of exemplary 1+1 TCE formats of the present disclosure in which a STEAP-1 scFv is connected to the N-terminus of a CD3 Fab. In FIG. 10A, an immunoglobulin variable heavy chain of a STEAP-1 scFv is connected to an immunoglobulin light chain of a CD3 Fab via Linker 1. In FIG. 10B, an immunoglobulin light chain of a STEAP-1 scFv is connected to an immunoglobulin light chain of a CD3-Fab via Linker 1.

FIGS. 11A-11B illustrate schematics of exemplary 2+1 TCE formats of the present disclosure in which a CD3 scFv is connected to the N-terminus of a first STEAP-1 Fab and the first STEAP-1 Fab is connected to the N-terminus of a second STEAP-1 Fab. In FIG. 11A, an immunoglobulin variable light chain of a CD3 scFv is connected to an immunoglobulin variable heavy chain of a first STEAP-1 Fab via Linker 1 and an immunoglobulin constant heavy chain of the first STEAP-1 Fab (CH1 chain) is connected to an immunoglobulin variable heavy chain of a second STEAP-1 Fab via Linker 2. In FIG. 11B, an immunoglobulin variable light chain of a CD3 scFv is connected to an immunoglobulin variable light chain of a first STEAP-1 Fab via Linker 1 and an immunoglobulin constant light chain (constant kappa light chain) of the first STEAP-1 Fab is connected to an immunoglobulin variable light chain of a second STEAP-1 Fab via Linker 2.

FIGS. 12A-12D illustrate schematics of exemplary 2+1 TCE formats of the present disclosure in which two STEAP-1 Fabs and one CD3 CrossFab are connected and where a first STEAP-1 Fab is connected to the N-terminus of a CD3 CrossFab and the C-terminus of a second STEAP-1 Fab is connected to the C-terminus of the CD3 CrossFab via antibody hinge containing linker 2. In FIGS. 12A-12D, an immunoglobulin constant heavy chain (CH1 chain) of a first STEAP-1 Fab is connected to the N-terminus an immunoglobulin variable light chain of a CD3 CrossFab, and an immunoglobulin heavy chain (CH1 chain) of a the CD3 CrossFab is connected to an immunoglobulin heavy chain (CH1 chain) of a second STEAP-1 Fab via an antibody hinge containing Linker 2. In FIG. 12B and FIG. 12D, the STEAP-1 Fabs include charge steering mutations (EE/RK) to guide pairing. In FIGS. 12C-12D, Linker 2 includes a charge steering mutation (E/R) to guide steering in the antibody hinge domain.

FIG. 13 illustrates a schematic of an exemplary 2+1 TCE format of the present disclosure having two STEAP-1 Fabs, a CD3 scFv, and a fragment crystallizable (Fc) region.

FIG. 14 illustrates the influence of TCE geometry on STEAP-1 TCE mediated killing of STEAP-1 positive LNCaP tumor cells in the presence of CD8+ T-cells.

FIG. 15 illustrates STEAP-1 TCE mediated killing of STEAP-1 positive LNCaP tumor cells in the presence of CD8+ T-cells.

FIG. 16 illustrates STEAP-1 TCE mediated killing of STEAP-1 positive LNCaP tumor cells in the presence of CD8+ T-cells.

FIG. 17 illustrates a 2+1 TCE format of the present disclosure.

FIGS. 18A-18C illustrate 1+1 TCE formats of the present disclosure.

FIG. 19 illustrates binding of 1+1 TCEs and 2+1 TCEs to STEAP-1 positive LNCaP tumor cells as measured by flow cytometry. Figure discloses “GGGGS” as SEQ ID NO: 223, “GGGGSGGGGS” as SEQ ID NO: 270, “GGGGSGGGGSGGGGS” as SEQ ID NO: 222, and “GGGGSGGGGSGGGGSGGGGSGGGGS” as SEQ ID NO: 272.

FIGS. 20A-20F illustrate binding domain geometries of 1+1 TCE and 2+1 TCE constructs of the present disclosure.

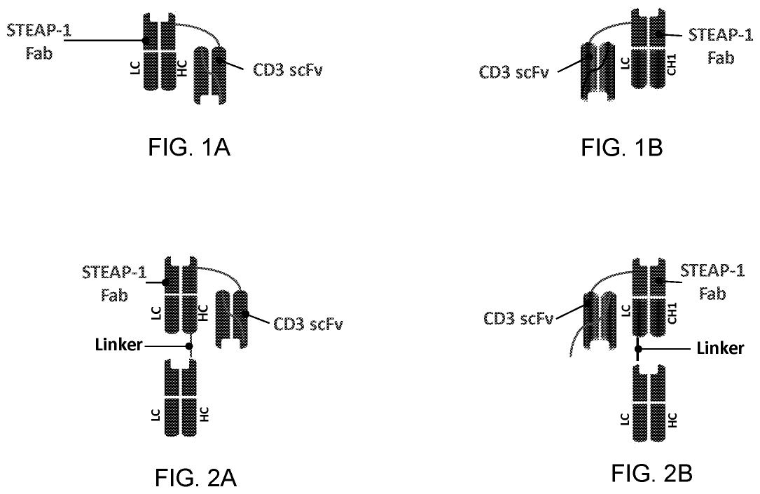

### DETAILED DESCRIPTION OF THE INVENTION

Multispecific antibodies combine the benefits of different binding specificities derived from two or more antibodies into a single composition. Multispecific antibodies for redirecting T cells to cancers have shown promise in both pre-clinical and clinical studies. This approach relies on binding of one antigen interacting portion of the antibody to a tumor-associated antigen or marker, while a second antigen interacting portion can bind to an effector cell antigen on a T cell, such as CD3, which then triggers cytotoxic activity.

One such tumor-associated antigen is six-transmembrane epithelial antigen of prostate 1 (STEAP-1). STEAP-1 is a membrane protein that is up-regulated in multiple cancer lines such as prostate, bladder, colon, ovarian, and Ewing sarcoma, among others. Given its increased expression in cancer tissues, STEAP-1 is a promising target for antibody-based cancer therapy.

Disclosed herein are various wild-type and mutated antibodies that bind to STEAP-1 and CD3. In some embodiments, the STEAP-1 binding domain is linked to the CD3 binding domain in a bispecific antibody format. In some embodiments, the STEAP-1 binding domain is in a Fab or Fab′ format, and the CD3 binding domain is a single-chain variable fragment (scFv). In some embodiments, the polypeptide or polypeptide complexes described herein are used in a method of treating cancer. In some embodiments, the cancer has cells that express or overexpress STEAP-1. In some embodiments, the cancer is sarcoma or a cancer of the prostate, breast, pancreas, bladder, gastrointestinal tract, testis, ovary, or cervix.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

As used herein, an “immunoglobulin light chain” includes at least the variable light domain and may include one or more constant light domains. As used herein, an “immunoglobulin heavy chain” includes at least the variable heavy domain and may include one or more constant heavy domains.

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′) 2 fragment's heavy chain disulfide bridge. Other chemical couplings of antibody fragments are also known.

A “CrossFab configuration” as used herein is a Fab molecule wherein the variable domains or the constant domains of the Fab heavy and light chain are exchanged (replaced by each other) (see, e.g., WO2009/080252A1 and WO2017/055388A2). For example, a Fab molecule in a CrossFab configuration may comprise a peptide chain composed of the variable light chain domain and the heavy chain constant domain (VL-CH1 in an N- to C-terminal direction), and a peptide chain composed of the variable heavy chain domain and the light chain constant domain (VH-CL in an N- to C-terminal direction).

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L₁, CDR-L₂, CDR-L₃). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L₁, FR-L₂, FR-L₃, and FR-L₄). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg NR and Rees AR, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Polypeptide or Polypeptide Complexes that Bind to STEAP-1 and CD3

### STEAP-1 Mutations

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226 (see Tables 1-2), and wherein the polypeptide or polypeptide complex comprises a second antibody or antigen binding thereof that binds to CD3.

TABLE 1
Anti-STEAP-1 Template Sequence

| | | SEQ ID |
|---|---|---|
| Description | Amino Acid Sequence | NO: |
| Anti-STEAP-1 Fab Heavy | EVQLVESGGGLVQPGGSLRLSCAVSGYSITSDYA | 1 |
| Chain (SBD-1/wt) | WNWVRQAPGKGLEWVGYISNSGSTSYNPSLKSR | |
| | FTISRDTSKNTLYLQMNSLRAEDTAVYYCARER | |
| | NYDYDDYYYAMDYWGQGTLVTVSSASTKGPSVF | |
| | PLAPSSKSTSGGTAALGCLVKDYFPEPVTVSWN | |
| | SGALTSGVHTFPAVLQSSGLYSLSSVVTVPSSS | |
| | LGTQTYICNVNHKPSNTKVDKKVEPKSC | |
| Anti-STEAP-1 Heavy | EVQLVESGGGLVQPGGSLRLSCAVSGYSITSDYA | 225 |
| Chain Variable Domain | WNWVRQAPGKGLEWVGYISNSGSTSYNPSLKSR | |
| (SBD-1/wt) | FTISRDTSKNTLYLQMNSLRAEDTAVYYCARER | |
| | NYDYDDYYYAMDYWGQGTLVTVSS | |
| Anti-STEAP-1 Fab Light | DIQMTQSPSSLSASVGDRVTITCKSSQSLLYRSN | 2 |
| Chain (SBD-1/wt) | QKNYLAWYQQKPGKAPKLLIYWASTRESGVPSRF | |
| | SGSGSGTDFTLTISSLQPEDFATYYCQQYYNYPR | |
| | TFGQGTKVEIKRTVAAPSVFIFPPSDEQLKSGTA | |
| | SVVCLLNNFYPREAKVQWKVDNALQSGNSQESV | |
| | TEQDSKDSTYSLSSTLTLSKADYEKHKVYACEVT | |
| | HQGLSSPVTKSFNRGEC | |
| Anti-STEAP-1 Light | DIQMTQSPSSLSASVGDRVTITCKSSQSLLYRSN | 226 |
| Chain Variable Domain | QKNYLAWYQQKPGKAPKLLIYWASTRESGVPSRF | |
| (SBD-1/wt) | SGSGSGTDFTLTISSLQPEDFATYYCQQYYNYPR | |
| | TFGQGTKVEIK | |

TABLE 2
Anti-STEAP-1 Mutations

| Chain | Position or Mutation |
|---|---|
| Exemplary amino acid positions | V24, Y27, W35, G45, T58, L64, |
| for mutation | S66, T74, E99, M111 |
| Heavy Chain | |
| (numbering corresponding to | |
| SEQ ID NO: 225) | |
| Exemplary amino acid positions | V24A, Y27F, W35M, G45C, T58I, |
| and mutations | L64V, S66G, T74N, E99D, M111F |
| Heavy Chain | |
| (numbering corresponding to | |
| SEQ ID NO: 225) | |
| Exemplary amino acid positions | Q3, S9, S12, A13, V15, D17, V19, |
| for mutation | T22, Q35, K48, A49, S66, P86, |
| Light Chain | F89, T91, Q106 |
| (numbering corresponding to | |
| SEQ ID NO: 226) | |
| Exemplary amino acid positions | Q3V, S9D, S12A, A13V, V15L, D17E, |
| and mutations | V19A, T22N, Q35N, K48Q, A49P, |
| Light Chain | S66D, P86A, F89V, T91V, Q106C |
| (numbering corresponding to | |
| SEQ ID NO: 226) | |

In some embodiments, the at least one amino acid mutation is selected from V24A, Y27F, W35M, G45C, T58I, L64V, S66G, T74N, E99D, and M111F wherein the amino acid position corresponds to that of SEQ ID NO: 225, and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226. In some embodiments, the at least one amino acid mutation is V24A wherein the amino acid position corresponds to that of SEQ ID NO: 225. In some embodiments, the at least one amino acid mutation is Y27F wherein the amino acid position corresponds to that of SEQ ID NO: 225. In some embodiments, the at least one amino acid mutation is W35M wherein the amino acid position corresponds to that of SEQ ID NO: 225. In some embodiments, the at least one amino acid mutation is G45C wherein the amino acid position corresponds to that of SEQ ID NO: 225. In some embodiments, the at least one amino acid mutation is T58I wherein the amino acid position corresponds to that of SEQ ID NO: 225. In some embodiments, the at least one amino acid mutation is L64V wherein the amino acid position corresponds to that of SEQ ID NO: 225. In some embodiments, the at least one amino acid mutation is S66G wherein the amino acid position corresponds to that of SEQ ID NO: 225. In some embodiments, the at least one amino acid mutation is T74N wherein the amino acid position corresponds to that of SEQ ID NO: 225. In some embodiments, the at least one amino acid mutation E99D wherein the amino acid position corresponds to that of SEQ ID NO: 225. In some embodiments, the at least one amino acid mutation is M111F wherein the amino acid position corresponds to that of SEQ ID NO: 225. In some embodiments, the at least one amino acid mutation is Q3V wherein the amino acid position corresponds to that of SEQ ID NO: 226. In some embodiments, the at least one amino acid mutation is S9D wherein the amino acid position corresponds to that of SEQ ID NO: 226. In some embodiments, the at least one amino acid mutation is S12A wherein the amino acid position corresponds to that of SEQ ID NO: 226. In some embodiments, the at least one amino acid mutation is A13V wherein the amino acid position corresponds to that of SEQ ID NO: 226. In some embodiments, the at least one amino acid mutation is V15L wherein the amino acid position corresponds to that of SEQ ID NO: 226. In some embodiments, the at least one amino acid mutation is D17E wherein the amino acid position corresponds to that of SEQ ID NO: 226. In some embodiments, the at least one amino acid mutation is V19A wherein the amino acid position corresponds to that of SEQ ID NO: 226. In some embodiments, the at least one amino acid mutation is T22N wherein the amino acid position corresponds to that of SEQ ID NO: 226. In some embodiments, the at least one amino acid mutation is Q35N wherein the amino acid position corresponds to that of SEQ ID NO: 226. In some embodiments, the at least one amino acid mutation is K48Q wherein the amino acid position corresponds to that of SEQ ID NO: 226. In some embodiments, the at least one amino acid mutation is A49P wherein the amino acid position corresponds to that of SEQ ID NO: 226. In some embodiments, the at least one amino acid mutation is S66D wherein the amino acid position corresponds to that of SEQ ID NO: 226. In some embodiments, the at least one amino acid mutation is P86A wherein the amino acid position corresponds to that of SEQ ID NO: 226. In some embodiments, the at least one amino acid mutation is F89V wherein the amino acid position corresponds to that of SEQ ID NO: 226. In some embodiments, the at least one amino acid mutation is T91V wherein the amino acid position corresponds to that of SEQ ID NO: 226. In some embodiments, the at least one amino acid mutation is Q35N wherein the amino acid position corresponds to that of SEQ ID NO: 226. In some embodiments, the at least one amino acid mutation is Q106C wherein the amino acid position corresponds to that of SEQ ID NO: 226.

In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226, and the immunoglobulin heavy chain comprises an amino acid sequence with at least 90% sequence identity to SEQ ID NO: 225, and the immunoglobulin light chain comprises an amino acid sequence with at least 90% sequence identity to SEQ ID NO: 226. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226, and the immunoglobulin heavy chain comprises an amino acid sequence with at least 92% sequence identity to SEQ ID NO: 225, and the immunoglobulin light chain comprises an amino acid sequence with at least 92% sequence identity to SEQ ID NO: 226. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226, and the immunoglobulin heavy chain comprises an amino acid sequence with at least 95% sequence identity to SEQ ID NO: 225, and the immunoglobulin light chain comprises an amino acid sequence with at least 95% sequence identity to SEQ ID NO: 226.

In some embodiments, the immunoglobulin heavy chain comprises mutations V24A, Y27F, T58I, and T74N, and at least 90% sequence identity to SEQ ID NO: 225. In some embodiments, the immunoglobulin heavy chain comprises mutations W35M and L64V and at least 90% sequence identity to SEQ ID NO: 225. In some embodiments, the immunoglobulin heavy chain comprises mutations V24A, Y27F, W35M, T58I, L64V, S66G, and T74N and at least 90% sequence identity to SEQ ID NO: 225. In some embodiments, the immunoglobulin heavy chain comprises mutations W35M, L64V, E99D, and M111F and at least 90% sequence identity to SEQ ID NO: 225. In some embodiments, the immunoglobulin heavy chain comprises mutations V24A, Y27F, W35M, L64V, T74N, E99D, and M111F and at least 90% sequence identity to SEQ ID NO: 225. In some embodiments, the immunoglobulin heavy chain comprises the G45C mutation and at least 90% sequence identity to SEQ ID NO: 225.

In some embodiments, the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226. In some embodiments, the immunoglobulin light chain comprises the Q35N mutation and at least 90% sequence identity to SEQ ID NO: 226. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin light chain comprises the Q106C mutation and at least 90% sequence identity to SEQ ID NO: 226.

In some embodiments, the immunoglobulin heavy chain comprises mutations V24A, Y27F, T58I, and T74N, and at least 90% sequence identity to SEQ ID NO: 225 and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226. In some embodiments, the immunoglobulin heavy chain comprises mutations V24A, Y27F, T58I, and T74N wherein the amino acid position corresponds to that of SEQ ID NO: 225, and the immunoglobulin light chain comprises mutations Q3V, S9D, S12A, A13V, V15L, D17E, V19A, T22N, K48Q, A49P, S66D, P86A, F89V, and T91V wherein the amino acid position corresponds to that of SEQ ID NO: 226.

In some embodiments, the immunoglobulin heavy chain comprises mutations W35M and L64V and at least 90% sequence identity to SEQ ID NO: 225 and the immunoglobulin light chain comprises the Q35N mutation and at least 90% sequence identity to SEQ ID NO: 226. In some embodiments, the immunoglobulin heavy chain comprises mutations W35M and L64V and at least 95% sequence identity to SEQ ID NO: 225 and the immunoglobulin light chain comprises the Q35N mutation and at least 95% sequence identity to SEQ ID NO: 226. In some embodiments, the immunoglobulin heavy chain comprises mutations W35M and L64V wherein the amino acid position corresponds to that of SEQ ID NO: 225, and the immunoglobulin light chain comprises the Q35N mutation wherein the amino acid position corresponds to that of SEQ ID NO: 226.

In some embodiments, the immunoglobulin heavy chain comprises mutations V24A, Y27F, W35M, T58I, L64V, S66G, and T74N and at least 90% sequence identity to SEQ ID NO: 225 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin heavy chain comprises mutations V24A, Y27F, W35M, T58I, L64V, S66G, and T74N wherein the amino acid position corresponds to that of SEQ ID NO: 225, and the immunoglobulin light chain comprises mutations Q3V, S9D, S12A, A13V, V15L, D17E, V19A, T22N, Q35N, K48Q, A49P, S66D, P86A, F89V, and T91V wherein the amino acid position corresponds to that of SEQ ID NO: 226.

In some embodiments, the immunoglobulin heavy chain comprises mutations W35M, L64V, E99D, and M111F and at least 90% sequence identity to SEQ ID NO: 225 and the immunoglobulin light chain comprises the Q35N mutation and at least 90% sequence identity to SEQ ID NO: 226. In some embodiments, the immunoglobulin heavy chain comprises mutations W35M, L64V, E99D, and M111F and at least 95% sequence identity to SEQ ID NO: 225 and the immunoglobulin light chain comprises the Q35N mutation and at least 95% sequence identity to SEQ ID NO: 226. In some embodiments, the immunoglobulin heavy chain comprises mutations W35M, L64V, E99D, and M111F wherein the amino acid position corresponds to that of SEQ ID NO: 225, and the immunoglobulin light chain comprises the Q35N mutation wherein the amino acid position corresponds to that of SEQ ID NO: 226.

In some embodiments, the immunoglobulin heavy chain comprises mutations V24A, Y27F, W35M, L64V, T74N, E99D, and M111F and at least 90% sequence identity to SEQ ID NO: 225 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226. In some embodiments, the immunoglobulin heavy chain comprises mutations V24A, Y27F, W35M, L64V, T74N, E99D, and M111F wherein the amino acid position corresponds to that of SEQ ID NO: 225, and the immunoglobulin light chain comprises mutations Q3V, S9D, S12A, A13V, V15L, D17E, V19A, T22N, Q35N, K48Q, A49P, S66D, P86A, F89V, and T91V wherein the amino acid position corresponds to that of SEQ ID NO: 226.

In some embodiments, the immunoglobulin heavy chain comprises the G45C mutation and at least 90% sequence identity to SEQ ID NO: 225 and the immunoglobulin light chain comprises the Q106C mutation and at least 90% sequence identity to SEQ ID NO: 226.

In some embodiments, the antibody or antigen binding fragment thereof comprises an engineered disulfide bond between the immunoglobulin heavy chain and the immunoglobulin light chain. In some embodiments, the antibody or antigen binding fragment thereof comprises an engineered disulfide bond between the cysteine at the position of the G45C mutation and the cysteine at the position of the Q106C mutation.

In some embodiments, the immunoglobulin light chain comprises complementarity determining regions (CDRs) CDR1-L, CDR2-L, and CDR3-L, and the immunoglobulin heavy chain comprises CDRs: CDR1-H, CDR2-H, and CDR3-H, wherein at least one of the CDRs comprises the amino acid sequence selected from SEQ ID NOs: 3-38.

TABLE 3
Anti-STEAP Heavy Chain CDR sequences as (according to Kabat)

| | | SEQ | | SEQ | | SEQ |
|---|---|---|---|---|---|---|
| Construct | | ID | | ID | | ID |
| Name | CDR1-H | NO: | CDR2-H | NO: | CDR3-H | NO: |
| HC CDR- | SDYAWN | 3 | YISNSGSTSYNPSLKS | 9 | ERNYDYDDYYYAMDY | 15 |
| wt | | | | | | |
| HC CDR- | SDYAWN | 4 | YISNSGSISYNPSLKS | 10 | ERNYDYDDYYYAMDY | 16 |
| T58I | | | | | | |
| HC CDR- | SDYAMN | 5 | YISNSGSTSYNPSVKS | 11 | ERNYDYDDYYYAMDY | 17 |
| W35M, | | | | | | |
| L64V | | | | | | |
| HC CDR- | SDYAMN | 6 | YISNSGSISYNPSVKG | 12 | ERNYDYDDYYYAMDY | 18 |
| W35M, | | | | | | |
| T58I, | | | | | | |
| L64V, | | | | | | |
| S66G | | | | | | |
| HC CDR- | SDYAMN | 7 | YISNSGSTSYNPSVKS | 13 | DRNYDYDDYYYAFDY | 19 |
| W35M, | | | | | | |
| L64V, | | | | | | |
| E99D, | | | | | | |
| M111F | | | | | | |
| HC CDR- | SDYAMN | 8 | YISNSGSTSYNPSVKS | 14 | DRNYDYDDYYYAFDY | 20 |
| W35M, | | | | | | |
| L64V, | | | | | | |
| E99D, | | | | | | |
| M111F | | | | | | |
| HC CDR | TYWIE | 227 | EILPGSGQTDFNEKFQG | 228 | WGYYGTRGYFNV | 229 |
| AM509 | | | | | | |

TABLE 4
Anti-STEAP Light Chain CDR sequences as (according to Kabat)

| | | SEQ | | SEQ | | SEQ |
|---|---|---|---|---|---|---|
| Construct | | ID | | ID | | ID |
| Name | CDR1-L | NO: | CDR2-L | NO: | CDR3-L | NO: |
| LC CDR- | KSSQSLLYRSNQKNYLA | 21 | WASTRES | 27 | QQYYNYPRT | 33 |
| wt | | | | | | |
| LC CDR- | KSSQSLLYRSNQKNYLA | 22 | WASTRES | 28 | QQYYNYPRT | 34 |
| wt | | | | | | |
| LC CDR- | KSSQSLLYRSNNKNYLA | 23 | WASTRES | 29 | QQYYNYPRT | 35 |
| Q35N | | | | | | |
| LC CDR- | KSSQSLLYRSNNKNYLA | 24 | WASTRES | 30 | QQYYNYPRT | 36 |
| Q35N | | | | | | |
| LC CDR- | KSSQSLLYRSNNKNYLA | 25 | WASTRES | 31 | QQYYNYPRT | 37 |
| Q35N | | | | | | |
| LC CDR- | KSSQSLLYRSNNKNYLA | 26 | WASTRES | 32 | QQYYNYPRT | 38 |
| Q35N | | | | | | |
| LC CDR | RASSSVSYMH | 230 | STSNLAS | 231 | QQRRSFPYT | 232 |
| AM509 | | | | | | |

In some embodiments, CDR1-H comprises the amino acid sequence selected from SEQ ID NOs: 3-8, CDR2-H comprises the amino acid sequence selected from SEQ ID NOs: 9-14, and CDR3-H comprises the amino acid sequence selected from SEQ ID NOs: 15-20. In some embodiments, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38. In some embodiments, CDR1-H comprises the amino acid sequence selected from SEQ ID NOs: 3-8, CDR2-H comprises the amino acid sequence selected from SEQ ID NOs: 9-14, CDR3-H comprises the amino acid sequence selected from SEQ ID NOs: 15-20, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38.

In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 261. In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 262.

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the polypeptide or polypeptide complex comprises a second antibody or antigen binding fragment thereof that binds to CD3.

In some embodiments, the immunoglobulin heavy chain and the immunoglobulin light chain comprise a set of six CDR sequences selected from the group consisting of: CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the immunoglobulin light chain comprises a variable domain of an immunoglobulin kappa (IgK) or immunoglobulin lambda (IgL) light chain. In some embodiments, the immunoglobulin heavy chain comprises a constant domain derived from an IgG1, IgG2, IgG3, or IgG4 heavy chain.

In some embodiments, the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; and the immunoglobulin heavy chain comprises an amino acid sequence of at least 95% sequence identity to SEQ ID NO: 233 and the immunoglobulin light chain comprises an amino acid sequence of at least 95% sequence identity to SEQ ID NO: 234. In some embodiments, the immunoglobulin heavy chain comprises an amino acid sequence of at least 99% sequence identity to SEQ ID NO: 233 and the immunoglobulin light chain comprises an amino acid sequence of at least 99% sequence identity to SEQ ID NO: 234. In some embodiments, the immunoglobulin heavy chain comprises the amino acid sequence of SEQ ID NO: 233 and the immunoglobulin light chain comprises the amino acid sequence of SEQ ID NO: 234. In some embodiments, the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34, and the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40. In some embodiments, the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40. In some embodiments, the immunoglobulin heavy chain comprises the amino acid sequence of SEQ ID NO: 39 and the immunoglobulin light chain comprises the amino acid sequence of SEQ ID NO: 40.

In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; and the immunoglobulin heavy chain comprises an amino acid sequence of at least 95% sequence identity to SEQ ID NO: 235 and the immunoglobulin light chain comprises an amino acid sequence of at least 95% sequence identity to SEQ ID NO: 236. In some embodiments, wherein the immunoglobulin heavy chain comprises an amino acid sequence of at least 99% sequence identity to SEQ ID NO: 235 and the immunoglobulin light chain comprises an amino acid sequence of at least 99% sequence identity to SEQ ID NO: 236. In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35, and the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42. In some embodiments, the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42. In some embodiments, the immunoglobulin heavy chain comprises the amino acid sequence of SEQ ID NO: 41 and the immunoglobulin light chain comprises the amino acid sequence of SEQ ID NO: 42.

In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; and the immunoglobulin heavy chain comprises an amino acid sequence of at least 95% sequence identity to SEQ ID NO: 237 and the immunoglobulin light chain comprises an amino acid sequence of at least 95% sequence identity to SEQ ID NO: 238. In some embodiments, the immunoglobulin heavy chain comprises an amino acid sequence of at least 99% sequence identity to SEQ ID NO: 237 and the immunoglobulin light chain comprises an amino acid sequence of at least 99% sequence identity to SEQ ID NO: 238. In some embodiments, the immunoglobulin heavy chain comprises the amino acid sequence of SEQ ID NO: 237 and the immunoglobulin light chain comprises the amino acid sequence of SEQ ID NO: 238. In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36, and the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44. In some embodiments, the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44. In some embodiments, the immunoglobulin heavy chain comprises the amino acid sequence of SEQ ID NO: 43 and the immunoglobulin light chain comprises the amino acid sequence of SEQ ID NO: 44.

In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and the immunoglobulin heavy chain comprises an amino acid sequence of at least 95% sequence identity to SEQ ID NO: 239 and the immunoglobulin light chain comprises an amino acid sequence of at least 95% sequence identity to SEQ ID NO: 240. In some embodiments, the immunoglobulin heavy chain comprises an amino acid sequence of at least 99% sequence identity to SEQ ID NO: 239 and the immunoglobulin light chain comprises an amino acid sequence of at least 99% sequence identity to SEQ ID NO: 240. In some embodiments, the immunoglobulin heavy chain comprises the amino acid sequence of SEQ ID NO: 239 and the immunoglobulin light chain comprises the amino acid sequence of SEQ ID NO: 240. In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37, and the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46. In some embodiments, the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46. In some embodiments, the immunoglobulin heavy chain comprises the amino acid sequence of SEQ ID NO: 45 and the immunoglobulin light chain comprises the amino acid sequence of SEQ ID NO: 46.

In some embodiments, the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and the immunoglobulin heavy chain comprises an amino acid sequence of at least 95% sequence identity to SEQ ID NO: 241 and the immunoglobulin light chain comprises an amino acid sequence of at least 95% sequence identity to SEQ ID NO: 242. In some embodiments, the immunoglobulin heavy chain comprises an amino acid sequence of at least 99% sequence identity to SEQ ID NO: 241 and the immunoglobulin light chain comprises an amino acid sequence of at least 99% sequence identity to SEQ ID NO: 242. In some embodiments, the immunoglobulin heavy chain comprises the amino acid sequence of SEQ ID NO: 241 and the immunoglobulin light chain comprises the amino acid sequence of SEQ ID NO: 242. In some embodiments, the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38, and the immunoglobulin heavy chain comprises an amino acid sequence of at least 95% sequence identity to SEQ ID NO: 47 and the immunoglobulin light chain comprises an amino acid sequence of at least 95% sequence identity to SEQ ID NO: 48. In some embodiments, the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48. In some embodiments, the immunoglobulin heavy chain comprises the amino acid sequence of SEQ ID NO: 47 and the immunoglobulin light chain comprises the amino acid sequence of SEQ ID NO: 48.

In some embodiments, the immunoglobulin heavy chain comprises an amino acid sequence of at least 95% sequence identity to SEQ ID NO: 243 and the immunoglobulin light chain comprises an amino acid sequence of at least 95% sequence identity to SEQ ID NO: 244. In some embodiments, the immunoglobulin heavy chain comprises an amino acid sequence of at least 99% sequence identity to SEQ ID NO: 243 and the immunoglobulin light chain comprises an amino acid sequence of at least 99% sequence identity to SEQ ID NO: 244. In some embodiments, the immunoglobulin heavy chain comprises the amino acid sequence of SEQ ID NO: 243 and the immunoglobulin light chain comprises the amino acid sequence of SEQ ID NO: 244. In some embodiments, the immunoglobulin heavy chain comprises an amino acid sequence of at least 95% sequence identity to SEQ ID NO: 255 and the immunoglobulin light chain comprises an amino acid sequence of at least 95% sequence identity to SEQ ID NO: 256. In some embodiments, the immunoglobulin heavy chain comprises an amino acid sequence of at least 99% sequence identity to SEQ ID NO: 255 and the immunoglobulin light chain comprises an amino acid sequence of at least 95% sequence identity to SEQ ID NO: 256. In some embodiments, the immunoglobulin heavy chain comprises the amino acid sequence of SEQ ID NO: 255 and the immunoglobulin light chain comprises the amino acid sequence of SEQ ID NO: 256.

In some embodiments, the immunoglobulin heavy chain comprises an amino acid sequence of at least 95% sequence identity to SEQ ID NO: 245 and the immunoglobulin light chain comprises an amino acid sequence of at least 95% sequence identity to SEQ ID NO: 246. In some embodiments, the immunoglobulin heavy chain comprises an amino acid sequence of at least 99% sequence identity to SEQ ID NO: 245 and the immunoglobulin light chain comprises an amino acid sequence of at least 99% sequence identity to SEQ ID NO: 246. In some embodiments, the immunoglobulin heavy chain comprises the amino acid sequence of SEQ ID NO: 245 and the immunoglobulin light chain comprises the amino acid sequence of SEQ ID NO: 246. In some embodiments, the immunoglobulin heavy chain comprises an amino acid sequence of at least 95% sequence identity to SEQ ID NO: 257 and the immunoglobulin light chain comprises an amino acid sequence of at least 95% sequence identity to SEQ ID NO: 258. In some embodiments, the immunoglobulin heavy chain comprises an amino acid sequence of at least 99% sequence identity to SEQ ID NO: 257 and the immunoglobulin light chain comprises an amino acid sequence of at least 95% sequence identity to SEQ ID NO: 258. In some embodiments, the immunoglobulin heavy chain comprises the amino acid sequence of SEQ ID NO: 257 and the immunoglobulin light chain comprises the amino acid sequence of SEQ ID NO: 258.

In some embodiments, the immunoglobulin heavy chain comprises an amino acid sequence of at least 95%, 98%, 99%, or 100% sequence identity to SEQ ID NO: 251 and the immunoglobulin light chain comprises an amino acid sequence of at least 95%, 98%, 99%, or 100% sequence identity to SEQ ID NO: 252. In some embodiments, the immunoglobulin heavy chain comprises an amino acid sequence of at least 95%, 98%, 99%, or 100% sequence identity to SEQ ID NO: 253 and the immunoglobulin light chain comprises an amino acid sequence of at least 95%, 98%, 99%, or 100% sequence identity to SEQ ID NO: 254.

In some embodiments, the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 255 and the immunoglobulin light chain comprises an amino acid sequence of at least 90% sequence identity to SEQ ID NO: 256. In some embodiments, the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 95% sequence identity to SEQ ID NO: 255 and the immunoglobulin light chain comprises an amino acid sequence of at least 95% sequence identity to SEQ ID NO: 256.

TABLE 5
Anti-STEAP-1 Variable Heavy Chain and Variable Light Chain Sequences

| Construct | | SEQ | | SEQ |
|---|---|---|---|---|
| Name/ | | ID | | ID |
| mutations | Heavy Chain | NO: | Light Chain | NO: |
| SBD-2/ | EVQLVESGGGLVQPGGSLRLSCAAS | 233 | DIVMTQSPDSLAVSLGE | 234 |
| HC: | GFSITSDYAWNWVRQAPGKGLEWVG | | RATINCKSSQSLLYRSN | |
| V24A, | YISNSGSISYNPSLKSRFTISRDNS | | QKNYLAWYQQKPGQPP | |
| Y27F, | KNTLYLQMNSLRAEDTAVYYCARER | | KLLIYWASTRESGVPDR | |
| T58I, | NYDYDDYYYAMDYWGQGTLVTVS | | FSGSGSGTDFTLTISSLQ | |
| T74N | S | | AEDVAVYYCQQYYNYP | |
| LC: Q3V, | | | RTFGQGTKVEIK | |
| S9D, | | | | |
| S12A, | | | | |
| A13V, | | | | |
| V15L, | | | | |
| D17E, | | | | |
| V19A, | | | | |
| T22N, | | | | |
| K48Q, | | | | |
| A49P, | | | | |
| S66D, | | | | |
| P86A, | | | | |
| F89V, | | | | |
| T91V | | | | |
| SBD-3/ | EVQLVESGGGLVQPGGSLRLSCAVS | 235 | DIQMTQSPSSLSASVGD | 236 |
| HC: | GYSITSDYAMNWVRQAPGKGLEW | | RVTITCKSSQSLLYRSN | |
| W35M, | VGYISNSGSTSYNPSVKSRFTISRDT | | NKNYLAWYQQKPGKA | |
| L64V | SKNTLYLQMNSLRAEDTAVYYCAR | | PKLLIYWASTRESGVPS | |
| LC: | ERNYDYDDYYYAMDYWGQGTLVT | | RFSGSGSGTDFTLTISSL | |
| Q35N | VSS | | QPEDFATYYCQQYYNY | |
| | | | PRTFGQGTKVEIK | |
| SBD-4/ | EVQLVESGGGLVQPGGSLRLSCAAS | 237 | DIVMTQSPDSLAVSLGE | 238 |
| HC: | GESITSDYAMNWVRQAPGKGLEWV | | RATINCKSSQSLLYRSN | |
| V24A, | GYISNSGSISYNPSVKGRFTISRDNS | | NKNYLAWYQQKPGQPP | |
| Y27F, | KNTLYLQMNSLRAEDTAVYYCARE | | KLLIYWASTRESGVPDR | |
| W35M, | RNYDYDDYYYAMDYWGQGTLVT | | FSGSGSGTDFTLTISSLQ | |
| T58I, | VSS | | AEDVAVYYCQQYYNYP | |
| L64V, | | | RTFGQGTKVEIK | |
| S66G, | | | | |
| T74N | | | | |
| LC: | | | | |
| Q3V, | | | | |
| S9D, | | | | |
| S12A, | | | | |
| A13V, | | | | |
| V15L, | | | | |
| D17E, | | | | |
| V19A, | | | | |
| T22N, | | | | |
| Q35N, | | | | |
| K48Q, | | | | |
| A49P, | | | | |
| S66D, | | | | |
| P86A, | | | | |
| F89V, | | | | |
| T91V | | | | |
| SBD-5/ | EVQLVESGGGLVQPGGSLRLSCAVS | 239 | DIQMTQSPSSLSASVGD | 240 |
| HC: | GYSITSDYAMNWVRQAPGKGLEW | | RVTITCKSSQSLLYRSN | |
| W35M, | VGYISNSGSTSYNPSVKSRFTISRDT | | NKNYLAWYQQKPGKA | |
| L64V, | SKNTLYLQMNSLRAEDTAVYYCAR | | PKLLIYWASTRESGVPS | |
| E99D, | DRNYDYDDYYYAFDYWGQGTLVT | | RFSGSGSGTDFTLTISSL | |
| M111F | VSS | | QPEDFATYYCQQYYNY | |
| LC: | | | PRTFGQGTKVEIK | |
| Q35N | | | | |
| SBD-6/ | EVQLVESGGGLVQPGGSLRLSCAAS | 241 | DIVMTQSPDSLAVSLGE | 242 |
| HC: | GESITSDYAMNWVRQAPGKGLEWV | | RATINCKSSQSLLYRSN | |
| V24A, | GYISNSGSTSYNPSVKSRFTISRDNS | | NKNYLAWYQQKPGQPP | |
| Y27F, | KNTLYLQMNSLRAEDTAVYYCARD | | KLLIYWASTRESGVPDR | |
| W35M, | RNYDYDDYYYAFDYWGQGTLVTV | | FSGSGSGTDFTLTISSLQ | |
| L64V, | SS | | AEDVAVYYCQQYYNYP | |
| T74N, | | | RTFGQGTKVEIK | |
| E99D, | | | | |
| M111F | | | | |
| LC: | | | | |
| Q3V, | | | | |
| S9D, | | | | |
| S12A, | | | | |
| A13V, | | | | |
| V15L, | | | | |
| D17E, | | | | |
| V19A, | | | | |
| T22N, | | | | |
| Q35N, | | | | |
| K48Q, | | | | |
| A49P, | | | | |
| S66D, | | | | |
| P86A, | | | | |
| F89V, | | | | |
| T91V | | | | |
| SBD-7 | EVQLVESGGGLVQPGGSLRLSCAVS | 243 | DIQMTQSPSSLSASVGD | 244 |
| | GYSITSDYAWNWVRQAPGKGLEWVG | | RVTITCKSSQSLLYRSN | |
| | YISNSGSTSYNPSLKSRFTISRDTS | | QKNYLAWYQQKPGKA | |
| | KNTLYLQMNSLRAEDTAVYYCARE | | PKLLIYWASTRESGVPS | |
| | RNYDYDDYYYAMDYWGQGTLVT | | RFSGSGSGTDFTLTISSL | |
| | VSS | | QPEDFATYYCQQYYNY | |
| | | | PRTFGQGTKVEIK | |
| SBD-8 | QVQLVQSGAEVKKPGASVKVSCKA | 245 | EIVLTQSPATLSLSPGER | 246 |
| | SGYTFSTYWIEWVRQAPGQRLEWM | | ATLSCRASSSVSYMHW | |
| | GEILPGSGQTDFNEKFQGRVTFTAD | | FQQKPGQAPRLLIYSTS | |
| | TSSDTAYMELSSLRSEDTAVYYCTR | | NLASGIPARFSGSGSGT | |
| | WGYYGTRGYFNVWGQGTLVTVSS | | DYTLTISSLEPEDFAVYY | |
| | | | CQQRRSFPYTFGQGTKL | |
| | | | EIK | |
| SBD-9 | EVQLVESGGGLVQPGGSLRLSCAVS | 247 | DIQMTQSPSSLSASVGD | 248 |
| | GYSITSDYAWNWVRQAPGKGLEW | | RVTITCKSSQSLLYRSN | |
| | VGYISNSGSTSYNPSLKSRFTISRDTS | | QKNYLAWYQQKPGKA | |
| | KNTLYLQMNSLRAEDTAVYYCARE | | PKLLIYWASTRESGVPS | |
| | RNYDYDDYYYAMDYWGQGTLVT | | RFSGSGSGTDFTLTISSL | |
| | VSS | | QPEDFATYYCQQYYNY | |
| | | | PRTFGQGTKVEIK | |
| SBD-10 | EVQLVESGGGLVQPGGSLRLSCAVS | 249 | DIQMTQSPSSLSASVGD | 250 |
| | GYSITSDYAWNWVRQAPGKGLEW | | RVTITCKSSQSLLYRSN | |
| | VGYISNSGSTSYNPSLKSRFTISRDTS | | QKNYLAWYQQKPGKA | |
| | KNTLYLQMNSLRAEDTAVYYCARE | | PKLLIYWASTRESGVPS | |
| | RNYDYDDYYYAMDYWGQGTLVT | | RFSGSGSGTDFTLTISSL | |
| | VSS | | QPEDFATYYCQQYYNY | |
| | | | PRTFGQGTKVEIK | |
| SBD-11/ | EVQLVESGGGLVQPGGSLRLSCAVS | 251 | DIQMTQSPSSLSASVGD | 252 |
| HC: G45C | GYSITSDYAWNWVRQAPGKCLEW | | RVTITCKSSQSLLYRSN | |
| LC: | VGYISNSGSTSYNPSLKSRFTISRDTS | | QKNYLAWYQQKPGKA | |
| Q106C | KNTLYLQMNSLRAEDTAVYYCARE | | PKLLIYWASTRESGVPS | |
| | RNYDYDDYYYAMDYWGQGTLVT | | RFSGSGSGTDFTLTISSL | |
| | VSS | | QPEDFATYYCQQYYNY | |
| | | | PRTFGCGTKVEIK | |
| SBD-12/ | EVQLVESGGGLVQPGGSLRLSCAVS | 253 | DIQMTQSPSSLSASVGD | 254 |
| HC: G45C | GYSITSDYAWNWVRQAPGKCLEW | | RVTITCKSSQSLLYRSN | |
| LC: | VGYISNSGSTSYNPSLKSRFTISRDTS | | QKNYLAWYQQKPGKA | |
| Q106C | KNTLYLQMNSLRAEDTAVYYCARE | | PKLLIYWASTRESGVPS | |
| | RNYDYDDYYYAMDYWGQGTLVT | | RFSGSGSGTDFTLTISSL | |
| | VSS | | QPEDFATYYCQQYYNY | |
| | | | PRTFGCGTKVEIK | |

TABLE 6
Anti-STEAP Fab Heavy Chain and Light Chain Sequences

| Construct | | SEQ | | SEQ |
|---|---|---|---|---|
| Name/ | | ID | | ID |
| mutations | Heavy Chain | NO: | Light Chain | NO: |
| SBD-2/ | EVQLVESGGGLVQPGGSLRLSCA | 39 | DIVMTQSPDSLAVSLGERA | 40 |
| HC: | ASGFSITSDYAWNWVRQAPGKGL | | TINCKSSQSLLYRSNQKNY | |
| V24A. | EWVGYISNSGSISYNPSLKSRFTI | | LAWYQQKPGQPPKLLIYW | |
| Y27F, | SRDNSKNTLYLQMNSLRAEDTAVY | | ASTRESGVPDRFSGSGSGT | |
| T58I, | YCARERNYDYDDYYYAMDYWG | | DFTLTISSLQAEDVAVYYC | |
| T74N | QGTLVTVSSASTKGPSVFPLAPSS | | QQYYNYPRTFGQGTKVEI | |
| LC: Q3V, | KSTSGGTAALGCLVKDYFPEPVTV | | KRTVAAPSVFIFPPSDEQLK | |
| S9D, | SWNSGALTSGVHTFPAVLQSSGL | | SGTASVVCLLNNFYPREAK | |
| S12A, | YSLSSVVTVPSSSLGTQTYICNVN | | VQWKVDNALQSGNSQESV | |
| A13V, | HKPSNTKVDKKVEPKSC | | TEQDSKDSTYSLSSTLTLS | |
| V15L, | | | KADYEKHKVYACEVTHQ | |
| D17E, | | | GLSSPVTKSFNRGEC | |
| V19A, | | | | |
| T22N, | | | | |
| K48Q, | | | | |
| A49P, | | | | |
| S66D, | | | | |
| P86A, | | | | |
| F89V, | | | | |
| T91V | | | | |
| SBD-3/ | EVQLVESGGGLVQPGGSLRLSCA | 41 | DIQMTQSPSSLSASVGDRV | 42 |
| HC: | VSGYSITSDYAMNWVRQAPGKGL | | TITCKSSQSLLYRSNNKNY | |
| W35M, | EWVGYISNSGSTSYNPSVKSRFT | | LAWYQQKPGKAPKLLIYW | |
| L64V | ISRDTSKNTLYLQMNSLRAEDTAV | | ASTRESGVPSRFSGSGSGT | |
| LC: | YYCARERNYDYDDYYYAMDYWGQ | | DFTLTISSLQPEDFATYYCQ | |
| Q35N | GTLVTVSSASTKGPSVFPLAPSS | | QYYNYPRTFGQGTKVEIK | |
| | KSTSGGTAALGCLVKDYFPEPVTV | | RTVAAPSVFIFPPSDEQLKS | |
| | SWNSGALTSGVHTFPAVLQSSGL | | GTASVVCLLNNFYPREAK | |
| | YSLSSVVTVPSSSLGTQTYICNVN | | VQWKVDNALQSGNSQESV | |
| | HKPSNTKVDKKVEPKSC | | TEQDSKDSTYSLSSTLTLS | |
| | | | KADYEKHKVYACEVTHQ | |
| | | | GLSSPVTKSFNRGEC | |
| SBD-4/ | EVQLVESGGGLVQPGGSLRLSCA | 43 | DIVMTQSPDSLAVSLGERA | 44 |
| HC: | ASGFSITSDYAMNWVRQAPGKGL | | TINCKSSQSLLYRSNNKNY | |
| V24A, | EWVGYISNSGSISYNPSVKGRFTIS | | LAWYQQKPGQPPKLLIYW | |
| Y27F, | RDNSKNTLYLQMNSLRAEDTAVY | | ASTRESGVPDRFSGSGSGT | |
| W35M, | YCARERNYDYDDYYYAMDYWG | | DFTLTISSLQAEDVAVYYC | |
| T58I, | QGTLVTVSSASTKGPSVFPLAPSS | | QQYYNYPRTFGQGTKVEI | |
| L64V, | KSTSGGTAALGCLVKDYFPEPVTV | | KRTVAAPSVFIFPPSDEQLK | |
| S66G, | SWNSGALTSGVHTFPAVLQSSGL | | SGTASVVCLLNNFYPREAK | |
| T74N | YSLSSVVTVPSSSLGTQTYICNVN | | VQWKVDNALQSGNSQESV | |
| LC: | HKPSNTKVDKKVEPKSC | | TEQDSKDSTYSLSSTLTLS | |
| Q3V, | | | KADYEKHKVYACEVTHQ | |
| S9D, | | | GLSSPVTKSFNRGEC | |
| S12A, | | | | |
| A13V, | | | | |
| V15L, | | | | |
| D17E, | | | | |
| V19A, | | | | |
| T22N, | | | | |
| Q35N, | | | | |
| K48Q, | | | | |
| A49P, | | | | |
| S66D, | | | | |
| P86A, | | | | |
| F89V, | | | | |
| T91V | | | | |
| SBD-5/ | EVQLVESGGGLVQPGGSLRLSCA | 45 | DIQMTQSPSSLSASVGDRV | 46 |
| HC: | VSGYSITSDYAMNWVRQAPGKGL | | TITCKSSQSLLYRSNNKNY | |
| W35M, | EWVGYISNSGSTSYNPSVKSRFTIS | | LAWYQQKPGKAPKLLIYW | |
| L64V, | RDTSKNTLYLQMNSLRAEDTAVY | | ASTRESGVPSRFSGSGSGT | |
| E99D, | YCARDRNYDYDDYYYAFDYWGQ | | DFTLTISSLQPEDFATYYCQ | |
| M111F | GTLVTVSSASTKGPSVFPLAPSSKS | | QYYNYPRTFGQGTKVEIK | |
| LC: | TSGGTAALGCLVKDYFPEPVTVS | | RTVAAPSVFIFPPSDEQLKS | |
| Q35N | WNSGALTSGVHTFPAVLQSSGLY | | GTASVVCLLNNFYPREAK | |
| | SLSSVVTVPSSSLGTQTYICNVNH | | VQWKVDNALQSGNSQESV | |
| | KPSNTKVDKKVEPKSC | | TEQDSKDSTYSLSSTLTLS | |
| | | | KADYEKHKVYACEVTHQ | |
| | | | GLSSPVTKSFNRGEC | |
| SBD-6/ | EVQLVESGGGLVQPGGSLRLSCA | 47 | DIVMTQSPDSLAVSLGERA | 48 |
| HC: | ASGFSITSDYAMNWVRQAPGKGL | | TINCKSSQSLLYRSNNKNY | |
| V24A, | EWVGYISNSGSTSYNPSVKSRFTIS | | LAWYQQKPGQPPKLLIYW | |
| Y27F, | RDNSKNTLYLQMNSLRAEDTAVY | | ASTRESGVPDRFSGSGSGT | |
| W35M, | YCARDRNYDYDDYYYAFDYWGQ | | DFTLTISSLQAEDVAVYYC | |
| L64V, | GTLVTVSSASTKGPSVFPLAPSSKS | | QQYYNYPRTFGQGTKVEI | |
| T74N, | TSGGTAALGCLVKDYFPEPVTVS | | KRTVAAPSVFIFPPSDEQLK | |
| E99D, | WNSGALTSGVHTFPAVLQSSGLY | | SGTASVVCLLNNFYPREAK | |
| M111F | SLSSVVTVPSSSLGTQTYICNVNH | | VQWKVDNALQSGNSQESV | |
| LC: | KPSNTKVDKKVEPKSC | | TEQDSKDSTYSLSSTLTLS | |
| Q3V, | | | KADYEKHKVYACEVTHQ | |
| S9D, | | | GLSSPVTKSFNRGEC | |
| S12A, | | | | |
| A13V, | | | | |
| V15L, | | | | |
| D17E, | | | | |
| V19A, | | | | |
| T22N, | | | | |
| Q35N, | | | | |
| K48Q, | | | | |
| A49P, | | | | |
| S66D, | | | | |
| P86A, | | | | |
| F89V, | | | | |
| T91 | | | | |
| SBD-7/ | EVQLVESGGGLVQPGGSLRLSCA | 255 | DIQMTQSPSSLSASVGDRV | 256 |
| EE/RK | VSGYSITSDYAWNWVRQAPGKGL | | TITCKSSQSLLYRSNQKNY | |
| HC: | EWVGYISNSGSTSYNPSLKSRFTIS | | LAWYQQKPGKAPKLLIYW | |
| K154E, | RDTSKNTLYLQMNSLRAEDTAVY | | ASTRESGVPSRFSGSGSGT | |
| K220E | YCARERNYDYDDYYYAMDYWG | | DFTLTISSLQPEDFATYYCQ | |
| LC: | QGTLVTVSSASTKGPSVFPLAPSS | | QYYNYPRTFGQGTKVEIK | |
| E129R, | KSTSGGTAALGCLVEDYFPEPVTV | | RTVAAPSVFIFPPSDRKLKS | |
| Q130K | SWNSGALTSGVHTFPAVLQSSGL | | GTASVVCLLNNFYPREAK | |
| | YSLSSVVTVPSSSLGTQTYICNVN | | VQWKVDNALQSGNSQESV | |
| | HKPSNGKVDEKVEPKSC | | TEQDSKDSTYSLSSTLTLS | |
| | | | KADYEKHKVYACEVTHQ | |
| | | | GLSSPVTKSFNRGEC | |
| SBD-8 | QVQLVQSGAEVKKPGASVKVSCK | 257 | EIVLTQSPATLSLSPGERAT | 258 |
| | ASGYTFSTYWIEWVRQAPGQRLE | | LSCRASSSVSYMHWFQQK | |
| | WMGEILPGSGQTDFNEKFQGRVT | | PGQAPRLLIYSTSNLASGIP | |
| | FTADTSSDTAYMELSSLRSEDTAV | | ARFSGSGSGTDYTLTISSLE | |
| | YYCTRWGYYGTRGYFNVWGQGT | | PEDFAVYYCQQRRSFPYTF | |
| | LVTVSSASTKGPSVFPLAPSSKSTS | | GQGTKLEIKRTVAAPSVFIF | |
| | GGTAALGCLVKDYFPEPVTVSWN | | PPSDEQLKSGTASVVCLLN | |
| | SGALTSGVHTFPAVLQSSGLYSLS | | NFYPREAKVQWKVDNAL | |
| | SVVTVPSSSLGTQTYICNVNHKPS | | QSGNSQESVTEQDSKDSTY | |
| | NTKVDKKVEPKSC | | SLSSTLTLSKADYEKHKVY | |
| | | | ACEVTHQGLSSPVTKSFNR | |
| | | | GEC | |

In some embodiments, the immunoglobulin heavy chain comprises an amino acid sequence of at least 99% sequence identity to SEQ ID NO: 49 and the immunoglobulin light chain comprises an amino acid sequence of at least 99% sequence identity to SEQ ID NO: 50. In some embodiments, the immunoglobulin heavy chain comprises an amino acid sequence of at least 99% sequence identity to SEQ ID NO: 51 and the immunoglobulin light chain comprises an amino acid sequence of at least 99% sequence identity to SEQ ID NO: 52. In some embodiments, the immunoglobulin heavy chain comprises the amino acid sequence of SEQ ID NO: 51 and the immunoglobulin light chain comprises the amino acid sequence of SEQ ID NO: 52. In some embodiments, the immunoglobulin heavy chain comprises an amino acid sequence of at least 99% sequence identity to SEQ ID NO: 53 and the immunoglobulin light chain comprises an amino acid sequence of at least 99% sequence identity to SEQ ID NO: 54. In some embodiments, the immunoglobulin heavy chain comprises the amino acid sequence of SEQ ID NO: 53 and the immunoglobulin light chain comprises the amino acid sequence of SEQ ID NO: 54. In some embodiments, the immunoglobulin heavy chain comprises an amino acid sequence of at least 99% sequence identity to SEQ ID NO: 55 and the immunoglobulin light chain comprises an amino acid sequence of at least 99% sequence identity to SEQ ID NO: 56. In some embodiments, the immunoglobulin heavy chain comprises the amino acid sequence of SEQ ID NO: 55 and the immunoglobulin light chain comprises the amino acid sequence of SEQ ID NO: 56. In some embodiments, the immunoglobulin heavy chain comprises an amino acid sequence of at least 99% sequence identity to SEQ ID NO: 57 and the immunoglobulin light chain comprises an amino acid sequence of at least 99% sequence identity to SEQ ID NO: 58. In some embodiments, the immunoglobulin heavy chain comprises the amino acid sequence of SEQ ID NO: 57 and the immunoglobulin light chain comprises the amino acid sequence of SEQ ID NO: 58. In some embodiments, the immunoglobulin heavy chain comprises an amino acid sequence of at least 99% sequence identity to SEQ ID NO: 59 and the immunoglobulin light chain comprises an amino acid sequence of at least 99% sequence identity to SEQ ID NO: 60. In some embodiments, the immunoglobulin heavy chain comprises the amino acid sequence of SEQ ID NO: 59 and the immunoglobulin light chain comprises the amino acid sequence of SEQ ID NO: 60.

TABLE 7
Heavy Chain and Light Chain

| Construct | | SEQ | | SEQ |
|---|---|---|---|---|
| Name/ | | ID | | ID |
| mutations | Heavy Chain | NO: | Light Chain | NO: |
| IgG-1/ | EVQLVESGGGLVQPGGSLRLSCA | 49 | DIQMTQSPSSLSASVGDRVTI | 50 |
| wt | VSGYSITSDYAWNWVRQAPGKGL | | TCKSSQSLLYRSNQKNYLA | |
| | EWVGYISNSGSTSYNPSLKSRFTIS | | WYQQKPGKAPKLLIYWAST | |
| | RDTSKNTLYLQMNSLRAEDTAVY | | RESGVPSRFSGSGSGTDFTLT | |
| | YCARERNYDYDDYYYAMDYWG | | ISSLQPEDFATYYCQQYYNY | |
| | QGTLVTVSSASTKGPSVFPLAPSS | | PRTFGQGTKVEIKRTVAAPS | |
| | KSTSGGTAALGCLVKDYFPEPVTV | | VFIFPPSDEQLKSGTASVVCL | |
| | SWNSGALTSGVHTFPAVLQSSGL | | LNNFYPREAKVQWKVDNAL | |
| | YSLSSVVTVPSSSLGTQTYICNVN | | QSGNSQESVTEQDSKDSTYS | |
| | HKPSNTKVDKKVEPKSCDKTHTC | | LSSTLTLSKADYEKHKVYAC | |
| | PPCPAPELLGGPSVFLFPPKPKDTL | | EVTHQGLSSPVTKSFNRGEC | |
| | MISRTPEVTCVVVDVSHEDPEVKF | | | |
| | NWYVDGVEVHNAKTKPREEQYN | | | |
| | STYRVVSVLTVLHQDWLNGKEYK | | | |
| | CKVSNKALPAPIEKTISKAKGQPR | | | |
| | EPQVYTLPPSRDELTKNQVSLTCL | | | |
| | VKGFYPSDIAVEWESNGQPENNY | | | |
| | KTTPPVLDSDGSFFLYSKLTVDKS | | | |
| | RWQQGNVFSCSVMHEALHNHYT | | | |
| | QKSLSLSPGK | | | |
| IgG-2/ | EVQLVESGGGLVQPGGSLRLSCA | 51 | DIVMTQSPDSLAVSLGERATI | 52 |
| HC: | ASGFSITSDYAWNWVRQAPGKGL | | NCKSSQSLLYRSNQKNYLA | |
| V24A, | EWVGYISNSGSISYNPSLKSRFTIS | | WYQQKPGQPPKLLIYWAST | |
| Y27F, | RDNSKNTLYLQMNSLRAEDTAVY | | RESGVPDRFSGSGSGTDFTL | |
| T58I, | YCARERNYDYDDYYYAMDYWG | | TISSLQAEDVAVYYCQQYYN | |
| T74N | QGTLVTVSSASTKGPSVFPLAPSS | | YPRTFGQGTKVEIKRTVAAP | |
| LC: | KSTSGGTAALGCLVKDYFPEPVTV | | SVFIFPPSDEQLKSGTASVVC | |
| Q3V, | SWNSGALTSGVHTFPAVLQSSGL | | LLNNFYPREAKVQWKVDNA | |
| S9D, | YSLSSVVTVPSSSLGTQTYICNVN | | LQSGNSQESVTEQDSKDSTY | |
| S12A, | HKPSNTKVDKKVEPKSCDKTHTC | | SLSSTLTLSKADYEKHKVYA | |
| A13V, | PPCPAPELLGGPSVFLFPPKPKDTL | | CEVTHQGLSSPVTKSFNRGE | |
| V15L, | MISRTPEVTCVVVDVSHEDPEVKF | | C | |
| D17E, | NWYVDGVEVHNAKTKPREEQYN | | | |
| V19A, | STYRVVSVLTVLHQDWLNGKEYK | | | |
| T22N, | CKVSNKALPAPIEKTISKAKGQPR | | | |
| K48Q, | EPQVYTLPPSRDELTKNQVSLTCL | | | |
| A49P, | VKGFYPSDIAVEWESNGQPENNY | | | |
| S66D, | KTTPPVLDSDGSFFLYSKLTVDKS | | | |
| P86A, | RWQQGNVFSCSVMHEALHNHYT | | | |
| F89V, | QKSLSLSPGK | | | |
| T91V | | | | |
| IgG-3/ | EVQLVESGGGLVQPGGSLRLSCA | 53 | DIQMTQSPSSLSASVGDRVTI | 54 |
| HC: | VSGYSITSDYAMNWVRQAPGKGL | | TCKSSQSLLYRSNNKNYLA | |
| W35M, | EWVGYISNSGSTSYNPSVKSRFTIS | | WYQQKPGKAPKLLIYWAST | |
| L64V | RDTSKNTLYLQMNSLRAEDTAVY | | RESGVPSRFSGSGSGTDFTLT | |
| LC: | YCARERNYDYDDYYYAMDYWG | | ISSLQPEDFATYYCQQYYNY | |
| Q35N | QGTLVTVSSASTKGPSVFPLAPSS | | PRTFGQGTKVEIKRTVAAPS | |
| | KSTSGGTAALGCLVKDYFPEPVTV | | VFIFPPSDEQLKSGTASVVCL | |
| | SWNSGALTSGVHTFPAVLQSSGL | | LNNFYPREAKVQWKVDNAL | |
| | YSLSSVVTVPSSSLGTQTYICNVN | | QSGNSQESVTEQDSKDSTYS | |
| | HKPSNTKVDKKVEPKSCDKTHTC | | LSSTLTLSKADYEKHKVYAC | |
| | PPCPAPELLGGPSVFLFPPKPKDTL | | EVTHQGLSSPVTKSFNRGEC | |
| | MISRTPEVTCVVVDVSHEDPEVKF | | | |
| | NWYVDGVEVHNAKTKPREEQYN | | | |
| | STYRVVSVLTVLHQDWLNGKEYK | | | |
| | CKVSNKALPAPIEKTISKAKGQPR | | | |
| | EPQVYTLPPSRDELTKNQVSLTCL | | | |
| | VKGFYPSDIAVEWESNGQPENNY | | | |
| | KTTPPVLDSDGSFFLYSKLTVDKS | | | |
| | RWQQGNVFSCSVMHEALHNHYT | | | |
| | QKSLSLSPGK | | | |
| IgG-4/ | EVQLVESGGGLVQPGGSLRLSCA | 55 | DIVMTQSPDSLAVSLGERATI | 56 |
| HC: | ASGFSITSDYAMNWVRQAPGKGL | | NCKSSQSLLYRSNNKNYLA | |
| V24A, | EWVGYISNSGSISYNPSVKGRFTIS | | WYQQKPGQPPKLLIYWAST | |
| Y27F, | RDNSKNTLYLQMNSLRAEDTAVY | | RESGVPDRFSGSGSGTDFTL | |
| W35M, | YCARERNYDYDDYYYAMDYWG | | TISSLQAEDVAVYYCQQYYN | |
| T58I, | QGTLVTVSSASTKGPSVFPLAPSS | | YPRTFGQGTKVEIKRTVAAP | |
| L64V, | KSTSGGTAALGCLVKDYFPEPVTV | | SVFIFPPSDEQLKSGTASVVC | |
| S66G, | SWNSGALTSGVHTFPAVLQSSGL | | LLNNFYPREAKVQWKVDNA | |
| T74N | YSLSSVVTVPSSSLGTQTYICNVN | | LQSGNSQESVTEQDSKDSTY | |
| LC: | HKPSNTKVDKKVEPKSCDKTHTC | | SLSSTLTLSKADYEKHKVYA | |
| Q3V, | PPCPAPELLGGPSVFLFPPKPKDTL | | CEVTHQGLSSPVTKSFNRGE | |
| S9D, | MISRTPEVTCVVVDVSHEDPEVKF | | C | |
| S12A, | NWYVDGVEVHNAKTKPREEQYN | | | |
| A13V, | STYRVVSVLTVLHQDWLNGKEYK | | | |
| V15L, | CKVSNKALPAPIEKTISKAKGQPR | | | |
| D17E, | EPQVYTLPPSRDELTKNQVSLTCL | | | |
| V19A, | VKGFYPSDIAVEWESNGQPENNY | | | |
| T22N, | KTTPPVLDSDGSFFLYSKLTVDKS | | | |
| Q35N, | RWQQGNVFSCSVMHEALHNHYT | | | |
| K48Q, | QKSLSLSPGK | | | |
| A49P, | | | | |
| S66D, | | | | |
| P86A, | | | | |
| F89V, | | | | |
| T91V | | | | |
| IgG-5/ | EVQLVESGGGLVQPGGSLRLSCA | 57 | DIQMTQSPSSLSASVGDRVT | 58 |
| HC: | VSGYSITSDYAMNWVRQAPGKGL | | ITCKSSQSLLYRSNNKNYLA | |
| W35M, | EWVGYISNSGSTSYNPSVKSRFTI | | WYQQKPGKAPKLLIYWASTR | |
| L64V, | SRDTSKNTLYLQMNSLRAEDTAVY | | ESGVPSRFSGSGSGTDFTLT | |
| E99D, | YCARDRNYDYDDYYYAFDYWGQ | | ISSLQPEDFATYYCQQYYNY | |
| M111F | GTLVTVSSASTKGPSVFPLAPSSKS | | PRTFGQGTKVEIKRTVAAPS | |
| LC: | TSGGTAALGCLVKDYFPEPVTVS | | VFIFPPSDEQLKSGTASVVC | |
| Q35N | WNSGALTSGVHTFPAVLQSSGLY | | LLNNFYPREAKVQWKVDNAL | |
| | SLSSVVTVPSSSLGTQTYICNVNH | | QSGNSQESVTEQDSKDSTYS | |
| | KPSNTKVDKKVEPKSCDKTHTCPP | | LSSTLTLSKADYEKHKVYAC | |
| | CPAPELLGGPSVFLFPPKPKDTLMI | | EVTHQGLSSPVTKSFNRGEC | |
| | SRTPEVTCVVVDVSHEDPEVKFN | | | |
| | WYVDGVEVHNAKTKPREEQYNS | | | |
| | TYRVVSVLTVLHQDWLNGKEYK | | | |
| | CKVSNKALPAPIEKTISKAKGQPR | | | |
| | EPQVYTLPPSRDELTKNQVSLTCL | | | |
| | VKGFYPSDIAVEWESNGQPENNY | | | |
| | KTTPPVLDSDGSFFLYSKLTVDKS | | | |
| | RWQQGNVFSCSVMHEALHNHYT | | | |
| | QKSLSLSPGK | | | |
| IgG-6/ | EVQLVESGGGLVQPGGSLRLSCA | 59 | DIVMTQSPDSLAVSLGERAT | 60 |
| HC: | ASGFSITSDYAMNWVRQAPGKGLE | | INCKSSQSLLYRSNNKNYLA | |
| V24A, | WVGYISNSGSTSYNPSVKSRFTIS | | WYQQKPGQPPKLLIYWAST | |
| Y27F, | RDNSKNTLYLQMNSLRAEDTAVY | | RESGVPDRFSGSGSGTDFTL | |
| W35M, | YCARDRNYDYDDYYYAFDYWGQ | | TISSLQAEDVAVYYCQQYYN | |
| L64V, | GTLVTVSSASTKGPSVFPLAPSSKS | | YPRTFGQGTKVEIKRTVAAP | |
| T74N, | TSGGTAALGCLVKDYFPEPVTVS | | SVFIFPPSDEQLKSGTASVV | |
| E99D, | WNSGALTSGVHTFPAVLQSSGLY | | CLLNNFYPREAKVQWKVDNA | |
| M111F | SLSSVVTVPSSSLGTQTYICNVNH | | LQSGNSQESVTEQDSKDSTY | |
| LC: | KPSNTKVDKKVEPKSCDKTHTCPP | | SLSSTLTLSKADYEKHKVYA | |
| Q3V, | CPAPELLGGPSVFLFPPKPKDTLMI | | CEVTHQGLSSPVTKSFNRGE | |
| S9D, | SRTPEVTCVVVDVSHEDPEVKFN | | C | |
| S12A, | WYVDGVEVHNAKTKPREEQYNS | | | |
| A13V, | TYRVVSVLTVLHQDWLNGKEYK | | | |
| V15L, | CKVSNKALPAPIEKTISKAKGQPR | | | |
| D17E, | EPQVYTLPPSRDELTKNQVSLTCL | | | |
| V19A, | VKGFYPSDIAVEWESNGQPENNY | | | |
| T22N, | KTTPPVLDSDGSFFLYSKLTVDKS | | | |
| Q35N, | RWQQGNVFSCSVMHEALHNHYT | | | |
| K48Q, | QKSLSLSPGK | | | |
| A49P, | | | | |
| S66D, | | | | |
| P86A, | | | | |
| F89V, | | | | |
| T91V | | | | |

In some embodiments, the polypeptide or polypeptide complex comprises an amino acid sequence of at least 95%, 98%, or 99% sequence identity to SEQ ID NO: 259. In some embodiments, the polypeptide or polypeptide complex comprises an amino acid sequence of at least 95%, 98%, or 99% sequence identity to SEQ ID NO: 260. In some embodiments, the polypeptide or polypeptide complex comprises an amino acid sequence of at least 95%, 98%, 99%, or 100% sequence identity to SEQ ID NO: 261. In some embodiments, the polypeptide or polypeptide complex comprises an amino acid sequence of at least 95%, 98%, 99%, or 100% sequence identity to SEQ ID NO: 262. In some embodiments, the polypeptide or polypeptide complex comprises an amino acid sequence of at least 95%, 98%, 99%, or 100% sequence identity to SEQ ID NO: 263. In some embodiments, the polypeptide or polypeptide complex comprises an amino acid sequence of at least 95%, 98%, 99%, or 100% sequence identity to SEQ ID NO: 264.

TABLE 8
Anti-STEAP-1 scFv Sequences

| | | SEQ |
|---|---|---|
| Construct | | ID |
| Description | Amino Acid Sequence (N to C) | NO: |
| SBD-9 | EVQLVESGGGLVQPGGSLRLSCAVSGYSITSDYAWNWVRQAPGKGL | 259 |
| Vh-L-Vl | EWVGYISNSGSTSYNPSLKSRFTISRDTSKNTLYLQMNSLRAEDTAVY | |
| wt | YCARERNYDYDDYYYAMDYWGQGTLVTVSSGGGGSGGGGSGGGG | |
| | SDIQMTQSPSSLSASVGDRVTITCKSSQSLLYRSNQKNYLAWYQQKP | |
| | GKAPKLLIYWASTRESGVPSRFSGSGSGTDFTLTISSLQPEDFATYYCQ | |
| | QYYNYPRTFGQGTKVEIK | |
| SBD-10 | DIQMTQSPSSLSASVGDRVTITCKSSQSLLYRSNQKNYLAWYQQKPG | 260 |
| Vl-L-Vh | KAPKLLIYWASTRESGVPSRFSGSGSGTDFTLTISSLQPEDFATYYCQQ | |
| wt | YYNYPRTFGQGTKVEIKGGGGSGGGGSGGGGSEVQLVESGGGLVQP | |
| | GGSLRLSCAVSGYSITSDYAWNWVRQAPGKGLEWVGYISNSGSTSYN | |
| | PSLKSRFTISRDTSKNTLYLQMNSLRAEDTAVYYCARERNYDYDDYY | |
| | YAMDYWGQGTLVTVSS | |
| SBD-11 | EVQLVESGGGLVQPGGSLRLSCAVSGYSITSDYAWNWVRQAPGKCL | 261 |
| Vh-L-V1 | EWVGYISNSGSTSYNPSLKSRFTISRDTSKNTLYLQMNSLRAEDTAVY | |
| disulfide, | YCARERNYDYDDYYYAMDYWGQGTLVTVSSGGGGSGGGGSGGGG | |
| C44:C100 | SDIQMTQSPSSLSASVGDRVTITCKSSQSLLYRSNQKNYLAWYQQKP | |
| | GKAPKLLIYWASTRESGVPSRFSGSGSGTDFTLTISSLQPEDFATYYCQ | |
| | QYYNYPRTFGCGTKVEIK | |
| SBD-12 | DIQMTQSPSSLSASVGDRVTITCKSSQSLLYRSNQKNYLAWYQQKPG | 262 |
| VI-L-Vh, | KAPKLLIYWASTRESGVPSRFSGSGSGTDFTLTISSLQPEDFATYYCQQ | |
| Disulfide, | YYNYPRTFGCGTKVEIKGGGGSGGGGSGGGGSEVQLVESGGGLVQP | |
| C44:C100 | GGSLRLSCAVSGYSITSDYAWNWVRQAPGKCLEWVGYISNSGSTSYN | |
| | PSLKSRFTISRDTSKNTLYLQMNSLRAEDTAVYYCARERNYDYDDYY | |
| | YAMDYWGQGTLVTVSS | |
| SBD-13 | QVQLVQSGAEVKKPGASVKVSCKASGYTFSTYWIEWVRQAPGQRLE | 263 |
| scFv | WMGEILPGSGQTDFNEKFQGRVTFTADTSSDTAYMELSSLRSEDTAV | |
| (Vh-Vl) | YYCTRWGYYGTRGYFNVWGQGTLVTVSSGGGGSGGGGSGGGGSEI | |
| | VLTQSPATLSLSPGERATLSCRASSSVSYMHWFQQKPGQAPRLLIYST | |
| | SNLASGIPARFSGSGSGTDYTLTISSLEPEDFAVYYCQQRRSFPYTFGQ | |
| | GTKLEIK | |
| SBD-14 | EIVLTQSPATLSLSPGERATLSCRASSSVSYMHWFQQKPGQAPRLLIYS | 264 |
| scFv | TSNLASGIPARFSGSGSGTDYTLTISSLEPEDFAVYYCQQRRSFPYTFG | |
| (Vl-Vh) | QGTKLEIKGGGGSGGGGSGGGGSQVQLVQSGAEVKKPGASVKVSCK | |
| | ASGYTFSTYWIEWVRQAPGQRLEWMGEILPGSGQTDFNEKFQGRVTF | |
| | TADTSSDTAYMELSSLRSEDTAVYYCTRWGYYGTRGYFNVWGQGTL | |
| | VTVSS | |

In some embodiments, the polypeptide or polypeptide complex that comprises the antibody or antigen binding fragment thereof that binds to STEAP-1 is a monoclonal antibody. In some embodiments, the polypeptide or polypeptide complex that comprises the antibody or antigen binding fragment thereof that binds to STEAP-1 is a monospecific antibody, bispecific antibody, or trispecific antibody. In some embodiments, the antibody or antigen binding fragment thereof that binds to STEAP-1 comprises a Fab, Fab′, (Fab′)₂ or a single chain variable fragment (scFv).

In some embodiments, the polypeptide or polypeptide complex comprises more than one antibody or antigen binding fragment thereof that binds to STEAP-1. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁). In some embodiments, L₁ connects an immunoglobulin heavy chain of the Fab or Fab′ that binds STEAP-1 with another immunoglobulin heavy chain of the second Fab or Fab′ that binds STEAP-1. In some embodiments, L₁ connects an immunoglobulin light chain of the Fab or Fab′ that binds STEAP-1 with another immunoglobulin light chain of the second Fab or Fab′ that binds STEAP-1. In some embodiments, L₁ connects an immunoglobulin light chain of the Fab or Fab′ that binds STEAP-1 with an immunoglobulin heavy chain of the second Fab or Fab′ that binds STEAP-1. In some embodiments, L₁ comprises an amino acid sequence selected from any one of SEQ ID NOs: 222-224, 270-273, and 371.

### CD3 Binding Domain

In some embodiments, the second antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83; wherein CDR2-L comprises the amino acid sequence of GTK; wherein CDR3-L comprises the amino acid sequence of Z₁-Z₂-W-Z₃-Z₄-Z₅-Z₆-W-Z₇-Z₈ or SEQ ID NO: 82; wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 61 or SEQ ID NO: 64; wherein CDR2-H comprises the amino acid sequence of SEQ ID NO: 62; wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63; and wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A. In some embodiments, Z₁ is V, G, L, I, or A; Z₂ is L, V, I, or A; Z₃ is Y, W, F, or A; Z₄ is S, G, T, or A; Z₅ is N, Q, D, E, or A; Z₆ is R, K, or A; Z₇ is V, G, L, I, or A; Z₈ is F, Y, W, or A; Z₉ is V, G, L, I, or A; Z₁₀ is R, K, or A; Z₁₂ is G, S, T, or A; Z₁₃ is F, Y, W, or A; Z₁₄ is G, S, T, or A; Z₁₅ is N, Q, D, E, or A; Z₁₆ is S, G, T, or A; Z₁₇ is Y, W, F, or A; Z₁₈ is I, V, L, or A; Z₁₉ is S, G, T, or A; Z₂₀ is W, Y, F, or A; and Z₂₁ is Y, W, F, or A. In some embodiments, Z₈ is F. In some embodiments, Z₁₀ is R; Z₁₁ is H; Z₁₃ is F; Z₁₈ is I; Z₁₉ is S; and Z₂₀ is W.

TABLE 9
Anti-CD3 heavy chain complementarity
determining regions (CDRs) as based on
IMGT CDR numbering system.

| Construct | Amino Acid Sequence | SEQ ID |
|---|---|---|
| Description | (N to C) | NO: |
| CDR1-H | GFTFNKYA | 61 |
| CDR2-H | IRSKYNNYAT | 62 |
| CDR3-H | VRHGNFGNSYISYWAY | 63 |
| CDR3-H | VRHGNFGNSYVSYWAY | 124 |
| CDR1-H | GFTFQKYA | 64 |
| CDR3-H V99A | ARHGNFGNSYISYWAY | 65 |
| CDR3-H R100A | VAHGNFGNSYISYWAY | 66 |
| CDR3-H H101A | VRAGNFGNSYISYWAY | 67 |
| CDR3-HG102A | VRHANFGNSYISYWAY | 68 |
| CDR3-HN103A | VRHGAFGNSYISYWAY | 69 |
| CDR3-H F104A | VRHGNAGNSYISYWAY | 70 |
| CDR3-HG105A | VRHGNFANSYISYWAY | 71 |
| CDR3-HN106A | VRHGNFGASYISYWAY | 72 |
| CDR3-H S107A | VRHGNFGNAYISYWAY | 73 |
| CDR3-H Y108A | VRHGNFGNSAISYWAY | 74 |
| CDR3-H I109A | VRHGNFGNSYASYWAY | 75 |
| CDR3-H S110A | VRHGNFGNSYIAYWAY | 76 |
| CDR3-H Y111A | VRHGNFGNSYISAWAY | 77 |
| CDR3-H W112A | VRHGNFGNSYISYAAY | 78 |
| CDR3-H Y114A | VRHGNFGNSYISYWAA | 79 |

TABLE 10
Anti-CD3 light chain complementarity
determining regions (CDRs) as based
on IMGT CDR numbering system.

| Construct | Amino Acid Sequence | SEQ ID |
|---|---|---|
| Description | (N to C) | NO: |
| CDR1-L | TGAVTSGNY | 80 |
| CDR2-L | GTK | |
| CDR3-L | VLWYSNRWV | 82 |
| CDR1-L | TGAVTSANY | 83 |
| CDR3-L V231A | ALWYSNRWV | 84 |
| CDR3-L L232A | VAWYSNRWV | 85 |
| CDR3-L W233A | VLAYSNRWV | 86 |
| CDR3-L Y234A | VLWASNRWV | 87 |
| CDR3-L S235A | VLWYANRWV | 88 |
| CDR3-L N236A | VLWYSARWV | 89 |
| CDR3-L R237A | VLWYSNAWV | 90 |
| CDR3-L W238A | VLWYSNRAV | 91 |
| CDR3-L V239A | VLWYSNRWA | 92 |
| CDR3-L F240A | VLWYSNRWVA | 93 |

In some embodiments, CDR1-H of the second antibody or antigen binding fragment thereof that binds to CD3 comprises the amino acid sequence of SEQ ID NO: 61 or SEQ ID NO: 64; CDR2-H of the second antibody or antigen binding fragment thereof that binds to CD3 comprises the amino acid sequence of SEQ ID NO: 62; CDR3-H of the second antibody or antigen binding fragment thereof that binds to CD3 comprises the amino acid sequence selected from SEQ ID NOs: 63, 65-79, and 124; CDR1-L of the second antibody or antigen binding fragment thereof that binds to CD3 comprises the amino acid sequence of SEQ ID NO: 80 or SEQ ID NO: 83; CDR2-L of the second antibody or antigen binding fragment thereof that binds to CD3 comprises the amino acid sequence of GTK; and CDR3-L of the second antibody or antigen binding fragment thereof that binds to CD3 comprises the amino acid sequence selected from SEQ ID NOs: 82 and 84-93.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: SEQ ID NO: 81, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 65, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 66, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 67, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 68, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 69, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 70, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 71, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 72, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 73, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 74, CDR1-L: SEQ ID NO: 80, CDR2-L: GTK, and CDR3-L: SEQ ID NO: 82. In some embodiments, the second antibody or antigen binding fragment thereof that binds to CD3 comprises the 75, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 76, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 77, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 78, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 79, CDR1-L: SEQ ID NO: 80, CDR2-L: GTK, and CDR3-L: SEQ ID NO: 82.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 84. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 85. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 86. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 87. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 88. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 89. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 90. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 91. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 92. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 93. In some embodiments, the second antibody or antigen binding fragment thereof that binds to CD3 comprises the following set of six CDRs: CDR1-H: SEQ ID NO: 64, CDR2-H: SEQ ID NO: 62, CDR3-H: SEQ ID NO: 124, CDR1-L: SEQ ID NO: 83, CDR2-L: GTK, and CDR3-L: SEQ ID NO: 84.

In some embodiments, the second antibody or antigen binding fragment thereof that binds to CD3 comprises a variable domain of an immunoglobulin kappa (IgK) or immunoglobulin lambda (IgL) light chain. In some embodiments, the second antibody or antigen binding fragment thereof that binds to CD3 comprises a constant domain of an IgG1, IgG2, IgG3, or IgG4 heavy chain. In some embodiments, the second antibody or antigen binding fragment thereof that binds to CD3 comprises amino acid sequences with at least 90%, 95%, 96%, 97%, 98%, 99%, or 100% sequence identity to SEQ ID NO: 94 and SEQ ID NO: 95. In some embodiments, the second antibody or antigen binding fragment thereof that binds to CD3 comprises amino acid sequences with at least 90%, 95%, 96%, 97%, 98%, 99%, or 100% identity to SEQ ID NO: 266 and SEQ ID NO: 267. In some embodiments, the second antibody or antigen binding fragment thereof that binds to CD3 comprises amino acid sequences with at least 90%, 95%, 96%, 97%, 98%, or 100% identity to SEQ ID NO: 268 and SEQ ID NO: 269. In some embodiments, the second antibody or antigen binding fragment thereof that binds to CD3 comprises a Fab, Fab′, (Fab′)₂ or a single chain variable fragment (scFv). In some embodiments, the second antibody or antigen binding fragment thereof that binds to CD3 comprises the scFv. In some embodiments, the second antibody or antigen binding fragment thereof that binds to CD3 comprises the Fab.

TABLE 11
Anti-CD3 heavy chain and light chain sequences and scFvs

| | | SEQ |
|---|---|---|
| | | ID |
| Construct Description | Amino Acid Sequence (N to C) | NO: |
| CD3 light chain | QTVVTQEPSLTVSPGGTVTLTCGSSTGAVTSGNYP | 94 |
| variable region | NWVQQKPGQAPRGLIGGTKFLAPGTPARFSGSLLG | |
| | GKAALTLSGVQPEDEAEYYCVLWYSNRWVFGGGT | |
| | KLTVL | |
| CD3 heavy chain | EVQLVESGGGLVQPGGSLKLSCAASGFTFNKYAMN | 95 |
| variable region | WVRQAPGKGLEWVARIRSKYNNYATYYADSVKDR | |
| | FTISRDDSKNTAYLQMNNLKTEDTAVYYCVRHGNF | |
| | GNSYISYWAYWGQGTLVTVSS | |
| CD3 scFv (SP34.185) | EVQLVESGGGLVQPGGSLKLSCAASGFTFNKYAM | 96 |
| Vh-VI | NWVRQAPGKGLEWVARIRSKYNNYATYYADSVK | |
| | DRFTISRDDSKNTAYLQMNNLKTEDTAVYYCVRH | |
| | GNFGNSYISYWAYWGQGTLVTVSSGGGGSGGGGS | |
| | GGGGSQTVVTQEPSLTVSPGGTVTLTCGSSTGAVTS | |
| | GNYPNWVQQKPGQAPRGLIGGTKFLAPGTPARFSG | |
| | SLLGGKAALTLSGVQPEDEAEYYCVLWYSNRWVF | |
| | GGGTKLTVL | |
| CD3 scFv V99A | EVQLVESGGGLVQPGGSLKLSCAASGFTFNKYAMN | 97 |
| | WVRQAPGKGLEWVARIRSKYNNYATYYADSVKDR | |
| | FTISRDDSKNTAYLQMNNLKTEDTAVYYCARHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv R100A | EVQLVESGGGLVQPGGSLKLSCAASGFTFNKYAMN | 98 |
| | WVRQAPGKGLEWVARIRSKYNNYATYYADSVKDR | |
| | FTISRDDSKNTAYLQMNNLKTEDTAVYYCVAHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv H101A | EVQLVESGGGLVQPGGSLKLSCAASGFTFNKYAMN | 99 |
| | WVRQAPGKGLEWVARIRSKYNNYATYYADSVKDR | |
| | FTISRDDSKNTAYLQMNNLKTEDTAVYYCVRAGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv G102A | EVQLVESGGGLVQPGGSLKLSCAASGFTFNKYAMN | 100 |
| | WVRQAPGKGLEWVARIRSKYNNYATYYADSVKDR | |
| | FTISRDDSKNTAYLQMNNLKTEDTAVYYCVRHAN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv N103A | EVQLVESGGGLVQPGGSLKLSCAASGFTFNKYAMN | 101 |
| | WVRQAPGKGLEWVARIRSKYNNYATYYADSVKDR | |
| | FTISRDDSKNTAYLQMNNLKTEDTAVYYCVRHGA | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv F104A | EVQLVESGGGLVQPGGSLKLSCAASGFTFNKYAMN | 102 |
| | WVRQAPGKGLEWVARIRSKYNNYATYYADSVKDR | |
| | FTISRDDSKNTAYLQMNNLKTEDTAVYYCVRHGN | |
| | AGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv G105A | EVQLVESGGGLVQPGGSLKLSCAASGFTFNKYAMN | 103 |
| | WVRQAPGKGLEWVARIRSKYNNYATYYADSVKDR | |
| | FTISRDDSKNTAYLQMNNLKTEDTAVYYCVRHGN | |
| | FANSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv N106A | EVQLVESGGGLVQPGGSLKLSCAASGFTFNKYAMN | 104 |
| | WVRQAPGKGLEWVARIRSKYNNYATYYADSVKDR | |
| | FTISRDDSKNTAYLQMNNLKTEDTAVYYCVRHGN | |
| | FGASYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv S107A | EVQLVESGGGLVQPGGSLKLSCAASGFTFNKYAMN | 105 |
| | WVRQAPGKGLEWVARIRSKYNNYATYYADSVKDR | |
| | FTISRDDSKNTAYLQMNNLKTEDTAVYYCVRHGN | |
| | FGNAYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv Y108A | EVQLVESGGGLVQPGGSLKLSCAASGFTFNKYAMN | 106 |
| | WVRQAPGKGLEWVARIRSKYNNYATYYADSVKDR | |
| | FTISRDDSKNTAYLQMNNLKTEDTAVYYCVRHGN | |
| | FGNSAISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv I109A | EVQLVESGGGLVQPGGSLKLSCAASGFTFNKYAMN | 107 |
| | WVRQAPGKGLEWVARIRSKYNNYATYYADSVKDR | |
| | FTISRDDSKNTAYLQMNNLKTEDTAVYYCVRHGN | |
| | FGNSYASYWAYWGQGTLVTVSSGGGGSGGGGSG | |
| | GGGSQTVVTQEPSLTVSPGGTVTLTCGSSTGAVTSG | |
| | NYPNWVQQKPGQAPRGLIGGTKFLAPGTPARFSGS | |
| | LLGGKAALTLSGVQPEDEAEYYCVLWYSNRWVFG | |
| | GGTKLTVL | |
| CD3 scFv S110A | EVQLVESGGGLVQPGGSLKLSCAASGFTFNKYAMN | 108 |
| | WVRQAPGKGLEWVARIRSKYNNYATYYADSVKDR | |
| | FTISRDDSKNTAYLQMNNLKTEDTAVYYCVRHGN | |
| | FGNSYIAYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv Y111A | EVQLVESGGGLVQPGGSLKLSCAASGFTFNKYAMN | 109 |
| | WVRQAPGKGLEWVARIRSKYNNYATYYADSVKDR | |
| | FTISRDDSKNTAYLQMNNLKTEDTAVYYCVRHGN | |
| | FGNSYISAWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv W112A | EVQLVESGGGLVQPGGSLKLSCAASGFTFNKYAMN | 110 |
| | WVRQAPGKGLEWVARIRSKYNNYATYYADSVKDR | |
| | FTISRDDSKNTAYLQMNNLKTEDTAVYYCVRHGN | |
| | FGNSYISYAAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv Y114A | EVQLVESGGGLVQPGGSLKLSCAASGFTFNKYAMN | 111 |
| | WVRQAPGKGLEWVARIRSKYNNYATYYADSVKDR | |
| | FTISRDDSKNTAYLQMNNLKTEDTAVYYCVRHGN | |
| | FGNSYISYWAAWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVFGG | |
| | GTKLTVL | |
| CD3 scFv V231A | EVQLVESGGGLVQPGGSLKLSCAASGFTFNKYAMN | 112 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCALWYSNRWVFGG | |
| | GTKLTVL | |
| CD3 scFv L232A | EVQLVESGGGLVQPGGSLKLSCAASGFTFNKYAMN | 113 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVAWYSNRWVFGG | |
| | GTKLTVL | |
| CD3 scFv W233A | EVQLVESGGGLVQPGGSLKLSCAASGFTFNKYAMN | 114 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLAYSNRWVFGGG | |
| | TKLTVL | |
| CD3 scFv Y234A | EVQLVESGGGLVQPGGSLKLSCAASGFTFNKYAMN | 115 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWASNRWVFGG | |
| | GTKLTVL | |
| CD3 scFv S235A | EVQLVESGGGLVQPGGSLKLSCAASGFTFNKYAMN | 116 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYANRWVFGG | |
| | GTKLTVL | |
| CD3 scFv N236A | EVQLVESGGGLVQPGGSLKLSCAASGFTFNKYAMN | 117 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSARWVFGG | |
| | GTKLTVL | |
| CD3 scFv R237A | EVQLVESGGGLVQPGGSLKLSCAASGFTFNKYAMN | 118 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNAWVFGG | |
| | GTKLTVL | |
| CD3 scFv W238A | EVQLVESGGGLVQPGGSLKLSCAASGFTFNKYAMN | 119 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRAVFGGG | |
| | TKLTVL | |
| CD3 scFv V239A | EVQLVESGGGLVQPGGSLKLSCAASGFTFNKYAMN | 120 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWAFGG | |
| | GTKLTVL | |
| CD3 scFv F240A | EVQLVESGGGLVQPGGSLKLSCAASGFTFNKYAMN | 121 |
| | WVRQAPGKGLEWVARIRSKYNNYATYYADSVKDR | |
| | FTISRDDSKNTAYLQMNNLKTEDTAVYYCVRHGN | |
| | FGNSYISYWAYWGQGTLVTVSSGGGGSGGGGSGG | |
| | GGSQTVVTQEPSLTVSPGGTVTLTCGSSTGAVTSGN | |
| | YPNWVQQKPGQAPRGLIGGTKFLAPGTPARFSGSLL | |
| | GGKAALTLSGVQPEDEAEYYCVLWYSNRWVAGG | |
| | GTKLTVL | |
| CD3 scFv | EVQLVESGGGLVQPGGSLKLSCAASGFTFNKYAMN | 122 |
| (mutations relative to SEQ ID | WVRQASGKGLEWVGRIRSKYNNYATYYADSVKDR | |
| NO: 96: P41S, A49G, N87S, | FTISRDDSKNTAYLQMNSLKTEDTAVYYCVRHGNF | |
| L150F, T151S, G163R, P175A, | GNSYISYWAYWGQGTLVTVSSGGGGSGGGGSGGG | |
| K181T, T200V, A202D, L208I, | GSQTVVTQEPSFSVSPGGTVTLTCRSSTGAVTSGNY | |
| G211N, L217I, S218T, V220A, | ANWVQQTPGQAPRGLIGGTKFLAPGVPDRFSGSILG | |
| P222A, E223D, A226S, E227D ) | NKAALTITGAQADDESDYYCVLWYSNRWVFGGGT | |
| | KLTVL | |
| CD3 scFv | EVQLVESGGGLVQPGGSLKLSCAASGFTFQKYAMN | 123 |
| (mutations relative to SEQ ID | WVRQAPGKGLEWVARIRSKYNNYATYYADSVKDR | |
| NO: 96: N30Q, I109V, G172A, | FTISRDDSKNTAYLQMNNLKTEDTAVYYCVRHGNF | |
| V231A) | GNSYVSYWAYWGQGTLVTVSSGGGGSGGGGSGG | |
| Vh-V1 | GGSQTVVTQEPSLTVSPGGTVTLTCGSSTGAVTSAN | |
| | YPNWVQQKPGQAPRGLIGGTKFLAPGTPARFSGSLL | |
| | GGKAALTLSGVQPEDEAEYYCALWYSNRWVFGGG | |
| | TKLTVL | |
| CD3 scFv | EVQLVESGGGLVQPGGSLRLSCAASGFTFSTYAMN | 265 |
| Vh-V1 | WVRQAPGKGLEWVGRIRSKYNNYATYYADSVKGR | |
| | FTISRDDSKNTLYLQMNSLRAEDTAVYYCVRHGNF | |
| | GDSYVSWFAYWGQGTLVTVSSGKPGSGKPGSGKP | |
| | GSGKPGSQAVVTQEPSLTVSPGGTVTLTCGSSTGAV | |
| | TTSNYANWVQQKPGKSPRGLIGGTNKRAPGVPARF | |
| | SGSLLGGKAALTISGAQPEDEADYYCALWYSNHW | |
| | VFGGGTKLTVL | |

TABLE 12
Anti-CD3 Fab Heavy and Light Chain Sequences

| Construct | | SEQ | | SEQ |
|---|---|---|---|---|
| Name/ | | ID | | ID |
| mutations | Heavy Chain | NO: | Light Chain | NO: |
| CD3 | EVQLVESGGGLVQPGGSLKLSC | 266 | QTVVTQEPSLTVSPGGTVTLT | 267 |
| Fab/wt | AASGFTFNKYAMNWVRQAPGK | | CGSSTGAVTSGNYPNWVQQK | |
| | GLEWVARIRSKYNNYATYYADS | | PGQAPRGLIGGTKFLAPGTPA | |
| | VKDRFTISRDDSKNTAYLQMNN | | RFSGSLLGGKAALTLSGVQPE | |
| | LKTEDTAVYYCVRHGNFGNSYI | | DEAEYYCVLWYSNRWVFGG | |
| | SYWAYWGQGTLVTVSSASTKG | | GTKLTVLGQPKAAPSVTLFPP | |
| | PSVFPLAPSSKSTSGGTAALGCL | | SSEELQANKATLVCLISDFYP | |
| | VKDYFPEPVTVSWNSGALTSGV | | GAVTVAWKADSSPVKAGVE | |
| | HTFPAVLQSSGLYSLSSVVTVPS | | TTTPSKQSNNKYAASSYLSLT | |
| | SSLGTQTYICNVNHKPSNTKVD | | PEQWKSHRSYSCQVTHEGST | |
| | KKVEPKSC | | VEKTVAPTECS | |
| CD3 | QTVVTQEPSLTVSPGGTVTLTCG | 268 | EVQLVESGGGLVQPGGSLKL | 269 |
| CrossFab/ | SSTGAVTSGNYPNWVQQKPGQ | | SCAASGFTFNKYAMNWVRQ | |
| HC: | APRGLIGGTKFLAPGTPARFSGS | | APGKGLEWVARIRSKYNNYA | |
| ‘SS’ | LLGGKAALTLSGVQPEDEAEYY | | TYYADSVKDRFTISRDDSKNT | |
| inserted | CVLWYSNRWVFGGGTKLTVLS | | AYLQMNNLKTEDTAVYYCV | |
| between | SASTKGPSVFPLAPSSKSTSGGT | | RHGNFGNSYISYWAYWGQG | |
| VL-CH1 | AALGCLVKDYFPEPVTVSWNSG | | TLVTVSSASVAAPSVFIFPPSD | |
| junction | ALTSGVHTFPAVLQSSGLYSLSS | | EQLKSGTASVVCLLNNFYPRE | |
| LC: | VVTVPSSSLGTQTYICNVNHKPS | | AKVQWKVDNALQSGNSQES | |
| RT | NTKVDGKVEPKSC | | VTEQDSKDSTYSLSSTLTLSK | |
| replaced | | | ADYEKHKVYACEVTHQGLSS | |
| with ‘AS’ | | | PVTKSFNRGEC | |
| in | | | | |
| constant | | | | |
| kappa | | | | |
| light chain | | | | |

In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 96. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 97. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 98. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 99. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 100. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 101. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 102. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 103. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 104. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 105. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 106. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 107. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 108. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 109. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 110. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 111. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 112. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 113. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 114. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 115. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 116. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 117. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 118. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 119. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 120. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 121. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 122. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 123.

### Connections

In some embodiments, the antibody or antigen binding fragment thereof that binds to STEAP-1 is a Fab or Fab′, and the second antibody or antigen binding fragment thereof that binds to CD3 is an scFv and the Fab or Fab′ is connected to the scFv by a linker (L₂).

STEAP-1 Fab/CD3 scFv

In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin light chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin light chain.

### STEAP-1 Fab/CD3 Fab

In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to STEAP-1 is connected to the Fab or Fab′ that binds to CD3 by a linker (L₂). In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin light chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin light chain of the Fab or Fab′ that binds to STEAP-1.

STEAP-1 scFv/CD3 Fab

In some embodiments, the antibody or antigen binding fragment thereof that binds to STEAP-1 is an scFv, and the second antibody or antigen binding fragment thereof that binds to CD3 is a Fab or Fab′ and the scFv that binds to STEAP-1 is connected to the Fab or Fab′ that binds to CD3 by a linker (L₂). In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin light chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin light chain of the scFv that binds to STEAP-1.

STEAP-1 scFv

In some embodiments, the antibody or antigen binding fragment thereof that binds to STEAP-1 is an scFv, and wherein a C-terminus of the scFv that binds to STEAP-1 is connected to an N-terminus of the second antibody or antigen binding fragment thereof that binds to CD3 via a linker (L₂). In some embodiments, the antibody or antigen binding fragment thereof that binds to STEAP-1 is an scFv, and wherein an N-terminus of the scFv that binds to STEAP-1 is connected to a C-terminus of the second antibody or antigen binding fragment thereof that binds to CD3 via a linker (L₂). In some embodiments, a C-terminus of the immunoglobulin light chain of the scFv that binds to STEAP-1 is connected to an N-terminus of the immunoglobulin heavy chain of the scFv that binds to STEAP-1 via a linker. In some embodiments, a C-terminus of the immunoglobulin heavy chain of the scFv that binds to STEAP-1 is connected to an N-terminus of the immunoglobulin light chain that binds to STEAP-1 via a linker.

### STEAP-1 Binding Domains

In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to an N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to the C terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the C terminus of one of the two Fab or Fab′ that are connected by L₁.

In some embodiments, L₁ connects an immunoglobulin heavy chain of the Fab or Fab′ that binds to STEAP-1 with an immunoglobulin light chain of the second antibody or antigen binding fragment that binds to CD3, and L₂ connects an immunoglobulin heavy chain of the second antibody or antigen binding fragment that binds to CD3 with another immunoglobulin heavy chain of the second Fab or Fab′ that binds to STEAP-1.

### Linker

In some embodiments, L₂ comprises an amino acid sequence according to any one of SEQ ID NOs: 222-224, 270-272, and 371.

TABLE 13
Linker Sequences (L₁ or L₂)

| Construct | Amino Acid Sequence | SEQ ID |
|---|---|---|
| Description | (N to C) | NO: |
| Linker | GGGGSGGGGSGGGGS | 222 |
| Linker | GGGGS | 223 |
| Linker | GGGGSGGGS | 224 |
| Linker | GGGGSGGGGS | 270 |
| Linker | GGGGSGGGGSGGGGSGGGGS | 271 |
| Linker | GGGGSGGGGSGGGGSGGGGSGGGGS | 272 |
| Linker | DGGGGSGGGGS | 371 |

### T-Cell Engager (TCE) Sequences

In some embodiments, the polypeptide or polypeptide complex comprises an amino acid sequence that comprises the immunoglobulin light chain that binds to STEAP-1 is selected from SEQ ID NOs: 126-149 and an amino acid sequence that comprises the immunoglobulin heavy chain that binds to STEAP-1 is selected from SEQ ID NOs: 174-197. In some embodiments, the polypeptide or polypeptide complex comprises the amino acid sequences of SEQ ID NO: 126 and SEQ ID NO: 174. In some embodiments, the polypeptide complex comprises the amino acid sequences of SEQ ID NO: 127 and SEQ ID NO: 175.

In some embodiments, the polypeptide complex comprises amino acid sequences with at least 90%, 95%, 96%, 97%, 98%, 99%, or 100% sequence identity to SEQ ID NO: 128 and SEQ ID NO: 176. In some embodiments, the polypeptide complex comprises the amino acid sequences of SEQ ID NO: 128 and SEQ ID NO: 176. In some embodiments, the polypeptide complex comprises amino acid sequences with at least 90%, 95%, 96%, 97%, 98%, 99%, or 100% sequence identity to SEQ ID NO: 129 and SEQ ID NO: 177. In some embodiments, the polypeptide complex comprises the amino acid sequences of SEQ ID NO: 129 and SEQ ID NO: 177. In some embodiments, the polypeptide complex comprises amino acid sequences with at least 90%, 95%, 96%, 97%, 98%, 99%, or 100% sequence identity to SEQ ID NO: 130 and SEQ ID NO: 178. In some embodiments, the polypeptide complex comprises the amino acid sequences of SEQ ID NO: 130 and SEQ ID NO: 178. In some embodiments, the polypeptide complex comprises amino acid sequences with at least 90%, 95%, 96%, 97%, 98%, 99%, or 100% sequence identity to SEQ ID NO: 131 and SEQ ID NO: 179. In some embodiments, the polypeptide complex comprises the amino acid sequences of SEQ ID NO: 131 and SEQ ID NO: 179. In some embodiments, the polypeptide complex comprises amino acid sequences with at least 90%, 95%, 96%, 97%, 98%, 99%, or 100% sequence identity to SEQ ID NO: 132 and SEQ ID NO: 180. In some embodiments, the polypeptide complex comprises the amino acid sequences of SEQ ID NO: 132 and SEQ ID NO: 180. In some embodiments, the polypeptide complex comprises amino acid sequences with at least 90%, 95%, 96%, 97%, 98%, 99%, or 100% sequence identity to SEQ ID NO: 133 and SEQ ID NO: 181. In some embodiments, the polypeptide complex comprises the amino acid sequences of SEQ ID NO: 133 and SEQ ID NO: 181. In some embodiments, the polypeptide complex comprises amino acid sequences with at least 90%, 95%, 96%, 97%, 98%, 99%, or 100% sequence identity to SEQ ID NO: 134 and SEQ ID NO: 182. In some embodiments, the polypeptide complex comprises the amino acid sequences of SEQ ID NO: 134 and SEQ ID NO: 182. In some embodiments, the polypeptide complex comprises amino acid sequences with at least 90%, 95%, 96%, 97%, 98%, 99%, or 100% sequence identity to SEQ ID NO: 135 and SEQ ID NO: 183. In some embodiments, the polypeptide complex comprises the amino acid sequences of SEQ ID NO: 135 and SEQ ID NO: 183. In some embodiments, the polypeptide complex comprises amino acid sequences with at least 90%, 95%, 96%, 97%, 98%, 99%, or 100% sequence identity to SEQ ID NO: 136 and SEQ ID NO: 184. In some embodiments, the polypeptide complex comprises the amino acid sequences of SEQ ID NO: 136 and SEQ ID NO: 184. In some embodiments, the polypeptide complex comprises amino acid sequences with at least 90%, 95%, 96%, 97%, 98%, 99%, or 100% sequence identity to SEQ ID NO: 137 and SEQ ID NO: 185. In some embodiments, the polypeptide complex comprises the amino acid sequences of SEQ ID NO: 137 and SEQ ID NO: 185.

In some embodiments, the polypeptide complex comprises amino acid sequences with at least 90%, 95%, 96%, 97%, 98%, 99%, or 100% sequence identity to SEQ ID NO: 138 and SEQ ID NO: 186. In some embodiments, the polypeptide complex comprises the amino acid sequences of SEQ ID NO: 138 and SEQ ID NO: 186. In some embodiments, the polypeptide complex comprises amino acid sequences with at least 90%, 95%, 96%, 97%, 98%, 99%, or 100% sequence identity to SEQ ID NO: 139 and SEQ ID NO: 187. In some embodiments, the polypeptide complex comprises the amino acid sequences of SEQ ID NO: 139 and SEQ ID NO: 187. In some embodiments, the polypeptide complex comprises amino acid sequences with at least 90%, 95%, 96%, 97%, 98%, 99%, or 100% sequence identity to SEQ ID NO: 140 and SEQ ID NO: 188. In some embodiments, the polypeptide complex comprises the amino acid sequences of SEQ ID NO: 140 and SEQ ID NO: 188. In some embodiments, the polypeptide complex comprises amino acid sequences with at least 90%, 95%, 96%, 97%, 98%, 99%, or 100% sequence identity to SEQ ID NO: 141 and SEQ ID NO: 189. In some embodiments, the polypeptide complex comprises the amino acid sequences of SEQ ID NO: 141 and SEQ ID NO: 189. In some embodiments, the polypeptide complex comprises amino acid sequences with at least 90%, 95%, 96%, 97%, 98%, 99%, or 100% sequence identity to SEQ ID NO: 142 and SEQ ID NO: 190. In some embodiments, the polypeptide complex comprises the amino acid sequences of SEQ ID NO: 142 and SEQ ID NO: 190. In some embodiments, the polypeptide complex comprises amino acid sequences with at least 90%, 95%, 96%, 97%, 98%, 99%, or 100% sequence identity to SEQ ID NO: 143 and SEQ ID NO: 191. In some embodiments, the polypeptide complex comprises the amino acid sequences of SEQ ID NO: 143 and SEQ ID NO: 191. In some embodiments, the polypeptide complex comprises amino acid sequences with at least 90%, 95%, 96%, 97%, 98%, 99%, or 100% sequence identity to SEQ ID NO: 144 and SEQ ID NO: 192. In some embodiments, the polypeptide complex comprises the amino acid sequences of SEQ ID NO: 144 and SEQ ID NO: 192. In some embodiments, the polypeptide complex comprises amino acid sequences with at least 90%, 95%, 96%, 97%, 98%, 99%, or 100% sequence identity to SEQ ID NO: 145 and SEQ ID NO: 193. In some embodiments, the polypeptide complex comprises the amino acid sequences of SEQ ID NO: 145 and SEQ ID NO: 193. In some embodiments, the polypeptide complex comprises amino acid sequences with at least 90%, 95%, 96%, 97%, 98%, 99%, or 100% sequence identity to SEQ ID NO: 146 and SEQ ID NO: 194. In some embodiments, the polypeptide complex comprises the amino acid sequences of SEQ ID NO: 146 and SEQ ID NO: 194. In some embodiments, the polypeptide complex comprises amino acid sequences with at least 90%, 95%, 96%, 97%, 98%, 99%, or 100% sequence identity to SEQ ID NO: 147 and SEQ ID NO: 195. In some embodiments, the polypeptide complex comprises the amino acid sequences of SEQ ID NO: 147 and SEQ ID NO: 195. In some embodiments, the polypeptide complex comprises amino acid sequences with at least 90%, 95%, 96%, 97%, 98%, 99%, or 100% sequence identity to SEQ ID NO: 148 and SEQ ID NO: 196. In some embodiments, the polypeptide complex comprises the amino acid sequences of SEQ ID NO: 148 and SEQ ID NO: 196. In some embodiments, the polypeptide complex comprises amino acid sequences with at least 90%, 95%, 96%, 97%, 98%, 99%, or 100% sequence identity to SEQ ID NO: 149 and SEQ ID NO: 197. In some embodiments, the polypeptide complex comprises the amino acid sequences of SEQ ID NO: 149 and SEQ ID NO: 197.

In some embodiments, the polypeptide complex comprises amino acid sequences with at least 90%, 95%, 96%, 97%, 98%, 99%, or 100% sequence identity to SEQ ID NO: 273 and SEQ ID NO: 299. In some embodiments, the polypeptide complex comprises the amino acid sequences of SEQ ID NO: 273 and SEQ ID NO: 299. In some embodiments, the polypeptide complex comprises amino acid sequences with at least 90%, 95%, 96%, 97%, 98%, 99%, or 100% sequence identity to SEQ ID NO: 274 and SEQ ID NO: 300. In some embodiments, the polypeptide complex comprises the amino acid sequences of SEQ ID NO: 274 and SEQ ID NO: 300. In some embodiments, the polypeptide complex comprises amino acid sequences with at least 90%, 95%, 96%, 97%, 98%, 99%, or 100% sequence identity to SEQ ID NO: 275 and SEQ ID NO: 301. In some embodiments, the polypeptide complex comprises the amino acid sequences of SEQ ID NO: 275 and SEQ ID NO: 301. In some embodiments, the polypeptide complex comprises amino acid sequences with at least 90%, 95%, 96%, 97%, 98%, 99%, or 100% sequence identity to SEQ ID NO: 276 and SEQ ID NO: 302. In some embodiments, the polypeptide complex comprises the amino acid sequences of SEQ ID NO: 276 and SEQ ID NO: 302. In some embodiments, the polypeptide complex comprises amino acid sequences with at least 90%, 95%, 96%, 97%, 98%, 99%, or 100% sequence identity to SEQ ID NO: 277 and SEQ ID NO: 372. In some embodiments, the polypeptide complex comprises the amino acid sequences of SEQ ID NO: 277 and SEQ ID NO: 372. In some embodiments, the polypeptide complex comprises amino acid sequences with at least 90%, 95%, 96%, 97%, 98%, 99%, or 100% sequence identity to SEQ ID NO: 278 and SEQ ID NO: 303. In some embodiments, the polypeptide complex comprises the amino acid sequences of SEQ ID NO: 278 and SEQ ID NO: 303. In some embodiments, the polypeptide complex comprises amino acid sequences with at least 90%, 95%, 96%, 97%, 98%, 99%, or 100% sequence identity to SEQ ID NO: 279 and SEQ ID NO: 304. In some embodiments, the polypeptide complex comprises the amino acid sequences of SEQ ID NO: 279 and SEQ ID NO: 304. In some embodiments, the polypeptide complex comprises amino acid sequences with at least 90%, 95%, 96%, 97%, 98%, 99%, or 100% sequence identity to SEQ ID NO: 280 and SEQ ID NO: 305. In some embodiments, the polypeptide complex comprises the amino acid sequences of SEQ ID NO: 280 and SEQ ID NO: 305. In some embodiments, the polypeptide complex comprises amino acid sequences with at least 90%, 95%, 96%, 97%, 98%, 99%, or 100% sequence identity to SEQ ID NO: 281 and SEQ ID NO: 306. In some embodiments, the polypeptide complex comprises the amino acid sequences of SEQ ID NO: 281 and SEQ ID NO: 306. In some embodiments, the polypeptide complex comprises amino acid sequences with at least 90%, 95%, 96%, 97%, 98%, 99%, or 100% sequence identity to SEQ ID NO: 282 and SEQ ID NO: 307. In some embodiments, the polypeptide complex comprises the amino acid sequences of SEQ ID NO: 282 and SEQ ID NO: 307. In some embodiments, the polypeptide complex comprises amino acid sequences with at least 90%, 95%, 96%, 97%, 98%, 99%, or 100% sequence identity to SEQ ID NO: 283 and SEQ ID NO: 308. In some embodiments, the polypeptide complex comprises the amino acid sequences of SEQ ID NO: 283 and SEQ ID NO: 308. In some embodiments, the polypeptide complex comprises amino acid sequences with at least 90%, 95%, 96%, 97%, 98%, 99%, or 100% sequence identity to SEQ ID NO: 284 and SEQ ID NO: 309. In some embodiments, the polypeptide complex comprises the amino acid sequences of SEQ ID NO: 284 and SEQ ID NO: 309. In some embodiments, the polypeptide complex comprises amino acid sequences with at least 90%, 95%, 96%, 97%, 98%, 99%, or 100% sequence identity to SEQ ID NO: 285 and SEQ ID NO: 310. In some embodiments, the polypeptide complex comprises the amino acid sequences of SEQ ID NO: 285 and SEQ ID NO: 310. In some embodiments, the polypeptide complex comprises amino acid sequences with at least 90%, 95%, 96%, 97%, 98%, 99%, or 100% sequence identity to SEQ ID NO: 286 and SEQ ID NO: 311. In some embodiments, the polypeptide complex comprises the amino acid sequences of SEQ ID NO: 286 and SEQ ID NO: 311. In some embodiments, the polypeptide complex comprises amino acid sequences with at least 90%, 95%, 96%, 97%, 98%, 99%, or 100% sequence identity to SEQ ID NO: 287 and SEQ ID NO: 312. In some embodiments, the polypeptide complex comprises the amino acid sequences of SEQ ID NO: 287 and SEQ ID NO: 312. In some embodiments, the polypeptide complex comprises amino acid sequences with at least 90%, 95%, 96%, 97%, 98%, 99%, or 100% sequence identity to SEQ ID NO: 288 and SEQ ID NO: 313. In some embodiments, the polypeptide complex comprises the amino acid sequences of SEQ ID NO: 288 and SEQ ID NO: 313. In some embodiments, the polypeptide complex comprises amino acid sequences with at least 90%, 95%, 96%, 97%, 98%, 99%, or 100% sequence identity to SEQ ID NO: 289 and SEQ ID NO: 314. In some embodiments, the polypeptide complex comprises the amino acid sequences of SEQ ID NO: 289 and SEQ ID NO: 314. In some embodiments, the polypeptide complex comprises amino acid sequences with at least 90%, 95%, 96%, 97%, 98%, 99%, or 100% sequence identity to SEQ ID NO: 290 and SEQ ID NO: 315. In some embodiments, the polypeptide complex comprises the amino acid sequences of SEQ ID NO: 290 and SEQ ID NO: 315. In some embodiments, the polypeptide complex comprises amino acid sequences with at least 90%, 95%, 96%, 97%, 98%, 99%, or 100% sequence identity to SEQ ID NO: 291 and SEQ ID NO: 316. In some embodiments, the polypeptide complex comprises the amino acid sequences of SEQ ID NO: 291 and SEQ ID NO: 316. In some embodiments, the polypeptide complex comprises amino acid sequences with at least 90%, 95%, 96%, 97%, 98%, 99%, or 100% sequence identity to SEQ ID NO: 292 and SEQ ID NO: 317. In some embodiments, the polypeptide complex comprises the amino acid sequences of SEQ ID NO: 292 and SEQ ID NO: 317. In some embodiments, the polypeptide complex comprises amino acid sequences with at least 90%, 95%, 96%, 97%, 98%, 99%, or 100% sequence identity to SEQ ID NO: 293 and SEQ ID NO: 318. In some embodiments, the polypeptide complex comprises the amino acid sequences of SEQ ID NO: 293 and SEQ ID NO: 318. In some embodiments, the polypeptide complex comprises amino acid sequences with at least 90%, 95%, 96%, 97%, 98%, 99%, or 100% sequence identity to SEQ ID NO: 294 and SEQ ID NO: 319. In some embodiments, the polypeptide complex comprises the amino acid sequences of SEQ ID NO: 294 and SEQ ID NO: 319. In some embodiments, the polypeptide complex comprises amino acid sequences with at least 90%, 95%, 96%, 97%, 98%, 99%, or 100% sequence identity to SEQ ID NO: 295 and SEQ ID NO: 320. In some embodiments, the polypeptide complex comprises the amino acid sequences of SEQ ID NO: 295 and SEQ ID NO: 320. In some embodiments, the polypeptide complex comprises amino acid sequences with at least 90%, 95%, 96%, 97%, 98%, 99%, or 100% sequence identity to SEQ ID NO: 296 and SEQ ID NO: 321. In some embodiments, the polypeptide complex comprises the amino acid sequences of SEQ ID NO: 296 and SEQ ID NO: 321. In some embodiments, the polypeptide complex comprises amino acid sequences with at least 90%, 95%, 96%, 97%, 98%, 99%, or 100% sequence identity to SEQ ID NO: 297 and SEQ ID NO: 322. In some embodiments, the polypeptide complex comprises the amino acid sequences of SEQ ID NO: 297 and SEQ ID NO: 322. In some embodiments, the polypeptide complex comprises amino acid sequences with at least 90%, 95%, 96%, 97%, 98%, 99%, or 100% sequence identity to SEQ ID NO: 298 and SEQ ID NO: 323. In some embodiments, the polypeptide complex comprises the amino acid sequences of SEQ ID NO: 298 and SEQ ID NO: 323.

In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 128-137, 139-149, 283-284, 287-288, and 291-292, and an amino acid sequence, that comprises the immunoglobulin heavy chain that binds to STEAP-1, with at least 95%, 98%, 99%, or 100% sequence identity to any one of SEQ ID NOs: 176-185, 187-197, 308-309, 312-313, and 316-317. In some embodiments, the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 126-127, 138, 273-282, 285-286, and 289-290, and an amino acid sequence, that comprises the immunoglobulin heavy chain that binds to STEAP-1, with at least 95%, 98%, or 99% sequence identity to any one of SEQ ID NOs: 174-175, 186, 299-307, 310-311, and 314-315.

In some embodiments, the polypeptide or polypeptide complex comprises an amino acid sequence, that comprises the immunoglobulin light chain that binds to STEAP-1, according to SEQ ID NO: 283 and an amino acid sequence, that comprises the immunoglobulin heavy chain that binds to STEAP-1, according to SEQ ID NO: 308. In some embodiments, the polypeptide complex comprises amino acid sequences with at least 90%, 95%, 96%, 97%, 98%, 99%, or 100% sequence identity to SEQ ID NO: 283 and SEQ ID NO: 308. In some embodiments, the polypeptide complex comprises the amino acid sequences of SEQ ID NO: 283 and SEQ ID NO: 308.

In some embodiments, the polypeptide or polypeptide complex comprises an amino acid sequence that comprises the immunoglobulin light chain that binds to STEAP-1 according to SEQ ID NO: 284 and an amino acid sequence that comprises the immunoglobulin heavy chain that binds to STEAP-1 according to SEQ ID NO: 309. In some embodiments, the polypeptide complex comprises amino acid sequences with at least 90%, 95%, 96%, 97%, 98%, 99%, or 100% sequence identity to SEQ ID NO: 284 and SEQ ID NO: 309. In some embodiments, the polypeptide complex comprises the amino acid sequences of SEQ ID NO: 284 and SEQ ID NO: 309.

In some embodiments, the polypeptide or polypeptide complex comprises an amino acid sequence that comprises the immunoglobulin light chain that binds to STEAP-1 according to SEQ ID NO: 287 and an amino acid sequence that comprises the immunoglobulin heavy chain that binds to STEAP-1 according to SEQ ID NO: 312. In some embodiments, the polypeptide complex comprises amino acid sequences with at least 90%, 95%, 96%, 97%, 98%, 99%, or 100% sequence identity to SEQ ID NO: 287 and SEQ ID NO: 312. In some embodiments, the polypeptide complex comprises the amino acid sequences of SEQ ID NO: 287 and SEQ ID NO: 312.

In some embodiments, the polypeptide or polypeptide complex comprises an amino acid sequence that comprises the immunoglobulin light chain that binds to STEAP-1 according to SEQ ID NO: 288 and an amino acid sequence that comprises the immunoglobulin heavy chain that binds to STEAP-1 according to SEQ ID NO: 313. In some embodiments, the polypeptide complex comprises amino acid sequences with at least 90%, 95%, 96%, 97%, 98%, 99%, or 100% sequence identity to SEQ ID NO: 288 and SEQ ID NO: 313. In some embodiments, the polypeptide complex comprises the amino acid sequences of SEQ ID NO: 288 and SEQ ID NO: 313.

In some embodiments, the polypeptide or polypeptide complex comprises an amino acid sequence that comprises the immunoglobulin light chain that binds to STEAP-1 according to SEQ ID NO: 291 and an amino acid sequence that comprises the immunoglobulin heavy chain that binds to STEAP-1 according to SEQ ID NO: 316. In some embodiments, the polypeptide complex comprises amino acid sequences with at least 90%, 95%, 96%, 97%, 98%, 99%, or 100% sequence identity to SEQ ID NO: 291 and SEQ ID NO: 316. In some embodiments, the polypeptide complex comprises the amino acid sequences of SEQ ID NO: 291 and SEQ ID NO: 316.

In some embodiments, the polypeptide or polypeptide complex comprises an amino acid sequence that comprises the immunoglobulin light chain that binds to STEAP-1 according to SEQ ID NO: 292 and an amino acid sequence that comprises the immunoglobulin heavy chain that binds to STEAP-1 according to SEQ ID NO: 317. In some embodiments, the polypeptide complex comprises amino acid sequences with at least 90%, 95%, 96%, 97%, 98%, 99%, or 100% sequence identity to SEQ ID NO: 292 and SEQ ID NO: 317. In some embodiments, the polypeptide complex comprises the amino acid sequences of SEQ ID NO: 292 and SEQ ID NO: 317.

In some embodiments, the polypeptide complex comprises amino acid sequences with at least 90%, 95%, 96%, 97%, 98%, 99%, or 100% sequence identity to any one of SEQ ID NOs: 126-173, 273-298, and 330-342 and SEQ ID NOs: 174-221, 299-323, 343-355, and 372.

TABLE 14
Anti-STEAP-1 and Anti-CD3 T-cell engager (TCE) sequencesP

| | | CD3 | | | | |
|---|---|---|---|---|---|---|
| | STEAP-1 | mutations | | | | |
| | mutations | (LC) | Amino Acid Sequence | SEQ | Amino acid sequence | SEQ |
| Name/ | (LC) and | and | (containing | ID | (containing Fab | ID |
| Format | (HC) | (HC) | Fab light chain) | NO: | heavy chain) | NO: |
| TCE-1/ | (None) and | (None) | DIQMTQSPSSLSASVGDRV | 126 | EVQLVESGGGLVQPG | 174 |
| (1 + 1) | (None) | and | TITCKSSQSLLYRSNQKNY | | GSLKLSCAASGFTFNK | |
| | | (None) | LAWYQQKPGKAPKLLIYW | | YAMNWVRQAPGKGL | |
| | | | ASTRESGVPSRFSGSGSGT | | EWVARIRSKYNNYAT | |
| | | | DFTLTISSLQPEDFATYYC | | YYADSVKDRFTISRDD | |
| | | | QQYYNYPRTFGQGTKVEI | | SKNTAYLQMNNLKTE | |
| | | | KRTVAAPSVFIFPPSDEQL | | DTAVYYCVRHGNFGN | |
| | | | KSGTASVVCLLNNFYPRE | | SYISYWAYWGQGTLV | |
| | | | AKVQWKVDNALQSGNSQ | | TVSSGGGGSGGGGSG | |
| | | | ESVTEQDSKDSTYSLSSTL | | GGGSQTVVTQEPSLTV | |
| | | | TLSKADYEKHKVYACEVT | | SPGGTVTLTCGSSTGA | |
| | | | HQGLSSPVTKSFNRGEC | | VTSGNYPNWVQQKPG | |
| | | | | | QAPRGLIGGTKFLAPG | |
| | | | | | TPARFSGSLLGGKAAL | |
| | | | | | TLSGVQPEDEAEYYCV | |
| | | | | | LWYSNRWVFGGGTKL | |
| | | | | | TVLGGGGSEVQLVES | |
| | | | | | GGGLVQPGGSLRLSCA | |
| | | | | | VSGYSITSDYAWNWV | |
| | | | | | RQAPGKGLEWVGYIS | |
| | | | | | NSGSTSYNPSLKSRFTI | |
| | | | | | SRDTSKNTLYLQMNSL | |
| | | | | | RAEDTAVYYCARERN | |
| | | | | | YDYDDYYYAMDYWG | |
| | | | | | QGTLVTVSSASTKGPS | |
| | | | | | VFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE-2/ | (None) and | (None) | EVQLVESGGGLVQPGGSL | 127 | EVQLVESGGGLVQPG | 175 |
| (1 + 1) | (None) | and | KLSCAASGFTFNKYAMN | | GSLRLSCAVSGYSITSD | |
| | | (None) | WVRQAPGKGLEWVARIRS | | YAWNWVRQAPGKGL | |
| | | | KYNNYATYYADSVKDRFT | | EWVGYISNSGSTSYNP | |
| | | | ISRDDSKNTAYLQMNNLK | | SLKSRFTISRDTSKNTL | |
| | | | TEDTAVYYCVRHGNFGNS | | YLQMNSLRAEDTAVY | |
| | | | YISYWAYWGQGTLVTVSS | | YCARERNYDYDDYYY | |
| | | | GGGGSGGGGSGGGGSQTV | | AMDYWGQGTLVTVSS | |
| | | | VTQEPSLTVSPGGTVTLTC | | ASTKGPSVFPLAPSSKS | |
| | | | GSSTGAVTSGNYPNWVQQ | | TSGGTAALGCLVKDY | |
| | | | KPGQAPRGLIGGTKFLAPG | | FPEPVTVSWNSGALTS | |
| | | | TPARFSGSLLGGKAALTLS | | GVHTFPAVLQSSGLYS | |
| | | | GVQPEDEAEYYCVLWYSN | | LSSVVTVPSSSLGTQT | |
| | | | RWVFGGGTKLTVLGGGGS | | YICNVNHKPSNTKVD | |
| | | | DIQMTQSPSSLSASVGDRV | | KKVEPKSC | |
| | | | TITCKSSQSLLYRSNQKNY | | | |
| | | | LAWYQQKPGKAPKLLIYW | | | |
| | | | ASTRESGVPSRFSGSGSGT | | | |
| | | | DFTLTISSLQPEDFATYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE-3/ | (Q3V, | (None) | DIVMTQSPDSLAVSLGERA | 128 | EVQLVESGGGLVQPG | 176 |
| (1 + 1) | S9D, | and | TINCKSSQSLLYRSNQKNY | | GSLKLSCAASGFTFNK | |
| | S12A, | (None) | LAWYQQKPGQPPKLLIYW | | YAMNWVRQAPGKGL | |
| | A13V, | | ASTRESGVPDRFSGSGSGT | | EWVARIRSKYNNYAT | |
| | V15L, | | DFTLTISSLQAEDVAVYYC | | YYADSVKDRFTISRDD | |
| | D17E, | | QQYYNYPRTFGQGTKVEI | | SKNTAYLQMNNLKTE | |
| | V19A, | | KRTVAAPSVFIFPPSDEQL | | DTAVYYCVRHGNFGN | |
| | T22N, | | KSGTASVVCLLNNFYPRE | | SYISYWAYWGQGTLV | |
| | K48Q, | | AKVQWKVDNALQSGNSQ | | TVSSGGGGSGGGGSG | |
| | A49P, | | ESVTEQDSKDSTYSLSSTL | | GGGSQTVVTQEPSLTV | |
| | S66D, | | TLSKADYEKHKVYACEVT | | SPGGTVTLTCGSSTGA | |
| | P86A, | | HQGLSSPVTKSFNRGEC | | VTSGNYPNWVQQKPG | |
| | F89V, | | | | QAPRGLIGGTKFLAPG | |
| | T91V) | | | | TPARFSGSLLGGKAAL | |
| | AND | | | | TLSGVQPEDEAEYYCV | |
| | (V24A, | | | | LWYSNRWVFGGGTKL | |
| | Y27F, | | | | TVLGGGGSEVQLVES | |
| | T58I, | | | | GGGLVQPGGSLRLSCA | |
| | T74N) | | | | ASGFSITSDYAWNWV | |
| | | | | | RQAPGKGLEWVGYIS | |
| | | | | | NSGSISYNPSLKSRFTIS | |
| | | | | | RDNSKNTLYLQMNSL | |
| | | | | | RAEDTAVYYCARERN | |
| | | | | | YDYDDYYYAMDYWG | |
| | | | | | QGTLVTVSSASTKGPS | |
| | | | | | VFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE-4/ | (Q3V, | (None) | EVQLVESGGGLVQPGGSL | 129 | EVQLVESGGGLVQPG | 177 |
| (1 + 1) | S9D, | and | KLSCAASGFTFNKYAMN | | GSLRLSCAASGFSITSD | |
| | S12A, | (None) | WVRQAPGKGLEWVARIRS | | YAWNWVRQAPGKGL | |
| | A13V, | | KYNNYATYYADSVKDRFT | | EWVGYISNSGSISYNPS | |
| | V15L, | | ISRDDSKNTAYLQMNNLK | | LKSRFTISRDNSKNTL | |
| | D17E, | | TEDTAVYYCVRHGNFGNS | | YLQMNSLRAEDTAVY | |
| | V19A, | | YISYWAYWGQGTLVTVSS | | YCARERNYDYDDYYY | |
| | T22N, | | GGGGSGGGGSGGGGSQTV | | AMDYWGQGTLVTVSS | |
| | K48Q, | | VTQEPSLTVSPGGTVTLTC | | ASTKGPSVFPLAPSSKS | |
| | A49P, | | GSSTGAVTSGNYPNWVQQ | | TSGGTAALGCLVKDY | |
| | S66D, | | KPGQAPRGLIGGTKFLAPG | | FPEPVTVSWNSGALTS | |
| | P86A, | | TPARFSGSLLGGKAALTLS | | GVHTFPAVLQSSGLYS | |
| | F89V, | | GVQPEDEAEYYCVLWYSN | | LSSVVTVPSSSLGTQT | |
| | T91V AND | | RWVFGGGTKLTVLGGGGS | | YICNVNHKPSNTKVD | |
| | (V24A, | | DIVMTQSPDSLAVSLGERA | | KKVEPKSC | |
| | Y27F, | | TINCKSSQSLLYRSNQKNY | | | |
| | T58I | | LAWYQQKPGQPPKLLIYW | | | |
| | T74N) | | ASTRESGVPDRFSGSGSGT | | | |
| | | | DFTLTISSLQAEDVAVYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE-5/ | (Q35N) | (None) | DIQMTQSPSSLSASVGDRV | 130 | EVQLVESGGGLVQPG | 178 |
| (1 + 1) | AND | and | TITCKSSQSLLYRSNNKNY | | GSLKLSCAASGFTFNK | |
| | (W35M, | (None) | LAWYQQKPGKAPKLLIYW | | YAMNWVRQAPGKGL | |
| | L64V) | | ASTRESGVPSRFSGSGSGT | | EWVARIRSKYNNYAT | |
| | | | DFTLTISSLQPEDFATYYC | | YYADSVKDRFTISRDD | |
| | | | QQYYNYPRTFGQGTKVEI | | SKNTAYLQMNNLKTE | |
| | | | KRTVAAPSVFIFPPSDEQL | | DTAVYYCVRHGNFGN | |
| | | | KSGTASVVCLLNNFYPRE | | SYISYWAYWGQGTLV | |
| | | | AKVQWKVDNALQSGNSQ | | TVSSGGGGSGGGGSG | |
| | | | ESVTEQDSKDSTYSLSSTL | | GGGSQTVVTQEPSLTV | |
| | | | TLSKADYEKHKVYACEVT | | SPGGTVTLTCGSSTGA | |
| | | | HQGLSSPVTKSFNRGEC | | VTSGNYPNWVQQKPG | |
| | | | | | QAPRGLIGGTKFLAPG | |
| | | | | | TPARFSGSLLGGKAAL | |
| | | | | | TLSGVQPEDEAEYYCV | |
| | | | | | LWYSNRWVFGGGTKL | |
| | | | | | TVLGGGGSEVQLVES | |
| | | | | | GGGLVQPGGSLRLSCA | |
| | | | | | VSGYSITSDYAMNWV | |
| | | | | | RQAPGKGLEWVGYIS | |
| | | | | | NSGSTSYNPSVKSRFTI | |
| | | | | | SRDTSKNTLYLQMNSL | |
| | | | | | RAEDTAVYYCARERN | |
| | | | | | YDYDDYYYAMDYWG | |
| | | | | | QGTLVTVSSASTKGPS | |
| | | | | | VFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE-6/ | (Q35N) | (None) | EVQLVESGGGLVQPGGSL | 131 | EVQLVESGGGLVQPG | 179 |
| (1 + 1) | AND | and | KLSCAASGFTFNKYAMN | | GSLRLSCAVSGYSITSD | |
| | (W35M, | (None) | WVRQAPGKGLEWVARIRS | | YAMNWVRQAPGKGL | |
| | L64V) | | KYNNYATYYADSVKDRFT | | EWVGYISNSGSTSYNP | |
| | | | ISRDDSKNTAYLQMNNLK | | SVKSRFTISRDTSKNTL | |
| | | | TEDTAVYYCVRHGNFGNS | | YLQMNSLRAEDTAVY | |
| | | | YISYWAYWGQGTLVTVSS | | YCARERNYDYDDYYY | |
| | | | GGGGSGGGGSGGGGSQTV | | AMDYWGQGTLVTVSS | |
| | | | VTQEPSLTVSPGGTVTLTC | | ASTKGPSVFPLAPSSKS | |
| | | | GSSTGAVTSGNYPNWVQQ | | TSGGTAALGCLVKDY | |
| | | | KPGQAPRGLIGGTKFLAPG | | FPEPVTVSWNSGALTS | |
| | | | TPARFSGSLLGGKAALTLS | | GVHTFPAVLQSSGLYS | |
| | | | GVQPEDEAEYYCVLWYSN | | LSSVVTVPSSSLGTQT | |
| | | | RWVFGGGTKLTVLGGGGS | | YICNVNHKPSNTKVD | |
| | | | DIQMTQSPSSLSASVGDRV | | KKVEPKSC | |
| | | | TITCKSSQSLLYRSNNKNY | | | |
| | | | LAWYQQKPGKAPKLLIYW | | | |
| | | | ASTRESGVPSRFSGSGSGT | | | |
| | | | DFTLTISSLQPEDFATYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE-7/ | (Q3V, | (None) | DIVMTQSPDSLAVSLGERA | 132 | EVQLVESGGGLVQPG | 180 |
| (1 + 1) | S9D, | and | TINCKSSQSLLYRSNNKNY | | GSLKLSCAASGFTFNK | |
| | S12A, | (None) | LAWYQQKPGQPPKLLIYW | | YAMNWVRQAPGKGL | |
| | A13V, | | ASTRESGVPDRFSGSGSGT | | EWVARIRSKYNNYAT | |
| | V15L, | | DFTLTISSLQAEDVAVYYC | | YYADSVKDRFTISRDD | |
| | D17E, | | QQYYNYPRTFGQGTKVEI | | SKNTAYLQMNNLKTE | |
| | V19A, | | KRTVAAPSVFIFPPSDEQL | | DTAVYYCVRHGNFGN | |
| | T22N, | | KSGTASVVCLLNNFYPRE | | SYISYWAYWGQGTLV | |
| | Q35N, | | AKVQWKVDNALQSGNSQ | | TVSSGGGGSGGGGSG | |
| | K48Q, | | ESVTEQDSKDSTYSLSSTL | | GGGSQTVVTQEPSLTV | |
| | A49P, | | TLSKADYEKHKVYACEVT | | SPGGTVTLTCGSSTGA | |
| | S66D, | | HQGLSSPVTKSFNRGEC | | VTSGNYPNWVQQKPG | |
| | P86A, | | | | QAPRGLIGGTKFLAPG | |
| | F89V, | | | | TPARFSGSLLGGKAAL | |
| | T91V) | | | | TLSGVQPEDEAEYYCV | |
| | AND | | | | LWYSNRWVFGGGTKL | |
| | (V24A, | | | | TVLGGGGSEVQLVES | |
| | Y27F, | | | | GGGLVQPGGSLRLSCA | |
| | W35M, | | | | ASGFSITSDYAMNWV | |
| | T58I, | | | | RQAPGKGLEWVGYIS | |
| | L64V, | | | | NSGSISYNPSVKGRFTI | |
| | S66G, | | | | SRDNSKNTLYLQMNS | |
| | T74N) | | | | LRAEDTAVYYCARER | |
| | | | | | NYDYDDYYYAMDYW | |
| | | | | | GQGTLVTVSSASTKGP | |
| | | | | | SVFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE-8/ | (Q3V, | (None) | EVQLVESGGGLVQPGGSL | 133 | EVQLVESGGGLVQPG | 181 |
| (1 + 1) | S9D, | and | KLSCAASGFTFNKYAMN | | GSLRLSCAASGFSITSD | |
| | S12A, | (None) | WVRQAPGKGLEWVARIRS | | YAMNWVRQAPGKGL | |
| | A13V, | | KYNNYATYYADSVKDRFT | | EWVGYISNSGSISYNPS | |
| | V15L, | | ISRDDSKNTAYLQMNNLK | | VKGRFTISRDNSKNTL | |
| | D17E, | | TEDTAVYYCVRHGNFGNS | | YLQMNSLRAEDTAVY | |
| | V19A, | | YISYWAYWGQGTLVTVSS | | YCARERNYDYDDYYY | |
| | T22N, | | GGGGSGGGGSGGGGSQTV | | AMDYWGQGTLVTVSS | |
| | Q35N, | | VTQEPSLTVSPGGTVTLTC | | ASTKGPSVFPLAPSSKS | |
| | K48Q, | | GSSTGAVTSGNYPNWVQQ | | TSGGTAALGCLVKDY | |
| | A49P, | | KPGQAPRGLIGGTKFLAPG | | FPEPVTVSWNSGALTS | |
| | S66D, | | | | GVHTFPAVLQSSGLYS | |
| | P86A, | | | | LSSVVTVPSSSLGTQT | |
| | F89V, | | | | YICNVNHKPSNTKVD | |
| | T91V) | | | | KKVEPKSC | |
| | AND | | | | | |
| | (V24A, | | | | | |
| | Y27F, | | | | | |
| | W35M, | | | | | |
| | T58I, | | | | | |
| | L64V, | | | | | |
| | S66G, | | | | | |
| | T74N) | | | | | |
| TCE-9/ | (Q35N) | (None) | TPARFSGSLLGGKAALTLS | 134 | EVQLVESGGGLVQPG | 182 |
| (1 + 1) | AND | and | GVQPEDEAEYYCVLWYSN | | GSLKLSCAASGFTFNK | |
| | (W35M, | (None) | RWVFGGGTKLTVLGGGGS | | YAMNWVRQAPGKGL | |
| | L64V, | | DIVMTQSPDSLAVSLGERA | | EWVARIRSKYNNYAT | |
| | E99D, | | TINCKSSQSLLYRSNNKNY | | YYADSVKDRFTISRDD | |
| | M111F) | | LAWYQQKPGQPPKLLIYW | | SKNTAYLQMNNLKTE | |
| | | | ASTRESGVPDRFSGSGSGT | | DTAVYYCVRHGNFGN | |
| | | | DFTLTISSLQAEDVAVYYC | | SYISYWAYWGQGTLV | |
| | | | QQYYNYPRTFGQGTKVEI | | TVSSGGGGSGGGGSG | |
| | | | KRTVAAPSVFIFPPSDEQL | | GGGSQTVVTQEPSLTV | |
| | | | KSGTASVVCLLNNFYPRE | | SPGGTVTLTCGSSTGA | |
| | | | AKVQWKVDNALQSGNSQ | | VTSGNYPNWVQQKPG | |
| | | | ESVTEQDSKDSTYSLSSTL | | QAPRGLIGGTKFLAPG | |
| | | | TLSKADYEKHKVYACEVT | | TPARFSGSLLGGKAAL | |
| | | | HQGLSSPVTKSFNRGEC | | TLSGVQPEDEAEYYCV | |
| | | | DIQMTQSPSSLSASVGDRV | | LWYSNRWVFGGGTKL | |
| | | | TITCKSSQSLLYRSNNKNY | | TVLGGGGSEVQLVES | |
| | | | LAWYQQKPGKAPKLLIYW | | GGGLVQPGGSLRLSCA | |
| | | | ASTRESGVPSRFSGSGSGT | | VSGYSITSDYAMNWV | |
| | | | DFTLTISSLQPEDFATYYC | | RQAPGKGLEWVGYIS | |
| | | | QQYYNYPRTFGQGTKVEI | | NSGSTSYNPSVKSRFTI | |
| | | | KRTVAAPSVFIFPPSDEQL | | SRDTSKNTLYLQMNSL | |
| | | | KSGTASVVCLLNNFYPRE | | RAEDTAVYYCARDRN | |
| | | | AKVQWKVDNALQSGNSQ | | YDYDDYYYAFDYWG | |
| | | | ESVTEQDSKDSTYSLSSTL | | QGTLVTVSSASTKGPS | |
| | | | TLSKADYEKHKVYACEVT | | VFPLAPSSKSTSGGTA | |
| | | | HQGLSSPVTKSFNRGEC | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE- | (Q35N) | (None) | EVQLVESGGGLVQPGGSL | 135 | EVQLVESGGGLVQPG | 183 |
| 10/ | AND | and | KLSCAASGFTFNKYAMN | | GSLRLSCAVSGYSITSD | |
| (1 + 1) | (W35M, | (None) | WVRQAPGKGLEWVARIRS | | YAMNWVRQAPGKGL | |
| | L64V, | | KYNNYATYYADSVKDRFT | | EWVGYISNSGSTSYNP | |
| | E99D, | | ISRDDSKNTAYLQMNNLK | | SVKSRFTISRDTSKNTL | |
| | M111F) | | TEDTAVYYCVRHGNFGNS | | YLQMNSLRAEDTAVY | |
| | | | YISYWAYWGQGTLVTVSS | | YCARDRNYDYDDYY | |
| | | | GGGGSGGGGSGGGGSQTV | | YAFDYWGQGTLVTVS | |
| | | | VTQEPSLTVSPGGTVTLTC | | SASTKGPSVFPLAPSSK | |
| | | | GSSTGAVTSGNYPNWVQQ | | STSGGTAALGCLVKD | |
| | | | KPGQAPRGLIGGTKFLAPG | | YFPEPVTVSWNSGALT | |
| | | | TPARFSGSLLGGKAALTLS | | SGVHTFPAVLQSSGLY | |
| | | | GVQPEDEAEYYCVLWYSN | | SLSSVVTVPSSSLGTQT | |
| | | | RWVFGGGTKLTVLGGGGS | | YICNVNHKPSNTKVD | |
| | | | DIQMTQSPSSLSASVGDRV | | KKVEPKSC | |
| | | | TITCKSSQSLLYRSNNKNY | | | |
| | | | LAWYQQKPGKAPKLLIYW | | | |
| | | | ASTRESGVPSRFSGSGSGT | | | |
| | | | DFTLTISSLQPEDFATYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE- | (Q3V, | (None) | DIVMTQSPDSLAVSLGERA | 136 | EVQLVESGGGLVQPG | 184 |
| 11/ | S9D, | and | TINCKSSQSLLYRSNNKNY | | GSLKLSCAASGFTFNK | |
| (1 + 1) | S12A, | (None) | LAWYQQKPGQPPKLLIYW | | YAMNWVRQAPGKGL | |
| | A13V, | | ASTRESGVPDRFSGSGSGT | | EWVARIRSKYNNYAT | |
| | V15L, | | DFTLTISSLQAEDVAVYYC | | YYADSVKDRFTISRDD | |
| | D17E, | | QQYYNYPRTFGQGTKVEI | | SKNTAYLQMNNLKTE | |
| | V19A, | | KRTVAAPSVFIFPPSDEQL | | DTAVYYCVRHGNFGN | |
| | T22N, | | KSGTASVVCLLNNFYPRE | | SYISYWAYWGQGTLV | |
| | Q35N, | | AKVQWKVDNALQSGNSQ | | TVSSGGGGSGGGGSG | |
| | K48Q, | | ESVTEQDSKDSTYSLSSTL | | GGGSQTVVTQEPSLTV | |
| | A49P, | | TLSKADYEKHKVYACEVT | | SPGGTVTLTCGSSTGA | |
| | S66D, | | HQGLSSPVTKSFNRGEC | | VTSGNYPNWVQQKPG | |
| | P86A, | | | | QAPRGLIGGTKFLAPG | |
| | F89V, | | | | TPARFSGSLLGGKAAL | |
| | T91V) | | | | TLSGVQPEDEAEYYCV | |
| | AND | | | | LWYSNRWVFGGGTKL | |
| | (V24A, | | | | TVLGGGGSEVQLVES | |
| | Y27F, | | | | GGGLVQPGGSLRLSCA | |
| | W35M, | | | | ASGFSITSDYAMNWV | |
| | L64V, | | | | RQAPGKGLEWVGYIS | |
| | T74N, | | | | NSGSTSYNPSVKSRFTI | |
| | E99D, | | | | SRDNSKNTLYLQMNS | |
| | M111F) | | | | LRAEDTAVYYCARDR | |
| | | | | | NYDYDDYYYAFDYW | |
| | | | | | GQGTLVTVSSASTKGP | |
| | | | | | SVFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE- | (Q3V, | (None) | EVQLVESGGGLVQPGGSL | 137 | EVQLVESGGGLVQPG | 185 |
| 12/ | S9D, | and | KLSCAASGFTFNKYAMN | | GSLRLSCAASGFSITSD | |
| (1 + 1) | S12A, | (None) | WVRQAPGKGLEWVARIRS | | YAMNWVRQAPGKGL | |
| | A13V, | | KYNNYATYYADSVKDRFT | | EWVGYISNSGSTSYNP | |
| | V15L, | | ISRDDSKNTAYLQMNNLK | | SVKSRFTISRDNSKNTL | |
| | D17E, | | TEDTAVYYCVRHGNFGNS | | YLQMNSLRAEDTAVY | |
| | V19A, | | YISYWAYWGQGTLVTVSS | | YCARDRNYDYDDYY | |
| | T22N, | | GGGGSGGGGSGGGGSQTV | | YAFDYWGQGTLVTVS | |
| | Q35N, | | VTQEPSLTVSPGGTVTLTC | | SASTKGPSVFPLAPSSK | |
| | K48Q, | | GSSTGAVTSGNYPNWVQQ | | STSGGTAALGCLVKD | |
| | A49P, | | KPGQAPRGLIGGTKFLAPG | | YFPEPVTVSWNSGALT | |
| | S66D, | | TPARFSGSLLGGKAALTLS | | SGVHTFPAVLQSSGLY | |
| | P86A, | | GVQPEDEAEYYCVLWYSN | | SLSSVVTVPSSSLGTQT | |
| | F89V, | | RWVFGGGTKLTVLGGGGS | | YICNVNHKPSNTKVD | |
| | T91V) | | DIVMTQSPDSLAVSLGERA | | KKVEPKSC | |
| | AND | | TINCKSSQSLLYRSNNKNY | | | |
| | (V24A, | | LAWYQQKPGQPPKLLIYW | | | |
| | Y27F, | | ASTRESGVPDRFSGSGSGT | | | |
| | W35M, | | DFTLTISSLQAEDVAVYYC | | | |
| | L64V, | | QQYYNYPRTFGQGTKVEI | | | |
| | T74N, | | KRTVAAPSVFIFPPSDEQL | | | |
| | E99D, | | KSGTASVVCLLNNFYPRE | | | |
| | M111F) | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE- | (None) and | (N30Q, | DIQMTQSPSSLSASVGDRV | 138 | EVQLVESGGGLVQPG | 186 |
| 13/ | (None) | I109V, | TITCKSSQSLLYRSNQKNY | | GSLKLSCAASGFTFQK | |
| (1 + 1) | | G172A, | LAWYQQKPGKAPKLLIYW | | YAMNWVRQAPGKGL | |
| | | V231A) | ASTRESGVPSRFSGSGSGT | | EWVARIRSKYNNYAT | |
| | | AND | DFTLTISSLQPEDFATYYC | | YYADSVKDRFTISRDD | |
| | | (N30Q, | QQYYNYPRTFGQGTKVEI | | SKNTAYLQMNNLKTE | |
| | | I109V, | KRTVAAPSVFIFPPSDEQL | | DTAVYYCVRHGNFGN | |
| | | G172A, | KSGTASVVCLLNNFYPRE | | SYVSYWAYWGQGTL | |
| | | V231A) | AKVQWKVDNALQSGNSQ | | VTVSSGGGGSGGGGS | |
| | | | ESVTEQDSKDSTYSLSSTL | | GGGGSQTVVTQEPSLT | |
| | | | TLSKADYEKHKVYACEVT | | VSPGGTVTLTCGSSTG | |
| | | | HQGLSSPVTKSFNRGEC | | AVTSANYPNWVQQKP | |
| | | | | | GQAPRGLIGGTKFLAP | |
| | | | | | GTPARFSGSLLGGKAA | |
| | | | | | LTLSGVQPEDEAEYYC | |
| | | | | | ALWYSNRWVFGGGT | |
| | | | | | KLTVLGGGGSEVQLV | |
| | | | | | ESGGGLVQPGGSLRLS | |
| | | | | | CAVSGYSITSDYAWN | |
| | | | | | WVRQAPGKGLEWVG | |
| | | | | | YISNSGSTSYNPSLKSR | |
| | | | | | FTISRDTSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE- | (None) and | (N30Q, | EVQLVESGGGLVQPGGSL | 139 | EVQLVESGGGLVQPG | 187 |
| 14/ | (None) | I109V, | KLSCAASGFTFQKYAMN | | GSLRLSCAVSGYSITSD | |
| (1 + 1) | | G172A, | WVRQAPGKGLEWVARIRS | | YAWNWVRQAPGKGL | |
| | | V231A) | KYNNYATYYADSVKDRFT | | EWVGYISNSGSTSYNP | |
| | | AND | ISRDDSKNTAYLQMNNLK | | SLKSRFTISRDTSKNTL | |
| | | (N30Q, | TEDTAVYYCVRHGNFGNS | | YLQMNSLRAEDTAVY | |
| | | I109V, | YVSYWAYWGQGTLVTVS | | YCARERNYDYDDYYY | |
| | | G172A, | SGGGGSGGGGSGGGGSQT | | AMDYWGQGTLVTVSS | |
| | | V231A) | VVTQEPSLTVSPGGTVTLT | | ASTKGPSVFPLAPSSKS | |
| | | | CGSSTGAVTSANYPNWVQ | | TSGGTAALGCLVKDY | |
| | | | QKPGQAPRGLIGGTKFLAP | | FPEPVTVSWNSGALTS | |
| | | | GTPARFSGSLLGGKAALTL | | GVHTFPAVLQSSGLYS | |
| | | | SGVQPEDEAEYYCALWYS | | LSSVVTVPSSSLGTQT | |
| | | | NRWVFGGGTKLTVLGGG | | YICNVNHKPSNTKVD | |
| | | | GSDIQMTQSPSSLSASVGD | | KKVEPKSC | |
| | | | RVTITCKSSQSLLYRSNQK | | | |
| | | | NYLAWYQQKPGKAPKLLI | | | |
| | | | YWASTRESGVPSRFSGSGS | | | |
| | | | GTDFTLTISSLQPEDFATY | | | |
| | | | YCQQYYNYPRTFGQGTKV | | | |
| | | | EIKRTVAAPSVFIFPPSDEQ | | | |
| | | | LKSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE- | (Q3V, | (N30Q, | DIVMTQSPDSLAVSLGERA | 140 | EVQLVESGGGLVQPG | 188 |
| 15/ | S9D, | I109V, | TINCKSSQSLLYRSNQKNY | | GSLKLSCAASGFTFQK | |
| (1 + 1) | S12A, | G172A, | LAWYQQKPGQPPKLLIYW | | YAMNWVRQAPGKGL | |
| | A13V, | V231A) | ASTRESGVPDRFSGSGSGT | | EWVARIRSKYNNYAT | |
| | V15L, | AND | DFTLTISSLQAEDVAVYYC | | YYADSVKDRFTISRDD | |
| | D17E, | (N30Q, | QQYYNYPRTFGQGTKVEI | | SKNTAYLQMNNLKTE | |
| | V19A, | I109V, | KRTVAAPSVFIFPPSDEQL | | DTAVYYCVRHGNFGN | |
| | T22N, | G172A, | KSGTASVVCLLNNFYPRE | | SYVSYWAYWGQGTL | |
| | K48Q, | V231A) | AKVQWKVDNALQSGNSQ | | VTVSSGGGGSGGGGS | |
| | A49P, | | ESVTEQDSKDSTYSLSSTL | | GGGGSQTVVTQEPSLT | |
| | S66D, | | TLSKADYEKHKVYACEVT | | VSPGGTVTLTCGSSTG | |
| | P86A, | | HQGLSSPVTKSFNRGEC | | AVTSANYPNWVQQKP | |
| | F89V, | | | | GQAPRGLIGGTKFLAP | |
| | T91V) | | | | GTPARFSGSLLGGKAA | |
| | AND | | | | LTLSGVQPEDEAEYYC | |
| | (V24A, | | | | ALWYSNRWVFGGGT | |
| | Y27F, | | | | KLTVLGGGGSEVQLV | |
| | T58I, | | | | ESGGGLVQPGGSLRLS | |
| | T74N) | | | | CAASGFSITSDYAWN | |
| | | | | | WVRQAPGKGLEWVG | |
| | | | | | YISNSGSISYNPSLKSR | |
| | | | | | FTISRDNSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE- | (Q3V, | (N30Q, | EVQLVESGGGLVQPGGSL | 141 | EVQLVESGGGLVQPG | 189 |
| 16/ | S9D, | I109V, | KLSCAASGFTFQKYAMN | | GSLRLSCAASGFSITSD | |
| (1 + 1) | S12A, | G172A, | WVRQAPGKGLEWVARIRS | | YAWNWVRQAPGKGL | |
| | A13V, | V231A) | KYNNYATYYADSVKDRFT | | EWVGYISNSGSISYNPS | |
| | V15L, | AND | ISRDDSKNTAYLQMNNLK | | LKSRFTISRDNSKNTL | |
| | D17E, | (N30Q, | TEDTAVYYCVRHGNFGNS | | YLQMNSLRAEDTAVY | |
| | V19A, | I109V, | YVSYWAYWGQGTLVTVS | | YCARERNYDYDDYYY | |
| | T22N, | G172A, | SGGGGSGGGGSGGGGSQT | | AMDYWGQGTLVTVSS | |
| | K48Q, | V231A) | VVTQEPSLTVSPGGTVTLT | | ASTKGPSVFPLAPSSKS | |
| | A49P, | | CGSSTGAVTSANYPNWVQ | | TSGGTAALGCLVKDY | |
| | S66D | | QKPGQAPRGLIGGTKFLAP | | FPEPVTVSWNSGALTS | |
| | P86A, | | GTPARFSGSLLGGKAALTL | | GVHTFPAVLQSSGLYS | |
| | F89V, | | SGVQPEDEAEYYCALWYS | | LSSVVTVPSSSLGTQT | |
| | T91V,) | | NRWVFGGGTKLTVLGGG | | YICNVNHKPSNTKVD | |
| | AND | | GSDIVMTQSPDSLAVSLGE | | KKVEPKSC | |
| | (V24A, | | RATINCKSSQSLLYRSNQK | | | |
| | Y27F, | | NYLAWYQQKPGQPPKLLI | | | |
| | T58I, | | YWASTRESGVPDRFSGSG | | | |
| | T74N) | | SGTDFTLTISSLQAEDVAV | | | |
| | | | YYCQQYYNYPRTFGQGTK | | | |
| | | | VEIKRTVAAPSVFIFPPSDE | | | |
| | | | QLKSGTASVVCLLNNFYP | | | |
| | | | REAKVQWKVDNALQSGN | | | |
| | | | SQESVTEQDSKDSTYSLSS | | | |
| | | | TLTLSKADYEKHKVYACE | | | |
| | | | VTHQGLSSPVTKSFNRGEC | | | |
| TCE- | (Q35N) | (N30Q, | DIQMTQSPSSLSASVGDRV | 142 | EVQLVESGGGLVQPG | 190 |
| 17/ | AND | I109V, | TITCKSSQSLLYRSNNKNY | | GSLKLSCAASGFTFQK | |
| (1 + 1) | (W35M, | G172A, | LAWYQQKPGKAPKLLIYW | | YAMNWVRQAPGKGL | |
| | L64V) | V231A) | ASTRESGVPSRFSGSGSGT | | EWVARIRSKYNNYAT | |
| | | AND | DFTLTISSLQPEDFATYYC | | YYADSVKDRFTISRDD | |
| | | (N30Q, | QQYYNYPRTFGQGTKVEI | | SKNTAYLQMNNLKTE | |
| | | I109V, | KRTVAAPSVFIFPPSDEQL | | DTAVYYCVRHGNFGN | |
| | | G172A, | KSGTASVVCLLNNFYPRE | | SYVSYWAYWGQGTL | |
| | | V231A) | AKVQWKVDNALQSGNSQ | | VTVSSGGGGSGGGGS | |
| | | | ESVTEQDSKDSTYSLSSTL | | GGGGSQTVVTQEPSLT | |
| | | | TLSKADYEKHKVYACEVT | | VSPGGTVTLTCGSSTG | |
| | | | HQGLSSPVTKSFNRGEC | | AVTSANYPNWVQQKP | |
| | | | | | GQAPRGLIGGTKFLAP | |
| | | | | | GTPARFSGSLLGGKAA | |
| | | | | | LTLSGVQPEDEAEYYC | |
| | | | | | ALWYSNRWVFGGGT | |
| | | | | | KLTVLGGGGSEVQLV | |
| | | | | | ESGGGLVQPGGSLRLS | |
| | | | | | CAVSGYSITSDYAMN | |
| | | | | | WVRQAPGKGLEWVG | |
| | | | | | YISNSGSTSYNPSVKSR | |
| | | | | | FTISRDTSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE- | (Q35N) | (N30Q, | EVQLVESGGGLVQPGGSL | 143 | EVQLVESGGGLVQPG | 191 |
| 18/ | AND | I109V, | KLSCAASGFTFQKYAMN | | GSLRLSCAVSGYSITSD | |
| (1 + 1) | (W35M, | G172A, | WVRQAPGKGLEWVARIRS | | YAMNWVRQAPGKGL | |
| | L64V) | V231A) | KYNNYATYYADSVKDRFT | | EWVGYISNSGSTSYNP | |
| | | AND | ISRDDSKNTAYLQMNNLK | | SVKSRFTISRDTSKNTL | |
| | | (N30Q, | TEDTAVYYCVRHGNFGNS | | YLQMNSLRAEDTAVY | |
| | | I109V, | YVSYWAYWGQGTLVTVS | | YCARERNYDYDDYYY | |
| | | G172A, | SGGGGSGGGGSGGGGSQT | | AMDYWGQGTLVTVSS | |
| | | V231A) | VVTQEPSLTVSPGGTVTLT | | ASTKGPSVFPLAPSSKS | |
| | | | CGSSTGAVTSANYPNWVQ | | TSGGTAALGCLVKDY | |
| | | | QKPGQAPRGLIGGTKFLAP | | FPEPVTVSWNSGALTS | |
| | | | GTPARFSGSLLGGKAALTL | | GVHTFPAVLQSSGLYS | |
| | | | SGVQPEDEAEYYCALWYS | | LSSVVTVPSSSLGTQT | |
| | | | NRWVFGGGTKLTVLGGG | | YICNVNHKPSNTKVD | |
| | | | GSDIQMTQSPSSLSASVGD | | KKVEPKSC | |
| | | | RVTITCKSSQSLLYRSNNK | | | |
| | | | NYLAWYQQKPGKAPKLLI | | | |
| | | | YWASTRESGVPSRFSGSGS | | | |
| | | | GTDFTLTISSLQPEDFATY | | | |
| | | | YCQQYYNYPRTFGQGTKV | | | |
| | | | EIKRTVAAPSVFIFPPSDEQ | | | |
| | | | LKSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE- | (Q3V, | (N30Q, | DIVMTQSPDSLAVSLGERA | 144 | EVQLVESGGGLVQPG | 192 |
| 19/ | S9D, | I109V, | TINCKSSQSLLYRSNNKNY | | GSLKLSCAASGFTFQK | |
| (1 + 1) | S12A, | G172A, | LAWYQQKPGQPPKLLIYW | | YAMNWVRQAPGKGL | |
| | A13V, | V231A) | ASTRESGVPDRFSGSGSGT | | EWVARIRSKYNNYAT | |
| | V15L, | AND | DFTLTISSLQAEDVAVYYC | | YYADSVKDRFTISRDD | |
| | D17E, | (N30Q, | QQYYNYPRTFGQGTKVEI | | SKNTAYLQMNNLKTE | |
| | V19A, | I109V, | KRTVAAPSVFIFPPSDEQL | | DTAVYYCVRHGNFGN | |
| | T22N, | G172A, | KSGTASVVCLLNNFYPRE | | SYVSYWAYWGQGTL | |
| | Q35N, | V231A) | AKVQWKVDNALQSGNSQ | | VTVSSGGGGSGGGGS | |
| | K48Q, | | ESVTEQDSKDSTYSLSSTL | | GGGGSQTVVTQEPSLT | |
| | A49P, | | TLSKADYEKHKVYACEVT | | VSPGGTVTLTCGSSTG | |
| | S66D, | | HQGLSSPVTKSFNRGEC | | AVTSANYPNWVQQKP | |
| | P86A, | | | | GQAPRGLIGGTKFLAP | |
| | F89V, | | | | GTPARFSGSLLGGKAA | |
| | T91V) | | | | LTLSGVQPEDEAEYYC | |
| | AND | | | | ALWYSNRWVFGGGT | |
| | (V24A, | | | | KLTVLGGGGSEVQLV | |
| | Y27F, | | | | ESGGGLVQPGGSLRLS | |
| | W35M, | | | | CAASGFSITSDYAMN | |
| | T58I, | | | | WVRQAPGKGLEWVG | |
| | L64V, | | | | YISNSGSISYNPSVKGR | |
| | S66G, | | | | FTISRDNSKNTLYLQM | |
| | T74N) | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE- | (Q3V, | (N30Q, | EVQLVESGGGLVQPGGSL | 145 | EVQLVESGGGLVQPG | 193 |
| 20/ | S9D, | I109V, | KLSCAASGFTFQKYAMN | | GSLRLSCAASGFSITSD | |
| (1 + 1) | S12A, | G172A, | WVRQAPGKGLEWVARIRS | | YAMNWVRQAPGKGL | |
| | A13V | V231A) | KYNNYATYYADSVKDRFT | | EWVGYISNSGSISYNPS | |
| | V15L, | AND | ISRDDSKNTAYLQMNNLK | | VKGRFTISRDNSKNTL | |
| | D17E, | (N30Q, | TEDTAVYYCVRHGNFGNS | | YLQMNSLRAEDTAVY | |
| | V19A, | I109V, | YVSYWAYWGQGTLVTVS | | YCARERNYDYDDYYY | |
| | T22N, | G172A, | SGGGGSGGGGSGGGGSQT | | AMDYWGQGTLVTVSS | |
| | Q35N, | V231A) | VVTQEPSLTVSPGGTVTLT | | ASTKGPSVFPLAPSSKS | |
| | K48Q, | | CGSSTGAVTSANYPNWVQ | | TSGGTAALGCLVKDY | |
| | A49P, | | QKPGQAPRGLIGGTKFLAP | | FPEPVTVSWNSGALTS | |
| | S66D, | | GTPARFSGSLLGGKAALTL | | GVHTFPAVLQSSGLYS | |
| | P86A, | | SGVQPEDEAEYYCALWYS | | LSSVVTVPSSSLGTQT | |
| | F89V, | | NRWVFGGGTKLTVLGGG | | YICNVNHKPSNTKVD | |
| | T91V) | | GSDIVMTQSPDSLAVSLGE | | KKVEPKSC | |
| | AND | | RATINCKSSQSLLYRSNNK | | | |
| | (V24A, | | NYLAWYQQKPGQPPKLLI | | | |
| | Y27F, | | YWASTRESGVPDRFSGSG | | | |
| | W35M, | | SGTDFTLTISSLQAEDVAV | | | |
| | T58I, | | YYCQQYYNYPRTFGQGTK | | | |
| | L64V, | | VEIKRTVAAPSVFIFPPSDE | | | |
| | S66G, | | QLKSGTASVVCLLNNFYP | | | |
| | T74N) | | REAKVQWKVDNALQSGN | | | |
| | | | SQESVTEQDSKDSTYSLSS | | | |
| | | | TLTLSKADYEKHKVYACE | | | |
| | | | VTHQGLSSPVTKSFNRGEC | | | |
| TCE- | (Q35N) | (N30Q, | DIQMTQSPSSLSASVGDRV | 146 | EVQLVESGGGLVQPG | 194 |
| 21/ | AND | I109V, | TITCKSSQSLLYRSNNKNY | | GSLKLSCAASGFTFQK | |
| (1 + 1) | (W35M, | G172A, | LAWYQQKPGKAPKLLIYW | | YAMNWVRQAPGKGL | |
| | L64V, | V231A) | ASTRESGVPSRFSGSGSGT | | EWVARIRSKYNNYAT | |
| | E99D, | AND | DFTLTISSLQPEDFATYYC | | YYADSVKDRFTISRDD | |
| | M111F) | (N30Q, | QQYYNYPRTFGQGTKVEI | | SKNTAYLQMNNLKTE | |
| | | I109V, | KRTVAAPSVFIFPPSDEQL | | DTAVYYCVRHGNFGN | |
| | | G172A, | KSGTASVVCLLNNFYPRE | | SYVSYWAYWGQGTL | |
| | | V231A) | AKVQWKVDNALQSGNSQ | | VTVSSGGGGSGGGGS | |
| | | | ESVTEQDSKDSTYSLSSTL | | GGGGSQTVVTQEPSLT | |
| | | | TLSKADYEKHKVYACEVT | | VSPGGTVTLTCGSSTG | |
| | | | HQGLSSPVTKSFNRGEC | | AVTSANYPNWVQQKP | |
| | | | | | GQAPRGLIGGTKFLAP | |
| | | | | | GTPARFSGSLLGGKAA | |
| | | | | | LTLSGVQPEDEAEYYC | |
| | | | | | ALWYSNRWVFGGGT | |
| | | | | | KLTVLGGGGSEVQLV | |
| | | | | | ESGGGLVQPGGSLRLS | |
| | | | | | CAVSGYSITSDYAMN | |
| | | | | | WVRQAPGKGLEWVG | |
| | | | | | YISNSGSTSYNPSVKSR | |
| | | | | | FTISRDTSKNTLYLQM | |
| | | | | | NSLRAEDTAVYYCAR | |
| | | | | | DRNYDYDDYYYAFDY | |
| | | | | | WGQGTLVTVSSASTK | |
| | | | | | GPSVFPLAPSSKSTSGG | |
| | | | | | TAALGCLVKDYFPEPV | |
| | | | | | TVSWNSGALTSGVHT | |
| | | | | | FPAVLQSSGLYSLSSV | |
| | | | | | VTVPSSSLGTQTYICN | |
| | | | | | VNHKPSNTKVDKKVE | |
| | | | | | PKSC | |
| TCE- | (Q35N) | (N30Q, | EVQLVESGGGLVQPGGSL | 147 | EVQLVESGGGLVQPG | 195 |
| 22/ | AND | I109V, | KLSCAASGFTFQKYAMN | | GSLRLSCAVSGYSITSD | |
| (1 + 1) | (W35M, | G172A, | WVRQAPGKGLEWVARIRS | | YAMNWVRQAPGKGL | |
| | L64V, | V231A) | KYNNYATYYADSVKDRFT | | EWVGYISNSGSTSYNP | |
| | E99D, | AND | ISRDDSKNTAYLQMNNLK | | SVKSRFTISRDTSKNTL | |
| | M111F) | (N30Q, | TEDTAVYYCVRHGNFGNS | | YLQMNSLRAEDTAVY | |
| | | I109V, | YVSYWAYWGQGTLVTVS | | YCARDRNYDYDDYY | |
| | | G172A, | SGGGGSGGGGSGGGGSQT | | YAFDYWGQGTLVTVS | |
| | | V231A) | VVTQEPSLTVSPGGTVTLT | | SASTKGPSVFPLAPSSK | |
| | | | CGSSTGAVTSANYPNWVQ | | STSGGTAALGCLVKD | |
| | | | QKPGQAPRGLIGGTKFLAP | | YFPEPVTVSWNSGALT | |
| | | | GTPARFSGSLLGGKAALTL | | SGVHTFPAVLQSSGLY | |
| | | | SGVQPEDEAEYYCALWYS | | SLSSVVTVPSSSLGTQT | |
| | | | NRWVFGGGTKLTVLGGG | | YICNVNHKPSNTKVD | |
| | | | GSDIQMTQSPSSLSASVGD | | KKVEPKSC | |
| | | | RVTITCKSSQSLLYRSNNK | | | |
| | | | NYLAWYQQKPGKAPKLLI | | | |
| | | | YWASTRESGVPSRFSGSGS | | | |
| | | | GTDFTLTISSLQPEDFATY | | | |
| | | | YCQQYYNYPRTFGQGTKV | | | |
| | | | EIKRTVAAPSVFIFPPSDEQ | | | |
| | | | LKSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGEC | | | |
| TCE- | (Q3V, | (N30Q, | DIVMTQSPDSLAVSLGERA | 148 | EVQLVESGGGLVQPG | 196 |
| 23/ | S9D, | I109V, | TINCKSSQSLLYRSNNKNY | | GSLKLSCAASGFTFQK | |
| (1 + 1) | S12A, | G172A, | LAWYQQKPGQPPKLLIYW | | YAMNWVRQAPGKGL | |
| | A13V, | V231A) | ASTRESGVPDRFSGSGSGT | | EWVARIRSKYNNYAT | |
| | V15L, | AND | DFTLTISSLQAEDVAVYYC | | YYADSVKDRFTISRDD | |
| | D17E, | (N30Q, | QQYYNYPRTFGQGTKVEI | | SKNTAYLQMNNLKTE | |
| | V19A, | I109V, | KRTVAAPSVFIFPPSDEQL | | DTAVYYCVRHGNFGN | |
| | T22N, | G172A, | KSGTASVVCLLNNFYPRE | | SYVSYWAYWGQGTL | |
| | Q35N, | V231A) | AKVQWKVDNALQSGNSQ | | VTVSSGGGGSGGGGS | |
| | K48Q, | | ESVTEQDSKDSTYSLSSTL | | GGGGSQTVVTQEPSLT | |
| | A49P, | | TLSKADYEKHKVYACEVT | | VSPGGTVTLTCGSSTG | |
| | S66D, | | HQGLSSPVTKSFNRGEC | | AVTSANYPNWVQQKP | |
| | P86A, | | | | GQAPRGLIGGTKFLAP | |
| | F89V, | | | | GTPARFSGSLLGGKAA | |
| | T91V) | | | | LTLSGVQPEDEAEYYC | |
| | AND | | | | ALWYSNRWVFGGGT | |
| | (V24A, | | | | KLTVLGGGGSEVQLV | |
| | Y27F, | | | | ESGGGLVQPGGSLRLS | |
| | W35M, | | | | CAASGFSITSDYAMN | |
| | L64V, | | | | WVRQAPGKGLEWVG | |
| | T74N, | | | | YISNSGSTSYNPSVKSR | |
| | E99D, | | | | FTISRDNSKNTLYLQM | |
| | M111F) | | | | NSLRAEDTAVYYCAR | |
| | | | | | DRNYDYDDYYYAFDY | |
| | | | | | WGQGTLVTVSSASTK | |
| | | | | | GPSVFPLAPSSKSTSGG | |
| | | | | | TAALGCLVKDYFPEPV | |
| | | | | | TVSWNSGALTSGVHT | |
| | | | | | FPAVLQSSGLYSLSSV | |
| | | | | | VTVPSSSLGTQTYICN | |
| | | | | | VNHKPSNTKVDKKVE | |
| | | | | | PKSC | |
| TCE- | (Q3V, | (N30Q, | EVQLVESGGGLVQPGGSL | 149 | EVQLVESGGGLVQPG | 197 |
| 24/ | S9D, | I109V, | KLSCAASGFTFQKYAMN | | GSLRLSCAASGFSITSD | |
| (1 + 1) | S12A, | G172A, | WVRQAPGKGLEWVARIRS | | YAMNWVRQAPGKGL | |
| | A13V, | V231A) | KYNNYATYYADSVKDRFT | | EWVGYISNSGSTSYNP | |
| | V15L, | AND | ISRDDSKNTAYLQMNNLK | | SVKSRFTISRDNSKNTL | |
| | D17E, | (N30Q, | TEDTAVYYCVRHGNFGNS | | YLQMNSLRAEDTAVY | |
| | V19A, | I109V, | YVSYWAYWGQGTLVTVS | | YCARDRNYDYDDYY | |
| | T22N, | G172A, | SGGGGSGGGGSGGGGSQT | | YAFDYWGQGTLVTVS | |
| | Q35N, | V231A) | VVTQEPSLTVSPGGTVTLT | | SASTKGPSVFPLAPSSK | |
| | K48Q, | | CGSSTGAVTSANYPNWVQ | | STSGGTAALGCLVKD | |
| | A49P, | | QKPGQAPRGLIGGTKFLAP | | YFPEPVTVSWNSGALT | |
| | S66D, | | GTPARFSGSLLGGKAALTL | | SGVHTFPAVLQSSGLY | |
| | P86A, | | SGVQPEDEAEYYCALWYS | | SLSSVVTVPSSSLGTQT | |
| | F89V, | | NRWVFGGGTKLTVLGGG | | YICNVNHKPSNTKVD | |
| | T91V) | | GSDIVMTQSPDSLAVSLGE | | KKVEPKSC | |
| | AND | | RATINCKSSQSLLYRSNNK | | | |
| | (V24A, | | NYLAWYQQKPGQPPKLLI | | | |
| | Y27F, | | YWASTRESGVPDRFSGSG | | | |
| | W35M, | | SGTDFTLTISSLQAEDVAV | | | |
| | L64V, | | YYCQQYYNYPRTFGQGTK | | | |
| | T74N, | | VEIKRTVAAPSVFIFPPSDE | | | |
| | E99D, | | QLKSGTASVVCLLNNFYP | | | |
| | M111F) | | REAKVQWKVDNALQSGN | | | |
| | | | SQESVTEQDSKDSTYSLSS | | | |
| | | | TLTLSKADYEKHKVYACE | | | |
| | | | VTHQGLSSPVTKSFNRGEC | | | |
| TCE-49 | (None) | (None) | QTVVTQEPSLTVSPGGTVT | 273 | EVQLVESGGGLVQPG | 299 |
| (1 + 1) | AND | AND | LTCGSSTGAVTSGNYPNW | | GSLRLSCAVSGYSITSD | |
| N-term | (None) | None) | VQQKPGQAPRGLIGGTKF | | YAWNWVRQAPGKGL | |
| SBD-9 | | | LAPGTPARFSGSLLGGKAA | | EWVGYISNSGSTSYNP | |
| scFv | | | LTLSGVQPEDEAEYYCVL | | SLKSRFTISRDTSKNTL | |
| (Vh- | | | WYSNRWVFGGGTKLTVL | | YLQMNSLRAEDTAVY | |
| Vl); | | | GQPKAAPSVTLFPPSSEEL | | YCARERNYDYDDYYY | |
| CD3 | | | QANKATLVCLISDFYPGA | | AMDYWGQGTLVTVSS | |
| Fab; | | | VTVAWKADSSPVKAGVET | | GGGGSGGGGSGGGGS | |
| GGGG | | | TTPSKQSNNKYAASSYLSL | | DIQMTQSPSSLSASVG | |
| SGGG | | | TPEQWKSHRSYSCQVTHE | | DRVTITCKSSQSLLYRS | |
| GS | | | GSTVEKTVAPTECS | | NQKNYLAWYQQKPG | |
| linker | | | | | KAPKLLIYWASTRESG | |
| (SEQ | | | | | VPSRFSGSGSGTDFTLT | |
| ID NO: | | | | | ISSLQPEDFATYYCQQ | |
| 270) | | | | | YYNYPRTFGQGTKVEI | |
| | | | | | KGGGGSGGGGSEVQL | |
| | | | | | VESGGGLVQPGGSLKL | |
| | | | | | SCAASGFTFNKYAMN | |
| | | | | | WVRQAPGKGLEWVA | |
| | | | | | RIRSKYNNYATYYADS | |
| | | | | | VKDRFTISRDDSKNTA | |
| | | | | | YLQMNNLKTEDTAVY | |
| | | | | | YCVRHGNFGNSYISY | |
| | | | | | WAYWGQGTLVTVSSA | |
| | | | | | STKGPSVFPLAPSSKST | |
| | | | | | SGGTAALGCLVKDYF | |
| | | | | | PEPVTVSWNSGALTSG | |
| | | | | | VHTFPAVLQSSGLYSL | |
| | | | | | SSVVTVPSSSLGTQTYI | |
| | | | | | CNVNHKPSNTKVDKK | |
| | | | | | VEPKSC | |
| TCE-50 | (None) | (None) | EVQLVESGGGLVQPGGSL | 274 | EVQLVESGGGLVQPG | 300 |
| (1 + 1) | AND | AND | RLSCAVSGYSITSDYAWN | | GSLKLSCAASGFTFNK | |
| N-term | (None) | (None) | WVRQAPGKGLEWVGYIS | | YAMNWVRQAPGKGL | |
| SBD-9 | | | NSGSTSYNPSLKSRFTISRD | | EWVARIRSKYNNYAT | |
| scFv; | | | TSKNTLYLQMNSLRAEDT | | YYADSVKDRFTISRDD | |
| CD3 | | | AVYYCARERNYDYDDYY | | SKNTAYLQMNNLKTE | |
| Fab; | | | YAMDYWGQGTLVTVSSG | | DTAVYYCVRHGNFGN | |
| GGGG | | | GGGSGGGGSGGGGSDIQM | | SYISYWAYWGQGTLV | |
| SGGG | | | TQSPSSLSASVGDRVTITC | | TVSSASTKGPSVFPLAP | |
| GS | | | KSSQSLLYRSNQKNYLAW | | SSKSTSGGTAALGCLV | |
| linker | | | YQQKPGKAPKLLIYWAST | | KDYFPEPVTVSWNSG | |
| (SEQ | | | RESGVPSRFSGSGSGTDFT | | ALTSGVHTFPAVLQSS | |
| ID NO: | | | LTISSLQPEDFATYYCQQY | | GLYSLSSVVTVPSSSL | |
| 270) | | | YNYPRTFGQGTKVEIKGG | | GTQTYICNVNHKPSNT | |
| | | | GGSGGGGSQTVVTQEPSL | | KVDKKVEPKSC | |
| | | | TVSPGGTVTLTCGSSTGAV | | | |
| | | | TSGNYPNWVQQKPGQAPR | | | |
| | | | GLIGGTKFLAPGTPARFSG | | | |
| | | | SLLGGKAALTLSGVQPED | | | |
| | | | EAEYYCVLWYSNRWVFG | | | |
| | | | GGTKLTVLGQPKAAPSVT | | | |
| | | | LFPPSSEELQANKATLVCLI | | | |
| | | | SDFYPGAVTVAWKADSSP | | | |
| | | | VKAGVETTTPSKQSNNKY | | | |
| | | | AASSYLSLTPEQWKSHRS | | | |
| | | | YSCQVTHEGSTVEKTVAP | | | |
| | | | TECS | | | |
| TCE-51 | (None) | (None) | QTVVTQEPSLTVSPGGTVT | 275 | DIQMTQSPSSLSASVG | 301 |
| (1 + 1) | AND | AND | LTCGSSTGAVTSGNYPNW | | DRVTITCKSSQSLLYRS | |
| N-term | (None) | (None) | VQQKPGQAPRGLIGGTKF | | NQKNYLAWYQQKPG | |
| SBD-10 | | | LAPGTPARFSGSLLGGKAA | | KAPKLLIYWASTRESG | |
| scFv; | | | LTLSGVQPEDEAEYYCVL | | VPSRFSGSGSGTDFTLT | |
| CD3 | | | WYSNRWVFGGGTKLTVL | | ISSLQPEDFATYYCQQ | |
| Fab; | | | GQPKAAPSVTLFPPSSEEL | | YYNYPRTFGQGTKVEI | |
| GGGG | | | QANKATLVCLISDFYPGA | | KGGGGSGGGGSGGGG | |
| SGGG | | | VTVAWKADSSPVKAGVET | | SEVQLVESGGGLVQPG | |
| GS | | | TTPSKQSNNKYAASSYLSL | | GSLRLSCAVSGYSITSD | |
| linker | | | TPEQWKSHRSYSCQVTHE | | YAWNWVRQAPGKGL | |
| (SEQ | | | GSTVEKTVAPTECS | | EWVGYISNSGSTSYNP | |
| ID NO: | | | | | SLKSRFTISRDTSKNTL | |
| 270) | | | | | YLQMNSLRAEDTAVY | |
| | | | | | YCARERNYDYDDYYY | |
| | | | | | AMDYWGQGTLVTVSS | |
| | | | | | GGGGSGGGGSEVQLV | |
| | | | | | ESGGGLVQPGGSLKLS | |
| | | | | | CAASGFTFNKYAMNW | |
| | | | | | VRQAPGKGLEWVARI | |
| | | | | | RSKYNNYATYYADSV | |
| | | | | | KDRFTISRDDSKNTAY | |
| | | | | | LQMNNLKTEDTAVYY | |
| | | | | | CVRHGNFGNSYISYW | |
| | | | | | AYWGQGTLVTVSSAS | |
| | | | | | TKGPSVFPLAPSSKSTS | |
| | | | | | GGTAALGCLVKDYFP | |
| | | | | | EPVTVSWNSGALTSG | |
| | | | | | VHTFPAVLQSSGLYSL | |
| | | | | | SSVVTVPSSSLGTQTYI | |
| | | | | | CNVNHKPSNTKVDKK | |
| | | | | | VEPKSC | |
| TCE-52 | (None) | (None) | DIQMTQSPSSLSASVGDRV | 276 | EVQLVESGGGLVQPG | 302 |
| (1 + 1) | AND | AND | TITCKSSQSLLYRSNQKNY | | GSLKLSCAASGFTFNK | |
| | (None) | (None) | LAWYQQKPGKAPKLLIYW | | YAMNWVRQAPGKGL | |
| | | | ASTRESGVPSRFSGSGSGT | | EWVARIRSKYNNYAT | |
| | | | DFTLTISSLQPEDFATYYC | | YYADSVKDRFTISRDD | |
| | | | QQYYNYPRTFGQGTKVEI | | SKNTAYLQMNNLKTE | |
| | | | KGGGGSGGGGSGGGGSEV | | DTAVYYCVRHGNFGN | |
| | | | QLVESGGGLVQPGGSLRL | | SYISYWAYWGQGTLV | |
| | | | SCAVSGYSITSDYAWNWV | | TVSSASTKGPSVFPLAP | |
| | | | RQAPGKGLEWVGYISNSG | | SSKSTSGGTAALGCLV | |
| | | | STSYNPSLKSRFTISRDTSK | | KDYFPEPVTVSWNSG | |
| | | | NTLYLQMNSLRAEDTAVY | | ALTSGVHTFPAVLQSS | |
| | | | YCARERNYDYDDYYYAM | | GLYSLSSVVTVPSSSL | |
| | | | DYWGQGTLVTVSSGGGGS | | GTQTYICNVNHKPSNT | |
| | | | GGGGSQTVVTQEPSLTVSP | | KVDKKVEPKSC | |
| | | | GGTVTLTCGSSTGAVTSG | | | |
| | | | NYPNWVQQKPGQAPRGLI | | | |
| | | | GGTKFLAPGTPARFSGSLL | | | |
| | | | GGKAALTLSGVQPEDEAE | | | |
| | | | YYCVLWYSNRWVFGGGT | | | |
| | | | KLTVLGQPKAAPSVTLFPP | | | |
| | | | SSEELQANKATLVCLISDF | | | |
| | | | YPGAVTVAWKADSSPVK | | | |
| | | | AGVETTTPSKQSNNKYAA | | | |
| | | | SSYLSLTPEQWKSHRSYSC | | | |
| N-term | | | QVTHEGSTVEKTVAPTECS | | | |
| SBD-10 | | | | | | |
| scFv; | | | | | | |
| CD3 | | | | | | |
| Fab; | | | | | | |
| GGGG | | | | | | |
| SGGG | | | | | | |
| GS | | | | | | |
| linker | | | | | | |
| (SEQ | | | | | | |
| ID NO: | | | | | | |
| 270) | | | | | | |
| TCE-53 | (None) | (None) | DIQMTQSPSSLSASVGDRV | 277 | EVQLVESGGGLVQPG | 372 |
| (1 + 1) | AND | AND | TITCKSSQSLLYRSNQKNY | | GSLKLSCAASGFTFNK | |
| C-term | (None) | (None) | LAWYQQKPGKAPKLLIYW | | YAMNWVRQAPGKGL | |
| SBD-1 | | | ASTRESGVPSRFSGSGSGT | | EWVARIRSKYNNYAT | |
| Fab; N- | | | DFTLTISSLQPEDFATYYC | | YYADSVKDRFTISRDD | |
| term | | | QQYYNYPRTFGQGTKVEI | | SKNTAYLQMNNLKTE | |
| CD3 | | | KRTVAAPSVFIFPPSDEQL | | DTAVYYCVRHGNFGN | |
| scFv | | | KSGTASVVCLLNNFYPRE | | SYISYWAYWGQGTLV | |
| (Vh- | | | AKVQWKVDNALQSGNSQ | | TVSSGGGGSGGGGSG | |
| Vl); | | | ESVTEQDSKDSTYSLSSTL | | GGGSQTVVTQEPSLTV | |
| GGGG | | | TLSKADYEKHKVYACEVT | | SPGGTVTLTCGSSTGA | |
| SGGG | | | HQGLSSPVTKSFNRGEC | | VTSGNYPNWVQQKPG | |
| GS | | | | | QAPRGLIGGTKFLAPG | |
| linker | | | | | TPARFSGSLLGGKAAL | |
| (SEQ | | | | | TLSGVQPEDEAEYYCV | |
| ID NO: | | | | | LWYSNRWVFGGGTKL | |
| 270) | | | | | TVLGGGGSGGGGSEV | |
| | | | | | QLVESGGGLVQPGGSL | |
| | | | | | RLSCAVSGYSITSDYA | |
| | | | | | WNWVRQAPGKGLEW | |
| | | | | | VGYISNSGSTSYNPSL | |
| | | | | | KSRFTISRDTSKNTLYL | |
| | | | | | QMNSLRAEDTAVYYC | |
| | | | | | ARERNYDYDDYYYA | |
| | | | | | MDYWGQGTLVTVSSA | |
| | | | | | STKGPSVFPLAPSSKST | |
| | | | | | SGGTAALGCLVKDYF | |
| | | | | | PEPVTVSWNSGALTSG | |
| | | | | | VHTFPAVLQSSGLYSL | |
| | | | | | SSVVTVPSSSLGTQTYI | |
| | | | | | CNVNHKPSNTKVDKK | |
| | | | | | VEPKSC | |
| TCE-54 | (None) | (None) | DIQMTQSPSSLSASVGDRV | 278 | EVQLVESGGGLVQPG | 303 |
| (1 + 1) | AND | AND | TITCKSSQSLLYRSNQKNY | | GSLKLSCAASGFTFNK | |
| C-term | (None) | (None) | LAWYQQKPGKAPKLLIYW | | YAMNWVRQAPGKGL | |
| SBD-1 | | | ASTRESGVPSRFSGSGSGT | | EWVARIRSKYNNYAT | |
| Fab; N- | | | DFTLTISSLQPEDFATYYC | | YYADSVKDRFTISRDD | |
| term | | | QQYYNYPRTFGQGTKVEI | | SKNTAYLQMNNLKTE | |
| CD3 | | | KRTVAAPSVFIFPPSDEQL | | DTAVYYCVRHGNFGN | |
| scFv | | | KSGTASVVCLLNNFYPRE | | SYISYWAYWGQGTLV | |
| (Vh- | | | AKVQWKVDNALQSGNSQ | | TVSSGGGGSGGGGSG | |
| Vl); | | | ESVTEQDSKDSTYSLSSTL | | GGGSQTVVTQEPSLTV | |
| GGGG | | | TLSKADYEKHKVYACEVT | | SPGGTVTLTCGSSTGA | |
| SGGG | | | HQGLSSPVTKSFNRGEC | | VTSGNYPNWVQQKPG | |
| GSGG | | | | | QAPRGLIGGTKFLAPG | |
| GGS | | | | | TPARFSGSLLGGKAAL | |
| linker | | | | | TLSGVQPEDEAEYYCV | |
| (SEQ | | | | | LWYSNRWVFGGGTKL | |
| ID NO: | | | | | TVLGGGGSGGGGSGG | |
| 222) | | | | | GGSEVQLVESGGGLV | |
| | | | | | QPGGSLRLSCAVSGYS | |
| | | | | | ITSDYAWNWVRQAPG | |
| | | | | | KGLEWVGYISNSGSTS | |
| | | | | | YNPSLKSRFTISRDTSK | |
| | | | | | NTLYLQMNSLRAEDT | |
| | | | | | AVYYCARERNYDYDD | |
| | | | | | YYYAMDYWGQGTLV | |
| | | | | | TVSSASTKGPSVFPLAP | |
| | | | | | SSKSTSGGTAALGCLV | |
| | | | | | KDYFPEPVTVSWNSG | |
| | | | | | ALTSGVHTFPAVLQSS | |
| | | | | | GLYSLSSVVTVPSSSL | |
| | | | | | GTQTYICNVNHKPSNT | |
| | | | | | KVDKKVEPKSC | |
| TCE-55 | (None) | (None) | EVQLVESGGGLVQPGGSL | 279 | EVQLVESGGGLVQPG | 304 |
| (1 + 1) | AND | AND | KLSCAASGFTFNKYAMN | | GSLRLSCAVSGYSITSD | |
| C-term | (None) | (None) | WVRQAPGKGLEWVARIRS | | YAWNWVRQAPGKGL | |
| SBD-1 | | | KYNNYATYYADSVKDRFT | | EWVGYISNSGSTSYNP | |
| Fab; N- | | | ISRDDSKNTAYLQMNNLK | | SLKSRFTISRDTSKNTL | |
| term | | | TEDTAVYYCVRHGNFGNS | | YLQMNSLRAEDTAVY | |
| CD3 | | | YISYWAYWGQGTLVTVSS | | YCARERNYDYDDYYY | |
| scFv | | | GGGGSGGGGSGGGGSQTV | | AMDYWGQGTLVTVSS | |
| (Vh- | | | VTQEPSLTVSPGGTVTLTC | | ASTKGPSVFPLAPSSKS | |
| Vl); | | | GSSTGAVTSGNYPNWVQQ | | TSGGTAALGCLVKDY | |
| GGGG | | | KPGQAPRGLIGGTKFLAPG | | FPEPVTVSWNSGALTS | |
| SGGG | | | TPARFSGSLLGGKAALTLS | | GVHTFPAVLQSSGLYS | |
| GS | | | GVQPEDEAEYYCVLWYSN | | LSSVVTVPSSSLGTQT | |
| linker | | | RWVFGGGTKLTVLGGGGS | | YICNVNHKPSNTKVD | |
| (SEQ | | | GGGGSDIQMTQSPSSLSAS | | KKVEPKSC | |
| ID NO: | | | VGDRVTITCKSSQSLLYRS | | | |
| 270) | | | NQKNYLAWYQQKPGKAP | | | |
| | | | KLLIYWASTRESGVPSRFS | | | |
| | | | GSGSGTDFTLTISSLQPEDF | | | |
| | | | ATYYCQQYYNYPRTFGQG | | | |
| | | | TKVEIKRTVAAPSVFIFPPS | | | |
| | | | DEQLKSGTASVVCLLNNF | | | |
| | | | YPREAKVQWKVDNALQS | | | |
| | | | GNSQESVTEQDSKDSTYSL | | | |
| | | | SSTLTLSKADYEKHKVYA | | | |
| | | | CEVTHQGLSSPVTKSFNRG | | | |
| | | | EC | | | |
| TCE-56 | (None) | (None) | EVQLVESGGGLVQPGGSL | 280 | EVQLVESGGGLVQPG | 305 |
| (1 + 1) | AND | AND | KLSCAASGFTFNKYAMN | | GSLRLSCAVSGYSITSD | |
| C-term | (None) | (None) | WVRQAPGKGLEWVARIRS | | YAWNWVRQAPGKGL | |
| SBD-1 | | | KYNNYATYYADSVKDRFT | | EWVGYISNSGSTSYNP | |
| Fab; N- | | | ISRDDSKNTAYLQMNNLK | | SLKSRFTISRDTSKNTL | |
| term | | | TEDTAVYYCVRHGNFGNS | | YLQMNSLRAEDTAVY | |
| CD3 | | | YISYWAYWGQGTLVTVSS | | YCARERNYDYDDYYY | |
| scFv | | | GGGGSGGGGSGGGGSQTV | | AMDYWGQGTLVTVSS | |
| (Vh- | | | VTQEPSLTVSPGGTVTLTC | | ASTKGPSVFPLAPSSKS | |
| Vl); | | | GSSTGAVTSGNYPNWVQQ | | TSGGTAALGCLVKDY | |
| GGGG | | | KPGQAPRGLIGGTKFLAPG | | FPEPVTVSWNSGALTS | |
| SGGG | | | TPARFSGSLLGGKAALTLS | | GVHTFPAVLQSSGLYS | |
| GSGG | | | GVQPEDEAEYYCVLWYSN | | LSSVVTVPSSSLGTQT | |
| GGS | | | RWVFGGGTKLTVLGGGGS | | YICNVNHKPSNTKVD | |
| linker | | | GGGGSGGGGSDIQMTQSP | | KKVEPKSC | |
| (SEQ | | | SSLSASVGDRVTITCKSSQ | | | |
| ID NO: | | | SLLYRSNQKNYLAWYQQ | | | |
| 222) | | | KPGKAPKLLIYWASTRESG | | | |
| | | | VPSRFSGSGSGTDFTLTISS | | | |
| | | | LQPEDFATYYCQQYYNYP | | | |
| | | | RTFGQGTKVEIKRTVAAPS | | | |
| | | | VFIFPPSDEQLKSGTASVV | | | |
| | | | CLLNNFYPREAKVQWKV | | | |
| | | | DNALQSGNSQESVTEQDS | | | |
| | | | KDSTYSLSSTLTLSKADYE | | | |
| | | | KHKVYACEVTHQGLSSPV | | | |
| | | | TKSFNRGEC | | | |
| TCE-57 | (None) | (None) | DIQMTQSPSSLSASVGDRV | 281 | EVQLVESGGGLVQPG | 306 |
| (1 + 1) | AND | AND | TITCKSSQSLLYRSNQKNY | | GSLRLSCAVSGYSITSD | |
| N-term | (None) | (None) | LAWYQQKPGKAPKLLIYW | | YAWNWVRQAPGKGL | |
| SBD-1 | | | ASTRESGVPSRFSGSGSGT | | EWVGYISNSGSTSYNP | |
| Fab; C- | | | DFTLTISSLQPEDFATYYC | | SLKSRFTISRDTSKNTL | |
| term | | | QQYYNYPRTFGQGTKVEI | | YLQMNSLRAEDTAVY | |
| CD3 | | | KRTVAAPSVFIFPPSDEQL | | YCARERNYDYDDYYY | |
| scFv | | | KSGTASVVCLLNNFYPRE | | AMDYWGQGTLVTVSS | |
| (Vh- | | | AKVQWKVDNALQSGNSQ | | ASTKGPSVFPLAPSSKS | |
| Vl); | | | ESVTEQDSKDSTYSLSSTL | | TSGGTAALGCLVKDY | |
| GGGG | | | TLSKADYEKHKVYACEVT | | FPEPVTVSWNSGALTS | |
| SGGG | | | HQGLSSPVTKSFNRGEC | | GVHTFPAVLQSSGLYS | |
| GS | | | | | LSSVVTVPSSSLGTQT | |
| linker | | | | | YICNVNHKPSNTKVD | |
| (SEQ | | | | | KKVEPKSCGGGGSGG | |
| ID NO: | | | | | GGSEVQLVESGGGLV | |
| 270) | | | | | QPGGSLKLSCAASGFT | |
| | | | | | FNKYAMNWVRQAPG | |
| | | | | | KGLEWVARIRSKYNN | |
| | | | | | YATYYADSVKDRFTIS | |
| | | | | | RDDSKNTAYLQMNNL | |
| | | | | | KTEDTAVYYCVRHGN | |
| | | | | | FGNSYISYWAYWGQG | |
| | | | | | TLVTVSSGGGGSGGG | |
| | | | | | GSGGGGSQTVVTQEPS | |
| | | | | | LTVSPGGTVTLTCGSS | |
| | | | | | TGAVTSGNYPNWVQQ | |
| | | | | | KPGQAPRGLIGGTKFL | |
| | | | | | APGTPARFSGSLLGGK | |
| | | | | | AALTLSGVQPEDEAEY | |
| | | | | | YCVLWYSNRWVFGG | |
| | | | | | GTKLTVL | |
| TCE-58 | (None) | (None) | DIQMTQSPSSLSASVGDRV | 282 | EVQLVESGGGLVQPG | 307 |
| (1 + 1) | AND | AND | TITCKSSQSLLYRSNQKNY | | GSLRLSCAVSGYSITSD | |
| N-term | (None) | (None) | LAWYQQKPGKAPKLLIYW | | YAWNWVRQAPGKGL | |
| SBD-1 | | | ASTRESGVPSRFSGSGSGT | | EWVGYISNSGSTSYNP | |
| Fab; C- | | | DFTLTISSLQPEDFATYYC | | SLKSRFTISRDTSKNTL | |
| term | | | QQYYNYPRTFGQGTKVEI | | YLQMNSLRAEDTAVY | |
| CD3 | | | KRTVAAPSVFIFPPSDEQL | | YCARERNYDYDDYYY | |
| scFv | | | KSGTASVVCLLNNFYPRE | | AMDYWGQGTLVTVSS | |
| (Vh- | | | AKVQWKVDNALQSGNSQ | | ASTKGPSVFPLAPSSKS | |
| Vl); | | | ESVTEQDSKDSTYSLSSTL | | TSGGTAALGCLVKDY | |
| GGGG | | | TLSKADYEKHKVYACEVT | | FPEPVTVSWNSGALTS | |
| SGGG | | | HQGLSSPVTKSFNRGECG | | GVHTFPAVLQSSGLYS | |
| GS | | | GGGSGGGGSEVQLVESGG | | LSSVVTVPSSSLGTQT | |
| linker | | | GLVQPGGSLKLSCAASGFT | | YICNVNHKPSNTKVD | |
| (SEQ | | | FNKYAMNWVRQAPGKGL | | KKVEPKSC | |
| ID NO: | | | EWVARIRSKYNNYATYYA | | | |
| 270) | | | DSVKDRFTISRDDSKNTAY | | | |
| | | | LQMNNLKTEDTAVYYCV | | | |
| | | | RHGNFGNSYISYWAYWG | | | |
| | | | QGTLVTVSSGGGGSGGGG | | | |
| | | | SGGGGSQTVVTQEPSLTVS | | | |
| | | | PGGTVTLTCGSSTGAVTSG | | | |
| | | | NYPNWVQQKPGQAPRGLI | | | |
| | | | GGTKFLAPGTPARFSGSLL | | | |
| | | | GGKAALTLSGVQPEDEAE | | | |
| | | | YYCVLWYSNRWVFGGGT | | | |
| | | | KLTVL | | | |
| TCE-59 | (Q106C) | (None) | QTVVTQEPSLTVSPGGTVT | 283 | DIQMTQSPSSLSASVG | 308 |
| (1 + 1) | AND | AND | LTCGSSTGAVTSGNYPNW | | DRVTITCKSSQSLLYRS | |
| N-term | (G45C) | (None) | VQQKPGQAPRGLIGGTKF | | NQKNYLAWYQQKPG | |
| SBD-12 | | | LAPGTPARFSGSLLGGKAA | | KAPKLLIYWASTRESG | |
| scFv | | | LTLSGVQPEDEAEYYCVL | | VPSRFSGSGSGTDFTLT | |
| (Vl- | | | WYSNRWVFGGGTKLTVL | | ISSLQPEDFATYYCQQ | |
| Vh); | | | GQPKAAPSVTLFPPSSEEL | | YYNYPRTFGCGTKVEI | |
| CD3 | | | QANKATLVCLISDFYPGA | | KGGGGSGGGGSGGGG | |
| Fab; | | | VTVAWKADSSPVKAGVET | | SEVQLVESGGGLVQPG | |
| GGGG | | | TTPSKQSNNKYAASSYLSL | | GSLRLSCAVSGYSITSD | |
| SGGG | | | TPEQWKSHRSYSCQVTHE | | YAWNWVRQAPGKCL | |
| GS | | | GSTVEKTVAPTECS | | EWVGYISNSGSTSYNP | |
| linker | | | | | SLKSRFTISRDTSKNTL | |
| (SEQ | | | | | YLQMNSLRAEDTAVY | |
| ID NO: | | | | | YCARERNYDYDDYYY | |
| 270) | | | | | AMDYWGQGTLVTVSS | |
| | | | | | GGGGSGGGGSEVQLV | |
| | | | | | ESGGGLVQPGGSLKLS | |
| | | | | | CAASGFTFNKYAMNW | |
| | | | | | VRQAPGKGLEWVARI | |
| | | | | | RSKYNNYATYYADSV | |
| | | | | | KDRFTISRDDSKNTAY | |
| | | | | | LQMNNLKTEDTAVYY | |
| | | | | | CVRHGNFGNSYISYW | |
| | | | | | AYWGQGTLVTVSSAS | |
| | | | | | TKGPSVFPLAPSSKSTS | |
| | | | | | GGTAALGCLVKDYFP | |
| | | | | | EPVTVSWNSGALTSG | |
| | | | | | VHTFPAVLQSSGLYSL | |
| | | | | | SSVVTVPSSSLGTQTYI | |
| | | | | | CNVNHKPSNTKVDKK | |
| | | | | | VEPKSC | |
| TCE-60 | (Q106C) | (None) | DIQMTQSPSSLSASVGDRV | 284 | EVQLVESGGGLVQPG | 309 |
| (1 + 1) | AND | AND | TITCKSSQSLLYRSNQKNY | | GSLKLSCAASGFTFNK | |
| N-term | (G45C) | (None) | LAWYQQKPGKAPKLLIYW | | YAMNWVRQAPGKGL | |
| SBD-12 | | | ASTRESGVPSRFSGSGSGT | | EWVARIRSKYNNYAT | |
| scFv | | | DFTLTISSLQPEDFATYYC | | YYADSVKDRFTISRDD | |
| (Vl- | | | QQYYNYPRTFGCGTKVEI | | SKNTAYLQMNNLKTE | |
| Vh); | | | KGGGGSGGGGSGGGGSEV | | DTAVYYCVRHGNFGN | |
| CD3 | | | QLVESGGGLVQPGGSLRL | | SYISYWAYWGQGTLV | |
| Fab; | | | SCAVSGYSITSDYAWNWV | | TVSSASTKGPSVFPLAP | |
| GGGG | | | RQAPGKCLEWVGYISNSG | | SSKSTSGGTAALGCLV | |
| SGGG | | | STSYNPSLKSRFTISRDTSK | | KDYFPEPVTVSWNSG | |
| GS | | | NTLYLQMNSLRAEDTAVY | | ALTSGVHTFPAVLQSS | |
| linker | | | YCARERNYDYDDYYYAM | | GLYSLSSVVTVPSSSL | |
| (SEQ | | | DYWGQGTLVTVSSGGGGS | | GTQTYICNVNHKPSNT | |
| ID NO: | | | GGGGSQTVVTQEPSLTVSP | | KVDKKVEPKSC | |
| 270) | | | GGTVTLTCGSSTGAVTSG | | | |
| | | | NYPNWVQQKPGQAPRGLI | | | |
| | | | GGTKFLAPGTPARFSGSLL | | | |
| | | | GGKAALTLSGVQPEDEAE | | | |
| | | | YYCVLWYSNRWVFGGGT | | | |
| | | | KLTVLGQPKAAPSVTLFPP | | | |
| | | | SSEELQANKATLVCLISDF | | | |
| | | | YPGAVTVAWKADSSPVK | | | |
| | | | AGVETTTPSKQSNNKYAA | | | |
| | | | SSYLSLTPEQWKSHRSYSC | | | |
| | | | QVTHEGSTVEKTVAPTECS | | | |
| TCE-61 | (None) | (None) | QTVVTQEPSLTVSPGGTVT | 285 | DIQMTQSPSSLSASVG | 310 |
| (1 + 1) | AND | AND | LTCGSSTGAVTSGNYPNW | | DRVTITCKSSQSLLYRS | |
| N-term | (None) | (None) | VQQKPGQAPRGLIGGTKF | | NQKNYLAWYQQKPG | |
| SBD-10 | | | LAPGTPARFSGSLLGGKAA | | KAPKLLIYWASTRESG | |
| scFv | | | LTLSGVQPEDEAEYYCVL | | VPSRFSGSGSGTDFTLT | |
| (Vl- | | | WYSNRWVFGGGTKLTVL | | ISSLQPEDFATYYCQQ | |
| Vh); | | | GQPKAAPSVTLFPPSSEEL | | YYNYPRTFGQGTKVEI | |
| CD3 | | | QANKATLVCLISDFYPGA | | KGGGGSGGGGSGGGG | |
| Fab; | | | VTVAWKADSSPVKAGVET | | SEVQLVESGGGLVQPG | |
| GGGG | | | TTPSKQSNNKYAASSYLSL | | GSLRLSCAVSGYSITSD | |
| S linker | | | TPEQWKSHRSYSCQVTHE | | YAWNWVRQAPGKGL | |
| (SEQ | | | GSTVEKTVAPTECS | | EWVGYISNSGSTSYNP | |
| ID NO: | | | | | SLKSRFTISRDTSKNTL | |
| 223) | | | | | YLQMNSLRAEDTAVY | |
| | | | | | YCARERNYDYDDYYY | |
| | | | | | AMDYWGQGTLVTVSS | |
| | | | | | GGGGSEVQLVESGGG | |
| | | | | | LVQPGGSLKLSCAASG | |
| | | | | | FTFNKYAMNWVRQAP | |
| | | | | | GKGLEWVARIRSKYN | |
| | | | | | NYATYYADSVKDRFTI | |
| | | | | | SRDDSKNTAYLQMNN | |
| | | | | | LKTEDTAVYYCVRHG | |
| | | | | | NFGNSYISYWAYWGQ | |
| | | | | | GTLVTVSSASTKGPSV | |
| | | | | | FPLAPSSKSTSGGTAA | |
| | | | | | LGCLVKDYFPEPVTVS | |
| | | | | | WNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE-62 | (None) | (None) | DIQMTQSPSSLSASVGDRV | 286 | EVQLVESGGGLVQPG | 311 |
| (1 + 1) | AND | AND | TITCKSSQSLLYRSNQKNY | | GSLKLSCAASGFTFNK | |
| N-term | (None) | (None) | LAWYQQKPGKAPKLLIYW | | YAMNWVRQAPGKGL | |
| SBD-10 | | | ASTRESGVPSRFSGSGSGT | | EWVARIRSKYNNYAT | |
| svFv | | | DFTLTISSLQPEDFATYYC | | YYADSVKDRFTISRDD | |
| (Vl- | | | QQYYNYPRTFGQGTKVEI | | SKNTAYLQMNNLKTE | |
| Vh); | | | KGGGGGGGGSGGGGSEV | | DTAVYYCVRHGNFGN | |
| CD3 | | | QLVESGGGLVQPGGSLRL | | SYISYWAYWGQGTLV | |
| Fab; | | | SCAVSGYSITSDYAWNWV | | TVSSASTKGPSVFPLAP | |
| GGGG | | | RQAPGKGLEWVGYISNSG | | SSKSTSGGTAALGCLV | |
| S linker | | | STSYNPSLKSRFTISRDTSK | | KDYFPEPVTVSWNSG | |
| (SEQ | | | NTLYLQMNSLRAEDTAVY | | ALTSGVHTFPAVLQSS | |
| ID NO: | | | YCARERNYDYDDYYYAM | | GLYSLSSVVTVPSSSL | |
| 223) | | | DYWGQGTLVTVSSGGGGS | | GTQTYICNVNHKPSNT | |
| | | | QTVVTQEPSLTVSPGGTVT | | KVDKKVEPKSC | |
| | | | LTCGSSTGAVTSGNYPNW | | | |
| | | | VQQKPGQAPRGLIGGTKF | | | |
| | | | LAPGTPARFSGSLLGGKAA | | | |
| | | | LTLSGVQPEDEAEYYCVL | | | |
| | | | WYSNRWVFGGGTKLTVL | | | |
| | | | GQPKAAPSVTLFPPSSEEL | | | |
| | | | QANKATLVCLISDFYPGA | | | |
| | | | VTVAWKADSSPVKAGVET | | | |
| | | | TTPSKQSNNKYAASSYLSL | | | |
| | | | TPEQWKSHRSYSCQVTHE | | | |
| | | | GSTVEKTVAPTECS | | | |
| TCE-63 | (Q106C) | (None) | QTVVTQEPSLTVSPGGTVT | 287 | DIQMTQSPSSLSASVG | 312 |
| (1 + 1) | AND | AND | LTCGSSTGAVTSGNYPNW | | DRVTITCKSSQSLLYRS | |
| N-term | (G45C) | (None) | VQQKPGQAPRGLIGGTKF | | NQKNYLAWYQQKPG | |
| SBD-12 | | | LAPGTPARFSGSLLGGKAA | | KAPKLLIYWASTRESG | |
| scFv | | | LTLSGVQPEDEAEYYCVL | | VPSRFSGSGSGTDFTLT | |
| (Vl- | | | WYSNRWVFGGGTKLTVL | | ISSLQPEDFATYYCQQ | |
| Vh); | | | GQPKAAPSVTLFPPSSEEL | | YYNYPRTFGCGTKVEI | |
| CD3 | | | QANKATLVCLISDFYPGA | | KGGGGSGGGGSGGGG | |
| Fab; | | | VTVAWKADSSPVKAGVET | | SEVQLVESGGGLVQPG | |
| GGGG | | | TTPSKQSNNKYAASSYLSL | | GSLRLSCAVSGYSITSD | |
| S linker | | | TPEQWKSHRSYSCQVTHE | | YAWNWVRQAPGKCL | |
| (SEQ | | | GSTVEKTVAPTECS | | EWVGYISNSGSTSYNP | |
| ID NO: | | | | | SLKSRFTISRDTSKNTL | |
| 223) | | | | | YLQMNSLRAEDTAVY | |
| | | | | | YCARERNYDYDDYYY | |
| | | | | | AMDYWGQGTLVTVSS | |
| | | | | | GGGGSEVQLVESGGG | |
| | | | | | LVQPGGSLKLSCAASG | |
| | | | | | FTFNKYAMNWVRQAP | |
| | | | | | GKGLEWVARIRSKYN | |
| | | | | | NYATYYADSVKDRFTI | |
| | | | | | SRDDSKNTAYLQMNN | |
| | | | | | LKTEDTAVYYCVRHG | |
| | | | | | NFGNSYISYWAYWGQ | |
| | | | | | GTLVTVSSASTKGPSV | |
| | | | | | FPLAPSSKSTSGGTAA | |
| | | | | | LGCLVKDYFPEPVTVS | |
| | | | | | WNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE-64 | (Q106C) | (None) | DIQMTQSPSSLSASVGDRV | 288 | EVQLVESGGGLVQPG | 313 |
| (1 + 1) | AND | AND | TITCKSSQSLLYRSNQKNY | | GSLKLSCAASGFTFNK | |
| N-term | (G45C) | (None) | LAWYQQKPGKAPKLLIYW | | YAMNWVRQAPGKGL | |
| SBD-12 | | | ASTRESGVPSRFSGSGSGT | | EWVARIRSKYNNYAT | |
| scFv | | | DFTLTISSLQPEDFATYYC | | YYADSVKDRFTISRDD | |
| (V1- | | | QQYYNYPRTFGCGTKVEI | | SKNTAYLQMNNLKTE | |
| Vh); | | | KGGGGSGGGGSGGGGSEV | | DTAVYYCVRHGNFGN | |
| CD3 | | | QLVESGGGLVQPGGSLRL | | SYISYWAYWGQGTLV | |
| Fab; | | | SCAVSGYSITSDYAWNWV | | TVSSASTKGPSVFPLAP | |
| GGGG | | | RQAPGKCLEWVGYISNSG | | SSKSTSGGTAALGCLV | |
| S linker | | | STSYNPSLKSRFTISRDTSK | | KDYFPEPVTVSWNSG | |
| (SEQ | | | NTLYLQMNSLRAEDTAVY | | ALTSGVHTFPAVLQSS | |
| ID NO: | | | YCARERNYDYDDYYYAM | | GLYSLSSVVTVPSSSL | |
| 223) | | | DYWGQGTLVTVSSGGGGS | | GTQTYICNVNHKPSNT | |
| | | | QTVVTQEPSLTVSPGGTVT | | KVDKKVEPKSC | |
| | | | LTCGSSTGAVTSGNYPNW | | | |
| | | | VQQKPGQAPRGLIGGTKF | | | |
| | | | LAPGTPARFSGSLLGGKAA | | | |
| | | | LTLSGVQPEDEAEYYCVL | | | |
| | | | WYSNRWVFGGGTKLTVL | | | |
| | | | GQPKAAPSVTLFPPSSEEL | | | |
| | | | QANKATLVCLISDFYPGA | | | |
| | | | VTVAWKADSSPVKAGVET | | | |
| | | | TTPSKQSNNKYAASSYLSL | | | |
| | | | TPEQWKSHRSYSCQVTHE | | | |
| | | | GSTVEKTVAPTECS | | | |
| TCE-65 | (None) | (None) | QTVVTQEPSLTVSPGGTVT | 289 | DIQMTQSPSSLSASVG | 314 |
| (1 + 1) | AND | AND | LTCGSSTGAVTSGNYPNW | | DRVTITCKSSQSLLYRS | |
| N-term | (None) | (None) | VQQKPGQAPRGLIGGTKF | | NQKNYLAWYQQKPG | |
| SBD-10 | | | LAPGTPARFSGSLLGGKAA | | KAPKLLIYWASTRESG | |
| svFv | | | LTLSGVQPEDEAEYYCVL | | VPSRFSGSGSGTDFTLT | |
| (Vl- | | | WYSNRWVFGGGTKLTVL | | ISSLQPEDFATYYCQQ | |
| Vh); | | | GQPKAAPSVTLFPPSSEEL | | YYNYPRTFGQGTKVEI | |
| CD3 | | | QANKATLVCLISDFYPGA | | KGGGGSGGGGSGGGG | |
| Fab; | | | VTVAWKADSSPVKAGVET | | SEVQLVESGGGLVQPG | |
| GGGG | | | TTPSKQSNNKYAASSYLSL | | GSLRLSCAVSGYSITSD | |
| SGGG | | | TPEQWKSHRSYSCQVTHE | | YAWNWVRQAPGKGL | |
| GSGG | | | GSTVEKTVAPTECS | | EWVGYISNSGSTSYNP | |
| GGS | | | | | SLKSRFTISRDTSKNTL | |
| linker | | | | | YLQMNSLRAEDTAVY | |
| (SEQ | | | | | YCARERNYDYDDYYY | |
| ID NO: | | | | | AMDYWGQGTLVTVSS | |
| 222) | | | | | GGGGSGGGGSGGGGS | |
| | | | | | EVQLVESGGGLVQPG | |
| | | | | | GSLKLSCAASGFTFNK | |
| | | | | | YAMNWVRQAPGKGL | |
| | | | | | EWVARIRSKYNNYAT | |
| | | | | | YYADSVKDRFTISRDD | |
| | | | | | SKNTAYLQMNNLKTE | |
| | | | | | DTAVYYCVRHGNFGN | |
| | | | | | SYISYWAYWGQGTLV | |
| | | | | | TVSSASTKGPSVFPLAP | |
| | | | | | SSKSTSGGTAALGCLV | |
| | | | | | KDYFPEPVTVSWNSG | |
| | | | | | ALTSGVHTFPAVLQSS | |
| | | | | | GLYSLSSVVTVPSSSL | |
| | | | | | GTQTYICNVNHKPSNT | |
| | | | | | KVDKKVEPKSC | |
| TCE-66 | (None) | (None) | DIQMTQSPSSLSASVGDRV | 290 | EVQLVESGGGLVQPG | 315 |
| (1 + 1) | AND | AND | TITCKSSQSLLYRSNQKNY | | GSLKLSCAASGFTFNK | |
| N-term | (None) | (None) | LAWYQQKPGKAPKLLIYW | | YAMNWVRQAPGKGL | |
| SBD-10 | | | ASTRESGVPSRFSGSGSGT | | EWVARIRSKYNNYAT | |
| svFv | | | DFTLTISSLQPEDFATYYC | | YYADSVKDRFTISRDD | |
| (Vl- | | | QQYYNYPRTFGQGTKVEI | | SKNTAYLQMNNLKTE | |
| Vh); | | | KGGGGSGGGGSGGGGSEV | | DTAVYYCVRHGNFGN | |
| CD3 | | | QLVESGGGLVQPGGSLRL | | SYISYWAYWGQGTLV | |
| Fab; | | | SCAVSGYSITSDYAWNWV | | TVSSASTKGPSVFPLAP | |
| GGGG | | | RQAPGKGLEWVGYISNSG | | SSKSTSGGTAALGCLV | |
| SGGG | | | STSYNPSLKSRFTISRDTSK | | KDYFPEPVTVSWNSG | |
| GSGG | | | NTLYLQMNSLRAEDTAVY | | ALTSGVHTFPAVLQSS | |
| GGS | | | YCARERNYDYDDYYYAM | | GLYSLSSVVTVPSSSL | |
| linker | | | DYWGQGTLVTVSSGGGGS | | GTQTYICNVNHKPSNT | |
| (SEQ | | | GGGGSGGGGSQTVVTQEP | | KVDKKVEPKSC | |
| ID NO: | | | SLTVSPGGTVTLTCGSSTG | | | |
| 222) | | | AVTSGNYPNWVQQKPGQ | | | |
| | | | APRGLIGGTKFLAPGTPAR | | | |
| | | | FSGSLLGGKAALTLSGVQP | | | |
| | | | EDEAEYYCVLWYSNRWV | | | |
| | | | FGGGTKLTVLGQPKAAPS | | | |
| | | | VTLFPPSSEELQANKATLV | | | |
| | | | CLISDFYPGAVTVAWKAD | | | |
| | | | SSPVKAGVETTTPSKQSNN | | | |
| | | | KYAASSYLSLTPEQWKSH | | | |
| | | | RSYSCQVTHEGSTVEKTV | | | |
| | | | APTECS | | | |
| TCE-67 | (Q106C) | (None) | QTVVTQEPSLTVSPGGTVT | 291 | DIQMTQSPSSLSASVG | 316 |
| (1 + 1) | AND | AND | LTCGSSTGAVTSGNYPNW | | DRVTITCKSSQSLLYRS | |
| N-term | (G45C) | (None) | VQQKPGQAPRGLIGGTKF | | NQKNYLAWYQQKPG | |
| SBD-12 | | | LAPGTPARFSGSLLGGKAA | | KAPKLLIYWASTRESG | |
| svFv | | | LTLSGVQPEDEAEYYCVL | | VPSRFSGSGSGTDFTLT | |
| (Vl- | | | WYSNRWVFGGGTKLTVL | | ISSLQPEDFATYYCQQ | |
| Vh); | | | GQPKAAPSVTLFPPSSEEL | | YYNYPRTFGCGTKVEI | |
| CD3 | | | QANKATLVCLISDFYPGA | | KGGGGSGGGGSGGGG | |
| Fab; | | | VTVAWKADSSPVKAGVET | | SEVQLVESGGGLVQPG | |
| GGGG | | | TTPSKQSNNKYAASSYLSL | | GSLRLSCAVSGYSITSD | |
| SGGG | | | TPEQWKSHRSYSCQVTHE | | YAWNWVRQAPGKCL | |
| GSGG | | | GSTVEKTVAPTECS | | EWVGYISNSGSTSYNP | |
| GGS | | | | | SLKSRFTISRDTSKNTL | |
| linker | | | | | YLQMNSLRAEDTAVY | |
| (SEQ | | | | | YCARERNYDYDDYYY | |
| ID NO: | | | | | AMDYWGQGTLVTVSS | |
| 222) | | | | | GGGGSGGGGSGGGGS | |
| | | | | | EVQLVESGGGLVQPG | |
| | | | | | GSLKLSCAASGFTFNK | |
| | | | | | YAMNWVRQAPGKGL | |
| | | | | | EWVARIRSKYNNYAT | |
| | | | | | YYADSVKDRFTISRDD | |
| | | | | | SKNTAYLQMNNLKTE | |
| | | | | | DTAVYYCVRHGNFGN | |
| | | | | | SYISYWAYWGQGTLV | |
| | | | | | TVSSASTKGPSVFPLAP | |
| | | | | | SSKSTSGGTAALGCLV | |
| | | | | | KDYFPEPVTVSWNSG | |
| | | | | | ALTSGVHTFPAVLQSS | |
| | | | | | GLYSLSSVVTVPSSSL | |
| | | | | | GTQTYICNVNHKPSNT | |
| | | | | | KVDKKVEPKSC | |
| TCE-68 | (Q106C) | (None) | DIQMTQSPSSLSASVGDRV | 292 | EVQLVESGGGLVQPG | 317 |
| (1 + 1) | AND | AND | TITCKSSQSLLYRSNQKNY | | GSLKLSCAASGFTFNK | |
| N-term | (G45C) | (None) | LAWYQQKPGKAPKLLIYW | | YAMNWVRQAPGKGL | |
| SBD-12 | | | ASTRESGVPSRFSGSGSGT | | EWVARIRSKYNNYAT | |
| svFv | | | DFTLTISSLQPEDFATYYC | | YYADSVKDRFTISRDD | |
| (Vl- | | | QQYYNYPRTFGCGTKVEI | | SKNTAYLQMNNLKTE | |
| Vh); | | | KGGGGGGGGSGGGGSEV | | DTAVYYCVRHGNFGN | |
| CD3 | | | QLVESGGGLVQPGGSLRL | | SYISYWAYWGQGTLV | |
| Fab; | | | SCAVSGYSITSDYAWNWV | | TVSSASTKGPSVFPLAP | |
| GGGG | | | RQAPGKCLEWVGYISNSG | | SSKSTSGGTAALGCLV | |
| SGGG | | | STSYNPSLKSRFTISRDTSK | | KDYFPEPVTVSWNSG | |
| GSGG | | | NTLYLQMNSLRAEDTAVY | | ALTSGVHTFPAVLQSS | |
| GGS | | | YCARERNYDYDDYYYAM | | GLYSLSSVVTVPSSSL | |
| linker | | | DYWGQGTLVTVSSGGGGS | | GTQTYICNVNHKPSNT | |
| (SEQ | | | GGGGSGGGGSQTVVTQEP | | KVDKKVEPKSC | |
| ID NO: | | | SLTVSPGGTVTLTCGSSTG | | | |
| 222) | | | AVTSGNYPNWVQQKPGQ | | | |
| | | | APRGLIGGTKFLAPGTPAR | | | |
| | | | FSGSLLGGKAALTLSGVQP | | | |
| | | | EDEAEYYCVLWYSNRWV | | | |
| | | | FGGGTKLTVLGQPKAAPS | | | |
| | | | VTLFPPSSEELQANKATLV | | | |
| | | | CLISDFYPGAVTVAWKAD | | | |
| | | | SSPVKAGVETTTPSKQSNN | | | |
| | | | KYAASSYLSLTPEQWKSH | | | |
| | | | RSYSCQVTHEGSTVEKTV | | | |
| | | | APTECS | | | |
| TCE-71 | | (None) | QTVVTQEPSLTVSPGGTVT | 293 | QVQLVQSGAEVKKPG | 318 |
| (1 + 1) | | AND | LTCGSSTGAVTSGNYPNW | | ASVKVSCKASGYTFST | |
| SBD-13 | | (None) | VQQKPGQAPRGLIGGTKF | | YWIEWVRQAPGQRLE | |
| scFv | | | LAPGTPARFSGSLLGGKAA | | WMGEILPGSGQTDFNE | |
| (Vh- | | | LTLSGVQPEDEAEYYCVL | | KFQGRVTFTADTSSDT | |
| Vl); | | | WYSNRWVFGGGTKLTVL | | AYMELSSLRSEDTAVY | |
| CD3 | | | GQPKAAPSVTLFPPSSEEL | | YCTRWGYYGTRGYFN | |
| Fab; | | | QANKATLVCLISDFYPGA | | VWGQGTLVTVSSGGG | |
| GGGG | | | VTVAWKADSSPVKAGVET | | GSGGGGSGGGGSEIVL | |
| SGGG | | | TTPSKQSNNKYAASSYLSL | | TQSPATLSLSPGERATL | |
| GS | | | TPEQWKSHRSYSCQVTHE | | SCRASSSVSYMHWFQ | |
| linker | | | GSTVEKTVAPTECS | | QKPGQAPRLLIYSTSN | |
| (SEQ | | | | | LASGIPARFSGSGSGTD | |
| ID NO: | | | | | YTLTISSLEPEDFAVYY | |
| 270) | | | | | CQQRRSFPYTFGQGTK | |
| | | | | | LEIKGGGGSGGGGSEV | |
| | | | | | QLVESGGGLVQPGGSL | |
| | | | | | KLSCAASGFTFNKYA | |
| | | | | | MNWVRQAPGKGLEW | |
| | | | | | VARIRSKYNNYATYY | |
| | | | | | ADSVKDRFTISRDDSK | |
| | | | | | NTAYLQMNNLKTEDT | |
| | | | | | AVYYCVRHGNFGNSY | |
| | | | | | ISYWAYWGQGTLVTV | |
| | | | | | SSASTKGPSVFPLAPSS | |
| | | | | | KSTSGGTAALGCLVK | |
| | | | | | DYFPEPVTVSWNSGAL | |
| | | | | | TSGVHTFPAVLQSSGL | |
| | | | | | YSLSSVVTVPSSSLGT | |
| | | | | | QTYICNVNHKPSNTKV | |
| | | | | | DKKVEPKSC | |
| TCE-72 | | (None) | QVQLVQSGAEVKKPGASV | 294 | EVQLVESGGGLVQPG | 319 |
| (1 + 1) | | AND | KVSCKASGYTFSTYWIEW | | GSLKLSCAASGFTFNK | |
| SBD-13 | | (None) | VRQAPGQRLEWMGEILPG | | YAMNWVRQAPGKGL | |
| scFv | | | SGQTDFNEKFQGRVTFTA | | EWVARIRSKYNNYAT | |
| (Vh- | | | DTSSDTAYMELSSLRSEDT | | YYADSVKDRFTISRDD | |
| V1); | | | AVYYCTRWGYYGTRGYF | | SKNTAYLQMNNLKTE | |
| CD3 | | | NVWGQGTLVTVSSGGGGS | | DTAVYYCVRHGNFGN | |
| Fab; | | | GGGGSGGGGSEIVLTQSPA | | SYISYWAYWGQGTLV | |
| GGGG | | | TLSLSPGERATLSCRASSS | | TVSSASTKGPSVFPLAP | |
| SGGG | | | VSYMHWFQQKPGQAPRL | | SSKSTSGGTAALGCLV | |
| GS | | | LIYSTSNLASGIPARFSGSG | | KDYFPEPVTVSWNSG | |
| linker | | | SGTDYTLTISSLEPEDFAV | | ALTSGVHTFPAVLQSS | |
| (SEQ | | | YYCQQRRSFPYTFGQGTK | | GLYSLSSVVTVPSSSL | |
| ID NO: | | | LEIKGGGGSGGGGSQTVV | | GTQTYICNVNHKPSNT | |
| 270) | | | TQEPSLTVSPGGTVTLTCG | | KVDKKVEPKSC | |
| | | | SSTGAVTSGNYPNWVQQK | | | |
| | | | PGQAPRGLIGGTKFLAPGT | | | |
| | | | PARFSGSLLGGKAALTLSG | | | |
| | | | VQPEDEAEYYCVLWYSNR | | | |
| | | | WVFGGGTKLTVLGQPKA | | | |
| | | | APSVTLFPPSSEELQANKA | | | |
| | | | TLVCLISDFYPGAVTVAW | | | |
| | | | KADSSPVKAGVETTTPSK | | | |
| | | | QSNNKYAASSYLSLTPEQ | | | |
| | | | WKSHRSYSCQVTHEGSTV | | | |
| | | | EKTVAPTECS | | | |
| TCE-73 | | (None) | QTVVTQEPSLTVSPGGTVT | 295 | EIVLTQSPATLSLSPGE | 320 |
| (1 + 1) | | AND | LTCGSSTGAVTSGNYPNW | | RATLSCRASSSVSYMH | |
| SBD-14 | | (None) | VQQKPGQAPRGLIGGTKF | | WFQQKPGQAPRLLIYS | |
| scFv | | | LAPGTPARFSGSLLGGKAA | | TSNLASGIPARFSGSGS | |
| (V1- | | | LTLSGVQPEDEAEYYCVL | | GTDYTLTISSLEPEDFA | |
| Vh); | | | WYSNRWVFGGGTKLTVL | | VYYCQQRRSFPYTFGQ | |
| CD3 | | | GQPKAAPSVTLFPPSSEEL | | GTKLEIKGGGGSGGG | |
| Fab; | | | QANKATLVCLISDFYPGA | | GSGGGGSQVQLVQSG | |
| GGGG | | | VTVAWKADSSPVKAGVET | | AEVKKPGASVKVSCK | |
| SGGG | | | TTPSKQSNNKYAASSYLSL | | ASGYTFSTYWIEWVR | |
| GS | | | TPEQWKSHRSYSCQVTHE | | QAPGQRLEWMGEILP | |
| (SEQ | | | GSTVEKTVAPTECS | | GSGQTDFNEKFQGRV | |
| ID NO: | | | | | TFTADTSSDTAYMELS | |
| 270) | | | | | SLRSEDTAVYYCTRW | |
| linker | | | | | GYYGTRGYFNVWGQ | |
| | | | | | GTLVTVSSGGGGSGG | |
| | | | | | GGSEVQLVESGGGLV | |
| | | | | | QPGGSLKLSCAASGFT | |
| | | | | | FNKYAMNWVRQAPG | |
| | | | | | KGLEWVARIRSKYNN | |
| | | | | | YATYYADSVKDRFTIS | |
| | | | | | RDDSKNTAYLQMNNL | |
| | | | | | KTEDTAVYYCVRHGN | |
| | | | | | FGNSYISYWAYWGQG | |
| | | | | | TLVTVSSASTKGPSVF | |
| | | | | | PLAPSSKSTSGGTAAL | |
| | | | | | GCLVKDYFPEPVTVS | |
| | | | | | WNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| TCE-74 | | (None) | EIVLTQSPATLSLSPGERAT | 296 | EVQLVESGGGLVQPG | 321 |
| SBD-14 | | AND | LSCRASSSVSYMHWFQQK | | GSLKLSCAASGFTFNK | |
| scFv | | (None) | PGQAPRLLIYSTSNLASGIP | | YAMNWVRQAPGKGL | |
| (Vl- | | | ARFSGSGSGTDYTLTISSLE | | EWVARIRSKYNNYAT | |
| Vh); | | | PEDFAVYYCQQRRSFPYTF | | YYADSVKDRFTISRDD | |
| CD3 | | | GQGTKLEIKGGGGSGGGG | | SKNTAYLQMNNLKTE | |
| Fab; | | | SGGGGSQVQLVQSGAEVK | | DTAVYYCVRHGNFGN | |
| GGGG | | | KPGASVKVSCKASGYTFS | | SYISYWAYWGQGTLV | |
| SGGG | | | TYWIEWVRQAPGQRLEW | | TVSSASTKGPSVFPLAP | |
| GS | | | MGEILPGSGQTDFNEKFQG | | SSKSTSGGTAALGCLV | |
| linker | | | RVTFTADTSSDTAYMELSS | | KDYFPEPVTVSWNSG | |
| (SEQ | | | LRSEDTAVYYCTRWGYY | | ALTSGVHTFPAVLQSS | |
| ID NO: | | | GTRGYFNVWGQGTLVTVS | | GLYSLSSVVTVPSSSL | |
| 270) | | | SGGGGSGGGGSQTVVTQE | | GTQTYICNVNHKPSNT | |
| | | | PSLTVSPGGTVTLTCGSST | | KVDKKVEPKSC | |
| | | | GAVTSGNYPNWVQQKPG | | | |
| | | | QAPRGLIGGTKFLAPGTPA | | | |
| | | | RFSGSLLGGKAALTLSGV | | | |
| | | | QPEDEAEYYCVLWYSNR | | | |
| | | | WVFGGGTKLTVLGQPKA | | | |
| | | | APSVTLFPPSSEELQANKA | | | |
| | | | TLVCLISDFYPGAVTVAW | | | |
| | | | KADSSPVKAGVETTTPSK | | | |
| | | | QSNNKYAASSYLSLTPEQ | | | |
| | | | WKSHRSYSCQVTHEGSTV | | | |
| | | | EKTVAPTECS | | | |
| TCE-75 | | (None) | EIVLTQSPATLSLSPGERAT | 297 | QVQLVQSGAEVKKPG | 322 |
| (1 + 1) | | AND | LSCRASSSVSYMHWFQQK | | ASVKVSCKASGYTFST | |
| N-term | | (None) | PGQAPRLLIYSTSNLASGIP | | YWIEWVRQAPGQRLE | |
| SBD-8 | | | ARFSGSGSGTDYTLTISSLE | | WMGEILPGSGQTDFNE | |
| Fab; | | | PEDFAVYYCQQRRSFPYTF | | KFQGRVTFTADTSSDT | |
| CD3 | | | GQGTKLEIKRTVAAPSVFI | | AYMELSSLRSEDTAVY | |
| scFv | | | FPPSDEQLKSGTASVVCLL | | YCTRWGYYGTRGYFN | |
| (Vh- | | | NNFYPREAKVQWKVDNA | | VWGQGTLVTVSSAST | |
| Vl); | | | LQSGNSQESVTEQDSKDST | | KGPSVFPLAPSSKSTSG | |
| GGGG | | | YSLSSTLTLSKADYEKHKV | | GTAALGCLVKDYFPEP | |
| SGGG | | | YACEVTHQGLSSPVTKSFN | | VTVSWNSGALTSGVH | |
| GS | | | RGEC | | TFPAVLQSSGLYSLSS | |
| linker | | | | | VVTVPSSSLGTQTYIC | |
| (SEQ | | | | | NVNHKPSNTKVDKKV | |
| ID NO: | | | | | EPKSCGGGGSGGGGSE | |
| 270) | | | | | VQLVESGGGLVQPGG | |
| | | | | | SLKLSCAASGFTFNKY | |
| | | | | | AMNWVRQAPGKGLE | |
| | | | | | WVARIRSKYNNYATY | |
| | | | | | YADSVKDRFTISRDDS | |
| | | | | | KNTAYLQMNNLKTED | |
| | | | | | TAVYYCVRHGNFGNS | |
| | | | | | YISYWAYWGQGTLVT | |
| | | | | | VSSGGGGSGGGGSGG | |
| | | | | | GGSQTVVTQEPSLTVS | |
| | | | | | PGGTVTLTCGSSTGAV | |
| | | | | | TSGNYPNWVQQKPGQ | |
| | | | | | APRGLIGGTKFLAPGT | |
| | | | | | PARFSGSLLGGKAALT | |
| | | | | | LSGVQPEDEAEYYCVL | |
| | | | | | WYSNRWVFGGGTKLT | |
| | | | | | VL | |
| TCE-76 | | (None) | EIVLTQSPATLSLSPGERAT | 298 | QVQLVQSGAEVKKPG | 323 |
| (1 + 1) | | AND | LSCRASSSVSYMHWFQQK | | ASVKVSCKASGYTFST | |
| N-term | | (None) | PGQAPRLLIYSTSNLASGIP | | YWIEWVRQAPGQRLE | |
| SBD-8 | | | ARFSGSGSGTDYTLTISSLE | | WMGEILPGSGQTDFNE | |
| Fab; | | | PEDFAVYYCQQRRSFPYTF | | KFQGRVTFTADTSSDT | |
| CD3 | | | GQGTKLEIKRTVAAPSVFI | | AYMELSSLRSEDTAVY | |
| scFv | | | FPPSDEQLKSGTASVVCLL | | YCTRWGYYGTRGYFN | |
| (Vh- | | | NNFYPREAKVQWKVDNA | | VWGQGTLVTVSSAST | |
| Vl); | | | LQSGNSQESVTEQDSKDST | | KGPSVFPLAPSSKSTSG | |
| GGGG | | | YSLSSTLTLSKADYEKHKV | | GTAALGCLVKDYFPEP | |
| SGGG | | | YACEVTHQGLSSPVTKSFN | | VTVSWNSGALTSGVHT | |
| GS | | | RGECGGGGSGGGGSEVQL | | FPAVLQSSGLYSLSS | |
| linker | | | VESGGGLVQPGGSLKLSC | | VVTVPSSSLGTQTYIC | |
| (SEQ | | | AASGFTFNKYAMNWVRQ | | NVNHKPSNTKVDKKV | |
| ID NO: | | | APGKGLEWVARIRSKYNN | | EPKSC | |
| 270) | | | YATYYADSVKDRFTISRD | | | |
| | | | DSKNTAYLQMNNLKTEDT | | | |
| | | | AVYYCVRHGNFGNSYISY | | | |
| | | | WAYWGQGTLVTVSSGGG | | | |
| | | | GSGGGGSGGGGSQTVVTQ | | | |
| | | | EPSLTVSPGGTVTLTCGSS | | | |
| | | | TGAVTSGNYPNWVQQKP | | | |
| | | | GQAPRGLIGGTKFLAPGTP | | | |
| | | | ARFSGSLLGGKAALTLSG | | | |
| | | | VQPEDEAEYYCVLWYSNR | | | |
| | | | WVFGGGTKLTVL | | | |
| TCE- | (None) and | (None) | DIQMTQSPSSLSASVGDRV | 150 | EVQLVESGGGLVQPG | 198 |
| 25/ | (None) | and | TITCKSSQSLLYRSNQKNY | | GSLKLSCAASGFTFNK | |
| (2 + 1) | | (None) | LAWYQQKPGKAPKLLIYW | | YAMNWVRQAPGKGL | |
| | | | ASTRESGVPSRFSGSGSGT | | EWVARIRSKYNNYAT | |
| | | | DFTLTISSLQPEDFATYYC | | YYADSVKDRFTISRDD | |
| | | | QQYYNYPRTFGQGTKVEI | | SKNTAYLQMNNLKTE | |
| | | | KRTVAAPSVFIFPPSDEQL | | DTAVYYCVRHGNFGN | |
| | | | KSGTASVVCLLNNFYPRE | | SYISYWAYWGQGTLV | |
| | | | AKVQWKVDNALQSGNSQ | | TVSSGGGGSGGGGSG | |
| | | | ESVTEQDSKDSTYSLSSTL | | GGGSQTVVTQEPSLTV | |
| | | | TLSKADYEKHKVYACEVT | | SPGGTVTLTCGSSTGA | |
| | | | HQGLSSPVTKSFNRGEC | | VTSGNYPNWVQQKPG | |
| | | | | | QAPRGLIGGTKFLAPG | |
| | | | | | TPARFSGSLLGGKAAL | |
| | | | | | TLSGVQPEDEAEYYCV | |
| | | | | | LWYSNRWVFGGGTKL | |
| | | | | | TVLGGGGSEVQLVES | |
| | | | | | GGGLVQPGGSLRLSCA | |
| | | | | | VSGYSITSDYAWNWV | |
| | | | | | RQAPGKGLEWVGYIS | |
| | | | | | NSGSTSYNPSLKSRFTI | |
| | | | | | SRDTSKNTLYLQMNSL | |
| | | | | | RAEDTAVYYCARERN | |
| | | | | | YDYDDYYYAMDYWG | |
| | | | | | QGTLVTVSSASTKGPS | |
| | | | | | VFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| | | | | | DGGGGSGGGGSEVQL | |
| | | | | | VESGGGLVQPGGSLRL | |
| | | | | | SCAVSGYSITSDYAWN | |
| | | | | | WVRQAPGKGLEWVG | |
| | | | | | YISNSGSTSYNPSLKSR | |
| | | | | | FTISRDTSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE- | (None) and | (None) | EVQLVESGGGLVQPGGSL | 151 | EVQLVESGGGLVQPG | 199 |
| 26/ | (None) | and | KLSCAASGFTFNKYAMN | | GSLRLSCAVSGYSITSD | |
| (2 + 1) | | (None) | WVRQAPGKGLEWVARIRS | | YAWNWVRQAPGKGL | |
| | | | KYNNYATYYADSVKDRFT | | EWVGYISNSGSTSYNP | |
| | | | ISRDDSKNTAYLQMNNLK | | SLKSRFTISRDTSKNTL | |
| | | | TEDTAVYYCVRHGNFGNS | | YLQMNSLRAEDTAVY | |
| | | | YISYWAYWGQGTLVTVSS | | YCARERNYDYDDYYY | |
| | | | GGGGSGGGGSGGGGSQTV | | AMDYWGQGTLVTVSS | |
| | | | VTQEPSLTVSPGGTVTLTC | | ASTKGPSVFPLAPSSKS | |
| | | | GSSTGAVTSGNYPNWVQQ | | TSGGTAALGCLVKDY | |
| | | | KPGQAPRGLIGGTKFLAPG | | FPEPVTVSWNSGALTS | |
| | | | TPARFSGSLLGGKAALTLS | | GVHTFPAVLQSSGLYS | |
| | | | GVQPEDEAEYYCVLWYSN | | LSSVVTVPSSSLGTQT | |
| | | | RWVFGGGTKLTVLGGGGS | | YICNVNHKPSNTKVD | |
| | | | DIQMTQSPSSLSASVGDRV | | KKVEPKSC | |
| | | | TITCKSSQSLLYRSNQKNY | | | |
| | | | LAWYQQKPGKAPKLLIYW | | | |
| | | | ASTRESGVPSRFSGSGSGT | | | |
| | | | DFTLTISSLQPEDFATYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGECG | | | |
| | | | GGGSGGGGSDIQMTQSPSS | | | |
| | | | LSASVGDRVTITCKSSQSL | | | |
| | | | LYRSNQKNYLAWYQQKP | | | |
| | | | GKAPKLLIYWASTRESGVP | | | |
| | | | SRFSGSGSGTDFTLTISSLQ | | | |
| | | | PEDFATYYCQQYYNYPRT | | | |
| | | | FGQGTKVEIKRTVAAPSVF | | | |
| | | | IFPPSDEQLKSGTASVVCL | | | |
| | | | LNNFYPREAKVQWKVDN | | | |
| | | | ALQSGNSQESVTEQDSKD | | | |
| | | | STYSLSSTLTLSKADYEKH | | | |
| | | | KVYACEVTHQGLSSPVTK | | | |
| | | | SFNRGEC | | | |
| TCE- | (Q3V, | (None) | DIVMTQSPDSLAVSLGERA | 152 | EVQLVESGGGLVQPG | 200 |
| 27/ | S9D, | and | TINCKSSQSLLYRSNQKNY | | GSLKLSCAASGFTFNK | |
| (2 + 1) | S12A, | (None) | LAWYQQKPGQPPKLLIYW | | YAMNWVRQAPGKGL | |
| | A13V, | | ASTRESGVPDRFSGSGSGT | | EWVARIRSKYNNYAT | |
| | V15L, | | DFTLTISSLQAEDVAVYYC | | YYADSVKDRFTISRDD | |
| | D17E, | | QQYYNYPRTFGQGTKVEI | | SKNTAYLQMNNLKTE | |
| | V19A, | | KRTVAAPSVFIFPPSDEQL | | DTAVYYCVRHGNFGN | |
| | T22N, | | KSGTASVVCLLNNFYPRE | | SYISYWAYWGQGTLV | |
| | K48Q, | | AKVQWKVDNALQSGNSQ | | TVSSGGGGSGGGGSG | |
| | A49P, | | ESVTEQDSKDSTYSLSSTL | | GGGSQTVVTQEPSLTV | |
| | S66D, | | TLSKADYEKHKVYACEVT | | SPGGTVTLTCGSSTGA | |
| | P86A, | | HQGLSSPVTKSFNRGEC | | VTSGNYPNWVQQKPG | |
| | F89V, | | | | QAPRGLIGGTKFLAPG | |
| | T91V) | | | | TPARFSGSLLGGKAAL | |
| | AND | | | | TLSGVQPEDEAEYYCV | |
| | (V24A, | | | | LWYSNRWVFGGGTKL | |
| | Y27F, | | | | TVLGGGGSEVQLVES | |
| | T58I, | | | | GGGLVQPGGSLRLSCA | |
| | T74N) | | | | ASGFSITSDYAWNWV | |
| | | | | | RQAPGKGLEWVGYIS | |
| | | | | | NSGSISYNPSLKSRFTIS | |
| | | | | | RDNSKNTLYLQMNSL | |
| | | | | | RAEDTAVYYCARERN | |
| | | | | | YDYDDYYYAMDYWG | |
| | | | | | QGTLVTVSSASTKGPS | |
| | | | | | VFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| | | | | | DGGGGSGGGGSEVQL | |
| | | | | | VESGGGLVQPGGSLRL | |
| | | | | | SCAASGFSITSDYAWN | |
| | | | | | WVRQAPGKGLEWVG | |
| | | | | | YISNSGSISYNPSLKSR | |
| | | | | | FTISRDNSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE- | (Q3V, | (None) | EVQLVESGGGLVQPGGSL | 153 | EVQLVESGGGLVQPG | 201 |
| 28/ | S9D, | and | KLSCAASGFTFNKYAMN | | GSLRLSCAASGFSITSD | |
| (2 + 1) | S12A, | (None) | WVRQAPGKGLEWVARIRS | | YAWNWVRQAPGKGL | |
| | A13V, | | KYNNYATYYADSVKDRFT | | EWVGYISNSGSISYNPS | |
| | V15L, | | ISRDDSKNTAYLQMNNLK | | LKSRFTISRDNSKNTL | |
| | D17E, | | TEDTAVYYCVRHGNFGNS | | YLQMNSLRAEDTAVY | |
| | V19A, | | YISYWAYWGQGTLVTVSS | | YCARERNYDYDDYYY | |
| | T22N, | | GGGGSGGGGSGGGGSQTV | | AMDYWGQGTLVTVSS | |
| | K48Q, | | VTQEPSLTVSPGGTVTLTC | | ASTKGPSVFPLAPSSKS | |
| | A49P, | | GSSTGAVTSGNYPNWVQQ | | TSGGTAALGCLVKDY | |
| | S66D, | | KPGQAPRGLIGGTKFLAPG | | FPEPVTVSWNSGALTS | |
| | P86A, | | TPARFSGSLLGGKAALTLS | | GVHTFPAVLQSSGLYS | |
| | F89V, | | GVQPEDEAEYYCVLWYSN | | LSSVVTVPSSSLGTQT | |
| | T91V) | | RWVFGGGTKLTVLGGGGS | | YICNVNHKPSNTKVD | |
| | AND | | DIVMTQSPDSLAVSLGERA | | KKVEPKSC | |
| | (V24A, | | TINCKSSQSLLYRSNQKNY | | | |
| | Y27F, | | LAWYQQKPGQPPKLLIYW | | | |
| | T58I, | | ASTRESGVPDRFSGSGSGT | | | |
| | T74N) | | DFTLTISSLQAEDVAVYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGECG | | | |
| TCE- | | | GGGSGGGGSDIVMTQSPD | | | |
| 29/ | | | SLAVSLGERATINCKSSQS | | | |
| (2 + 1) | | | LLYRSNQKNYLAWYQQK | | | |
| | | | PGQPPKLLIYWASTRESGV | | | |
| | | | PDRFSGSGSGTDFTLTISSL | | | |
| | | | QAEDVAVYYCQQYYNYP | | | |
| | | | RTFGQGTKVEIKRTVAAPS | | | |
| | | | VFIFPPSDEQLKSGTASVV | | | |
| | | | CLLNNFYPREAKVQWKV | | | |
| | | | DNALQSGNSQESVTEQDS | | | |
| | | | KDSTYSLSSTLTLSKADYE | | | |
| | | | KHKVYACEVTHQGLSSPV | | | |
| | | | TKSFNRGEC | | | |
| | (Q35N) | (None) | DIQMTQSPSSLSASVGDRV | 154 | EVQLVESGGGLVQPG | 202 |
| | and | and | TITCKSSQSLLYRSNNKNY | | GSLKLSCAASGFTFNK | |
| | (W35M, | (None) | LAWYQQKPGKAPKLLIYW | | YAMNWVRQAPGKGL | |
| | L64V) | | ASTRESGVPSRFSGSGSGT | | EWVARIRSKYNNYAT | |
| | | | DFTLTISSLQPEDFATYYC | | YYADSVKDRFTISRDD | |
| | | | QQYYNYPRTFGQGTKVEI | | SKNTAYLQMNNLKTE | |
| | | | KRTVAAPSVFIFPPSDEQL | | DTAVYYCVRHGNFGN | |
| | | | KSGTASVVCLLNNFYPRE | | SYISYWAYWGQGTLV | |
| | | | AKVQWKVDNALQSGNSQ | | TVSSGGGGSGGGGSG | |
| | | | ESVTEQDSKDSTYSLSSTL | | GGGSQTVVTQEPSLTV | |
| | | | TLSKADYEKHKVYACEVT | | SPGGTVTLTCGSSTGA | |
| | | | HQGLSSPVTKSFNRGEC | | VTSGNYPNWVQQKPG | |
| | | | | | QAPRGLIGGTKFLAPG | |
| | | | | | TPARFSGSLLGGKAAL | |
| | | | | | TLSGVQPEDEAEYYCV | |
| | | | | | LWYSNRWVFGGGTKL | |
| | | | | | TVLGGGGSEVQLVES | |
| | | | | | GGGLVQPGGSLRLSCA | |
| | | | | | VSGYSITSDYAMNWV | |
| | | | | | RQAPGKGLEWVGYIS | |
| | | | | | NSGSTSYNPSVKSRFTI | |
| | | | | | SRDTSKNTLYLQMNSL | |
| | | | | | RAEDTAVYYCARERN | |
| | | | | | YDYDDYYYAMDYWG | |
| | | | | | QGTLVTVSSASTKGPS | |
| | | | | | VFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| | | | | | DGGGGSGGGGSEVQL | |
| | | | | | VESGGGLVQPGGSLRL | |
| | | | | | SCAVSGYSITSDYAMN | |
| | | | | | WVRQAPGKGLEWVG | |
| | | | | | YISNSGSTSYNPSVKSR | |
| | | | | | FTISRDTSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE- | (Q35N) | (None) | EVQLVESGGGLVQPGGSL | 155 | EVQLVESGGGLVQPG | 203 |
| 30/ | AND | and | KLSCAASGFTFNKYAMN | | GSLRLSCAVSGYSITSD | |
| (2 + 1) | (W35M, | (None) | WVRQAPGKGLEWVARIRS | | YAMNWVRQAPGKGL | |
| | L64V) | | KYNNYATYYADSVKDRFT | | EWVGYISNSGSTSYNP | |
| | | | ISRDDSKNTAYLQMNNLK | | SVKSRFTISRDTSKNTL | |
| | | | TEDTAVYYCVRHGNFGNS | | YLQMNSLRAEDTAVY | |
| | | | YISYWAYWGQGTLVTVSS | | YCARERNYDYDDYYY | |
| | | | GGGGSGGGGSGGGGSQTV | | AMDYWGQGTLVTVSS | |
| | | | VTQEPSLTVSPGGTVTLTC | | ASTKGPSVFPLAPSSKS | |
| | | | GSSTGAVTSGNYPNWVQQ | | TSGGTAALGCLVKDY | |
| | | | KPGQAPRGLIGGTKFLAPG | | FPEPVTVSWNSGALTS | |
| | | | TPARFSGSLLGGKAALTLS | | GVHTFPAVLQSSGLYS | |
| | | | GVQPEDEAEYYCVLWYSN | | LSSVVTVPSSSLGTQT | |
| | | | RWVFGGGTKLTVLGGGGS | | YICNVNHKPSNTKVD | |
| | | | DIQMTQSPSSLSASVGDRV | | KKVEPKSC | |
| | | | TITCKSSQSLLYRSNNKNY | | | |
| | | | LAWYQQKPGKAPKLLIYW | | | |
| | | | ASTRESGVPSRFSGSGSGT | | | |
| | | | DFTLTISSLQPEDFATYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGECG | | | |
| | | | GGGSGGGGSDIQMTQSPSS | | | |
| | | | LSASVGDRVTITCKSSQSL | | | |
| | | | LYRSNNKNYLAWYQQKP | | | |
| | | | GKAPKLLIYWASTRESGVP | | | |
| | | | SRFSGSGSGTDFTLTISSLQ | | | |
| | | | PEDFATYYCQQYYNYPRT | | | |
| | | | FGQGTKVEIKRTVAAPSVF | | | |
| | | | IFPPSDEQLKSGTASVVCL | | | |
| | | | LNNFYPREAKVQWKVDN | | | |
| | | | ALQSGNSQESVTEQDSKD | | | |
| | | | STYSLSSTLTLSKADYEKH | | | |
| | | | KVYACEVTHQGLSSPVTK | | | |
| | | | SFNRGEC | | | |
| TCE- | (Q3V, | (None) | DIVMTQSPDSLAVSLGERA | 156 | EVQLVESGGGLVQPG | 204 |
| 31/ | S9D, | and | TINCKSSQSLLYRSNNKNY | | GSLKLSCAASGFTFNK | |
| (2 + 1) | S12A, | (None) | LAWYQQKPGQPPKLLIYW | | YAMNWVRQAPGKGL | |
| | A13V, | | ASTRESGVPDRFSGSGSGT | | EWVARIRSKYNNYAT | |
| | V15L, | | DFTLTISSLQAEDVAVYYC | | YYADSVKDRFTISRDD | |
| | D17E, | | QQYYNYPRTFGQGTKVEI | | SKNTAYLQMNNLKTE | |
| | V19A, | | KRTVAAPSVFIFPPSDEQL | | DTAVYYCVRHGNFGN | |
| | T22N, | | KSGTASVVCLLNNFYPRE | | SYISYWAYWGQGTLV | |
| | Q35N, | | AKVQWKVDNALQSGNSQ | | TVSSGGGGSGGGGSG | |
| | K48Q, | | ESVTEQDSKDSTYSLSSTL | | GGGSQTVVTQEPSLTV | |
| | A49P, | | TLSKADYEKHKVYACEVT | | SPGGTVTLTCGSSTGA | |
| | S66D, | | HQGLSSPVTKSFNRGEC | | VTSGNYPNWVQQKPG | |
| | P86A, | | | | QAPRGLIGGTKFLAPG | |
| | F89V, | | | | TPARFSGSLLGGKAAL | |
| | T91V) | | | | TLSGVQPEDEAEYYCV | |
| | AND | | | | LWYSNRWVFGGGTKL | |
| | (V24A, | | | | TVLGGGGSEVQLVES | |
| | Y27F, | | | | GGGLVQPGGSLRLSCA | |
| | W35M, | | | | ASGFSITSDYAMNWV | |
| | T58I, | | | | RQAPGKGLEWVGYIS | |
| | L64V, | | | | NSGSISYNPSVKGRFTI | |
| | S66G, | | | | SRDNSKNTLYLQMNS | |
| | T74N) | | | | LRAEDTAVYYCARER | |
| | | | | | NYDYDDYYYAMDYW | |
| | | | | | GQGTLVTVSSASTKGP | |
| | | | | | SVFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| | | | | | DGGGGSGGGGSEVQL | |
| | | | | | VESGGGLVQPGGSLRL | |
| | | | | | SCAASGFSITSDYAMN | |
| | | | | | WVRQAPGKGLEWVG | |
| | | | | | YISNSGSISYNPSVKGR | |
| | | | | | FTISRDNSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSC | |
| TCE | (Q3V, | (None) | EVQLVESGGGLVQPGGSL | 157 | EVQLVESGGGLVQPG | 205 |
| 32/ | S9D, | and | KLSCAASGFTFNKYAMN | | GSLRLSCAASGFSITSD | |
| (2 + 1) | S12A, | (None) | WVRQAPGKGLEWVARIRS | | YAMNWVRQAPGKGL | |
| | A13V, | | KYNNYATYYADSVKDRFT | | EWVGYISNSGSISYNPS | |
| | V15L, | | ISRDDSKNTAYLQMNNLK | | VKGRFTISRDNSKNTL | |
| | D17E, | | TEDTAVYYCVRHGNFGNS | | YLQMNSLRAEDTAVY | |
| | V19A, | | YISYWAYWGQGTLVTVSS | | YCARERNYDYDDYYY | |
| | T22N, | | GGGGSGGGGSGGGGSQTV | | AMDYWGQGTLVTVSS | |
| | Q35N | | VTQEPSLTVSPGGTVTLTC | | ASTKGPSVFPLAPSSKS | |
| | K48Q, | | GSSTGAVTSGNYPNWVQQ | | TSGGTAALGCLVKDY | |
| | A49P, | | KPGQAPRGLIGGTKFLAPG | | FPEPVTVSWNSGALTS | |
| | S66D, | | TPARFSGSLLGGKAALTLS | | GVHTFPAVLQSSGLYS | |
| | P86A, | | GVQPEDEAEYYCVLWYSN | | LSSVVTVPSSSLGTQT | |
| | F89V, | | RWVFGGGTKLTVLGGGGS | | YICNVNHKPSNTKVD | |
| | T91V) | | DIVMTQSPDSLAVSLGERA | | KKVEPKSC | |
| | AND | | TINCKSSQSLLYRSNNKNY | | | |
| | (V24A, | | LAWYQQKPGQPPKLLIYW | | | |
| | Y27F, | | ASTRESGVPDRFSGSGSGT | | | |
| | W35M, | | DFTLTISSLQAEDVAVYYC | | | |
| | T58I, | | QQYYNYPRTFGQGTKVEI | | | |
| | L64V, | | KRTVAAPSVFIFPPSDEQL | | | |
| | S66G, | | KSGTASVVCLLNNFYPRE | | | |
| | T74N) | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGECG | | | |
| | | | GGGSGGGGSDIVMTQSPD | | | |
| | | | SLAVSLGERATINCKSSQS | | | |
| | | | LLYRSNNKNYLAWYQQK | | | |
| | | | PGQPPKLLIYWASTRESGV | | | |
| | | | PDRFSGSGSGTDFTLTISSL | | | |
| | | | QAEDVAVYYCQQYYNYP | | | |
| | | | RTFGQGTKVEIKRTVAAPS | | | |
| | | | VFIFPPSDEQLKSGTASVV | | | |
| | | | CLLNNFYPREAKVQWKV | | | |
| | | | DNALQSGNSQESVTEQDS | | | |
| | | | KDSTYSLSSTLTLSKADYE | | | |
| | | | KHKVYACEVTHQGLSSPV | | | |
| | | | TKSFNRGEC | | | |
| TCE- | (Q35N) | (None) | DIQMTQSPSSLSASVGDRV | 158 | EVQLVESGGGLVQPG | 206 |
| 33/ | AND | and | TITCKSSQSLLYRSNNKNY | | GSLKLSCAASGFTFNK | |
| 2 + 1) | (W35M, | (None) | LAWYQQKPGKAPKLLIYW | | YAMNWVRQAPGKGL | |
| | L64V, | | ASTRESGVPSRFSGSGSGT | | EWVARIRSKYNNYAT | |
| | E99D, | | DFTLTISSLQPEDFATYYC | | YYADSVKDRFTISRDD | |
| | M111F) | | QQYYNYPRTFGQGTKVEI | | SKNTAYLQMNNLKTE | |
| | | | KRTVAAPSVFIFPPSDEQL | | DTAVYYCVRHGNFGN | |
| | | | KSGTASVVCLLNNFYPRE | | SYISYWAYWGQGTLV | |
| | | | AKVQWKVDNALQSGNSQ | | TVSSGGGGSGGGGSG | |
| | | | ESVTEQDSKDSTYSLSSTL | | GGGSQTVVTQEPSLTV | |
| | | | TLSKADYEKHKVYACEVT | | SPGGTVTLTCGSSTGA | |
| | | | HQGLSSPVTKSFNRGEC | | VTSGNYPNWVQQKPG | |
| | | | | | QAPRGLIGGTKFLAPG | |
| | | | | | TPARFSGSLLGGKAAL | |
| | | | | | TLSGVQPEDEAEYYCV | |
| | | | | | LWYSNRWVFGGGTKL | |
| | | | | | TVLGGGGSEVQLVES | |
| | | | | | GGGLVQPGGSLRLSCA | |
| | | | | | VSGYSITSDYAMNWV | |
| | | | | | RQAPGKGLEWVGYIS | |
| | | | | | NSGSTSYNPSVKSRFTI | |
| | | | | | SRDTSKNTLYLQMNSL | |
| | | | | | RAEDTAVYYCARDRN | |
| | | | | | YDYDDYYYAFDYWG | |
| | | | | | QGTLVTVSSASTKGPS | |
| | | | | | VFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| | | | | | DGGGGSGGGGSEVQL | |
| | | | | | VESGGGLVQPGGSLRL | |
| | | | | | SCAVSGYSITSDYAMN | |
| | | | | | WVRQAPGKGLEWVG | |
| | | | | | YISNSGSTSYNPSVKSR | |
| | | | | | FTISRDTSKNTLYLQM | |
| | | | | | NSLRAEDTAVYYCAR | |
| | | | | | DRNYDYDDYYYAFDY | |
| | | | | | WGQGTLVTVSSASTK | |
| | | | | | GPSVFPLAPSSKSTSGG | |
| | | | | | TAALGCLVKDYFPEPV | |
| | | | | | TVSWNSGALTSGVHT | |
| | | | | | FPAVLQSSGLYSLSSV | |
| | | | | | VTVPSSSLGTQTYICN | |
| | | | | | VNHKPSNTKVDKKVE | |
| | | | | | PKSC | |
| TCE- | (Q35N) | (None) | EVQLVESGGGLVQPGGSL | 159 | EVQLVESGGGLVQPG | 207 |
| 34/ | AND | and | KLSCAASGFTFNKYAMN | | GSLRLSCAVSGYSITSD | |
| (2 + 1) | (W35M, | (None) | WVRQAPGKGLEWVARIRS | | YAMNWVRQAPGKGL | |
| | L64V, | | KYNNYATYYADSVKDRFT | | EWVGYISNSGSTSYNP | |
| | E99D, | | ISRDDSKNTAYLQMNNLK | | SVKSRFTISRDTSKNTL | |
| | M111F) | | TEDTAVYYCVRHGNFGNS | | YLQMNSLRAEDTAVY | |
| | | | YISYWAYWGQGTLVTVSS | | YCARDRNYDYDDYY | |
| | | | GGGGSGGGGSGGGGSQTV | | YAFDYWGQGTLVTVS | |
| | | | VTQEPSLTVSPGGTVTLTC | | SASTKGPSVFPLAPSSK | |
| | | | GSSTGAVTSGNYPNWVQQ | | STSGGTAALGCLVKD | |
| | | | KPGQAPRGLIGGTKFLAPG | | YFPEPVTVSWNSGALT | |
| | | | TPARFSGSLLGGKAALTLS | | SGVHTFPAVLQSSGLY | |
| | | | GVQPEDEAEYYCVLWYSN | | SLSSVVTVPSSSLGTQT | |
| | | | RWVFGGGTKLTVLGGGGS | | YICNVNHKPSNTKVD | |
| | | | DIQMTQSPSSLSASVGDRV | | KKVEPKSC | |
| | | | TITCKSSQSLLYRSNNKNY | | | |
| | | | LAWYQQKPGKAPKLLIYW | | | |
| | | | ASTRESGVPSRFSGSGSGT | | | |
| | | | DFTLTISSLQPEDFATYYC | | | |
| | | | QQYYNYPRTFGQGTKVEI | | | |
| | | | KRTVAAPSVFIFPPSDEQL | | | |
| | | | KSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGECG | | | |
| | | | GGGSGGGGSDIQMTQSPSS | | | |
| | | | LSASVGDRVTITCKSSQSL | | | |
| | | | LYRSNNKNYLAWYQQKP | | | |
| | | | GKAPKLLIYWASTRESGVP | | | |
| | | | SRFSGSGSGTDFTLTISSLQ | | | |
| | | | PEDFATYYCQQYYNYPRT | | | |
| | | | FGQGTKVEIKRTVAAPSVF | | | |
| | | | IFPPSDEQLKSGTASVVCL | | | |
| | | | LNNFYPREAKVQWKVDN | | | |
| | | | ALQSGNSQESVTEQDSKD | | | |
| | | | STYSLSSTLTLSKADYEKH | | | |
| | | | KVYACEVTHQGLSSPVTK | | | |
| | | | SFNRGEC | | | |
| TCE- | (Q3V, | (None) | DIVMTQSPDSLAVSLGERA | 160 | EVQLVESGGGLVQPG | 208 |
| 35/ | S9D, | and | TINCKSSQSLLYRSNNKNY | | GSLKLSCAASGFTFNK | |
| (2 + 1) | S12A, | (None) | LAWYQQKPGQPPKLLIYW | | YAMNWVRQAPGKGL | |
| | A13V, | | ASTRESGVPDRFSGSGSGT | | EWVARIRSKYNNYAT | |
| | V15L, | | DFTLTISSLQAEDVAVYYC | | YYADSVKDRFTISRDD | |
| | D17E, | | QQYYNYPRTFGQGTKVEI | | SKNTAYLQMNNLKTE | |
| | V19A, | | KRTVAAPSVFIFPPSDEQL | | DTAVYYCVRHGNFGN | |
| | T22N, | | KSGTASVVCLLNNFYPRE | | SYISYWAYWGQGTLV | |
| | Q35N, | | AKVQWKVDNALQSGNSQ | | TVSSGGGGSGGGGSG | |
| | K48Q, | | ESVTEQDSKDSTYSLSSTL | | GGGSQTVVTQEPSLTV | |
| | A49P, | | TLSKADYEKHKVYACEVT | | SPGGTVTLTCGSSTGA | |
| | S66D, | | HQGLSSPVTKSFNRGEC | | VTSGNYPNWVQQKPG | |
| | P86A, | | | | QAPRGLIGGTKFLAPG | |
| | F89V, | | | | TPARFSGSLLGGKAAL | |
| | T91V) | | | | TLSGVQPEDEAEYYCV | |
| | AND | | | | LWYSNRWVFGGGTKL | |
| | (V24A, | | | | TVLGGGGSEVQLVES | |
| | Y27F, | | | | GGGLVQPGGSLRLSCA | |
| | W35M, | | | | ASGFSITSDYAMNWV | |
| | L64V, | | | | RQAPGKGLEWVGYIS | |
| | T74N, | | | | NSGSTSYNPSVKSRFTI | |
| | E99D, | | | | SRDNSKNTLYLQMNS | |
| | M111F) | | | | LRAEDTAVYYCARDR | |
| | | | | | NYDYDDYYYAFDYW | |
| | | | | | GQGTLVTVSSASTKGP | |
| | | | | | SVFPLAPSSKSTSGGTA | |
| | | | | | ALGCLVKDYFPEPVTV | |
| | | | | | SWNSGALTSGVHTFPA | |
| | | | | | VLQSSGLYSLSSVVTV | |
| | | | | | PSSSLGTQTYICNVNH | |
| | | | | | KPSNTKVDKKVEPKSC | |
| | | | | | DGGGGSGGGGSEVQL | |
| | | | | | VESGGGLVQPGGSLRL | |
| | | | | | SCAASGFSITSDYAMN | |
| | | | | | WVRQAPGKGLEWVG | |
| | | | | | YISNSGSTSYNPSVKSR | |
| | | | | | FTISRDNSKNTLYLQM | |
| | | | | | NSLRAEDTAVYYCAR | |
| | | | | | DRNYDYDDYYYAFDY | |
| | | | | | WGQGTLVTVSSASTK | |
| | | | | | GPSVFPLAPSSKSTSGG | |
| | | | | | TAALGCLVKDYFPEPV | |
| | | | | | TVSWNSGALTSGVHT | |
| | | | | | FPAVLQSSGLYSLSSV | |
| | | | | | VTVPSSSLGTQTYICN | |
| | | | | | VNHKPSNTKVDKKVE | |
| | | | | | PKSC | |
| TCE- | (Q3V, | (None) | EVQLVESGGGLVQPGGSL | 161 | EVQLVESGGGLVQPG | 209 |
| 36/ | S9D, | and | KLSCAASGFTFNKYAMN | | GSLRLSCAASGFSITSD | |
| (2 + 1) | S12A, | (None) | WVRQAPGKGLEWVARIRS | | YAMNWVRQAPGKGL | |
| | A13V, | | KYNNYATYYADSVKDRFT | | EWVGYISNSGSTSYNP | |
| | V15L, | | ISRDDSKNTAYLQMNNLK | | SVKSRFTISRDNSKNTL | |
| | D17E, | | TEDTAVYYCVRHGNFGNS | | YLQMNSLRAEDTAVY | |
| | V19A, | | YISYWAYWGQGTLVTVSS | | YCARDRNYDYDDYY | |
| | T22N, | | GGGGSGGGGGGGGSQTV | | YAFDYWGQGTLVTVS | |
| | Q35N, | | VTQEPSLTVSPGGTVTLTC | | SASTKGPSVFPLAPSSK | |
| | K48Q, | | GSSTGAVTSGNYPNWVQQ | | STSGGTAALGCLVKD | |
| | A49P, | | KPGQAPRGLIGGTKFLAPG | | YFPEPVTVSWNSGALT | |
| | S66D, | | TPARFSGSLLGGKAALTLS | | SGVHTFPAVLQSSGLY | |
| | P86A, | | GVQPEDEAEYYCVLWYSN | | SLSSVVTVPSSSLGTQT | |
| | F89V, | | RWVFGGGTKLTVLGGGGS | | YICNVNHKPSNTKVD | |
| | T91V) | | DIVMTQSPDSLAVSLGERA | | KKVEPKSC | |
| | AND | | TINCKSSQSLLYRSNNKNY | | | |
| | (V24A, | | LAWYQQKPGQPPKLLIYW | | | |
| | Y27F, | | ASTRESGVPDRFSGSGSGT | | | |
| | W35M, | | DFTLTISSLQAEDVAVYYC | | | |
| | L64V, | | QQYYNYPRTFGQGTKVEI | | | |
| | T74N, | | KRTVAAPSVFIFPPSDEQL | | | |
| | E99D, | | KSGTASVVCLLNNFYPRE | | | |
| | M111F) | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGECG | | | |
| | | | GGGSGGGGSDIVMTQSPD | | | |
| | | | SLAVSLGERATINCKSSQS | | | |
| | | | LLYRSNNKNYLAWYQQK | | | |
| | | | PGQPPKLLIYWASTRESGV | | | |
| | | | PDRFSGSGSGTDFTLTISSL | | | |
| | | | QAEDVAVYYCQQYYNYP | | | |
| | | | RTFGQGTKVEIKRTVAAPS | | | |
| | | | VFIFPPSDEQLKSGTASVV | | | |
| | | | CLLNNFYPREAKVQWKV | | | |
| | | | DNALQSGNSQESVTEQDS | | | |
| | | | KDSTYSLSSTLTLSKADYE | | | |
| | | | KHKVYACEVTHQGLSSPV | | | |
| | | | TKSFNRGEC | | | |
| TCE- | (None) and | (N30Q, | DIQMTQSPSSLSASVGDRV | 162 | EVQLVESGGGLVQPG | 210 |
| 37/ | (None) | I109V, | TITCKSSQSLLYRSNQKNY | | GSLKLSCAASGFTFQK | |
| (2 + 1) | | G172A, | LAWYQQKPGKAPKLLIYW | | YAMNWVRQAPGKGL | |
| | | V231A) | ASTRESGVPSRFSGSGSGT | | EWVARIRSKYNNYAT | |
| | | AND | DFTLTISSLQPEDFATYYC | | YYADSVKDRFTISRDD | |
| | | (N30Q, | QQYYNYPRTFGQGTKVEI | | SKNTAYLQMNNLKTE | |
| | | I109V, | KRTVAAPSVFIFPPSDEQL | | DTAVYYCVRHGNFGN | |
| | | G172A, | KSGTASVVCLLNNFYPRE | | SYVSYWAYWGQGTL | |
| | | V231A) | AKVQWKVDNALQSGNSQ | | VTVSSGGGGSGGGGS | |
| | | | ESVTEQDSKDSTYSLSSTL | | GGGGSQTVVTQEPSLT | |
| | | | TLSKADYEKHKVYACEVT | | VSPGGTVTLTCGSSTG | |
| | | | HQGLSSPVTKSFNRGEC | | AVTSANYPNWVQQKP | |
| | | | | | GQAPRGLIGGTKFLAP | |
| | | | | | GTPARFSGSLLGGKAA | |
| | | | | | LTLSGVQPEDEAEYYC | |
| | | | | | ALWYSNRWVFGGGT | |
| | | | | | KLTVLGGGGSEVQLV | |
| | | | | | ESGGGLVQPGGSLRLS | |
| | | | | | CAVSGYSITSDYAWN | |
| | | | | | WVRQAPGKGLEWVG | |
| | | | | | YISNSGSTSYNPSLKSR | |
| | | | | | FTISRDTSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSCDGGGGSGGGG | |
| | | | | | SEVQLVESGGGLVQPG | |
| | | | | | GSLRLSCAVSGYSITSD | |
| | | | | | YAWNWVRQAPGKGL | |
| | | | | | EWVGYISNSGSTSYNP | |
| | | | | | SLKSRFTISRDTSKNTL | |
| | | | | | YLQMNSLRAEDTAVY | |
| | | | | | YCARERNYDYDDYYY | |
| | | | | | AMDYWGQGTLVTVSS | |
| | | | | | ASTKGPSVFPLAPSSKS | |
| | | | | | TSGGTAALGCLVKDY | |
| | | | | | FPEPVTVSWNSGALTS | |
| | | | | | GVHTFPAVLQSSGLYS | |
| | | | | | LSSVVTVPSSSLGTQT | |
| | | | | | YICNVNHKPSNTKVD | |
| | | | | | KKVEPKSC | |
| TCE- | (None) and | (N30Q, | EVQLVESGGGLVQPGGSL | 163 | EVQLVESGGGLVQPG | 211 |
| 38/ | (None) | I109V, | KLSCAASGFTFQKYAMN | | GSLRLSCAVSGYSITSD | |
| (2 + 1) | | G172A, | WVRQAPGKGLEWVARIRS | | YAWNWVRQAPGKGL | |
| | | V231A) | KYNNYATYYADSVKDRFT | | EWVGYISNSGSTSYNP | |
| | | AND | ISRDDSKNTAYLQMNNLK | | SLKSRFTISRDTSKNTL | |
| | | (N30Q, | TEDTAVYYCVRHGNFGNS | | YLQMNSLRAEDTAVY | |
| | | I109V, | YVSYWAYWGQGTLVTVS | | YCARERNYDYDDYYY | |
| | | G172A, | SGGGGSGGGGSGGGGSQT | | AMDYWGQGTLVTVSS | |
| | | V231A) | VVTQEPSLTVSPGGTVTLT | | ASTKGPSVFPLAPSSKS | |
| | | | CGSSTGAVTSANYPNWVQ | | TSGGTAALGCLVKDY | |
| | | | QKPGQAPRGLIGGTKFLAP | | FPEPVTVSWNSGALTS | |
| | | | GTPARFSGSLLGGKAALTL | | GVHTFPAVLQSSGLYS | |
| | | | SGVQPEDEAEYYCALWYS | | LSSVVTVPSSSLGTQT | |
| | | | NRWVFGGGTKLTVLGGG | | YICNVNHKPSNTKVD | |
| | | | GSDIQMTQSPSSLSASVGD | | KKVEPKSC | |
| | | | RVTITCKSSQSLLYRSNQK | | | |
| | | | NYLAWYQQKPGKAPKLLI | | | |
| | | | YWASTRESGVPSRFSGSGS | | | |
| | | | GTDFTLTISSLQPEDFATY | | | |
| | | | YCQQYYNYPRTFGQGTKV | | | |
| | | | EIKRTVAAPSVFIFPPSDEQ | | | |
| | | | LKSGTASVVCLLNNFYPRE | | | |
| | | | AKVQWKVDNALQSGNSQ | | | |
| | | | ESVTEQDSKDSTYSLSSTL | | | |
| | | | TLSKADYEKHKVYACEVT | | | |
| | | | HQGLSSPVTKSFNRGECG | | | |
| | | | GGGSGGGGSDIQMTQSPSS | | | |
| | | | LSASVGDRVTITCKSSQSL | | | |
| | | | LYRSNQKNYLAWYQQKP | | | |
| | | | GKAPKLLIYWASTRESGVP | | | |
| | | | SRFSGSGSGTDFTLTISSLQ | | | |
| | | | PEDFATYYCQQYYNYPRT | | | |
| | | | FGQGTKVEIKRTVAAPSVF | | | |
| | | | IFPPSDEQLKSGTASVVCL | | | |
| | | | LNNFYPREAKVQWKVDN | | | |
| | | | ALQSGNSQESVTEQDSKD | | | |
| | | | STYSLSSTLTLSKADYEKH | | | |
| | | | KVYACEVTHQGLSSPVTK | | | |
| | | | SFNRGEC | | | |
| TCE- | (Q3V, | (N30Q, | DIVMTQSPDSLAVSLGERA | 164 | EVQLVESGGGLVQPG | 212 |
| 39/ | S9D, | I109V, | TINCKSSQSLLYRSNQKNY | | GSLKLSCAASGFTFQK | |
| (2 + 1) | S12A, | G172A, | LAWYQQKPGQPPKLLIYW | | YAMNWVRQAPGKGL | |
| | A13V, | V231A) | ASTRESGVPDRFSGSGSGT | | EWVARIRSKYNNYAT | |
| | V15L, | AND | DFTLTISSLQAEDVAVYYC | | YYADSVKDRFTISRDD | |
| | D17E, | (N30Q, | QQYYNYPRTFGQGTKVEI | | SKNTAYLQMNNLKTE | |
| | V19A, | I109V, | KRTVAAPSVFIFPPSDEQL | | DTAVYYCVRHGNFGN | |
| | T22N, | G172A, | KSGTASVVCLLNNFYPRE | | SYVSYWAYWGQGTL | |
| | K48Q, | V231A) | AKVQWKVDNALQSGNSQ | | VTVSSGGGGSGGGGS | |
| | A49P, | | ESVTEQDSKDSTYSLSSTL | | GGGGSQTVVTQEPSLT | |
| | S66D, | | TLSKADYEKHKVYACEVT | | VSPGGTVTLTCGSSTG | |
| | P86A, | | HQGLSSPVTKSFNRGEC | | AVTSANYPNWVQQKP | |
| | F89V, | | | | GQAPRGLIGGTKFLAP | |
| | T91V) | | | | GTPARFSGSLLGGKAA | |
| | AND | | | | LTLSGVQPEDEAEYYC | |
| | (V24A, | | | | ALWYSNRWVFGGGT | |
| | Y27F, | | | | KLTVLGGGGSEVQLV | |
| | T58I, | | | | ESGGGLVQPGGSLRLS | |
| | T74N) | | | | CAASGFSITSDYAWN | |
| | | | | | WVRQAPGKGLEWVG | |
| | | | | | YISNSGSISYNPSLKSR | |
| | | | | | FTISRDNSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSCDGGGGSGGGG | |
| | | | | | SEVQLVESGGGLVQPG | |
| | | | | | GSLRLSCAASGFSITSD | |
| | | | | | YAWNWVRQAPGKGL | |
| | | | | | EWVGYISNSGSISYNPS | |
| | | | | | LKSRFTISRDNSKNTL | |
| | | | | | YLQMNSLRAEDTAVY | |
| | | | | | YCARERNYDYDDYYY | |
| | | | | | AMDYWGQGTLVTVSS | |
| | | | | | ASTKGPSVFPLAPSSKS | |
| | | | | | TSGGTAALGCLVKDY | |
| | | | | | FPEPVTVSWNSGALTS | |
| | | | | | GVHTFPAVLQSSGLYS | |
| | | | | | LSSVVTVPSSSLGTQT | |
| | | | | | YICNVNHKPSNTKVD | |
| | | | | | KKVEPKSC | |
| TCE- | (Q3V, | (N30Q, | EVQLVESGGGLVQPGGSL | 165 | EVQLVESGGGLVQPG | 213 |
| 40/ | S9D, | I109V, | KLSCAASGFTFQKYAMN | | GSLRLSCAASGFSITSD | |
| (2 + 1) | S12A, | G172A, | WVRQAPGKGLEWVARIRS | | YAWNWVRQAPGKGL | |
| | A13V, | V231A) | KYNNYATYYADSVKDRFT | | EWVGYISNSGSISYNPS | |
| | V15L, | AND | ISRDDSKNTAYLQMNNLK | | LKSRFTISRDNSKNTL | |
| | D17E, | (N30Q, | TEDTAVYYCVRHGNFGNS | | YLQMNSLRAEDTAVY | |
| | V19A, | I109V, | YVSYWAYWGQGTLVTVS | | YCARERNYDYDDYYY | |
| | T22N, | G172A, | SGGGGSGGGGSGGGGSQT | | AMDYWGQGTLVTVSS | |
| | K48Q, | V231A) | VVTQEPSLTVSPGGTVTLT | | ASTKGPSVFPLAPSSKS | |
| | A49P, | | CGSSTGAVTSANYPNWVQ | | TSGGTAALGCLVKDY | |
| | S66D, | | QKPGQAPRGLIGGTKFLAP | | FPEPVTVSWNSGALTS | |
| | P86A, | | GTPARFSGSLLGGKAALTL | | GVHTFPAVLQSSGLYS | |
| | F89V, | | SGVQPEDEAEYYCALWYS | | LSSVVTVPSSSLGTQT | |
| | T91V) | | NRWVFGGGTKLTVLGGG | | YICNVNHKPSNTKVD | |
| | AND | | GSDIVMTQSPDSLAVSLGE | | KKVEPKSC | |
| | (V24A, | | RATINCKSSQSLLYRSNQK | | | |
| | Y27F, | | NYLAWYQQKPGQPPKLLI | | | |
| | T58I | | YWASTRESGVPDRFSGSG | | | |
| | T74N) | | SGTDFTLTISSLQAEDVAV | | | |
| | | | YYCQQYYNYPRTFGQGTK | | | |
| | | | VEIKRTVAAPSVFIFPPSDE | | | |
| | | | QLKSGTASVVCLLNNFYP | | | |
| | | | REAKVQWKVDNALQSGN | | | |
| | | | SQESVTEQDSKDSTYSLSS | | | |
| | | | TLTLSKADYEKHKVYACE | | | |
| | | | VTHQGLSSPVTKSFNRGEC | | | |
| | | | GGGGSGGGGSDIVMTQSP | | | |
| | | | DSLAVSLGERATINCKSSQ | | | |
| | | | SLLYRSNQKNYLAWYQQ | | | |
| | | | KPGQPPKLLIYWASTRESG | | | |
| | | | VPDRFSGSGSGTDFTLTISS | | | |
| | | | LQAEDVAVYYCQQYYNY | | | |
| | | | PRTFGQGTKVEIKRTVAAP | | | |
| | | | SVFIFPPSDEQLKSGTASVV | | | |
| | | | CLLNNFYPREAKVQWKV | | | |
| | | | DNALQSGNSQESVTEQDS | | | |
| | | | KDSTYSLSSTLTLSKADYE | | | |
| | | | KHKVYACEVTHQGLSSPV | | | |
| | | | TKSFNRGEC | | | |
| TCE- | (Q35N) | (N30Q, | DIQMTQSPSSLSASVGDRV | 166 | EVQLVESGGGLVQPG | 214 |
| 41/ | AND | I109V, | TITCKSSQSLLYRSNNKNY | | GSLKLSCAASGFTFQK | |
| (2 + 1) | (W35M, | G172A, | LAWYQQKPGKAPKLLIYW | | YAMNWVRQAPGKGL | |
| | L64V) | V231A) | ASTRESGVPSRFSGSGSGT | | EWVARIRSKYNNYAT | |
| | | AND | DFTLTISSLQPEDFATYYC | | YYADSVKDRFTISRDD | |
| | | (N30Q, | QQYYNYPRTFGQGTKVEI | | SKNTAYLQMNNLKTE | |
| | | I109V, | KRTVAAPSVFIFPPSDEQL | | DTAVYYCVRHGNFGN | |
| | | G172A, | KSGTASVVCLLNNFYPRE | | SYVSYWAYWGQGTL | |
| | | V231A) | AKVQWKVDNALQSGNSQ | | VTVSSGGGGSGGGGS | |
| | | | ESVTEQDSKDSTYSLSSTL | | GGGGSQTVVTQEPSLT | |
| | | | TLSKADYEKHKVYACEVT | | VSPGGTVTLTCGSSTG | |
| | | | HQGLSSPVTKSFNRGEC | | AVTSANYPNWVQQKP | |
| | | | | | GQAPRGLIGGTKFLAP | |
| | | | | | GTPARFSGSLLGGKAA | |
| | | | | | LTLSGVQPEDEAEYYC | |
| | | | | | ALWYSNRWVFGGGT | |
| | | | | | KLTVLGGGGSEVQLV | |
| | | | | | ESGGGLVQPGGSLRLS | |
| | | | | | CAVSGYSITSDYAMN | |
| | | | | | WVRQAPGKGLEWVG | |
| | | | | | YISNSGSTSYNPSVKSR | |
| | | | | | FTISRDTSKNTLYLQM | |
| | | | | | NSLRAEDTAVYYCAR | |
| | | | | | ERNYDYDDYYYAMD | |
| | | | | | YWGQGTLVTVSSAST | |
| | | | | | KGPSVFPLAPSSKSTSG | |
| | | | | | GTAALGCLVKDYFPEP | |
| | | | | | VTVSWNSGALTSGVH | |
| | | | | | TFPAVLQSSGLYSLSS | |
| | | | | | VVTVPSSSLGTQTYIC | |
| | | | | | NVNHKPSNTKVDKKV | |
| | | | | | EPKSCDGGGGSGGGG | |
| | | | | | SEVQLVESGGGLVQPG | |
| | | | | | GSLRLSCAVSGYSITSD | |
| | | | | | YAMNWVRQAPGKGL | |
| | | | | | EWVGYISNSGSTSYNP | |
| | | | | | SVKSRFTISRDTSKNTL | |
| | | | | | YLQMNSLRAEDTAVY | |
| | | | | | YCARERNYDYDDYYY | |
| | | | | | AMDYWGQGTLVTVSS | |
| | | | | | ASTKGPSVFPLAPSSKS | |
| | | | | | TSGGTAALGCLVKDY | |
| | | | | | FPEPVTVSWNSGALTS | |
| | | | | | GVHTFPAVLQSSGLYS | |
| | | | | | LSSVVTVPSSSLGTQT | |
| | | | | | YICNVNHKPSNTKVD | |
| | | | | | KKVEPKSC | |
| TCE- | (Q35N) | (N30Q, | EVQLVESGGGLVQPGGSLKL | 167 | EVQLVESGGGLVQPGGSLRL | 215 |
| 42/ | AND | I109V, | SCAASGFTFQKYAMNWVRQA | | SCAVSGYSITSDYAMNWVRQ | |
| (2 + 1) | (W35M, | G172A, | PGKGLEWVARIRSKYNNYAT | | APGKGLEWVGYISNSGSTSY | |
| | L64V) | V231A) | YYADSVKDRFTISRDDSKNT | | NPSVKSRFTISRDTSKNTLY | |
| | | AND | AYLQMNNLKTEDTAVYYCVR | | LQMNSLRAEDTAVYYCARER | |
| | | (N30Q, | HGNFGNSYVSYWAYWGQGTL | | NYDYDDYYYAMDYWGQGTLV | |
| | | I109V, | VTVSSGGGGSGGGGSGGGGS | | TVSSASTKGPSVFPLAPSSK | |
| | | G172A, | QTVVTQEPSLTVSPGGTVTL | | STSGGTAALGCLVKDYFPEP | |
| | | V231A) | TCGSSTGAVTSANYPNWVQQ | | VTVSWNSGALTSGVHTFPAV | |
| | | | KPGQAPRGLIGGTKFLAPGT | | LQSSGLYSLSSVVTVPSSSL | |
| | | | PARFSGSLLGGKAALTLSGV | | GTQTYICNVNHKPSNTKVDK | |
| | | | QPEDEAEYYCALWYSNRWVF | | KVEPKSC | |
| | | | GGGTKLTVLGGGGSDIQMTQ | | | |
| | | | SPSSLSASVGDRVTITCKSS | | | |
| | | | QSLLYRSNNKNYLAWYQQKP | | | |
| | | | GKAPKLLIYWASTRESGVPS | | | |
| | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGECGGGGSG | | | |
| | | | GGGSDIQMTQSPSSLSASVG | | | |
| | | | DRVTITCKSSQSLLYRSNNK | | | |
| | | | NYLAWYQQKPGKAPKLLIYW | | | |
| | | | ASTRESGVPSRFSGSGSGTD | | | |
| | | | FTLTISSLQPEDFATYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGEC | | | |
| TCE- | (Q3V, | (N30Q, | DIVMTQSPDSLAVSLGERAT | 168 | EVQLVESGGGLVQPGGSLKL | 216 |
| 43/ | S9D, | I109V, | INCKSSQSLLYRSNNKNYLA | | SCAASGFTFQKYAMNWVRQA | |
| (2 + 1) | S12A, | G172A, | WYQQKPGQPPKLLIYWASTR | | PGKGLEWVARIRSKYNNYAT | |
| | A13V, | V231A) | ESGVPDRFSGSGSGTDFTLT | | YYADSVKDRFTISRDDSKNT | |
| | V15L, | AND | ISSLQAEDVAVYYCQQYYNY | | AYLQMNNLKTEDTAVYYCVR | |
| | D17E, | (N30Q, | PRTFGQGTKVEIKRTVAAPS | | HGNFGNSYVSYWAYWGQGTL | |
| | V19A, | I109V, | VFIFPPSDEQLKSGTASVVC | | VTVSSGGGGSGGGGSGGGGS | |
| | T22N, | G172A, | LLNNFYPREAKVQWKVDNAL | | QTVVTQEPSLTVSPGGTVTL | |
| | Q35N, | V231A) | QSGNSQESVTEQDSKDSTYS | | TCGSSTGAVTSANYPNWVQQ | |
| | K48Q, | | LSSTLTLSKADYEKHKVYAC | | KPGQAPRGLIGGTKFLAPGT | |
| | A49P, S66D, | | EVTHQGLSSPVTKSFNRGEC | | PARFSGSLLGGKAALTLSGV | |
| | P86A, | | | | QPEDEAEYYCALWYSNRWVF | |
| | F89V, | | | | GGGTKLTVLGGGGSEVQLVE | |
| | T91V) | | | | SGGGLVQPGGSLRLSCAASG | |
| | AND | | | | FSITSDYAMNWVRQAPGKGL | |
| | (V24A, | | | | EWVGYISNSGSISYNPSVKG | |
| | Y27F, | | | | RFTISRDNSKNTLYLQMNSL | |
| | W35M, | | | | RAEDTAVYYCARERNYDYDD | |
| | T58I, | | | | YYYAMDYWGQGTLVTVSSAS | |
| | L64V, | | | | TKGPSVFPLAPSSKSTSGGT | |
| | S66G, | | | | AALGCLVKDYFPEPVTVSWN | |
| | T74N) | | | | SGALTSGVHTFPAVLQSSGL | |
| | | | | | YSLSSVVTVPSSSLGTQTYI | |
| | | | | | CNVNHKPSNTKVDKKVEPKS | |
| | | | | | CDGGGGSGGGGSEVQLVESG | |
| | | | | | GGLVQPGGSLRLSCAASGFS | |
| | | | | | ITSDYAMNWVRQAPGKGLEW | |
| | | | | | VGYISNSGSISYNPSVKGRF | |
| | | | | | TISRDNSKNTLYLQMNSLRA | |
| | | | | | EDTAVYYCARERNYDYDDYY | |
| | | | | | YAMDYWGQGTLVTVSSASTK | |
| | | | | | GPSVFPLAPSSKSTSGGTAA | |
| | | | | | LGCLVKDYFPEPVTVSWNSG | |
| | | | | | ALTSGVHTFPAVLQSSGLYS | |
| | | | | | LSSVVTVPSSSLGTQTYICN | |
| | | | | | VNHKPSNTKVDKKVEPKSC | |
| TCE- | (Q3V, | (N30Q, | EVQLVESGGGLVQPGGSLKL | 169 | EVQLVESGGGLVQPGGSLRL | 217 |
| 44/ | S9D, | I109V, | SCAASGFTFQKYAMNWVRQA | | SCAASGFSITSDYAMNWVRQ | |
| (2 + 1) | S12A, | G172A, | PGKGLEWVARIRSKYNNYAT | | APGKGLEWVGYISNSGSISY | |
| | A13V, | V231A) | YYADSVKDRFTISRDDSKNT | | NPSVKGRFTISRDNSKNTLY | |
| | V15L, | AND | AYLQMNNLKTEDTAVYYCVR | | LQMNSLRAEDTAVYYCARER | |
| | D17E, | (N30Q, | HGNFGNSYVSYWAYWGQGTL | | NYDYDDYYYAMDYWGQGTLV | |
| | V19A, | I109V, | VTVSSGGGGSGGGGSGGGGS | | TVSSASTKGPSVFPLAPSSK | |
| | T22N, | G172A, | QTVVTQEPSLTVSPGGTVTL | | STSGGTAALGCLVKDYFPEP | |
| | Q35N, | V231A) | TCGSSTGAVTSANYPNWVQQ | | VTVSWNSGALTSGVHTFPAV | |
| | K48Q, | | KPGQAPRGLIGGTKFLAPGT | | LQSSGLYSLSSVVTVPSSSL | |
| | A49P, | | PARFSGSLLGGKAALTLSGV | | GTQTYICNVNHKPSNTKVDK | |
| | S66D, | | QPEDEAEYYCALWYSNRWVF | | KVEPKSC | |
| | P86A, | | GGGTKLTVLGGGGSDIVMTQ | | | |
| | F89V, | | SPDSLAVSLGERATINCKSS | | | |
| | T91V) | | QSLLYRSNNKNYLAWYQQKP | | | |
| | AND | | GQPPKLLIYWASTRESGVPD | | | |
| | (V24A, | | RFSGSGSGTDFTLTISSLQA | | | |
| | Y27F, | | EDVAVYYCQQYYNYPRTFGQ | | | |
| | W35M, | | GTKVEIKRTVAAPSVFIFPP | | | |
| | T58I, | | SDEQLKSGTASVVCLLNNFY | | | |
| | L64V, | | PREAKVQWKVDNALQSGNSQ | | | |
| | S66G, | | ESVTEQDSKDSTYSLSSTLT | | | |
| | T74N) | | LSKADYEKHKVYACEVTHQG | | | |
| | | | LSSPVTKSFNRGECGGGGSG | | | |
| | | | GGGSDIVMTQSPDSLAVSLG | | | |
| | | | ERATINCKSSQSLLYRSNNK | | | |
| | | | NYLAWYQQKPGQPPKLLIYW | | | |
| | | | ASTRESGVPDRFSGSGSGTD | | | |
| | | | FTLTISSLQAEDVAVYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGEC | | | |
| TCE- | (Q35N) | (N30Q, | DIQMTQSPSSLSASVGDRVT | 170 | EVQLVESGGGLVQPGGSLKL | 218 |
| 45/ | AND | I109V, | ITCKSSQSLLYRSNNKNYLA | | SCAASGFTFQKYAMNWVRQA | |
| (2 + 1) | (W35M, | G172A, | WYQQKPGKAPKLLIYWASTR | | PGKGLEWVARIRSKYNNYAT | |
| | L64V, | V231A) | ESGVPSRFSGSGSGTDFTLT | | YYADSVKDRFTISRDDSKNT | |
| | E99D, | AND | ISSLQPEDFATYYCQQYYNY | | AYLQMNNLKTEDTAVYYCVR | |
| | M111F) | (N30Q, | PRTFGQGTKVEIKRTVAAPS | | HGNFGNSYVSYWAYWGQGTL | |
| | | I109V, | VFIFPPSDEQLKSGTASVVC | | VTVSSGGGGSGGGGSGGGGS | |
| | | G172A, | LLNNFYPREAKVQWKVDNAL | | QTVVTQEPSLTVSPGGTVTL | |
| | | V231A) | QSGNSQESVTEQDSKDSTYS | | TCGSSTGAVTSANYPNWVQQ | |
| | | | LSSTLTLSKADYEKHKVYAC | | KPGQAPRGLIGGTKFLAPGT | |
| | | | EVTHQGLSSPVTKSFNRGEC | | PARFSGSLLGGKAALTLSGV | |
| | | | | | QPEDEAEYYCALWYSNRWVF | |
| | | | | | GGGTKLTVLGGGGSEVQLVE | |
| | | | | | SGGGLVQPGGSLRLSCAVSG | |
| | | | | | YSITSDYAMNWVRQAPGKGL | |
| | | | | | EWVGYISNSGSTSYNPSVKS | |
| | | | | | RFTISRDTSKNTLYLQMNSL | |
| | | | | | RAEDTAVYYCARDRNYDYDD | |
| | | | | | YYYAFDYWGQGTLVTVSSAS | |
| | | | | | TKGPSVFPLAPSSKSTSGGT | |
| | | | | | AALGCLVKDYFPEPVTVSWN | |
| | | | | | SGALTSGVHTFPAVLQSSGL | |
| | | | | | YSLSSVVTVPSSSLGTQTYI | |
| | | | | | CNVNHKPSNTKVDKKVEPKS | |
| | | | | | CDGGGGSGGGGSEVQLVESG | |
| | | | | | GGLVQPGGSLRLSCAVSGYS | |
| | | | | | ITSDYAMNWVRQAPGKGLEW | |
| | | | | | VGYISNSGSTSYNPSVKSRF | |
| | | | | | TISRDTSKNTLYLQMNSLRA | |
| | | | | | EDTAVYYCARDRNYDYDDYY | |
| | | | | | YAFDYWGQGTLVTVSSASTK | |
| | | | | | GPSVFPLAPSSKSTSGGTAA | |
| | | | | | LGCLVKDYFPEPVTVSWNSG | |
| | | | | | ALTSGVHTFPAVLQSSGLYS | |
| | | | | | LSSVVTVPSSSLGTQTYICN | |
| | | | | | VNHKPSNTKVDKKVEPKSC | |
| TCE- | (Q35N) | (N30Q, | EVQLVESGGGLVQPGGSLKL | 171 | EVQLVESGGGLVQPGGSLRL | 219 |
| 46/ | AND | I109V, | SCAASGFTFQKYAMNWVRQA | | SCAVSGYSITSDYAMNWVRQ | |
| (2 + 1) | (W35M, | G172A, | PGKGLEWVARIRSKYNNYAT | | APGKGLEWVGYISNSGSTSY | |
| | L64V, | V231A) | YYADSVKDRFTISRDDSKNT | | NPSVKSRFTISRDTSKNTLY | |
| | E99D, | AND | AYLQMNNLKTEDTAVYYCVR | | LQMNSLRAEDTAVYYCARDR | |
| | M111F) | (N30Q, | HGNFGNSYVSYWAYWGQGTL | | NYDYDDYYYAFDYWGQGTLV | |
| | | I109V, | VTVSSGGGGSGGGGSGGGGS | | TVSSASTKGPSVFPLAPSSK | |
| | | G172A, | QTVVTQEPSLTVSPGGTVTL | | STSGGTAALGCLVKDYFPEP | |
| | | V231A) | TCGSSTGAVTSANYPNWVQQ | | VTVSWNSGALTSGVHTFPAV | |
| | | | KPGQAPRGLIGGTKFLAPGT | | LQSSGLYSLSSVVTVPSSSL | |
| | | | PARFSGSLLGGKAALTLSGV | | GTQTYICNVNHKPSNTKVDK | |
| | | | QPEDEAEYYCALWYSNRWVF | | KVEPKSC | |
| | | | GGGTKLTVLGGGGSDIQMTQ | | | |
| | | | SPSSLSASVGDRVTITCKSS | | | |
| | | | QSLLYRSNNKNYLAWYQQKP | | | |
| | | | GKAPKLLIYWASTRESGVPS | | | |
| | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGECGGGGSG | | | |
| | | | GGGSDIQMTQSPSSLSASVG | | | |
| | | | DRVTITCKSSQSLLYRSNNK | | | |
| | | | NYLAWYQQKPGKAPKLLIYW | | | |
| | | | ASTRESGVPSRFSGSGSGTD | | | |
| | | | FTLTISSLQPEDFATYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGEC | | | |
| TCE- | (Q3V, | (N30Q, | DIVMTQSPDSLAVSLGERAT | 172 | EVQLVESGGGLVQPGGSLKL | 220 |
| 47/ | S9D, | I109V, | INCKSSQSLLYRSNNKNYLA | | SCAASGFTFQKYAMNWVRQA | |
| (2 + 1) | S12A, | G172A, | WYQQKPGQPPKLLIYWASTR | | PGKGLEWVARIRSKYNNYAT | |
| | A13V, | V231A) | ESGVPDRFSGSGSGTDFTLT | | YYADSVKDRFTISRDDSKNT | |
| | V15L, | AND | ISSLQAEDVAVYYCQQYYNY | | AYLQMNNLKTEDTAVYYCVR | |
| | D17E, | (N30Q, | PRTFGQGTKVEIKRTVAAPS | | HGNFGNSYVSYWAYWGQGTL | |
| | V19A, | I109V, | VFIFPPSDEQLKSGTASVVC | | VTVSSGGGGSGGGGSGGGGS | |
| | T22N, | G172A, | LLNNFYPREAKVQWKVDNAL | | QTVVTQEPSLTVSPGGTVTL | |
| | Q35N, | V231A) | QSGNSQESVTEQDSKDSTYS | | TCGSSTGAVTSANYPNWVQQ | |
| | K48Q, | | LSSTLTLSKADYEKHKVYAC | | KPGQAPRGLIGGTKFLAPGT | |
| | A49P, | | EVTHQGLSSPVTKSFNRGEC | | PARFSGSLLGGKAALTLSGV | |
| | S66D, | | | | QPEDEAEYYCALWYSNRWVF | |
| | P86A, | | | | GGGTKLTVLGGGGSEVQLVE | |
| | F89V, | | | | SGGGLVQPGGSLRLSCAASG | |
| | T91V) | | | | FSITSDYAMNWVRQAPGKGL | |
| | AND | | | | EWVGYISNSGSTSYNPSVKS | |
| | (V24A, | | | | RFTISRDNSKNTLYLQMNSL | |
| | Y27F, | | | | RAEDTAVYYCARDRNYDYDD | |
| | W35M, | | | | YYYAFDYWGQGTLVTVSSAS | |
| | L64V, | | | | TKGPSVFPLAPSSKSTSGGT | |
| | T74N, | | | | AALGCLVKDYFPEPVTVSWN | |
| | E99D, | | | | SGALTSGVHTFPAVLQSSGL | |
| | M111F) | | | | YSLSSVVTVPSSSLGTQTYI | |
| | | | | | CNVNHKPSNTKVDKKVEPKS | |
| | | | | | CDGGGGSGGGGSEVQLVESG | |
| | | | | | GGLVQPGGSLRLSCAASGFS | |
| | | | | | ITSDYAMNWVRQAPGKGLEW | |
| | | | | | VGYISNSGSTSYNPSVKSRF | |
| | | | | | TISRDNSKNTLYLQMNSLRA | |
| | | | | | EDTAVYYCARDRNYDYDDYY | |
| | | | | | YAFDYWGQGTLVTVSSASTK | |
| | | | | | GPSVFPLAPSSKSTSGGTAA | |
| | | | | | LGCLVKDYFPEPVTVSWNSG | |
| | | | | | ALTSGVHTFPAVLQSSGLYS | |
| | | | | | LSSVVTVPSSSLGTQTYICN | |
| | | | | | VNHKPSNTKVDKKVEPKSC | |
| TCE- | (Q3V, | (N30Q, | EVQLVESGGGLVQPGGSLKL | 173 | EVQLVESGGGLVQPGGSLRL | 221 |
| 48/ | S9D, | I109V, | SCAASGFTFQKYAMNWVRQA | | SCAASGFSITSDYAMNWVRQ | |
| (2 + 1) | S12A, | G172A, | PGKGLEWVARIRSKYNNYAT | | APGKGLEWVGYISNSGSTSY | |
| | A13V, | V231A) | YYADSVKDRFTISRDDSKNT | | NPSVKSRFTISRDNSKNTLY | |
| | V15L, | AND | AYLQMNNLKTEDTAVYYCVR | | LQMNSLRAEDTAVYYCARDR | |
| | D17E, | (N30Q, | HGNFGNSYVSYWAYWGQGTL | | NYDYDDYYYAFDYWGQGTLV | |
| | V19A, | I109V, | VTVSSGGGGSGGGGSGGGGS | | TVSSASTKGPSVFPLAPSSK | |
| | T22N, | G172A, | QTVVTQEPSLTVSPGGTVTL | | STSGGTAALGCLVKDYFPEP | |
| | Q35N, | V231A) | TCGSSTGAVTSANYPNWVQQ | | VTVSWNSGALTSGVHTFPAV | |
| | K48Q, | | KPGQAPRGLIGGTKFLAPGT | | LQSSGLYSLSSVVTVPSSSL | |
| | A49P, | | PARFSGSLLGGKAALTLSGV | | GTQTYICNVNHKPSNTKVDK | |
| | S66D, | | QPEDEAEYYCALWYSNRWVF | | KVEPKSC | |
| | P86A, | | GGGTKLTVLGGGGSDIVMTQ | | | |
| | F89V, | | SPDSLAVSLGERATINCKSS | | | |
| | T91V) | | QSLLYRSNNKNYLAWYQQKP | | | |
| | AND | | GQPPKLLIYWASTRESGVPD | | | |
| | (V24A, | | RFSGSGSGTDFTLTISSLQA | | | |
| | Y27F, | | EDVAVYYCQQYYNYPRTFGQ | | | |
| | W35M, | | GTKVEIKRTVAAPSVFIFPP | | | |
| | L64V, | | SDEQLKSGTASVVCLLNNFY | | | |
| | T74N, | | PREAKVQWKVDNALQSGNSQ | | | |
| | E99D, | | ESVTEQDSKDSTYSLSSTLT | | | |
| | M111F) | | LSKADYEKHKVYACEVTHQG | | | |
| | | | LSSPVTKSFNRGECGGGGSG | | | |
| | | | GGGSDIVMTQSPDSLAVSLG | | | |
| | | | ERATINCKSSQSLLYRSNNK | | | |
| | | | NYLAWYQQKPGQPPKLLIYW | | | |
| | | | ASTRESGVPDRFSGSGSGTD | | | |
| | | | FTLTISSLQAEDVAVYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGEC | | | |
| TCE-77 | (None) | (None) | DIQMTQSPSSLSASVGDRVT | 330 | EVQLVESGGGLVQPGGSLKL | 343 |
| [2 + 1] | AND | AND | ITCKSSQSLLYRSNQKNYLA | | SCAASGFTFNKYAMNWVRQA | |
| N-term | (R19S) | (None) | WYQQKPGKAPKLLIYWASTR | | PGKGLEWVARIRSKYNNYAT | |
| CD3 | | | ESGVPSRFSGSGSGTDFTLT | | YYADSVKDRFTISRDDSKNT | |
| scFv | | | ISSLQPEDFATYYCQQYYNY | | AYLQMNNLKTEDTAVYYCVR | |
| (Vh- | | | PRTFGQGTKVEIKRTVAAPS | | HGNFGNSYISYWAYWGQGTL | |
| Vl); L₁ | | | VFIFPPSDEQLKSGTASVVC | | VTVSSGGGGSGGGGSGGGGS | |
| GGGG | | | LLNNFYPREAKVQWKVDNAL | | QTVVTQEPSLTVSPGGTVTL | |
| S (SEQ | | | QSGNSQESVTEQDSKDSTYS | | TCGSSTGAVTSGNYPNWVQQ | |
| ID NO: | | | LSSTLTLSKADYEKHKVYAC | | KPGQAPRGLIGGTKFLAPGT | |
| 223); L₂ | | | EVTHQGLSSPVTKSFNRGEC | | PARFSGSLLGGKAALTLSGV | |
| DGGG | | | | | QPEDEAEYYCVLWYSNRWVF | |
| GSGG | | | | | GGGTKLTVLGGGGSEVQLVE | |
| GGS | | | | | SGGGLVQPGGSLSLSCAVSG | |
| (SEQ | | | | | YSITSDYAWNWVRQAPGKGL | |
| ID NO: | | | | | EWVGYISNSGSTSYNPSLKS | |
| 371) | | | | | RFTISRDTSKNTLYLQMNSL | |
| | | | | | RAEDTAVYYCARERNYDYDD | |
| | | | | | YYYAMDYWGQGTLVTVSSAS | |
| | | | | | TKGPSVFPLAPSSKSTSGGT | |
| | | | | | AALGCLVKDYFPEPVTVSWN | |
| | | | | | SGALTSGVHTFPAVLQSSGL | |
| | | | | | YSLSSVVTVPSSSLGTQTYI | |
| | | | | | CNVNHKPSNTKVDKKVEPKS | |
| | | | | | CDGGGGSGGGGSEVQLVESG | |
| | | | | | GGLVQPGGSLSLSCAVSGYS | |
| | | | | | ITSDYAWNWVRQAPGKGLEW | |
| | | | | | VGYISNSGSTSYNPSLKSRF | |
| | | | | | TISRDTSKNTLYLQMNSLRA | |
| | | | | | EDTAVYYCARERNYDYDDYY | |
| | | | | | YAMDYWGQGTLVTVSSASTK | |
| | | | | | GPSVFPLAPSSKSTSGGTAA | |
| | | | | | LGCLVKDYFPEPVTVSWNSG | |
| | | | | | ALTSGVHTFPAVLQSSGLYS | |
| | | | | | LSSVVTVPSSSLGTQTYICN | |
| | | | | | VNHKPSNTKVDKKVEPKSC | |
| TCE-78 | (None) | (None) | EVQLVESGGGLVQPGGSLKL | 331 | EVQLVESGGGLVQPGGSLSL | 344 |
| [2 + 1] | AND | AND | SCAASGFTFNKYAMNWVRQA | | SCAVSGYSITSDYAWNWVRQ | |
| N-term | (R19S) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv | | | AYLQMNNLKTEDTAVYYCVR | | LQMNSLRAEDTAVYYCARER | |
| (Vh- | | | HGNFGNSYISYWAYWGQGTL | | NYDYDDYYYAMDYWGQGTLV | |
| Vl); L₁ | | | VTVSSGGGGSGGGGSGGGGS | | TVSSASTKGPSVFPLAPSSK | |
| GGGG | | | QTVVTQEPSLTVSPGGTVTL | | STSGGTAALGCLVKDYFPEP | |
| S (SEQ | | | TCGSSTGAVTSGNYPNWVQQ | | VTVSWNSGALTSGVHTFPAV | |
| ID NO: | | | KPGQAPRGLIGGTKFLAPGT | | LQSSGLYSLSSVVTVPSSSL | |
| 223); L₂ | | | PARFSGSLLGGKAALTLSGV | | GTQTYICNVNHKPSNTKVDK | |
| GGGG | | | QPEDEAEYYCVLWYSNRWVF | | KVEPKSC | |
| SGGG | | | GGGTKLTVLGGGGSDIQMTQ | | | |
| GS | | | SPSSLSASVGDRVTITCKSS | | | |
| (SEQ | | | QSLLYRSNQKNYLAWYQQKP | | | |
| ID NO: | | | GKAPKLLIYWASTRESGVPS | | | |
| 270) | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGECGGGGSG | | | |
| | | | GGGSDIQMTQSPSSLSASVG | | | |
| | | | DRVTITCKSSQSLLYRSNQK | | | |
| | | | NYLAWYQQKPGKAPKLLIYW | | | |
| | | | ASTRESGVPSRFSGSGSGTD | | | |
| | | | FTLTISSLQPEDFATYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGEC | | | |
| TCE-79 | (None) | (None) | EVQLVESGGGLVQPGGSLKL | 332 | EVQLVESGGGLVQPGGSLRL | 345 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| S (SEQ | | | VTVSSGGGGSGGGGSGGGGS | | TVSSASTKGPSVFPLAPSSK | |
| ID NO: | | | QTVVTQEPSLTVSPGGTVTL | | STSGGTAALGCLVKDYFPEP | |
| 223); L₂ | | | TCGSSTGAVTSGNYPNWVQQ | | VTVSWNSGALTSGVHTFPAV | |
| GGGG | | | KPGQAPRGLIGGTKFLAPGT | | LQSSGLYSLSSVVTVPSSSL | |
| SGGG | | | PARFSGSLLGGKAALTLSGV | | GTQTYICNVNHKPSNTKVDK | |
| GSGG | | | QPEDEAEYYCVLWYSNRWVF | | KVEPKSC | |
| GGS | | | GGGTKLTVLGGGGSDIQMTQ | | | |
| (SEQ | | | SPSSLSASVGDRVTITCKSS | | | |
| ID NO: | | | QSLLYRSNQKNYLAWYQQKP | | | |
| 222) | | | GKAPKLLIYWASTRESGVPS | | | |
| | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGECGGGGSG | | | |
| | | | GGGSGGGGSDIQMTQSPSSL | | | |
| | | | SASVGDRVTITCKSSQSLLY | | | |
| | | | RSNQKNYLAWYQQKPGKAPK | | | |
| | | | LLIYWASTRESGVPSRFSGS | | | |
| | | | GSGTDFTLTISSLQPEDFAT | | | |
| | | | YYCQQYYNYPRTFGQGTKVE | | | |
| | | | IKRTVAAPSVFIFPPSDEQL | | | |
| | | | KSGTASVVCLLNNFYPREAK | | | |
| | | | VQWKVDNALQSGNSQESVTE | | | |
| | | | QDSKDSTYSLSSTLTLSKAD | | | |
| | | | YEKHKVYACEVTHQGLSSPV | | | |
| | | | TKSFNRGEC | | | |
| TCE-80 | (None) | (None) | EVQLVESGGGLVQPGGSLKL | 333 | EVQLVESGGGLVQPGGSLRL | 346 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| S (SEQ | | | VTVSSGGGGSGGGGSGGGGS | | TVSSASTKGPSVFPLAPSSK | |
| ID NO: | | | QTVVTQEPSLTVSPGGTVTL | | STSGGTAALGCLVKDYFPEP | |
| 223); L₂ | | | TCGSSTGAVTSGNYPNWVQQ | | VTVSWNSGALTSGVHTFPAV | |
| GGGG | | | KPGQAPRGLIGGTKFLAPGT | | LQSSGLYSLSSVVTVPSSSL | |
| SGGG | | | PARFSGSLLGGKAALTLSGV | | GTQTYICNVNHKPSNTKVDK | |
| GSGG | | | QPEDEAEYYCVLWYSNRWVF | | KVEPKSC | |
| GGSG | | | GGGTKLTVLGGGGSDIQMTQ | | | |
| GGGS | | | SPSSLSASVGDRVTITCKSS | | | |
| (SEQ | | | QSLLYRSNQKNYLAWYQQKP | | | |
| ID NO: | | | GKAPKLLIYWASTRESGVPS | | | |
| 271) | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGECGGGGSG | | | |
| | | | GGGSGGGGSGGGGSDIQMTQ | | | |
| | | | SPSSLSASVGDRVTITCKSS | | | |
| | | | QSLLYRSNQKNYLAWYQQKP | | | |
| | | | GKAPKLLIYWASTRESGVPS | | | |
| | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGEC | | | |
| TCE-81 | (None) | (None) | EVQLVESGGGLVQPGGSLKL | 334 | EVQLVESGGGLVQPGGSLRL | 347 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| S (SEQ | | | VTVSSGGGGSGGGGSGGGGS | | TVSSASTKGPSVFPLAPSSK | |
| ID NO: | | | QTVVTQEPSLTVSPGGTVTL | | STSGGTAALGCLVKDYFPEP | |
| 223); L₂ | | | TCGSSTGAVTSGNYPNWVQQ | | VTVSWNSGALTSGVHTFPAV | |
| GGGG | | | KPGQAPRGLIGGTKFLAPGT | | LQSSGLYSLSSVVTVPSSSL | |
| SGGG | | | PARFSGSLLGGKAALTLSGV | | GTQTYICNVNHKPSNTKVDK | |
| GSGG | | | QPEDEAEYYCVLWYSNRWVF | | KVEPKSC | |
| GGSG | | | GGGTKLTVLGGGGSDIQMTQ | | | |
| GGGS | | | SPSSLSASVGDRVTITCKSS | | | |
| GGGG | | | QSLLYRSNQKNYLAWYQQKP | | | |
| S (SEQ | | | GKAPKLLIYWASTRESGVPS | | | |
| ID NO: | | | RFSGSGSGTDFTLTISSLQP | | | |
| 272) | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGECGGGGSG | | | |
| | | | GGGSGGGGSGGGGSGGGGSD | | | |
| | | | IQMTQSPSSLSASVGDRVTI | | | |
| | | | TCKSSQSLLYRSNQKNYLAW | | | |
| | | | YQQKPGKAPKLLIYWASTRE | | | |
| | | | SGVPSRFSGSGSGTDFTLTI | | | |
| | | | SSLQPEDFATYYCQQYYNYP | | | |
| | | | RTFGQGTKVEIKRTVAAPSV | | | |
| | | | FIFPPSDEQLKSGTASVVCL | | | |
| | | | LNNFYPREAKVQWKVDNALQ | | | |
| | | | SGNSQESVTEQDSKDSTYSL | | | |
| | | | SSTLTLSKADYEKHKVYACE | | | |
| | | | VTHQGLSSPVTKSFNRGEC | | | |
| TCE-82 | (None) | (None) | EVQLVESGGGLVQPGGSLKL | 335 | EVQLVESGGGLVQPGGSLRL | 348 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GS | | | QTVVTQEPSLTVSPGGTVTL | | STSGGTAALGCLVKDYFPEP | |
| (SEQ | | | TCGSSTGAVTSGNYPNWVQQ | | VTVSWNSGALTSGVHTFPAV | |
| ID NO: | | | KPGQAPRGLIGGTKFLAPGT | | LQSSGLYSLSSVVTVPSSSL | |
| 270); L₂ | | | PARFSGSLLGGKAALTLSGV | | GTQTYICNVNHKPSNTKVDK | |
| GGGG | | | QPEDEAEYYCVLWYSNRWVF | | KVEPKSC | |
| SGGG | | | GGGTKLTVLGGGGSGGGGSD | | | |
| GS | | | IQMTQSPSSLSASVGDRVTI | | | |
| (SEQ | | | TCKSSQSLLYRSNQKNYLAW | | | |
| ID NO: | | | YQQKPGKAPKLLIYWASTRE | | | |
| 270) | | | SGVPSRFSGSGSGTDFTLTI | | | |
| | | | SSLQPEDFATYYCQQYYNYP | | | |
| | | | RTFGQGTKVEIKRTVAAPSV | | | |
| | | | FIFPPSDEQLKSGTASVVCL | | | |
| | | | LNNFYPREAKVQWKVDNALQ | | | |
| | | | SGNSQESVTEQDSKDSTYSL | | | |
| | | | SSTLTLSKADYEKHKVYACE | | | |
| | | | VTHQGLSSPVTKSFNRGECG | | | |
| | | | GGGSGGGGSDIQMTQSPSSL | | | |
| | | | SASVGDRVTITCKSSQSLLY | | | |
| | | | RSNQKNYLAWYQQKPGKAPK | | | |
| | | | LLIYWASTRESGVPSRFSGS | | | |
| | | | GSGTDFTLTISSLQPEDFAT | | | |
| | | | YYCQQYYNYPRTFGQGTKVE | | | |
| | | | IKRTVAAPSVFIFPPSDEQL | | | |
| | | | KSGTASVVCLLNNFYPREAK | | | |
| | | | VQWKVDNALQSGNSQESVTE | | | |
| | | | QDSKDSTYSLSSTLTLSKAD | | | |
| | | | YEKHKVYACEVTHQGLSSPV | | | |
| | | | TKSFNRGEC | | | |
| TCE-83 | (None) | (None) | EVQLVESGGGLVQPGGSLKL | 336 | EVQLVESGGGLVQPGGSLRL | 349 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GS | | | QTVVTQEPSLTVSPGGTVTL | | STSGGTAALGCLVKDYFPEP | |
| (SEQ | | | TCGSSTGAVTSGNYPNWVQQ | | VTVSWNSGALTSGVHTFPAV | |
| ID NO: | | | KPGQAPRGLIGGTKFLAPGT | | LQSSGLYSLSSVVTVPSSSL | |
| 270); L₂ | | | PARFSGSLLGGKAALTLSGV | | GTQTYICNVNHKPSNTKVDK | |
| GGGG | | | QPEDEAEYYCVLWYSNRWVF | | KVEPKSC | |
| SGGG | | | GGGTKLTVLGGGGSGGGGSD | | | |
| GSGG | | | IQMTQSPSSLSASVGDRVTI | | | |
| GGS | | | TCKSSQSLLYRSNQKNYLAW | | | |
| (SEQ | | | YQQKPGKAPKLLIYWASTRE | | | |
| ID NO: | | | SGVPSRFSGSGSGTDFTLTI | | | |
| 222) | | | SSLQPEDFATYYCQQYYNYP | | | |
| | | | RTFGQGTKVEIKRTVAAPSV | | | |
| | | | FIFPPSDEQLKSGTASVVCL | | | |
| | | | LNNFYPREAKVQWKVDNALQ | | | |
| | | | SGNSQESVTEQDSKDSTYSL | | | |
| | | | SSTLTLSKADYEKHKVYACE | | | |
| | | | VTHQGLSSPVTKSFNRGECG | | | |
| | | | GGGSGGGGSGGGGSDIQMTQ | | | |
| | | | SPSSLSASVGDRVTITCKSS | | | |
| | | | QSLLYRSNQKNYLAWYQQKP | | | |
| | | | GKAPKLLIYWASTRESGVPS | | | |
| | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGEC | | | |
| TCE-84 | (None) | (None) | EVQLVESGGGLVQPGGSLKL | 337 | EVQLVESGGGLVQPGGSLRL | 350 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GS | | | QTVVTQEPSLTVSPGGTVTL | | STSGGTAALGCLVKDYFPEP | |
| (SEQ | | | TCGSSTGAVTSGNYPNWVQQ | | VTVSWNSGALTSGVHTFPAV | |
| ID NO: | | | KPGQAPRGLIGGTKFLAPGT | | LQSSGLYSLSSVVTVPSSSL | |
| 270); L₂ | | | PARFSGSLLGGKAALTLSGV | | GTQTYICNVNHKPSNTKVDK | |
| GGGG | | | QPEDEAEYYCVLWYSNRWVF | | KVEPKSC | |
| SGGG | | | GGGTKLTVLGGGGSGGGGSD | | | |
| GSGG | | | IQMTQSPSSLSASVGDRVTI | | | |
| GGSG | | | TCKSSQSLLYRSNQKNYLAW | | | |
| GGGS | | | YQQKPGKAPKLLIYWASTRE | | | |
| (SEQ | | | SGVPSRFSGSGSGTDFTLTI | | | |
| ID NO: | | | SSLQPEDFATYYCQQYYNYP | | | |
| 271) | | | RTFGQGTKVEIKRTVAAPSV | | | |
| | | | FIFPPSDEQLKSGTASVVCL | | | |
| | | | LNNFYPREAKVQWKVDNALQ | | | |
| | | | SGNSQESVTEQDSKDSTYSL | | | |
| | | | SSTLTLSKADYEKHKVYACE | | | |
| | | | VTHQGLSSPVTKSFNRGECG | | | |
| | | | GGGSGGGGSGGGGSGGGGSD | | | |
| | | | IQMTQSPSSLSASVGDRVTI | | | |
| | | | TCKSSQSLLYRSNQKNYLAW | | | |
| | | | YQQKPGKAPKLLIYWASTRE | | | |
| | | | SGVPSRFSGSGSGTDFTLTI | | | |
| | | | SSLQPEDFATYYCQQYYNYP | | | |
| | | | RTFGQGTKVEIKRTVAAPSV | | | |
| | | | FIFPPSDEQLKSGTASVVCL | | | |
| | | | LNNFYPREAKVQWKVDNALQ | | | |
| | | | SGNSQESVTEQDSKDSTYSL | | | |
| | | | SSTLTLSKADYEKHKVYACE | | | |
| | | | VTHQGLSSPVTKSFNRGEC | | | |
| TCE-85 | (None) | (None) | EVQLVESGGGLVQPGGSLKL | 338 | EVQLVESGGGLVQPGGSLRL | 351 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GS | | | QTVVTQEPSLTVSPGGTVTL | | STSGGTAALGCLVKDYFPEP | |
| (SEQ | | | TCGSSTGAVTSGNYPNWVQQ | | VTVSWNSGALTSGVHTFPAV | |
| ID NO: | | | KPGQAPRGLIGGTKFLAPGT | | LQSSGLYSLSSVVTVPSSSL | |
| 270); L₂ | | | PARFSGSLLGGKAALTLSGV | | GTQTYICNVNHKPSNTKVDK | |
| GGGG | | | QPEDEAEYYCVLWYSNRWVF | | KVEPKSC | |
| SGGG | | | GGGTKLTVLGGGGSGGGGSD | | | |
| GSGG | | | IQMTQSPSSLSASVGDRVTI | | | |
| GGSG | | | TCKSSQSLLYRSNQKNYLAW | | | |
| GGGS | | | YQQKPGKAPKLLIYWASTRE | | | |
| GGGG | | | SGVPSRFSGSGSGTDFTLTI | | | |
| S (SEQ | | | SSLQPEDFATYYCQQYYNYP | | | |
| ID NO: | | | RTFGQGTKVEIKRTVAAPSV | | | |
| 272) | | | FIFPPSDEQLKSGTASVVCL | | | |
| | | | LNNFYPREAKVQWKVDNALQ | | | |
| | | | SGNSQESVTEQDSKDSTYSL | | | |
| | | | SSTLTLSKADYEKHKVYACE | | | |
| | | | VTHQGLSSPVTKSFNRGECG | | | |
| | | | GGGSGGGGSGGGGSGGGGSG | | | |
| | | | GGGSDIQMTQSPSSLSASVG | | | |
| | | | DRVTITCKSSQSLLYRSNQK | | | |
| | | | NYLAWYQQKPGKAPKLLIYW | | | |
| | | | ASTRESGVPSRFSGSGSGTD | | | |
| | | | FTLTISSLQPEDFATYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGEC | | | |
| TCE-86 | (None) | (None) | EVQLVESGGGLVQPGGSLKL | 339 | EVQLVESGGGLVQPGGSLRL | 352 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GSGG | | | QTVVTQEPSLTVSPGGTVTL | | STSGGTAALGCLVKDYFPEP | |
| GGS | | | TCGSSTGAVTSGNYPNWVQQ | | VTVSWNSGALTSGVHTFPAV | |
| (SEQ | | | KPGQAPRGLIGGTKFLAPGT | | LQSSGLYSLSSVVTVPSSSL | |
| ID NO: | | | PARFSGSLLGGKAALTLSGV | | GTQTYICNVNHKPSNTKVDK | |
| 222); L₂ | | | QPEDEAEYYCVLWYSNRWVF | | KVEPKSC | |
| GGGG | | | GGGTKLTVLGGGGSGGGGSG | | | |
| SGGG | | | GGGSDIQMTQSPSSLSASVG | | | |
| GS | | | DRVTITCKSSQSLLYRSNQK | | | |
| (SEQ | | | NYLAWYQQKPGKAPKLLIYW | | | |
| ID NO: | | | ASTRESGVPSRFSGSGSGTD | | | |
| 270) | | | FTLTISSLQPEDFATYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGECGGGGSGGGGSDIQMTQ | | | |
| | | | SPSSLSASVGDRVTITCKSS | | | |
| | | | QSLLYRSNQKNYLAWYQQKP | | | |
| | | | GKAPKLLIYWASTRESGVPS | | | |
| | | | RFSGSGSGTDFTLTISSLQP | | | |
| | | | EDFATYYCQQYYNYPRTFGQ | | | |
| | | | GTKVEIKRTVAAPSVFIFPP | | | |
| | | | SDEQLKSGTASVVCLLNNFY | | | |
| | | | PREAKVQWKVDNALQSGNSQ | | | |
| | | | ESVTEQDSKDSTYSLSSTLT | | | |
| | | | LSKADYEKHKVYACEVTHQG | | | |
| | | | LSSPVTKSFNRGEC | | | |
| TCE-87 | (None) | (None) | EVQLVESGGGLVQPGGSLKL | 340 | EVQLVESGGGLVQPGGSLRL | 353 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GSGG | | | QTVVTQEPSLTVSPGGTVTL | | STSGGTAALGCLVKDYFPEP | |
| GGS | | | TCGSSTGAVTSGNYPNWVQQ | | VTVSWNSGALTSGVHTFPAV | |
| (SEQ | | | KPGQAPRGLIGGTKFLAPGT | | LQSSGLYSLSSVVTVPSSSL | |
| ID NO: | | | PARFSGSLLGGKAALTLSGV | | GTQTYICNVNHKPSNTKVDK | |
| 222); L₂ | | | QPEDEAEYYCVLWYSNRWVF | | KVEPKSC | |
| GGGG | | | GGGTKLTVLGGGGSGGGGSG | | | |
| SGGG | | | GGGSDIQMTQSPSSLSASVG | | | |
| GSGG | | | DRVTITCKSSQSLLYRSNQK | | | |
| GGS | | | NYLAWYQQKPGKAPKLLIYW | | | |
| (SEQ | | | ASTRESGVPSRFSGSGSGTD | | | |
| ID NO: | | | FTLTISSLQPEDFATYYCQQ | | | |
| 222) | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGECGGGGSGGGGSGGGGSD | | | |
| | | | IQMTQSPSSLSASVGDRVTI | | | |
| | | | TCKSSQSLLYRSNQKNYLAW | | | |
| | | | YQQKPGKAPKLLIYWASTRE | | | |
| | | | SGVPSRFSGSGSGTDFTLTI | | | |
| | | | SSLQPEDFATYYCQQYYNYP | | | |
| | | | RTFGQGTKVEIKRTVAAPSV | | | |
| | | | FIFPPSDEQLKSGTASVVCL | | | |
| | | | LNNFYPREAKVQWKVDNALQ | | | |
| | | | SGNSQESVTEQDSKDSTYSL | | | |
| | | | SSTLTLSKADYEKHKVYACE | | | |
| | | | VTHQGLSSPVTKSFNRGEC | | | |
| TCE-88 | (None) | (None) | EVQLVESGGGLVQPGGSLKL | 341 | EVQLVESGGGLVQPGGSLRL | 354 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GSGG | | | QTVVTQEPSLTVSPGGTVTL | | STSGGTAALGCLVKDYFPEP | |
| GGS | | | TCGSSTGAVTSGNYPNWVQQ | | VTVSWNSGALTSGVHTFPAV | |
| (SEQ | | | KPGQAPRGLIGGTKFLAPGT | | LQSSGLYSLSSVVTVPSSSL | |
| ID NO: | | | PARFSGSLLGGKAALTLSGV | | GTQTYICNVNHKPSNTKVDK | |
| 222); L₂ | | | QPEDEAEYYCVLWYSNRWVF | | KVEPKSC | |
| GGGG | | | GGGTKLTVLGGGGSGGGGSG | | | |
| SGGG | | | GGGSDIQMTQSPSSLSASVG | | | |
| GSGG | | | DRVTITCKSSQSLLYRSNQK | | | |
| GGSG | | | NYLAWYQQKPGKAPKLLIYW | | | |
| GGGS | | | ASTRESGVPSRFSGSGSGTD | | | |
| (SEQ | | | FTLTISSLQPEDFATYYCQQ | | | |
| ID NO: | | | YYNYPRTFGQGTKVEIKRTV | | | |
| 271) | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGECGGGGSGGGGSGGGGSG | | | |
| | | | GGGSDIQMTQSPSSLSASVG | | | |
| | | | DRVTITCKSSQSLLYRSNQK | | | |
| | | | NYLAWYQQKPGKAPKLLIYW | | | |
| | | | ASTRESGVPSRFSGSGSGTD | | | |
| | | | FTLTISSLQPEDFATYYCQQ | | | |
| | | | YYNYPRTFGQGTKVEIKRTV | | | |
| | | | AAPSVFIFPPSDEQLKSGTA | | | |
| | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGEC | | | |
| TCE-89 | (None) | (None) | EVQLVESGGGLVQPGGSLKL | 342 | EVQLVESGGGLVQPGGSLRL | 355 |
| [2 + 1] | AND | AND | SCAASGFTFNKYAMNWVRQA | | SCAVSGYSITSDYAWNWVRQ | |
| N-term | (None) | (None) | PGKGLEWVARIRSKYNNYAT | | APGKGLEWVGYISNSGSTSY | |
| CD3 | | | YYADSVKDRFTISRDDSKNT | | NPSLKSRFTISRDTSKNTLY | |
| scFv; L₁ | | | AYLQMNNLKTEDTAVYYCVR | | LQMNSLRAEDTAVYYCARER | |
| GGGG | | | HGNFGNSYISYWAYWGQGTL | | NYDYDDYYYAMDYWGQGTLV | |
| SGGG | | | VTVSSGGGGSGGGGSGGGGS | | TVSSASTKGPSVFPLAPSSK | |
| GSGG | | | QTVVTQEPSLTVSPGGTVTL | | STSGGTAALGCLVKDYFPEP | |
| GGS | | | TCGSSTGAVTSGNYPNWVQQ | | VTVSWNSGALTSGVHTFPAV | |
| (SEQ | | | KPGQAPRGLIGGTKFLAPGT | | LQSSGLYSLSSVVTVPSSSL | |
| ID NO: | | | PARFSGSLLGGKAALTLSGV | | GTQTYICNVNHKPSNTKVDK | |
| 222); L₂ | | | QPEDEAEYYCVLWYSNRWVF | | KVEPKSC | |
| GGGG | | | GGGTKLTVLGGGGSGGGGSG | | | |
| SGGG | | | GGGSDIQMTQSPSSLSASVG | | | |
| GSGG | | | DRVTITCKSSQSLLYRSNQK | | | |
| GGSG | | | NYLAWYQQKPGKAPKLLIYW | | | |
| GGGS | | | ASTRESGVPSRFSGSGSGTD | | | |
| GGGG | | | FTLTISSLQPEDFATYYCQQ | | | |
| S (SEQ | | | YYNYPRTFGQGTKVEIKRTV | | | |
| ID NO: | | | AAPSVFIFPPSDEQLKSGTA | | | |
| 272) | | | SVVCLLNNFYPREAKVQWKV | | | |
| | | | DNALQSGNSQESVTEQDSKD | | | |
| | | | STYSLSSTLTLSKADYEKHK | | | |
| | | | VYACEVTHQGLSSPVTKSFN | | | |
| | | | RGECGGGGSGGGGSGGGGSG | | | |
| | | | GGGSGGGGSDIQMTQSPSSL | | | |
| | | | SASVGDRVTITCKSSQSLLY | | | |
| | | | RSNQKNYLAWYQQKPGKAPK | | | |
| | | | LLIYWASTRESGVPSRFSGS | | | |
| | | | GSGTDFTLTISSLQPEDFAT | | | |
| | | | YYCQQYYNYPRTFGQGTKVE | | | |
| | | | IKRTVAAPSVFIFPPSDEQL | | | |
| | | | KSGTASVVCLLNNFYPREAK | | | |
| | | | VQWKVDNALQSGNSQESVTE | | | |
| | | | QDSKDSTYSLSSTLTLSKAD | | | |
| | | | YEKHKVYACEVTHQGLSSPV | | | |
| | | | TKSFNRGEC | | | |

In some embodiments, the polypeptide or polypeptide complex comprises an amino acid sequence that comprises the immunoglobulin light chain that binds to STEAP-1 is selected from SEQ ID NOs: 150-173 and an amino acid sequence that comprises the immunoglobulin heavy chain that binds to STEAP-1 is selected from SEQ ID NOs: 198-221. In some embodiments, the polypeptide or polypeptide complex comprises the amino acid sequences of SEQ ID NO: 150 and SEQ ID NO: 198. In some embodiments, the polypeptide or polypeptide complex comprises the amino acid sequences of SEQ ID NO: 151 and SEQ ID NO: 199.

In some embodiments, the polypeptide complex comprises amino acid sequences with at least 90%, 95%, 96%, 97%, 98%, 99%, or 100% sequence identity to SEQ ID NO: 152 and SEQ ID NO: 200. In some embodiments, the polypeptide complex comprises the amino acid sequences of SEQ ID NO: 152 and SEQ ID NO: 200. In some embodiments, the polypeptide complex comprises amino acid sequences with at least 90%, 95%, 96%, 97%, 98%, 99%, or 100% sequence identity to SEQ ID NO: 153 and SEQ ID NO: 201. In some embodiments, the polypeptide complex comprises the amino acid sequences of SEQ ID NO: 153 and SEQ ID NO: 201. In some embodiments, the polypeptide complex comprises amino acid sequences with at least 90%, 95%, 96%, 97%, 98%, 99%, or 100% sequence identity to SEQ ID NO: 154 and SEQ ID NO: 202. In some embodiments, the polypeptide complex comprises the amino acid sequences of SEQ ID NO: 154 and SEQ ID NO: 202. In some embodiments, the polypeptide complex comprises amino acid sequences with at least 90%, 95%, 96%, 97%, 98%, 99%, or 100% sequence identity to SEQ ID NO: 155 and SEQ ID NO: 203. In some embodiments, the polypeptide complex comprises the amino acid sequences of SEQ ID NO: 155 and SEQ ID NO: 203. In some embodiments, the polypeptide complex comprises amino acid sequences with at least 90%, 95%, 96%, 97%, 98%, 99%, or 100% sequence identity to SEQ ID NO: 156 and SEQ ID NO: 204. In some embodiments, the polypeptide complex comprises the amino acid sequences of SEQ ID NO: 156 and SEQ ID NO: 204. In some embodiments, the polypeptide complex comprises amino acid sequences with at least 90%, 95%, 96%, 97%, 98%, 99%, or 100% sequence identity to SEQ ID NO: 157 and SEQ ID NO: 205. In some embodiments, the polypeptide complex comprises the amino acid sequences of SEQ ID NO: 157 and SEQ ID NO: 205. In some embodiments, the polypeptide complex comprises amino acid sequences with at least 90%, 95%, 96%, 97%, 98%, 99%, or 100% sequence identity to SEQ ID NO: 158 and SEQ ID NO: 206. In some embodiments, the polypeptide complex comprises the amino acid sequences of SEQ ID NO: 158 and SEQ ID NO: 206. In some embodiments, the polypeptide complex comprises amino acid sequences with at least 90%, 95%, 96%, 97%, 98%, 99%, or 100% sequence identity to SEQ ID NO: 159 and SEQ ID NO: 207. In some embodiments, the polypeptide complex comprises the amino acid sequences of SEQ ID NO: 159 and SEQ ID NO: 207. In some embodiments, the polypeptide complex comprises amino acid sequences with at least 90%, 95%, 96%, 97%, 98%, 99%, or 100% sequence identity to SEQ ID NO: 160 and SEQ ID NO: 208. In some embodiments, the polypeptide complex comprises the amino acid sequences of SEQ ID NO: 160 and SEQ ID NO: 208. In some embodiments, the polypeptide complex comprises amino acid sequences with at least 90%, 95%, 96%, 97%, 98%, 99%, or 100% sequence identity to SEQ ID NO: 161 and SEQ ID NO: 209. In some embodiments, the polypeptide complex comprises the amino acid sequences of SEQ ID NO: 161 and SEQ ID NO: 209. In some embodiments, the polypeptide complex comprises amino acid sequences with at least 90%, 95%, 96%, 97%, 98%, 99%, or 100% sequence identity to SEQ ID NO: 162 and SEQ ID NO: 210. In some embodiments, the polypeptide complex comprises the amino acid sequences of SEQ ID NO: 162 and SEQ ID NO: 210.

In some embodiments, the polypeptide complex comprises amino acid sequences with at least 90%, 95%, 96%, 97%, 98%, 99%, or 100% sequence identity to SEQ ID NO: 163 and SEQ ID NO: 211. In some embodiments, the polypeptide complex comprises the amino acid sequences of SEQ ID NO: 163 and SEQ ID NO: 211. In some embodiments, the polypeptide complex comprises amino acid sequences with at least 90%, 95%, 96%, 97%, 98%, 99%, or 100% sequence identity to SEQ ID NO: 164 and SEQ ID NO: 212. In some embodiments, the polypeptide complex comprises the amino acid sequences of SEQ ID NO: 164 and SEQ ID NO: 212. In some embodiments, the polypeptide complex comprises amino acid sequences with at least 90%, 95%, 96%, 97%, 98%, 99%, or 100% sequence identity to SEQ ID NO: 165 and SEQ ID NO: 213. In some embodiments, the polypeptide complex comprises the amino acid sequences of SEQ ID NO: 165 and SEQ ID NO: 213. In some embodiments, the polypeptide complex comprises amino acid sequences with at least 90%, 95%, 96%, 97%, 98%, 99%, or 100% sequence identity to SEQ ID NO: 166 and SEQ ID NO: 214. In some embodiments, the polypeptide complex comprises the amino acid sequences of SEQ ID NO: 166 and SEQ ID NO: 214. In some embodiments, the polypeptide complex comprises amino acid sequences with at least 90%, 95%, 96%, 97%, 98%, 99%, or 100% sequence identity to SEQ ID NO: 167 and SEQ ID NO: 215. In some embodiments, the polypeptide complex comprises the amino acid sequences of SEQ ID NO: 167 and SEQ ID NO: 215. In some embodiments, the polypeptide complex comprises amino acid sequences with at least 90%, 95%, 96%, 97%, 98%, 99%, or 100% sequence identity to SEQ ID NO: 168 and SEQ ID NO: 216. In some embodiments, the polypeptide complex comprises the amino acid sequences of SEQ ID NO: 168 and SEQ ID NO: 216. In some embodiments, the polypeptide complex comprises amino acid sequences with at least 90%, 95%, 96%, 97%, 98%, 99%, or 100% sequence identity to SEQ ID NO: 169 and SEQ ID NO: 217. In some embodiments, the polypeptide complex comprises the amino acid sequences of SEQ ID NO: 169 and SEQ ID NO: 217. In some embodiments, the polypeptide complex comprises amino acid sequences with at least 90%, 95%, 96%, 97%, 98%, 99%, or 100% sequence identity to SEQ ID NO: 170 and SEQ ID NO: 218. In some embodiments, the polypeptide complex comprises the amino acid sequences of SEQ ID NO: 170 and SEQ ID NO: 218. In some embodiments, the polypeptide complex comprises amino acid sequences with at least 90%, 95%, 96%, 97%, 98%, 99%, or 100% sequence identity to SEQ ID NO: 171 and SEQ ID NO: 219. In some embodiments, the polypeptide complex comprises the amino acid sequences of SEQ ID NO: 171 and SEQ ID NO: 219. In some embodiments, the polypeptide complex comprises amino acid sequences with at least 90%, 95%, 96%, 97%, 98%, 99%, or 100% sequence identity to SEQ ID NO: 172 and SEQ ID NO: 220. In some embodiments, the polypeptide complex comprises the amino acid sequences of SEQ ID NO: 172 and SEQ ID NO: 220. In some embodiments, the polypeptide complex comprises amino acid sequences with at least 90%, 95%, 96%, 97%, 98%, 99%, or 100% sequence identity to SEQ ID NO: 173 and SEQ ID NO: 221. In some embodiments, the polypeptide complex comprises the amino acid sequences of SEQ ID NO: 173 and SEQ ID NO: 221.

In some embodiments, the polypeptide complex comprises amino acid sequences with at least 90%, 95%, 96%, 97%, 98%, 99%, or 100% sequence identity to SEQ ID NO: 330 and SEQ ID NO: 343. In some embodiments, the polypeptide complex comprises the amino acid sequences of SEQ ID NO: 330 and SEQ ID NO: 343. In some embodiments, the polypeptide complex comprises amino acid sequences with at least 90%, 95%, 96%, 97%, 98%, 99%, or 100% sequence identity to SEQ ID NO: 331 and SEQ ID NO: 344. In some embodiments, the polypeptide complex comprises the amino acid sequences of SEQ ID NO: 331 and SEQ ID NO: 344. In some embodiments, the polypeptide complex comprises amino acid sequences with at least 90%, 95%, 96%, 97%, 98%, 99%, or 100% sequence identity to SEQ ID NO: 332 and SEQ ID NO: 345. In some embodiments, the polypeptide complex comprises the amino acid sequences of SEQ ID NO: 332 and SEQ ID NO: 345. In some embodiments, the polypeptide complex comprises amino acid sequences with at least 90%, 95%, 96%, 97%, 98%, 99%, or 100% sequence identity to SEQ ID NO: 333 and SEQ ID NO: 346. In some embodiments, the polypeptide complex comprises the amino acid sequences of SEQ ID NO: 333 and SEQ ID NO: 346. In some embodiments, the polypeptide complex comprises amino acid sequences with at least 90%, 95%, 96%, 97%, 98%, 99%, or 100% sequence identity to SEQ ID NO: 334 and SEQ ID NO: 347. In some embodiments, the polypeptide complex comprises the amino acid sequences of SEQ ID NO: 334 and SEQ ID NO: 347. In some embodiments, the polypeptide complex comprises amino acid sequences with at least 90%, 95%, 96%, 97%, 98%, 99%, or 100% sequence identity to SEQ ID NO: 335 and SEQ ID NO: 348. In some embodiments, the polypeptide complex comprises the amino acid sequences of SEQ ID NO: 335 and SEQ ID NO: 348. In some embodiments, the polypeptide complex comprises amino acid sequences with at least 90%, 95%, 96%, 97%, 98%, 99%, or 100% sequence identity to SEQ ID NO: 336 and SEQ ID NO: 349. In some embodiments, the polypeptide complex comprises the amino acid sequences of SEQ ID NO: 336 and SEQ ID NO: 349. In some embodiments, the polypeptide complex comprises amino acid sequences with at least 90%, 95%, 96%, 97%, 98%, 99%, or 100% sequence identity to SEQ ID NO: 337 and SEQ ID NO: 350. In some embodiments, the polypeptide complex comprises the amino acid sequences of SEQ ID NO: 337 and SEQ ID NO: 350. In some embodiments, the polypeptide complex comprises amino acid sequences with at least 90%, 95%, 96%, 97%, 98%, 99%, or 100% sequence identity to SEQ ID NO: 338 and SEQ ID NO: 351. In some embodiments, the polypeptide complex comprises the amino acid sequences of SEQ ID NO: 351 and SEQ ID NO: 351. In some embodiments, the polypeptide complex comprises amino acid sequences with at least 90%, 95%, 96%, 97%, 98%, 99%, or 100% sequence identity to SEQ ID NO: 339 and SEQ ID NO: 352. In some embodiments, the polypeptide complex comprises the amino acid sequences of SEQ ID NO: 339 and SEQ ID NO: 352. In some embodiments, the polypeptide complex comprises amino acid sequences with at least 90%, 95%, 96%, 97%, 98%, 99%, or 100% sequence identity to SEQ ID NO: 340 and SEQ ID NO: 353. In some embodiments, the polypeptide complex comprises the amino acid sequences of SEQ ID NO: 340 and SEQ ID NO: 353. In some embodiments, the polypeptide complex comprises amino acid sequences with at least 90%, 95%, 96%, 97%, 98%, 99%, or 100% sequence identity to SEQ ID NO: 341 and SEQ ID NO: 354. In some embodiments, the polypeptide complex comprises the amino acid sequences of SEQ ID NO: 341 and SEQ ID NO: 354. In some embodiments, the polypeptide complex comprises amino acid sequences with at least 90%, 95%, 96%, 97%, 98%, 99%, or 100% sequence identity to SEQ ID NO: 342 and SEQ ID NO: 355. In some embodiments, the polypeptide complex comprises the amino acid sequences of SEQ ID NO: 342 and SEQ ID NO: 355.

In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 152-161 and 164-173, and an amino acid sequence, that comprises the immunoglobulin heavy chain that binds to STEAP-1, with at least 95%, 98%, 99%, or 100% sequence identity to any one of SEQ ID NOs: 200-209 and 212-221. In some embodiments, the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 150-151, 162-163, and 330-342 and an amino acid sequence, that comprises the immunoglobulin heavy chain that binds to STEAP-1, with at least 95%, 98%, 99%, or 100% sequence identity to any one of SEQ ID NOs: 198-199, 210-211, and 343-355.

In some embodiments, the polypeptide or polypeptide complex comprises amino acid sequences with at least 95%, 98%, or 99% sequence identity to SEQ ID NOs: 324, 325, and 326. In some embodiments, the polypeptide or polypeptide complex comprises amino acid sequences with at least 95%, 98%, or 99% sequence identity to SEQ ID NOs: 327, 328, and 329. In some embodiments, the polypeptide or polypeptide complex comprises amino acid sequences with at least 95%, 98%, or 99% sequence identity to SEQ ID NOs: 356, 357, 358, and 359. In some embodiments, the polypeptide or polypeptide complex comprises amino acid sequences with at least 95%, 98%, or 99% sequence identity to SEQ ID NOs: 360, 361, 362, and 363. In some embodiments, the polypeptide or polypeptide complex comprises amino acid sequences with at least 95%, 98%, or 99% sequence identity to SEQ ID NOs: 364, 365, 366, and 367. In some embodiments, the polypeptide or polypeptide complex comprises amino acid sequences with at least 95%, 98%, or 99% sequence identity to SEQ ID NOs: 368, 369, and 370.

TABLE 15
Anti-STEAP-1 and Anti-CD3 T-cell engager (TCE) CrossFab Sequences (1 + 1)

| | Amino Acid | SEQ | Amino Acid | SEQ | Amino Acid | SEQ |
|---|---|---|---|---|---|---|
| Name/ | Sequence 1 | ID | Sequence 2 | ID | Sequence 3 | ID |
| Format | (N to C) | NO: | (N to C) | NO: | (N to C) | NO: |
| TCE-69 | DIQMTQSPSSLS | 324 | EVQLVESGGGLVQP | 325 | EVQLVESGGGLVQPGG | 326 |
| (1 + 1) | ASVGDRVTITCK | | GGSLKLSCAASGFTF | | SLRLSCAVSGYSITSDY | |
| N-term | SSQSLLYRSNQK | | NKYAMNWVRQAPG | | AWNWVRQAPGKGLEW | |
| SBD-7 | NYLAWYQQKPG | | KGLEWVARIRSKYN | | VGYISNSGSTSYNPSLK | |
| Fab; | KAPKLLIYWAST | | NYATYYADSVKDRF | | SRFTISRDTSKNTLYLQ | |
| CD3 | RESGVPSRFSGS | | TISRDDSKNTAYLQ | | MNSLRAEDTAVYYCAR | |
| CrossFab; | GSGTDFTLTISSL | | MNNLKTEDTAVYY | | ERNYDYDDYYYAMDY | |
| DGGG | QPEDFATYYCQ | | CVRHGNFGNSYISY | | WGQGTLVTVSSASTKG | |
| GSGG | QYYNYPRTFGQ | | WAYWGQGTLVTVS | | PSVFPLAPSSKSTSGGT | |
| GGS | GTKVEIKRTVAA | | SASVAAPSVFIFPPSD | | AALGCLVEDYFPEPVT | |
| linker | PSVFIFPPSDRKL | | EQLKSGTASVVCLL | | VSWNSGALTSGVHTFP | |
| (SEQ | KSGTASVVCLL | | NNFYPREAKVQWK | | AVLQSSGLYSLSSVVTV | |
| ID NO: | NNFYPREAKVQ | | VDNALQSGNSQESV | | PSSSLGTQTYICNVNHK | |
| 371) | WKVDNALQSGN | | TEQDSKDSTYSLSST | | PSNGKVDEKVEPKSCD | |
| STEAP-1 | SQESVTEQDSKD | | LTLSKADYEKHKVY | | GGGGSGGGGSQTVVTQ | |
| Mutations | STYSLSSTLTLSK | | ACEVTHQGLSSPVT | | EPSLTVSPGGTVTLTCG | |
| (LC; | ADYEKHKVYAC | | KSFNRGEC | | SSTGAVTSGNYPNWVQ | |
| HC): | EVTHQGLSSPVT | | | | QKPGQAPRGLIGGTKFL | |
| E129R, | KSFNRGEC | | | | APGTPARFSGSLLGGKA | |
| Q130K; | | | | | ALTLSGVQPEDEAEYY | |
| K154E, | | | | | CVLWYSNRWVFGGGT | |
| K220E | | | | | KLTVLSSASTKGPSVFP | |
| | | | | | LAPSSKSTSGGTAALGC | |
| | | | | | LVKDYFPEPVTVSWNS | |
| | | | | | GALTSGVHTFPAVLQSS | |
| | | | | | GLYSLSSVVTVPSSSLG | |
| | | | | | TQTYICNVNHKPSNTK | |
| | | | | | VDGKVEPKSC | |
| TCE-70 | DIQMTQSPSSLS | 327 | QTVVTQEPSLTVSPG | 328 | EVQL VESGGGLVQPGG | 329 |
| (1 + 1) | ASVGDRVTITCK | | GTVTLTCGSSTGAV | | SLRLSCAVSGYSITSDY | |
| N-term | SSQSLLYRSNQK | | TSGNYPNWVQQKP | | AWNWVRQAPGKGLEW | |
| SBD-1; | NYLAWYQQKPG | | GQAPRGLIGGTKFL | | VGYISNSGSTSYNPSLK | |
| CD3 | KAPKLLIYWAST | | APGTPARFSGSLLGG | | SRFTISRDTSKNTLYLQ | |
| CrossFab; | RESGVPSRFSGS | | KAALTLSGVQPEDE | | MNSLRAEDTAVYYCAR | |
| DGGG | GSGTDFTLTISSL | | AEYYCVLWYSNRW | | ERNYDYDDYYYAMDY | |
| GSGG | QPEDFATYYCQ | | VFGGGTKLTVLSSA | | WGQGTLVTVSSASTKG | |
| GGS | QYYNYPRTFGQ | | STKGPSVFPLAPSSK | | PSVFPLAPSSKSTSGGT | |
| linker | GTKVEIKRTVAA | | STSGGTAALGCLVK | | AALGCLVKDYFPEPVT | |
| (SEQ | PSVFIFPPSDEQL | | DYFPEPVTVSWNSG | | VSWNSGALTSGVHTFP | |
| ID NO: | KSGTASVVCLL | | ALTSGVHTFPAVLQ | | AVLQSSGLYSLSSVVTV | |
| 371) | NNFYPREAKVQ | | SSGLYSLSSVVTVPS | | PSSSLGTQTYICNVNHK | |
| | WKVDNALQSGN | | SSLGTQTYICNVNH | | PSNTKVDKKVEPKSCD | |
| | SQESVTEQDSKD | | KPSNTKVDKKVEPK | | GGGGSGGGGSEVQLVE | |
| | STYSLSSTLTLSK | | SC | | SGGGLVQPGGSLKLSC | |
| | ADYEKHKVYAC | | | | AASGFTFNKYAMNWV | |
| | EVTHQGLSSPVT | | | | RQAPGKGLEWVARIRS | |
| | KSFNRGEC | | | | KYNNYATYYADSVKD | |
| | | | | | RFTISRDDSKNTAYLQ | |
| | | | | | MNNLKTEDTAVYYCV | |
| | | | | | RHGNFGNSYISYWAYW | |
| | | | | | GQGTLVTVSSASVAAP | |
| | | | | | SVFIFPPSDEQLKSGTAS | |
| | | | | | VVCLLNNFYPREAKVQ | |
| | | | | | WKVDNALQSGNSQESV | |
| | | | | | TEQDSKDSTYSLSSTLT | |
| | | | | | LSKADYEKHKVYACEV | |
| | | | | | THQGLSSPVTKSFNRGE | |
| | | | | | C | |

TABLE 16
Anti-STEAP-1 and Anti-CD3 T-cell engager (TCE) CrossFab Sequences (2 + 1)

| | Amino Acid | SEQ | Amino Acid | SEQ | Amino Acid | SEQ | Amino Acid | SEQ |
|---|---|---|---|---|---|---|---|---|
| Name/ | Sequence 1 | ID | Sequence 2 | ID | Sequence 3 | ID | Sequence 4 | ID |
| Format | (N to C) | NO: | (Nt o C) | NO: | (N to C) | NO: | (N to C) | NO: |
| TCE-90 | DIQMTQSPS | 356 | EVQLVESGG | 357 | EVQLVESGGG | 358 | EVQLVESG | 359 |
| N-term | SLSASVGDR | | GLVQPGGSL | | LVQPGGSLKL | | GGLVQPGG | |
| SBD | VTITCKSSQS | | RLSCAVSGYS | | SCAASGFTFN | | SLRLSCAVS | |
| Fab; | LLYRSNQKN | | ITSDYAWNW | | KYAMNWVRQ | | GYSITSDYA | |
| C-term | YLAWYQQK | | VRQAPGKGL | | APGKGLEWV | | WNWVRQA | |
| CD3 | PGKAPKLLI | | EWVGYISNS | | ARIRSKYNNY | | PGKGLEWV | |
| CrossFab; | YWASTRESG | | GSTSYNPSLK | | ATYYADSVKD | | GYISNSGST | |
| L₁ = DG | VPSRFSGSG | | SRFTISRDTSK | | RFTISRDDSKN | | SYNPSLKSR | |
| GGGS | SGTDFTLTIS | | NTLYLQMNS | | TAYLQMNNL | | FTISRDTSK | |
| GGGG | SLQPEDFAT | | LRAEDTAVY | | KTEDTAVYYC | | NTLYLQMN | |
| S (SEQ | YYCQQYYN | | YCARERNYD | | VRHGNFGNSY | | SLRAEDTA | |
| ID NO: | YPRTFGQGT | | YDDYYYAM | | ISYWAYWGQ | | VYYCARER | |
| 371); | KVEIKRTVA | | DYWGQGTLV | | GTLVTVSSAS | | NYDYDDY | |
| Hinge = | APSVFIFPPS | | TVSSASTKGP | | VAAPSVFIFPP | | YYAMDYW | |
| DKTHT | DEQLKSGTA | | SVFPLAPSSK | | SDEQLKSGTA | | GQGTLVTV | |
| CPPCP | SVVCLLNNF | | STSGGTAALG | | SVVCLLNNFY | | SSASTKGPS | |
| APELL | YPREAKVQ | | CLVKDYFPEP | | PREAKVQWK | | VFPLAPSSK | |
| GG | WKVDNALQ | | VTVSWNSGA | | VDNALQSGNS | | STSGGTAA | |
| (SEQ | SGNSQESVT | | LTSGVHTFPA | | QESVTEQDSK | | LGCLVKDY | |
| ID NO: | EQDSKDSTY | | VLQSSGLYSL | | DSTYSLSSTLT | | FPEPVTVS | |
| 377); | SLSSTLTLSK | | SSVVTVPSSS | | LSKADYEKHK | | WNSGALTS | |
| CD3 | ADYEKHKV | | LGTQTYICNV | | VYACEVTHQG | | GVHTFPAV | |
| LC: | YACEVTHQ | | NHKPSNTKV | | LSSPVTKSFNR | | LQSSGLYSL | |
| Replaced | GLSSPVTKS | | DKKVEPKSC | | GEC | | SSVVTVPSS | |
| “RT” | FNRGEC | | DKTHTCPPCP | | | | SLGTQTYIC | |
| with | | | APELLGG | | | | NVNHKPSN | |
| “AS” in | | | | | | | TKVDKKVE | |
| constant | | | | | | | PKSCDGGG | |
| kappa | | | | | | | GSGGGGSQ | |
| light | | | | | | | TVVTQEPS | |
| chain; | | | | | | | LTVSPGGT | |
| CD3 | | | | | | | VTLTCGSST | |
| HC: | | | | | | | GAVTSGNY | |
| Inserted | | | | | | | PNWVQQKP | |
| “SS” | | | | | | | GQAPRGLI | |
| between | | | | | | | GGTKFLAP | |
| VL-CH1 | | | | | | | GTPARFSGS | |
| junction | | | | | | | LLGGKAAL | |
| | | | | | | | TLSGVQPE | |
| | | | | | | | DEAEYYCV | |
| | | | | | | | LWYSNRW | |
| | | | | | | | VFGGGTKL | |
| | | | | | | | TVLSSASTK | |
| | | | | | | | GPSVFPLAP | |
| | | | | | | | SSKSTSGGT | |
| | | | | | | | AALGCLVK | |
| | | | | | | | DYFPEPVT | |
| | | | | | | | VSWNSGAL | |
| | | | | | | | TSGVHTFP | |
| | | | | | | | AVLQSSGL | |
| | | | | | | | YSLSSVVT | |
| | | | | | | | VPSSSLGTQ | |
| | | | | | | | TYICNVNH | |
| | | | | | | | KPSNTKVD | |
| | | | | | | | KKVEPKSC | |
| | | | | | | | DKTHTCPP | |
| | | | | | | | CPAPELLG | |
| | | | | | | | G | |
| TCE-91 | DIQMTQSPS | 360 | EVQLVESGG | 361 | QTVVTQEPSL | 362 | EVQLVESG | 363 |
| N-term | SLSASVGDR | | GLVQPGGSL | | TVSPGGTVTL | | GGLVQPGG | |
| SBD | VTITCKSSQS | | RLSCAVSGYS | | TCGSSTGAVT | | SLRLSCAVS | |
| Fab; | LLYRSNQKN | | ITSDYAWNW | | SGNYPNWVQ | | GYSITSDYA | |
| C-term | YLAWYQQK | | VRQAPGKGL | | QKPGQAPRGL | | WNWVRQA | |
| CD3 | PGKAPKLLI | | EWVGYISNS | | IGGTKFLAPGT | | PGKGLEWV | |
| CrossFab; | YWASTRESG | | GSTSYNPSLK | | PARFSGSLLGG | | GYISNSGST | |
| L1 = DG | VPSRFSGSG | | SRFTISRDTSK | | KAALTLSGVQ | | SYNPSLKSR | |
| GGGS | SGTDFTLTIS | | NTLYLQMNS | | PEDEAEYYCV | | FTISRDTSK | |
| GGGG | SLQPEDFAT | | LRAEDTAVY | | LWYSNRWVF | | NTLYLQMN | |
| S (SEQ | YYCQQYYN | | YCARERNYD | | GGGTKLTVLS | | SLRAEDTA | |
| ID NO: | YPRTFGQGT | | YDDYYYAM | | SASTKGPSVFP | | VYYCARER | |
| 371); | KVEIKRTVA | | DYWGQGTLV | | LAPSSKSTSGG | | NYDYDDY | |
| D221E | APSVFIFPPS | | TVSSASTKGP | | TAALGCLVKD | | YYAMDYW | |
| Hinge = | DEQLKSGTA | | SVFPLAPSSK | | YFPEPVTVSW | | GQGTLVTV | |
| EKTHT | SVVCLLNNF | | STSGGTAALG | | NSGALTSGVH | | SSASTKGPS | |
| CPPCP | YPREAKVQ | | CLVKDYFPEP | | TFPAVLQSSGL | | VFPLAPSSK | |
| APELL | WKVDNALQ | | VTVSWNSGA | | YSLSSVVTVPS | | STSGGTAA | |
| GG | SGNSQESVT | | LTSGVHTFPA | | SSLGTQTYICN | | LGCLVKDY | |
| (SEQ | EQDSKDSTY | | VLQSSGLYSL | | VNHKPSNTKV | | FPEPVTVS | |
| ID NO: | SLSSTLTLSK | | SSVVTVPSSS | | DKKVEPKSC | | WNSGALTS | |
| 378), | ADYEKHKV | | LGTQTYICNV | | | | GVHTFPAV | |
| D221R | YACEVTHQ | | NHKPSNTKV | | | | LQSSGLYSL | |
| Hinge = | GLSSPVTKS | | DKKVEPKSC | | | | SSVVTVPSS | |
| RKTHT | FNRGEC | | EKTHTCPPCP | | | | SLGTQTYIC | |
| CPPCP | | | APELLGG | | | | NVNHKPSN | |
| APELL | | | | | | | TKVDKKVE | |
| GG | | | | | | | PKSCDGGG | |
| (SEQ | | | | | | | GSGGGGSE | |
| ID NO: | | | | | | | VQLVESGG | |
| 379); | | | | | | | GLVQPGGS | |
| CD3 | | | | | | | LKLSCAAS | |
| LC: | | | | | | | GFTFNKYA | |
| Inserted | | | | | | | MNWVRQA | |
| “SS” | | | | | | | PGKGLEWV | |
| between | | | | | | | ARIRSKYN | |
| VL-CH1 | | | | | | | NYATYYAD | |
| junction; | | | | | | | SVKDRFTIS | |
| CD3 | | | | | | | RDDSKNTA | |
| HC: | | | | | | | YLQMNNL | |
| Replaced | | | | | | | KTEDTAVY | |
| “RT” | | | | | | | YCVRHGNF | |
| with | | | | | | | GNSYISYW | |
| “AS” in | | | | | | | AYWGQGT | |
| constant | | | | | | | LVTVSSAS | |
| kappa | | | | | | | VAAPSVFIF | |
| light | | | | | | | PPSDEQLKS | |
| chain | | | | | | | GTASVVCL | |
| | | | | | | | LNNFYPRE | |
| | | | | | | | AKVQWKV | |
| | | | | | | | DNALQSGN | |
| | | | | | | | SQESVTEQ | |
| | | | | | | | DSKDSTYS | |
| | | | | | | | LSSTLTLSK | |
| | | | | | | | ADYEKHKV | |
| | | | | | | | YACEVTHQ | |
| | | | | | | | GLSSPVTKS | |
| | | | | | | | FNRGECRK | |
| | | | | | | | THTCPPCPA | |
| | | | | | | | PELLGG | |
| TCE-92 | DIQMTQSPS | 364 | EVQLVESGG | 365 | EVQLVESGGG | 366 | EVQLVESG | 367 |
| SBD | SLSASVGDR | | GLVQPGGSL | | LVQPGGSLKL | | GGLVQPGG | |
| Fab | VTITCKSSQS | | RLSCAVSGYS | | SCAASGFTFN | | SLRLSCAVS | |
| (EE/RK); | LLYRSNQKN | | ITSDYAWNW | | KYAMNWVRQ | | GYSITSDYA | |
| C-term | YLAWYQQK | | VRQAPGKGL | | APGKGLEWV | | WNWVRQA | |
| CD3 | PGKAPKLLI | | EWVGYISNS | | ARIRSKYNNY | | PGKGLEWV | |
| CrossFab; | YWASTRESG | | GSTSYNPSLK | | ATYYADSVKD | | GYISNSGST | |
| L1 = DG | VPSRFSGSG | | SRFTISRDTSK | | RFTISRDDSKN | | SYNPSLKSR | |
| GGGS | SGTDFTLTIS | | NTLYLQMNS | | TAYLQMNNL | | FTISRDTSK | |
| GGGG | SLQPEDFAT | | LRAEDTAVY | | KTEDTAVYYC | | NTLYLQMN | |
| S (SEQ | YYCQQYYN | | YCARERNYD | | VRHGNFGNSY | | SLRAEDTA | |
| ID NO: | YPRTFGQGT | | YDDYYYAM | | ISYWAYWGQ | | VYYCARER | |
| 371); | KVEIKRTVA | | DYWGQGTLV | | GTLVTVSSAS | | NYDYDDY | |
| D221E | APSVFIFPPS | | TVSSASTKGP | | VAAPSVFIFPP | | YYAMDYW | |
| Hinge = | DRKLKSGTA | | SVFPLAPSSK | | SDEQLKSGTA | | GQGTLVTV | |
| EKTHT | SVVCLLNNF | | STSGGTAALG | | SVVCLLNNFY | | SSASTKGPS | |
| CPPCP | YPREAKVQ | | CLVEDYFPEP | | PREAKVQWK | | VFPLAPSSK | |
| APELL | WKVDNALQ | | VTVSWNSGA | | VDNALQSGNS | | STSGGTAA | |
| GG | SGNSQESVT | | LTSGVHTFPA | | QESVTEQDSK | | LGCLVEDY | |
| (SEQ | EQDSKDSTY | | VLQSSGLYSL | | DSTYSLSSTLT | | FPEPVTVS | |
| ID NO: | SLSSTLTLSK | | SSVVTVPSSS | | LSKADYEKHK | | WNSGALTS | |
| 378), | ADYEKHKV | | LGTQTYICNV | | VYACEVTHQG | | GVHTFPAV | |
| D221R | YACEVTHQ | | NHKPSNTKV | | LSSPVTKSFNR | | LQSSGLYSL | |
| Hinge = | GLSSPVTKS | | DEKVEPKSCE | | GEC | | SSVVTVPSS | |
| RKTHT | FNRGEC | | KTHTCPPCPA | | | | SLGTQTYIC | |
| CPPCP | | | PELLGGGDY | | | | NVNHKPSN | |
| APELL | | | KDDDDK | | | | GKVDEKVE | |
| GG | | | | | | | PKSCDGGG | |
| (SEQ | | | | | | | GSGGGGSQ | |
| ID NO: | | | | | | | TVVTQEPS | |
| 379); | | | | | | | LTVSPGGT | |
| CD3 | | | | | | | VTLTCGSST | |
| LC: | | | | | | | GAVTSGNY | |
| Replaced | | | | | | | PNWVQQKP | |
| “RT” | | | | | | | GQAPRGLI | |
| with | | | | | | | GGTKFLAP | |
| “AS” in | | | | | | | GTPARFSGS | |
| constant | | | | | | | LLGGKAAL | |
| kappa | | | | | | | TLSGVQPE | |
| light | | | | | | | DEAEYYCV | |
| chain; | | | | | | | LWYSNRW | |
| CD3 | | | | | | | VFGGGTKL | |
| HC: | | | | | | | TVLSSASTK | |
| Inserted | | | | | | | GPSVFPLAP | |
| “SS” | | | | | | | SSKSTSGGT | |
| between | | | | | | | AALGCLVK | |
| VL- | | | | | | | DYFPEPVT | |
| CH1 | | | | | | | VSWNSGAL | |
| junction | | | | | | | TSGVHTFP | |
| | | | | | | | AVLQSSGL | |
| | | | | | | | YSLSSVVT | |
| | | | | | | | VPSSSLGTQ | |
| | | | | | | | TYICNVNH | |
| | | | | | | | KPSNGKVD | |
| | | | | | | | KKVEPKSC | |
| | | | | | | | RKTHTCPP | |
| | | | | | | | CPAPELLG | |
| | | | | | | | G | |
| TCE-93 | EIVLTQSPAT | 368 | QVQLVQSGA | 369 | | | QVQLVQSG | 370 |
| SBD | LSLSPGERA | | EVKKPGASV | | | | AEVKKPGA | |
| Fab; | TLSCRASSS | | KVSCKASGY | | | | SVKVSCKA | |
| CD3 | VSYMHWFQ | | TFSTYWIEW | | | | SGYTFSTY | |
| scFv; | QKPGQAPRL | | VRQAPGQRL | | | | WIEWVRQA | |
| L1 = | LIYSTSNLAS | | EWMGEILPGS | | | | PGQRLEW | |
| GGGG | GIPARFSGSG | | GQTDFNEKF | | | | MGEILPGS | |
| SGGG | SGTDYTLTIS | | QGRVTFTAD | | | | GQTDFNEK | |
| GS | SLEPEDFAV | | TSSDTAYME | | | | FQGRVTFT | |
| (SEQ | YYCQQRRSF | | LSSLRSEDTA | | | | ADTSSDTA | |
| ID NO: | PYTFGQGTK | | VYYCTRWGY | | | | YMELSSLR | |
| 270); | LEIKRTVAA | | YGTRGYFNV | | | | SEDTAVYY | |
| Hinge = | PSVFIFPPSD | | WGQGTLVTV | | | | CTRWGYY | |
| GGGG | EQLKSGTAS | | SSASTKGPSV | | | | GTRGYFNV | |
| SGGG | VVCLLNNFY | | FPLAPSSKSTS | | | | WGQGTLVT | |
| GSKTH | PREAKVQW | | GGTAALGCL | | | | VSSASTKG | |
| TCPPC | KVDNALQS | | VKDYFPEPVT | | | | PSVFPLAPS | |
| PAP | GNSQESVTE | | VSWNSGALT | | | | SKSTSGGT | |
| (SEQ | QDSKDSTYS | | SGVHTFPAVL | | | | AALGCLVK | |
| ID NO: | LSSTLTLSK | | QSSGLYSLSS | | | | DYFPEPVT | |
| 380); | ADYEKHKV | | VVTVPSSSLG | | | | VSWNSGAL | |
| Fc = | YACEVTHQ | | TQTYICNVNH | | | | TSGVHTFP | |
| PVAGP | GLSSPVTKS | | KPSDTKVDK | | | | AVLQSSGL | |
| SVFLF | FNRGEC | | KVEPKSCDK | | | | YSLSSVVT | |
| PPKPK | | | THTCPPCPAP | | | | VPSSSLGTQ | |
| DTLMI | | | PVAGPSVFLF | | | | TYICNVNH | |
| SRTPE | | | PPKPKDTLMI | | | | KPSNTKVD | |
| VTCV | | | SRTPEVTCVV | | | | KKVEPKSC | |
| VVDV | | | VDVKHEDPE | | | | GGGGSGGG | |
| KHEDP | | | VKFNWYVDG | | | | GSEVQLVE | |
| EVKFN | | | VEVHNAKTK | | | | SGGGLVQP | |
| WYVD | | | PCEEEYGSTY | | | | GGSLRLSC | |
| GVEV | | | RCVSVLTVL | | | | AASGFTFST | |
| HNAK | | | HQDWLNGKE | | | | YAMNWVR | |
| TKPCE | | | YKCKVSNKA | | | | QAPGKGLE | |
| EQYGS | | | LPAPIEKTISK | | | | WVGRIRSK | |
| TYRCV | | | AKGQPREPQ | | | | YNNYATYY | |
| SVLTV | | | VYTLPPSREE | | | | ADSVKGRF | |
| LHQD | | | MTKNQVSLT | | | | TISRDDSKN | |
| WLNG | | | CDVSGFYPSD | | | | TLYLQMNS | |
| KEYK | | | IAVEWESDG | | | | LRAEDTAV | |
| CKVSN | | | QPENNYKTTP | | | | YYCVRHGN | |
| KALPA | | | PVLDSDGSFF | | | | FGDSYVSW | |
| PIEKTI | | | LYSKLTVDKS | | | | FAYWGQG | |
| SKAK | | | RWEQGDVFS | | | | TLVTVSSG | |
| GQPRE | | | CSVMHEALH | | | | KPGSGKPG | |
| PQVYT | | | NHYTQKSLSL | | | | SGKPGSGK | |
| LPPSR | | | SPGK | | | | PGSQAVVT | |
| EQMT | | | | | | | QEPSLTVSP | |
| KNQV | | | | | | | GGTVTLTC | |
| KLTCL | | | | | | | GSSTGAVT | |
| VKGF | | | | | | | TSNYANW | |
| YPSDI | | | | | | | VQQKPGKS | |
| AVEW | | | | | | | PRGLIGGTN | |
| ESNGQ | | | | | | | KRAPGVPA | |
| PENNY | | | | | | | RFSGSLLG | |
| KTTPP | | | | | | | GKAALTIS | |
| VLDSD | | | | | | | GAQPEDEA | |
| GSFFL | | | | | | | DYYCALW | |
| YSKLT | | | | | | | YSNHWVFG | |
| VDKSR | | | | | | | GGTKLTVL | |
| WQQG | | | | | | | GGGGSGGG | |
| NVFSC | | | | | | | GSKTHTCP | |
| SVMH | | | | | | | PCPAPPVA | |
| EALHN | | | | | | | GPSVFLFPP | |
| HYTQ | | | | | | | KPKDTLMI | |
| KSLSL | | | | | | | SRTPEVTC | |
| SPGK | | | | | | | VVVDVKHE | |
| (SEQ | | | | | | | DPEVKFNW | |
| ID NO: | | | | | | | YVDGVEVH | |
| 381) | | | | | | | NAKTKPCE | |
| | | | | | | | EQYGSTYR | |
| | | | | | | | CVSVLTVL | |
| | | | | | | | HQDWLNG | |
| | | | | | | | KEYKCKVS | |
| | | | | | | | NKALPAPIE | |
| | | | | | | | KTISKAKG | |
| | | | | | | | QPREPQVY | |
| | | | | | | | TLPPSREQ | |
| | | | | | | | MTKNQVK | |
| | | | | | | | LTCLVKGF | |
| | | | | | | | YPSDIAVE | |
| | | | | | | | WESNGQPE | |
| | | | | | | | NNYKTTPP | |
| | | | | | | | VLDSDGSF | |
| | | | | | | | FLYSKLTV | |
| | | | | | | | DKSRWQQ | |
| | | | | | | | GNVFSCSV | |
| | | | | | | | MHEALHN | |
| | | | | | | | HYTQKSLS | |
| | | | | | | | LSPGK | |

Disclosed herein, in some embodiments, are polypeptide or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain, wherein the immunoglobulin heavy chain and the immunoglobulin light chain comprise a set of six CDR sequences according to: CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33, and wherein the polypeptide or polypeptide complex further comprises a second antibody or antigen binding fragment thereof that binds to CD3, wherein the second antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of SEQ ID NO: 81, CDR2-L comprises the amino acid sequence of SEQ ID NO: 81, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

In some embodiments, the antibody or antigen binding fragment thereof that binds to STEAP-1 comprises an amino acid sequence of at least 95% sequence identity to SEQ ID NO: 225 and an amino acid sequence of at least 95% sequence identity to SEQ ID NO: 226. In some embodiments, the antibody or antigen binding fragment thereof that binds to STEAP-1 comprises an amino acid sequence of at least 99% sequence identity to SEQ ID NO: 225 and an amino acid sequence of at least 99% sequence identity to SEQ ID NO: 226. In some embodiments, the antibody or antigen binding fragment thereof that binds to STEAP-1 comprises the amino acid sequence of SEQ ID NO: 225 and the amino acid sequence of SEQ ID NO: 226.

In some embodiments, the antibody or antigen binding fragment thereof that binds to STEAP-1 comprises an amino acid sequence of at least 95% sequence identity to SEQ ID NO: 1 and an amino acid sequence of at least 95% sequence identity to SEQ ID NO: 2. In some embodiments, the antibody or antigen binding fragment thereof that binds to STEAP-1 comprises an amino acid sequence of at least 99% sequence identity to SEQ ID NO: 1 and an amino acid sequence of at least 99% sequence identity to SEQ ID NO: 2. In some embodiments, the antibody or antigen binding fragment thereof that binds to STEAP-1 comprises the amino acid sequence of SEQ ID NO: 1 and the amino acid sequence of SEQ ID NO: 2. In some embodiments, the antibody or antigen binding fragment thereof that binds to STEAP-1 comprises a Fab, Fab′, (Fab′)₂ or a single chain variable fragment (scFv).

In some embodiments, the polypeptide or polypeptide complex comprises more than one antibody or antigen binding fragment thereof that binds to STEAP-1. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁). In some embodiments, L₁ connects an immunoglobulin heavy chain of the Fab or Fab′ that binds STEAP-1 with another immunoglobulin heavy chain of the second Fab or Fab′ that binds STEAP-1. In some embodiments, L₁ connects an immunoglobulin light chain of the Fab or Fab′ that binds STEAP-1 with another immunoglobulin light chain of the second Fab or Fab′ that binds STEAP-1. In some embodiments, L₁ connects an immunoglobulin light chain of the Fab or Fab′ that binds STEAP-1 with an immunoglobulin heavy chain of the second Fab or Fab′ that binds STEAP-1.

### CD3 Binding Domain

In some embodiments, CDR1-H of the second antibody or antigen binding fragment thereof that binds to CD3 comprises the amino acid sequence of SEQ ID NO: 61 or SEQ ID NO: 64; CDR2-H of the second antibody or antigen binding fragment thereof that binds to CD3 comprises the amino acid sequence of SEQ ID NO: 62; CDR3-H of the second antibody or antigen binding fragment thereof that binds to CD3 comprises the amino acid sequence selected from SEQ ID NOs: 63, 65-79, and 124; CDR1-L of the second antibody or antigen binding fragment thereof that binds to CD3 comprises the amino acid sequence of SEQ ID NO: 80 or SEQ ID NO: 83; CDR2-L of the second antibody or antigen binding fragment thereof that binds to CD3 comprises the amino acid sequence of GTK; and CDR3-L of the second antibody or antigen binding fragment thereof that binds to CD3 comprises the amino acid sequence selected from SEQ ID NOs: 82 and 84-93.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: SEQ ID NO: 81, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 65, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 66, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 67, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 68, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 69, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 70, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 71, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 72, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 73, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 74, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 75, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 76, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 77, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 78, CDR1-L: SEQ ID NO: 80, CDR2-L: GTK, and CDR3-L: SEQ ID NO: 82. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 79, CDR1-L: SEQ ID NO: 80, CDR2-L: GTK, and CDR3-L: SEQ ID NO: 82.

In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 84. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 85. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 86. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 87. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 88. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 89. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 90. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 91. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 92. In some embodiments, the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, and CDR3-L: SEQ ID NO: 93. In some embodiments, the second antibody or antigen binding fragment thereof that binds to CD3 comprises the following set of six CDRs: CDR1-H: SEQ ID NO: 64, CDR2-H: SEQ ID NO: 62, CDR3-H: SEQ ID NO: 124, CDR1-L: SEQ ID NO: 83, CDR2-L: GTK, and CDR3-L: SEQ ID NO: 84.

In some embodiments, the second antibody or antigen binding fragment thereof that binds to CD3 comprises a variable domain of an immunoglobulin kappa (IgK) or immunoglobulin lambda (IgL) light chain. In some embodiments, the second antibody or antigen binding fragment thereof that binds to CD3 comprises a constant domain of an IgG1, IgG2, IgG3, or IgG4 heavy chain.

In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 94 and SEQ ID NO: 95. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 266 and SEQ ID NO: 267. In some embodiments, the second antibody or antigen binding fragment thereof that binds to CD3 comprises an amino acid sequence with at least 90%, 95%, 96%, 97%, 98%, or 100% sequence identity to SEQ ID NO: 268 and SEQ ID NO: 269. In some embodiments, the second antibody or antigen binding fragment thereof that binds to CD3 comprises a Fab, Fab′, (Fab′)₂ or a single chain variable fragment (scFv). In some embodiments, the second antibody or antigen binding fragment thereof that binds to CD3 comprises the scFv. In some embodiments, the second antibody or antigen binding fragment thereof that binds to CD3 comprises the Fab.

In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 96. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 97. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 98. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 99. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 100. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 101. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 102. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 103. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 104. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 105. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 106. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 107. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 108. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 109. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 110. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 111. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 112. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 113. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 114. In some embodiments, the second antibody or antigen binding fragment thereof that binds to CD3 comprises an amino acid sequence with at least 90%, 95%, 96%, 97%, 98%, 99%, or 100% identity to SEQ ID NO: 115. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 116. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 117. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 118. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 119. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 120. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 121. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 122. In some embodiments, the second antibody or antigen binding fragment thereof that binds to CD3 comprises an amino acid sequence with at least 90%, 95%, 96%, 97%, 98%, 99%, or 100% sequence identity to SEQ ID NO: 123.

### Connections

STEAP-1 Fab/CD3 scFv

In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin heavy chain. In some embodiments, the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin light chain. In some embodiments, the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin light chain.

### STEAP-1 Fab/CD3 Fab

In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to STEAP-1 is connected to the Fab or Fab′ that binds to CD3 by a linker (L₂). In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin heavy chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin light chain of the Fab or Fab′ that binds to STEAP-1. In some embodiments, the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin light chain of the Fab or Fab′ that binds to STEAP-1.

STEAP-1 scFv/CD3 Fab

In some embodiments, the antibody or antigen binding fragment thereof that binds to STEAP-1 is an scFv, and the second antibody or antigen binding fragment thereof that binds to CD3 is a Fab or Fab′ and the scFv that binds to STEAP-1 is connected to the Fab or Fab′ that binds to CD3 by a linker (L₂). In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin heavy chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin light chain of the scFv that binds to STEAP-1. In some embodiments, the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin light chain of the scFv that binds to STEAP-1.

STEAP-1 scFv

In some embodiments, the antibody or antigen binding fragment thereof that binds to STEAP-1 is an scFv, and wherein a C-terminus of the scFv that binds to STEAP-1 is connected to an N-terminus of the second antibody or antigen binding fragment thereof that binds to CD3 via a linker (L₂). In some embodiments, the antibody or antigen binding fragment thereof that binds to STEAP-1 is an scFv, and wherein an N-terminus of the scFv that binds to STEAP-1 is connected to a C-terminus of the second antibody or antigen binding fragment thereof that binds to CD3 via a linker (L₂). In some embodiments, a C-terminus of the immunoglobulin light chain of the scFv that binds to STEAP-1 is connected to an N-terminus of the immunoglobulin heavy chain of the scFv that binds to STEAP-1 via a linker. In some embodiments, a C-terminus of the immunoglobulin heavy chain of the scFv that binds to STEAP-1 is connected to an N-terminus of the immunoglobulin light chain that binds to STEAP-1 via a linker.

### STEAP-1 Binding Domains

In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to an N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to the C terminus of one of the two connected Fab or Fab′ that are connected by L₁.

In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the N terminus of one of the two connected Fab or Fab′ that are connected by L₁. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the C terminus of one of the two Fab or Fab′ that are connected by L₁.

In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by the second antibody or antigen binding fragment thereof that binds to CD3, wherein the second antibody or antigen binding fragment thereof that binds to CD3 comprises a CrossFab configuration, and wherein the Fab or Fab′ that binds to STEAP-1 is connected to the second antibody or antigen binding fragment thereof that binds to CD3 via a linker (L₁) and the second Fab or Fab′ that binds to STEAP-1 is connected to the second antibody or antigen binding fragment thereof that binds to CD3 via a linker (L₂). In some embodiments, an immunoglobulin heavy chain of the Fab or Fab′ that binds to STEAP-1 with an immunoglobulin light chain of the second antibody or antigen binding fragment that binds to CD3, and L₂ connects an immunoglobulin heavy chain of the second antibody or antigen binding fragment that binds to CD3 with another immunoglobulin heavy chain of the second Fab or Fab′ that binds to STEAP-1.

### Linker

In some embodiments, L₁ or L₂ comprises an amino acid sequence according to any one of SEQ ID NOs: 222-224, 270-272, and 371.

### TCE Sequences

In some embodiments, the polypeptide or polypeptide complex comprises an amino acid sequence that comprises the immunoglobulin light chain that binds to STEAP-1 is selected from SEQ ID NOs: 126, 127, 138, and 139, and an amino acid sequence that comprises the immunoglobulin heavy chain that binds to STEAP-1 is selected from SEQ ID NOs: 174, 175, 186, and 187. In some embodiments, the polypeptide complex comprises amino acid sequences with at least 90%, 95%, 96%, 97%, 98%, 99%, or 100% sequence identity to SEQ ID NO: 126 and SEQ ID NO: 174. In some embodiments, the polypeptide complex comprises the amino acid sequences of SEQ ID NO: 126 and SEQ ID NO: 174. In some embodiments, the polypeptide complex comprises amino acid sequences with at least 90%, 95%, 96%, 97%, 98%, 99%, or 100% sequence identity to SEQ ID NO: 127 and SEQ ID NO: 175. In some embodiments, the polypeptide complex comprises the amino acid sequences of SEQ ID NO: 127 and SEQ ID NO: 175. In some embodiments, the polypeptide complex comprises amino acid sequences with at least 90%, 95%, 96%, 97%, 98%, 99%, or 100% sequence identity to SEQ ID NO: 138 and SEQ ID NO: 186. In some embodiments, the polypeptide complex comprises the amino acid sequences of SEQ ID NO: 138 and SEQ ID NO: 186. In some embodiments, the polypeptide complex comprises amino acid sequences with at least 90%, 95%, 96%, 97%, 98%, 99%, or 100% sequence identity to SEQ ID NO: 139 and SEQ ID NO: 187. In some embodiments, the polypeptide complex comprises the amino acid sequences of SEQ ID NO: 139 and SEQ ID NO: 187.

In some embodiments, the polypeptide or polypeptide complex comprises an amino acid sequence that comprises the immunoglobulin light chain that binds to STEAP-1 is selected from SEQ ID NOs: 150, 151, 162, and 163 and an amino acid sequence that comprises the immunoglobulin heavy chain that binds to STEAP-1 is selected from SEQ ID NOs: 198, 199, 210, and 211. In some embodiments, the polypeptide complex comprises amino acid sequences with at least 90%, 95%, 96%, 97%, 98%, 99%, or 100% sequence identity to SEQ ID NO: 150 and SEQ ID NO: 198. In some embodiments, the polypeptide complex comprises the amino acid sequences of SEQ ID NO: 150 and SEQ ID NO: 198. In some embodiments, the polypeptide complex comprises amino acid sequences with at least 90%, 95%, 96%, 97%, 98%, 99%, or 100% sequence identity to SEQ ID NO: 151 and SEQ ID NO: 199. In some embodiments, the polypeptide complex comprises the amino acid sequences of SEQ ID NO: 151 and SEQ ID NO: 199. In some embodiments, the polypeptide complex comprises amino acid sequences with at least 90%, 95%, 96%, 97%, 98%, 99%, or 100% sequence identity to SEQ ID NO: 162 and SEQ ID NO: 210. In some embodiments, the polypeptide complex comprises the amino acid sequences of SEQ ID NO: 162 and SEQ ID NO: 210. In some embodiments, the polypeptide complex comprises amino acid sequences with at least 90%, 95%, 96%, 97%, 98%, 99%, or 100% sequence identity to SEQ ID NO: 163 and SEQ ID NO: 211. In some embodiments, the polypeptide complex comprises the amino acid sequences of SEQ ID NO: 163 and SEQ ID NO: 211.

In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 126-127, 138-139, 273-282, 285-286, and 289-290, and an amino acid sequence, that comprises the immunoglobulin heavy chain that binds to STEAP-1, with at least 95%, 98%, 99%, or 100% sequence identity to any one of SEQ ID NOs: 174-175, 186-187, 299-302, 372, 303-307, 310-311, and 314-315.

In some embodiments, the polypeptide or polypeptide complex comprises an amino acid sequence, that comprises the immunoglobulin light chain that binds to STEAP-1, according to SEQ ID NO: 283 and an amino acid sequence, that comprises the immunoglobulin heavy chain that binds to STEAP-1, according to SEQ ID NO: 308. In some embodiments, the polypeptide complex comprises amino acid sequences with at least 90%, 95%, 96%, 97%, 98%, 99%, or 100% sequence identity to SEQ ID NO: 283 and SEQ ID NO: 308. In some embodiments, the polypeptide complex comprises the amino acid sequences of SEQ ID NO: 283 and SEQ ID NO: 308.

In some embodiments, the polypeptide or polypeptide complex comprises an amino acid sequence that comprises the immunoglobulin light chain that binds to STEAP-1 according to SEQ ID NO: 284 and an amino acid sequence that comprises the immunoglobulin heavy chain that binds to STEAP-1 according to SEQ ID NO: 309. In some embodiments, the polypeptide complex comprises amino acid sequences with at least 90%, 95%, 96%, 97%, 98%, 99%, or 100% sequence identity to SEQ ID NO: 284 and SEQ ID NO: 309. In some embodiments, the polypeptide complex comprises the amino acid sequences of SEQ ID NO: 284 and SEQ ID NO: 309.

In some embodiments, the polypeptide or polypeptide complex comprises an amino acid sequence that comprises the immunoglobulin light chain that binds to STEAP-1 according to SEQ ID NO: 287 and an amino acid sequence that comprises the immunoglobulin heavy chain that binds to STEAP-1 according to SEQ ID NO: 312. In some embodiments, the polypeptide complex comprises amino acid sequences with at least 90%, 95%, 96%, 97%, 98%, 99%, or 100% sequence identity to SEQ ID NO: 287 and SEQ ID NO: 312. In some embodiments, the polypeptide complex comprises the amino acid sequences of SEQ ID NO: 287 and SEQ ID NO: 312.

In some embodiments, the polypeptide or polypeptide complex comprises an amino acid sequence that comprises the immunoglobulin light chain that binds to STEAP-1 according to SEQ ID NO: 288 and an amino acid sequence that comprises the immunoglobulin heavy chain that binds to STEAP-1 according to SEQ ID NO: 313. In some embodiments, the polypeptide complex comprises amino acid sequences with at least 90%, 95%, 96%, 97%, 98%, 99%, or 100% sequence identity to SEQ ID NO: 288 and SEQ ID NO: 313. In some embodiments, the polypeptide complex comprises the amino acid sequences of SEQ ID NO: 288 and SEQ ID NO: 313.

In some embodiments, the polypeptide or polypeptide complex comprises an amino acid sequence that comprises the immunoglobulin light chain that binds to STEAP-1 according to SEQ ID NO: 291 and an amino acid sequence that comprises the immunoglobulin heavy chain that binds to STEAP-1 according to SEQ ID NO: 316. In some embodiments, the polypeptide complex comprises amino acid sequences with at least 90%, 95%, 96%, 97%, 98%, 99%, or 100% sequence identity to SEQ ID NO: 291 and SEQ ID NO: 316. In some embodiments, the polypeptide complex comprises the amino acid sequences of SEQ ID NO: 291 and SEQ ID NO: 316.

In some embodiments, the polypeptide or polypeptide complex comprises an amino acid sequence that comprises the immunoglobulin light chain that binds to STEAP-1 according to SEQ ID NO: 292 and an amino acid sequence that comprises the immunoglobulin heavy chain that binds to STEAP-1 according to SEQ ID NO: 317. In some embodiments, the polypeptide complex comprises amino acid sequences with at least 90%, 95%, 96%, 97%, 98%, 99%, or 100% sequence identity to SEQ ID NO: 292 and SEQ ID NO: 317. In some embodiments, the polypeptide complex comprises the amino acid sequences of SEQ ID NO: 292 and SEQ ID NO: 317.

In some embodiments, the polypeptide complex comprises amino acid sequences with at least 90%, 95%, 96%, 97%, 98%, 99%, or 100% sequence identity to any one of SEQ ID NOs: 126-173, 273-298, and 330-342 and SEQ ID NOs: 174-221, 299-323, and 343-355.

In some embodiments, the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 150-151, 162-163, and 330-342, and an amino acid sequence, that comprises the immunoglobulin heavy chain that binds to STEAP-1, with at least 95%, 98%, 99%, or 100% sequence identity to any one of SEQ ID NOs: 198-199, 210-211, and 343-355.

In some embodiments, the polypeptide or polypeptide complex comprises amino acid sequences with at least 95%, 98%, 99%, or 100% sequence identity to SEQ ID NOs: 327, 328, and 329. In some embodiments, the polypeptide or polypeptide complex comprises amino acid sequences with at least 95%, 98%, 99%, or 100% sequence identity to SEQ ID NOs: 366, 357, 358, and 359. In some embodiments, the polypeptide or polypeptide complex comprises amino acid sequences with at least 95%, 98%, 99%, or 100% sequence identity to SEQ ID NOs: 360, 361, 362, and 363.

### Modified Amino Acids

In some embodiments, the isolated polypeptide or polypeptide complex comprises a modified amino acid, a non-natural amino acid, a modified non-natural amino acid, or a combination thereof. In some embodiments, the non-natural amino acid comprises a D-amino acid. In some embodiments, the modified amino acid or modified non-natural amino acid comprises a post-translational modification.

### Pharmaceutical Compositions

Disclosed herein, in some embodiments, are pharmaceutical compositions comprising: (i) the polypeptide or polypeptide complex according to any embodiment disclosed herein, and (ii) a pharmaceutically acceptable excipient.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the polypeptide or polypeptide complex comprises a second antibody or antigen binding fragment thereof that binds to CD3.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; wherein the polypeptide or polypeptide complex further comprises a second antibody or antigen binding fragment thereof that binds to CD3 wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; and wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservatives, emulsifying agents and dispersing agents. Prevention of the action of microorganisms may be ensured by the inclusion of various antibacterial and antifungal agents.

The pharmaceutical composition may be in any suitable form, depending upon the desired method of administration. It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Isolated Recombinant Nucleic Acid Molecules

Disclosed herein, in some embodiments, are isolated recombinant nucleic acid molecules encoding the polypeptide or polypeptide complex of any embodiment disclosed herein.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the polypeptide or polypeptide complex comprises a second antibody or antigen binding fragment thereof that binds to CD3.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; wherein the polypeptide or polypeptide complex further comprises a second antibody or antigen binding fragment thereof that binds to CD3 wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; and wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

### Methods of Treatment

Disclosed herein, in some embodiments, are methods of treating cancer in a subject in need thereof comprising administering to the subject the polypeptide or polypeptide complex of any embodiment disclosed herein.

Disclosed herein, in some embodiments, are methods of treating cancer in a subject in need thereof comprising administering to the subject a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Disclosed herein, in some embodiments, are methods of treating cancer in a subject in need thereof comprising administering to the subject a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the polypeptide or polypeptide complex comprises a second antibody or antigen binding fragment thereof that binds to CD3.

Disclosed herein, in some embodiments, are methods of treating cancer in a subject in need thereof comprising administering to the subject a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; wherein the polypeptide or polypeptide complex further comprises a second antibody or antigen binding fragment thereof that binds to CD3 wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; and wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

In some embodiments, the cancer is sarcoma or a cancer of the prostate, breast, pancreas, bladder, gastrointestinal tract, testis, ovary, or cervix. In some embodiments, the cancer is prostate cancer or Ewing sarcoma.

In some embodiments, the method further comprises administering to the subject an immune checkpoint inhibitor. In some embodiments, the immune checkpoint inhibitor comprises an antibody or antigen binding fragment thereof that binds to PD-1.

### Production of Antibodies

In some embodiments, the recombinant antibodies or antigen binding fragments thereof as described herein are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

Disclosed herein, in some embodiments, are expression vectors comprising a nucleic acid encoding the polypeptide or polypeptide complex of any embodiment disclosed herein.

Disclosed herein, in some embodiments, are expression vectors comprising a nucleic acid encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Disclosed herein, in some embodiments, are expression vectors comprising a nucleic acid encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and wherein the polypeptide or polypeptide complex comprises a second antibody or antigen binding fragment thereof that binds to CD3.

Disclosed herein, in some embodiments, are expression vectors comprising a nucleic acid encoding a polypeptide or polypeptide complex an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; wherein the polypeptide or polypeptide complex further comprises a second antibody or antigen binding fragment thereof that binds to CD3 wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; and wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

In some embodiments, vectors include any suitable vectors derived from either eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or Hela cells. Exemplary cell-free systems include, but are not limited to, E. coli S30 Extract system, E. coli T7 S30 system, or PURExpress®.

### Host Cells

Disclosed herein, in some embodiments, are host cells comprising the polypeptide or polypeptide complex of any embodiment disclosed herein.

Disclosed herein, in some embodiments, are host cells comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Disclosed herein, in some embodiments, are host cells comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and wherein the polypeptide or polypeptide complex comprises a second antibody or antigen binding fragment thereof that binds to CD3.

Disclosed herein, in some embodiments, are host cells comprising a polypeptide or polypeptide complex an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; wherein the polypeptide or polypeptide complex further comprises a second antibody or antigen binding fragment thereof that binds to CD3 wherein the antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83, CDR2-L comprises the amino acid sequence of GTK, and CDR3-L comprises the amino acid sequence of Z₁-Z₂-W-Z₃-Z₄-Z₅-Z₆-W-Z₇-Z₈ or SEQ ID NO: 82, wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; and wherein CDR1-H comprises the amino acid sequence of SEQ ID NO: 61 or SEQ ID NO: 64, CDR2-H comprises the amino acid sequence of SEQ ID NO: 62, and CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63, wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5a, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomyces (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293 FT cell line, 293F cells, 293 H cells, CHO DG44 cells, CHO-S cells, CHO-K1 cells, FUT8 KO CHOK1, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per.C6 cells, T-REX™-293 cell line, T-REX™-CHO cell line, and T-REX™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a recombinant antibody or antigen binding fragment thereof of any embodiment disclosed herein.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Embodiments

Embodiment 1 comprises a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

Embodiment 2 comprises the polypeptide or polypeptide complex of embodiment 1, wherein the at least one amino acid mutation is selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226.

Embodiment 3 comprises the polypeptide or polypeptide complex of embodiments 1 or 2, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226.

Embodiment 4 comprises the polypeptide or polypeptide complex of any one of embodiments 1 to 3, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 225, and the immunoglobulin heavy chain comprises an amino acid sequence with at least 90% sequence identity to SEQ ID NO: 225, and the immunoglobulin light chain comprises an amino acid sequence with at least 90% sequence identity to SEQ ID NO: 226.

Embodiment 5 comprises the polypeptide or polypeptide complex of any one of the preceding embodiments, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226, and the immunoglobulin heavy chain comprises an amino acid sequence with at least 95% sequence identity to SEQ ID NO: 225, and the immunoglobulin light chain comprises an amino acid sequence with at least 95% sequence identity to SEQ ID NO: 226.

Embodiment 6 comprises the polypeptide or polypeptide complex of any one of embodiments 1 to 5, wherein the immunoglobulin heavy chain comprises mutations V24A, Y27F, T58I, and T74N, and at least 90% sequence identity to SEQ ID NO: 225.

Embodiment 7 comprises the polypeptide or polypeptide complex of any one of embodiments 1 to 5, wherein the immunoglobulin heavy chain comprises mutations W35M and L64V and at least 90% sequence identity to SEQ ID NO: 225.

Embodiment 8 comprises the polypeptide or polypeptide complex of any one of embodiments 1 to 5, wherein the immunoglobulin heavy chain comprises mutations V24A, Y27F, W35M, T58I, L64V, S66G, and T74N and at least 90% sequence identity to SEQ ID NO: 225.

Embodiment 9 comprises the polypeptide or polypeptide complex of any one of embodiments 1 to 5, wherein the immunoglobulin heavy chain comprises mutations W35M, L64V, E99D, and M111F and at least 90% sequence identity to SEQ ID NO: 225.

Embodiment 10 comprises the polypeptide or polypeptide complex of any one of embodiments 1 to 5, wherein the immunoglobulin heavy chain comprises mutations V24A, Y27F, W35M, L64V, T74N, E99D, and M111F and at least 90% sequence identity to SEQ ID NO: 225.

Embodiment 11 comprises the polypeptide or polypeptide complex of any one of embodiments 1 to 5, wherein the immunoglobulin heavy chain comprises the G45C mutation and at least 90% sequence identity to SEQ ID NO: 225.

Embodiment 12 comprises the polypeptide or polypeptide complex of any one of embodiments 1 to 11, wherein the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226.

Embodiment 13 comprises the polypeptide or polypeptide complex of any one of embodiments 1 to 11, wherein the immunoglobulin light chain comprises the Q35N mutation and at least 90% sequence identity to SEQ ID NO: 226.

Embodiment 14 comprises the polypeptide or polypeptide complex of any one of embodiments 1 to 11, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

Embodiment 15 comprises the polypeptide or polypeptide complex of any one of embodiments 1 to 11, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

Embodiment 16 comprises the polypeptide or polypeptide complex of any one of embodiments 1 to 11, wherein the immunoglobulin light chain comprises the Q106C mutation and at least 90% sequence identity to SEQ ID NO: 226.

Embodiment 17 comprises the polypeptide or polypeptide complex of any one of embodiments 1 to 5, wherein the immunoglobulin heavy chain comprises mutations V24A, Y27F, T58I, and T74N, and at least 90% sequence identity to SEQ ID NO: 225 and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226.

Embodiment 18 comprises the polypeptide or polypeptide complex of any one of embodiments 1 to 5, wherein the immunoglobulin heavy chain comprises mutations W35M and L64V and at least 90% sequence identity to SEQ ID NO: 225 and the immunoglobulin light chain comprises the Q35N mutation and at least 90% sequence identity to SEQ ID NO: 226.

Embodiment 19 comprises the polypeptide or polypeptide complex of any one of embodiments 1 to 5, wherein the immunoglobulin heavy chain comprises mutations V24A, Y27F, W35M, T58I, L64V, S66G, and T74N and at least 90% sequence identity to SEQ ID NO: 225 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

Embodiment 20 comprises the polypeptide or polypeptide complex of any one of embodiments 1 to 5, wherein the immunoglobulin heavy chain comprises mutations W35M, L64V, E99D, and M111F and at least 90% sequence identity to SEQ ID NO: 225 and the immunoglobulin light chain comprises the Q35N mutation and at least 90% sequence identity to SEQ ID NO: 226.

Embodiment 21 comprises the polypeptide or polypeptide complex of any one of embodiments 1 to 5, wherein the immunoglobulin heavy chain comprises mutations V24A, Y27F, W35M, L64V, T74N, E99D, and M111F and at least 90% sequence identity to SEQ ID NO: 225 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

Embodiment 22 comprises the polypeptide or polypeptide complex of any one of embodiments 1 to 5, wherein the immunoglobulin heavy chain comprises the G45C mutation and at least 90% sequence identity to SEQ ID NO: 225 and the immunoglobulin light chain comprises the Q106C mutation and at least 90% sequence identity to SEQ ID NO: 226.

Embodiment 23 comprises the polypeptide or polypeptide complex of embodiment any one of embodiments 1 to 5 and 22, wherein the antibody or antigen binding fragment thereof comprises an engineered disulfide bond between the immunoglobulin heavy chain and the immunoglobulin light chain.

Embodiment 24 comprises the polypeptide or polypeptide complex of embodiment 22, wherein the antibody or antigen binding fragment thereof comprises an engineered disulfide bond between the cysteine at the position of the G45C mutation and the cysteine at the position of the Q106C mutation.

Embodiment 25 comprises the polypeptide or polypeptide complex of any one of the preceding embodiments, wherein the immunoglobulin light chain comprises complementarity determining regions (CDRs) CDR1-L, CDR2-L, and CDR3-L, and the immunoglobulin heavy chain comprises CDRs: CDR1-H, CDR2-H, and CDR3-H, wherein at least one of the CDRs comprises the amino acid sequence selected from SEQ ID NOs: 3-38.

Embodiment 26 comprises the polypeptide or polypeptide complex of any one of the preceding embodiments, wherein CDR1-H comprises the amino acid sequence selected from SEQ ID NOs: 3-8; wherein CDR2-H comprises the amino acid sequence selected from SEQ ID NOs: 9-14; and wherein CDR3-H comprises the amino acid sequence selected from SEQ ID NOs: 15-20.

Embodiment 27 comprises the polypeptide or polypeptide complex of any one of the preceding embodiments, wherein CDR1-L comprises the amino acid sequence selected from SEQ ID NOs: 21-26; wherein CDR2-L comprises the amino acid sequence selected from SEQ ID NOs: 27-32; and wherein CDR3-L comprises the amino acid sequence selected from SEQ ID NOs: 33-38.

Embodiment 28 comprises the polypeptide or polypeptide complex of embodiment any one of the preceding embodiments, wherein CDR1-H comprises the amino acid sequence selected from SEQ ID NOs: 3-8; wherein CDR2-H comprises the amino acid sequence selected from SEQ ID NOs: 9-14; wherein CDR3-H comprises the amino acid sequence selected from SEQ ID NOs: 15-20; wherein CDR1-L comprises the amino acid sequence selected from SEQ ID NOs: 21-26; wherein CDR2-L comprises the amino acid sequence selected from SEQ ID NOs: 27-32; and wherein CDR3-L comprises the amino acid sequence selected from SEQ ID NOs: 33-38.

Embodiment 29 comprises the polypeptide or polypeptide complex of any one of embodiments 1 to 5 and 25 to 28, wherein the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 261.

Embodiment 30 comprises the polypeptide or polypeptide complex of any one of embodiments 1 to 5 and 25 to 28, wherein the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 262.

Embodiment 31 comprises a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and wherein the polypeptide or polypeptide complex comprises a second antibody or antigen binding fragment thereof that binds to CD3.

Embodiment 32 comprises the polypeptide or polypeptide complex of embodiment 1, wherein the immunoglobulin heavy chain and the immunoglobulin light chain comprise a set of six CDR sequences selected from the group consisting of: CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, CDR3-L: SEQ ID NO: 33; CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38.

Embodiment 33 comprises the polypeptide or polypeptide complex of any one of the preceding embodiments, wherein the immunoglobulin light chain comprises a variable domain of an immunoglobulin kappa (IgK) or immunoglobulin lambda (IgL) light chain.

Embodiment 34 comprises the polypeptide or polypeptide complex of any one of the preceding embodiments, wherein the immunoglobulin heavy chain comprises a constant domain derived from an IgG1, IgG2, IgG3, or IgG4 heavy chain.

Embodiment 35 comprises the polypeptide or polypeptide complex of embodiments 1 or 31, wherein the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; and wherein the immunoglobulin heavy chain comprises an amino acid sequence of at least 95% sequence identity to SEQ ID NO: 233 and the immunoglobulin light chain comprises an amino acid sequence of at least 95% sequence identity to SEQ ID NO: 234.

Embodiment 36 comprises the polypeptide or polypeptide complex of embodiment 35, wherein the immunoglobulin heavy chain comprises an amino acid sequence of at least 99% sequence identity to SEQ ID NO: 233 and the immunoglobulin light chain comprises an amino acid sequence of at least 99% sequence identity to SEQ ID NO: 234.

Embodiment 37 comprises the polypeptide or polypeptide complex of embodiment 35, wherein the immunoglobulin heavy chain comprises the amino acid sequence of SEQ ID NO: 233 and the immunoglobulin light chain comprises the amino acid sequence of SEQ ID NO: 234.

Embodiment 38 comprises the polypeptide or polypeptide complex of embodiments 1 or 31, wherein the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; and wherein the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40.

Embodiment 39 comprises the polypeptide or polypeptide complex of embodiment 38, wherein the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40.

Embodiment 40 comprises the polypeptide or polypeptide complex of embodiment 38, wherein the immunoglobulin heavy chain comprises the amino acid sequence of SEQ ID NO: 39 and the immunoglobulin light chain comprises the amino acid sequence of SEQ ID NO: 40.

Embodiment 41 comprises the polypeptide or polypeptide complex of embodiments 1 or 31, wherein the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; and wherein the immunoglobulin heavy chain comprises an amino acid sequence of at least 95% sequence identity to SEQ ID NO: 235 and the immunoglobulin light chain comprises an amino acid sequence of at least 95% sequence identity to SEQ ID NO: 236.

Embodiment 42 comprises the polypeptide or polypeptide complex of embodiment 41, wherein the immunoglobulin heavy chain comprises an amino acid sequence of at least 99% sequence identity to SEQ ID NO: 235 and the immunoglobulin light chain comprises an amino acid sequence of at least 99% sequence identity to SEQ ID NO: 236.

Embodiment 43 comprises the polypeptide or polypeptide complex of embodiment 41, wherein the immunoglobulin heavy chain comprises the amino acid sequence of SEQ ID NO: 235 and the immunoglobulin light chain comprises the amino acid sequence of SEQ ID NO: 236.

Embodiment 44 comprises the polypeptide or polypeptide complex of embodiments 1 or 31, wherein the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; and wherein the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42.

Embodiment 45 comprises the polypeptide or polypeptide complex of embodiment 44, wherein the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42.

Embodiment 46 comprises the polypeptide or polypeptide complex of embodiment 44, wherein the immunoglobulin heavy chain comprises the amino acid sequence of SEQ ID NO: 41 and the immunoglobulin light chain comprises the amino acid sequence of SEQ ID NO: 42.

Embodiment 47 comprises the polypeptide or polypeptide complex of embodiments 1 or 31, wherein the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; and wherein the immunoglobulin heavy chain comprises an amino acid sequence of at least 95% sequence identity to SEQ ID NO: 237 and the immunoglobulin light chain comprises an amino acid sequence of at least 95% sequence identity to SEQ ID NO: 238.

Embodiment 48 comprises the polypeptide or polypeptide complex of embodiment 47, wherein the immunoglobulin heavy chain comprises an amino acid sequence of at least 99% sequence identity to SEQ ID NO: 237 and the immunoglobulin light chain comprises an amino acid sequence of at least 99% sequence identity to SEQ ID NO: 238.

Embodiment 49 comprises the polypeptide or polypeptide complex of embodiment 47, wherein the immunoglobulin heavy chain comprises the amino acid sequence of SEQ ID NO: 237 and the immunoglobulin light chain comprises the amino acid sequence of SEQ ID NO: 238.

Embodiment 50 comprises the polypeptide or polypeptide complex of embodiments 1 or 31, wherein the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; and wherein the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44.

Embodiment 51 comprises the polypeptide or polypeptide complex of embodiment 50, wherein the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44.

Embodiment 52 comprises the polypeptide or polypeptide complex of embodiment 50, wherein the immunoglobulin heavy chain comprises the amino acid sequence of SEQ ID NO: 43 and the immunoglobulin light chain comprises the amino acid sequence of SEQ ID NO: 44.

Embodiment 53 comprises the polypeptide or polypeptide complex of embodiments 1 or 31, wherein the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and wherein the immunoglobulin heavy chain comprises an amino acid sequence of at least 95% sequence identity to SEQ ID NO: 239 and the immunoglobulin light chain comprises an amino acid sequence of at least 95% sequence identity to SEQ ID NO: 240.

Embodiment 54 comprises the polypeptide or polypeptide complex of embodiment 53, wherein the immunoglobulin heavy chain comprises an amino acid sequence of at least 99% sequence identity to SEQ ID NO: 239 and the immunoglobulin light chain comprises an amino acid sequence of at least 99% sequence identity to SEQ ID NO: 240.

Embodiment 55 comprises the polypeptide or polypeptide complex of embodiment 53, wherein the immunoglobulin heavy chain comprises the amino acid sequence of SEQ ID NO: 239 and the immunoglobulin light chain comprises the amino acid sequence of SEQ ID NO: 240.

Embodiment 56 comprises the polypeptide or polypeptide complex of embodiments 1 or 31, wherein the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and wherein the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46.

Embodiment 57 comprises the polypeptide or polypeptide complex of embodiment 56, wherein the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46.

Embodiment 58 comprises the polypeptide or polypeptide complex of embodiment 56, wherein the immunoglobulin heavy chain comprises the amino acid sequence of SEQ ID NO: 45 and the immunoglobulin light chain comprises the amino acid sequence of SEQ ID NO: 46.

Embodiment 59 comprises the polypeptide or polypeptide complex of embodiments 1 or 31, wherein the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and wherein the immunoglobulin heavy chain comprises an amino acid sequence of at least 95% sequence identity to SEQ ID NO: 241 and the immunoglobulin light chain comprises an amino acid sequence of at least 95% sequence identity to SEQ ID NO: 242.

Embodiment 60 comprises the polypeptide or polypeptide complex of embodiment 59, wherein the immunoglobulin heavy chain comprises an amino acid sequence of at least 99% sequence identity to SEQ ID NO: 241 and the immunoglobulin light chain comprises an amino acid sequence of at least 99% sequence identity to SEQ ID NO: 242.

Embodiment 61 comprises the polypeptide or polypeptide complex of embodiment 59, wherein the immunoglobulin heavy chain comprises the amino acid sequence of SEQ ID NO: 241 and the immunoglobulin light chain comprises the amino acid sequence of SEQ ID NO: 242.

Embodiment 62 comprises the polypeptide or polypeptide complex of embodiments 1 or 31, wherein the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and wherein the immunoglobulin heavy chain comprises an amino acid sequence of at least 95% sequence identity to SEQ ID NO: 47 and the immunoglobulin light chain comprises an amino acid sequence of at least 95% sequence identity to SEQ ID NO: 48.

Embodiment 63 comprises the polypeptide or polypeptide complex of embodiment 62, wherein the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48.

Embodiment 64 comprises the polypeptide or polypeptide complex of embodiment 62, wherein the immunoglobulin heavy chain comprises the amino acid sequence of SEQ ID NO: 47 and the immunoglobulin light chain comprises the amino acid sequence of SEQ ID NO: 48.

Embodiment 65 comprises the polypeptide or polypeptide complex of embodiments 1 or 31, wherein the immunoglobulin heavy chain comprises an amino acid sequence of at least 95%, 98%, 99%, or 100% sequence identity to SEQ ID NO: 251 and the immunoglobulin light chain comprises an amino acid sequence of at least 95%, 98%, 99%, or 100% sequence identity to SEQ ID NO: 252.

Embodiment 66 comprises the polypeptide or polypeptide complex of embodiments 1 or 31, wherein the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 255 and the immunoglobulin light chain comprises an amino acid sequence of at least 90% sequence identity to SEQ ID NO: 256.

Embodiment 67 comprises the polypeptide or polypeptide complex of embodiments 1 or 31, wherein the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 95% sequence identity to SEQ ID NO: 255 and the immunoglobulin light chain comprises an amino acid sequence of at least 95% sequence identity to SEQ ID NO: 256.

Embodiment 68 comprises the polypeptide or polypeptide complex of embodiments 1 or 31, wherein the immunoglobulin heavy chain comprises an amino acid sequence of at least 99% sequence identity to SEQ ID NO: 49 and the immunoglobulin light chain comprises an amino acid sequence of at least 99% sequence identity to SEQ ID NO: 50.

Embodiment 69 comprises the polypeptide or polypeptide complex of embodiments 1 or 31, wherein the immunoglobulin heavy chain comprises an amino acid sequence of at least 99% sequence identity to SEQ ID NO: 51 and the immunoglobulin light chain comprises an amino acid sequence of at least 99% sequence identity to SEQ ID NO: 52.

Embodiment 70 comprises the polypeptide or polypeptide complex of embodiments 1 or 31, wherein the immunoglobulin heavy chain comprises the amino acid sequence of SEQ ID NO: 51 and the immunoglobulin light chain comprises the amino acid sequence of SEQ ID NO: 52.

Embodiment 71 comprises the polypeptide or polypeptide complex of embodiments 1 or 31, wherein the immunoglobulin heavy chain comprises an amino acid sequence of at least 99% sequence identity to SEQ ID NO: 53 and the immunoglobulin light chain comprises an amino acid sequence of at least 99% sequence identity to SEQ ID NO: 54.

Embodiment 72 comprises the polypeptide or polypeptide complex of embodiments 1 or 31, wherein the immunoglobulin heavy chain comprises the amino acid sequence of SEQ ID NO: 53 and the immunoglobulin light chain comprises the amino acid sequence of SEQ ID NO: 54.

Embodiment 73 comprises the polypeptide or polypeptide complex of embodiments 1 or 31, wherein the immunoglobulin heavy chain comprises an amino acid sequence of at least 99% sequence identity to SEQ ID NO: 55 and the immunoglobulin light chain comprises an amino acid sequence of at least 99% sequence identity to SEQ ID NO: 56.

Embodiment 74 comprises the polypeptide or polypeptide complex of embodiments 1 or 31, wherein the immunoglobulin heavy chain comprises the amino acid sequence of SEQ ID NO: 55 and the immunoglobulin light chain comprises the amino acid sequence of SEQ ID NO: 56.

Embodiment 75 comprises the polypeptide or polypeptide complex of embodiments 1 or 31, wherein the immunoglobulin heavy chain comprises an amino acid sequence of at least 99% sequence identity to SEQ ID NO: 57 and the immunoglobulin light chain comprises an amino acid sequence of at least 99% sequence identity to SEQ ID NO: 58.

Embodiment 76 comprises the polypeptide or polypeptide complex of embodiments 1 or 31, wherein the immunoglobulin heavy chain comprises the amino acid sequence of SEQ ID NO: 57 and the immunoglobulin light chain comprises the amino acid sequence of SEQ ID NO: 58.

Embodiment 77 comprises the polypeptide or polypeptide complex of embodiments 1 or 31, wherein the immunoglobulin heavy chain comprises an amino acid sequence of at least 99% sequence identity to SEQ ID NO: 59 and the immunoglobulin light chain comprises an amino acid sequence of at least 99% sequence identity to SEQ ID NO: 60.

Embodiment 78 comprises the polypeptide or polypeptide complex of embodiments 1 or 31, wherein the immunoglobulin heavy chain comprises the amino acid sequence of SEQ ID NO: 59 and the immunoglobulin light chain comprises the amino acid sequence of SEQ ID NO: 60.

Embodiment 79 comprises the polypeptide or polypeptide complex of any one of the preceding embodiments, wherein the polypeptide or polypeptide complex that comprises the antibody or antigen binding fragment thereof that binds to STEAP-1 is a monoclonal antibody.

Embodiment 80 comprises the polypeptide or polypeptide complex of any one of the preceding embodiments, wherein the polypeptide or polypeptide complex that comprises the antibody or antigen binding fragment thereof that binds to STEAP-1 is a monospecific antibody, bispecific antibody, or trispecific antibody.

Embodiment 81 comprises the polypeptide or polypeptide complex of any one of the preceding embodiments, wherein the antibody or antigen binding fragment thereof that binds to STEAP-1 comprises a Fab, Fab′, (Fab′)₂ or a single chain variable fragment (scFv).

Embodiment 82 comprises the polypeptide or polypeptide complex of any one of the preceding embodiments, wherein the polypeptide or polypeptide complex comprises more than one antibody or antigen binding fragment thereof that binds to STEAP-1.

Embodiment 83 comprises the polypeptide or polypeptide complex of any one of the preceding claims, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁).

Embodiment 84 comprises the polypeptide or polypeptide complex of embodiment 83, wherein L₁ connects an immunoglobulin heavy chain of the Fab or Fab′ that binds STEAP-1 with another immunoglobulin heavy chain of the second Fab or Fab′ that binds STEAP-1.

Embodiment 85 comprises the polypeptide or polypeptide complex of embodiment 83, wherein L₁ connects an immunoglobulin light chain of the Fab or Fab′ that binds STEAP-1 with another immunoglobulin light chain of the second Fab or Fab′ that binds STEAP-1.

Embodiment 86 comprises the polypeptide or polypeptide complex of embodiment 83, wherein L₁ connects an immunoglobulin light chain of the Fab or Fab′ that binds STEAP-1 with an immunoglobulin heavy chain of the second Fab or Fab′ that binds STEAP-1.

Embodiment 87 comprises the polypeptide or polypeptide complex of any one of the preceding embodiments, wherein the second antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83; wherein CDR2-L comprises the amino acid sequence of GTK; wherein CDR3-L comprises the amino acid sequence of Z₁-Z₂-W-Z₃-Z₄-Z₅-Z₆-W-Z₇-Z₈ or SEQ ID NO: 82; wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 61 or SEQ ID NO: 64; wherein CDR2-H comprises the amino acid sequence of SEQ ID NO: 62; wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63; and wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

Embodiment 88 comprises the polypeptide or polypeptide complex of embodiment 87, wherein: Z₁ is V, G, L, I, or A; Z₂ is L, V, I, or A; Z₃ is Y, W, F, or A; Z₄ is S, G, T, or A; Z₅ is N, Q, D, E, or A; Z₆ is R, K, or A; Z₇ is V, G, L, I, or A; Z₈ is F, Y, W, or A; Z₉ is V, G, L, I, or A; Z₁₀ is R, K, or A; Z₁₂ is G, S, T, or A; Z₁₃ is F, Y, W, or A; Z₁₄ is G, S, T, or A; Z₁₅ is N, Q, D, E, or A; Z₁₆ is S, G, T, or A; Z₁₇ is Y, W, F, or A; Z₁₈ is I, V, L, or A; Z₁₉ is S, G, T, or A; Z₂₀ is W, Y, F, or A; and Z₂₁ is Y, W, F, or A.

Embodiment 89 comprises the polypeptide or polypeptide complex of embodiment 87, wherein: Z₈ is F.

Embodiment 90 comprises the polypeptide or polypeptide complex of embodiment 87, wherein: Z₁₀ is R; Z₁₁ is H; Z₁₃ is F; Z₁₈ is I; Z₁₉ is S; and Z₂₀ is W.

Embodiment 91 comprises the polypeptide or polypeptide complex of embodiment 87, wherein CDR1-H of the second antibody or antigen binding fragment thereof that binds to CD3 comprises the amino acid sequence of SEQ ID NO: 61 or SEQ ID NO: 64; wherein CDR2-H of the second antibody or antigen binding fragment thereof that binds to CD3 comprises the amino acid sequence of SEQ ID NO: 62; wherein CDR3-H of the second antibody or antigen binding fragment thereof that binds to CD3 comprises the amino acid sequence selected from SEQ ID NOs: 63, 65-79, and 124; wherein CDR1-L of the second antibody or antigen binding fragment thereof that binds to CD3 comprises the amino acid sequence of SEQ ID NO: 80 or SEQ ID NO: 83; wherein CDR2-L of the second antibody or antigen binding fragment thereof that binds to CD3 comprises the amino acid sequence of GTK; and wherein CDR3-L of the second antibody or antigen binding fragment thereof that binds to CD3 comprises the amino acid sequence selected from SEQ ID NOs: 82 and 84-93.

Embodiment 92 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 82.

Embodiment 93 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 65, CDR1-L: SEQ ID NO: 80, CDR2-L: GTK, CDR3-L: SEQ ID NO: 82.

Embodiment 94 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 66, CDR1-L: SEQ ID NO: 80, CDR2-L: GTK, CDR3-L: SEQ ID NO: 82.

Embodiment 95 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 67, CDR1-L: SEQ ID NO: 80, CDR2-L: GTK, CDR3-L: SEQ ID NO: 82.

Embodiment 96 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 68, CDR1-L: SEQ ID NO: 80, CDR2-L: GTK, CDR3-L: SEQ ID NO: 82.

Embodiment 97 comprises the polypeptide or polypeptide complex of any one of embodiments 1 to 87, wherein the second antibody or antigen binding fragment thereof that binds to CD3 comprises the 69, CDR1-L: SEQ ID NO: 80, CDR2-L: GTK, CDR3-L: SEQ ID NO: 82.

Embodiment 98 comprises the polypeptide or polypeptide complex of any one of embodiments 1 to 87, wherein the second antibody or antigen binding fragment thereof that binds to CD3 comprises the 70, CDR1-L: SEQ ID NO: 80, CDR2-L: GTK, CDR3-L: SEQ ID NO: 82.

Embodiment 99 comprises the polypeptide or polypeptide complex of any one of embodiments 1 to 87, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 71,
CDR1-L: SEQ ID NO: 80, CDR2-L: GTK, CDR3-L: SEQ ID NO: 82.

Embodiment 100 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 72, CDR1-L: SEQ ID NO: 80, CDR2-L: GTK, CDR3-L: SEQ ID NO: 82.

Embodiment 101 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 73, CDR1-L: SEQ ID NO: 80, CDR2-L: GTK, CDR3-L: SEQ ID NO: 82.

Embodiment 102 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 74, CDR1-L: SEQ ID NO: 80, CDR2-L: GTK, CDR3-L: SEQ ID NO: 82.

Embodiment 103 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 75, CDR1-L: SEQ ID NO: 80, CDR2-L: GTK, CDR3-L: SEQ ID NO: 82.

Embodiment 104 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 76, CDR1-L: SEQ ID NO: 80, CDR2-L: GTK, CDR3-L: SEQ ID NO: 82.

Embodiment 105 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 77, CDR1-L: SEQ ID NO: 80, CDR2-L: GTK, CDR3-L: SEQ ID NO: 82.

Embodiment 106 comprises the polypeptide or polypeptide complex of any one of embodiments 1 to 87, wherein the second antibody or antigen binding fragment thereof that binds to CD3 comprises the 78, CDR1-L: SEQ ID NO: 80, CDR2-L: GTK, CDR3-L: SEQ ID NO: 82.

Embodiment 107 comprises the polypeptide or polypeptide complex of any one of embodiments 1 to 87, wherein the second antibody or antigen binding fragment thereof that binds to CD3 comprises the 79, CDR1-L: SEQ ID NO: 80, CDR2-L: GTK, CDR3-L: SEQ ID NO: 82.

Embodiment 108 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 84.

Embodiment 109 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 85.

Embodiment 110 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 86.

Embodiment 111 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 87.

Embodiment 112 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 88.

Embodiment 113 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 89.

Embodiment 114 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 90.

Embodiment 115 comprises the polypeptide or polypeptide complex of any one of embodiments 1 to 87, wherein the second antibody or antigen binding fragment thereof that binds to CD3 comprises the 63, CDR1-L: SEQ ID NO: 80, CDR2-L: GTK, CDR3-L: SEQ ID NO: 91.

Embodiment 116 comprises the polypeptide or polypeptide complex of any one of embodiments 1 to 87, wherein the second antibody or antigen binding fragment thereof that binds to CD3 comprises the 63, CDR1-L: SEQ ID NO: 80, CDR2-L: GTK, CDR3-L: SEQ ID NO: 92.

Embodiment 117 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 93.

Embodiment 118 comprises the polypeptide or polypeptide complex of any one of embodiments 1 to 87, wherein the second antibody or antigen binding fragment thereof that binds to CD3 comprises the following set of six CDRs: CDR1-H: SEQ ID NO: 64, CDR2-H: SEQ ID NO: 62, CDR3-H: SEQ ID NO: 124, CDR1-L: SEQ ID NO: 83, CDR2-L: GTK, CDR3-L: SEQ ID NO: 84.

Embodiment 119 comprises the polypeptide or polypeptide complex of any one of the preceding embodiments, wherein the second antibody or antigen binding fragment thereof that binds to CD3 comprises a variable domain of an immunoglobulin kappa (IgK) or immunoglobulin lambda (IgL) light chain.

Embodiment 120 comprises the polypeptide or polypeptide complex of any one of the preceding embodiments, wherein the second antibody or antigen binding fragment thereof that binds to CD3 comprises a constant domain of an IgG1, IgG2, IgG3, or IgG4 heavy chain.

Embodiment 121 comprises the polypeptide or polypeptide complex of any one of embodiments 1 to 87, wherein the second antibody or antigen binding fragment thereof that binds to CD3 comprises amino acid sequences with at least 90%, 95%, 96%, 97%, 98%, 99%, or 100% sequence identity to SEQ ID NO: 94 and SEQ ID NO: 95.

Embodiment 122 comprises the polypeptide or polypeptide complex of any one of embodiments 1 to 87, wherein the second antibody or antigen binding fragment thereof that binds to CD3 comprises amino acid sequences with at least 90%, 95%, 96%, 97%, 98%, 99%, or 100% sequence identity to SEQ ID NO: 266 and SEQ ID NO: 267.

Embodiment 123 comprises the polypeptide or polypeptide complex of any one of embodiments 1 to 87, wherein the second antibody or antigen binding fragment thereof that binds to CD3 comprises amino acid sequences with at least 90%, 95%, 96%, 97%, 98%, or 100% sequence identity to SEQ ID NO: 268 and SEQ ID NO: 269.

Embodiment 124 comprises the polypeptide or polypeptide complex of any one of the preceding embodiments, wherein the second antibody or antigen binding fragment thereof that binds to CD3 comprises a Fab, Fab′, (Fab′)₂ or a single chain variable fragment (scFv).

Embodiment 125 comprises the polypeptide or polypeptide complex of embodiment 124, wherein the second antibody or antigen binding fragment thereof that binds to CD3 comprises the scFv.

Embodiment 126 comprises the polypeptide or polypeptide complex of embodiment 124, wherein the second antibody or antigen binding fragment thereof that binds to CD3 comprises the Fab.

Embodiment 127 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 96.

Embodiment 128 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 97.

Embodiment 129 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 98.

Embodiment 130 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 99.

Embodiment 131 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 100.

Embodiment 132 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 101.

Embodiment 133 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 102.

Embodiment 134 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 103.

Embodiment 135 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 104.

Embodiment 136 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 105.

Embodiment 137 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 106.

Embodiment 138 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 107.

Embodiment 139 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 108.

Embodiment 140 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 109.

Embodiment 141 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 110.

Embodiment 142 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 111.

Embodiment 143 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 112.

Embodiment 144 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 113.

Embodiment 145 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 114.

Embodiment 146 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 115.

Embodiment 147 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 116.

Embodiment 148 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 117.

Embodiment 149 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 118.

Embodiment 150 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 119.

Embodiment 151 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 120.

Embodiment 152 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 121.

Embodiment 153 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 122.

Embodiment 154 comprises the polypeptide or polypeptide complex of any one of embodiments 1 to 87, wherein the second antibody or antigen binding fragment thereof that binds to CD3 comprises an amino acid sequence with at least 90%, 95%, 96%, 97%, 98%, 99%, or 100% sequence identity to SEQ ID NO: 123.

Embodiment 155 comprises the polypeptide or polypeptide complex of any one of the preceding embodiments, wherein the antibody or antigen binding fragment thereof that binds to STEAP-1 is a Fab or Fab′, and the second antibody or antigen binding fragment thereof that binds to CD3 is an scFv and the Fab or Fab′ is connected to the scFv by a linker (L₂).

Embodiment 156 comprises the polypeptide or polypeptide complex of any one of the preceding embodiments, wherein the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′.

Embodiment 157 comprises the polypeptide or polypeptide complex of any one of embodiments 1 to 155, wherein the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′.

Embodiment 158 comprises the polypeptide or polypeptide complex of any one of embodiments 1 to 155, wherein the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin heavy chain.

Embodiment 159 comprises the polypeptide or polypeptide complex of any one of embodiments 1 to 155, wherein the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin heavy chain.

Embodiment 160 comprises the polypeptide or polypeptide complex of any one of embodiments 1 to 155, wherein the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin light chain.

Embodiment 161 comprises the polypeptide or polypeptide complex of any one of embodiments 1 to 155, wherein the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin light chain.

Embodiment 162 comprises the polypeptide or polypeptide complex of any one of embodiments 1 to 154, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to STEAP-1 is connected to the Fab or Fab′ that binds to CD3 by a linker (L₂).

Embodiment 163 comprises the polypeptide or polypeptide complex of any one of embodiments 1 to 154 and 162, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the Fab or Fab′ that binds to STEAP-1.

Embodiment 164 comprises the polypeptide or polypeptide complex of any one of embodiments 1 to 154 and 162, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the Fab or Fab′ that binds to STEAP-1.

Embodiment 165 comprises the polypeptide or polypeptide complex of any one of embodiments 1 to 154 and 162, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin heavy chain of the Fab or Fab′ that binds to STEAP-1.

Embodiment 166 comprises the polypeptide or polypeptide complex of any one of embodiments 1 to 154 and 162, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin heavy chain of the Fab or Fab′ that binds to STEAP-1.

Embodiment 167 comprises the polypeptide or polypeptide complex of any one of embodiments 1 to 154 and 162, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin light chain of the Fab or Fab′ that binds to STEAP-1.

Embodiment 168 comprises the polypeptide or polypeptide complex of any one of embodiments 1 to 154 and 162, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin light chain of the Fab or Fab′ that binds to STEAP-1.

Embodiment 169 comprises the polypeptide or polypeptide complex of any one of embodiments 1 to 154, wherein the antibody or antigen binding fragment thereof that binds to STEAP-1 is an scFv, and the second antibody or antigen binding fragment thereof that binds to CD3 is a Fab or Fab′ and the scFv that binds to STEAP-1 is connected to the Fab or Fab′ that binds to CD3 by a linker (L₂).

Embodiment 170 comprises the polypeptide or polypeptide complex of any one of embodiments 1 to 154 and 169, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the scFv that binds to STEAP-1.

Embodiment 171 comprises the polypeptide or polypeptide complex of any one of embodiments 1 to 154 and 169, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the scFv that binds to STEAP-1.

Embodiment 172 comprises the polypeptide or polypeptide complex of any one of embodiments 1 to 154 and 169, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin heavy chain of the scFv that binds to STEAP-1.

Embodiment 173 comprises the polypeptide or polypeptide complex of any one of embodiments 1 to 154 and 169, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin heavy chain of the scFv that binds to STEAP-1.

Embodiment 174 comprises the polypeptide or polypeptide complex of any one of embodiments 1 to 154 and 169, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin light chain of the scFv that binds to STEAP-1.

Embodiment 175 comprises the polypeptide or polypeptide complex of any one of embodiments 1 to 154 and 169, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin light chain of the scFv that binds to STEAP-1.

Embodiment 176 comprises the polypeptide or polypeptide complex of any one of embodiments 1 to 154, wherein the antibody or antigen binding fragment thereof that binds to STEAP-1 is an scFv, and wherein a C-terminus of the scFv that binds to STEAP-1 is connected to an N-terminus of the second antibody or antigen binding fragment thereof that binds to CD3 via a linker (L₂).

Embodiment 177 comprises the polypeptide or polypeptide complex of any one of embodiments 1 to 154, wherein the antibody or antigen binding fragment thereof that binds to STEAP-1 is an scFv, and wherein an N-terminus of the scFv that binds to STEAP-1 is connected to a C-terminus of the second antibody or antigen binding fragment thereof that binds to CD3 via a linker (L₂).

Embodiment 178 comprises the polypeptide or polypeptide complex of embodiment 177, wherein a C-terminus of the immunoglobulin light chain of the scFv that binds to STEAP-1 is connected to an N-terminus of the immunoglobulin heavy chain of the scFv that binds to STEAP-1 via a linker.

Embodiment 179 comprises the polypeptide or polypeptide complex of embodiment 177, wherein a C-terminus of the immunoglobulin heavy chain of the scFv that binds to STEAP-1 is connected to an N-terminus of the immunoglobulin light chain that binds to STEAP-1 via a linker.

Embodiment 180 comprises the polypeptide or polypeptide complex of any one of embodiments 1 to 154, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to an N terminus of one of the two connected Fab or Fab′ that are connected by L₁.

Embodiment 181 comprises the polypeptide or polypeptide complex of any one of embodiments 1 to 154, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to the C terminus of one of the two connected Fab or Fab′ that are connected by L₁.

Embodiment 182 comprises the polypeptide or polypeptide complex of any one of embodiments 1 to 154, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the N terminus of one of the two connected Fab or Fab′ that are connected by L₁.

Embodiment 183 comprises the polypeptide or polypeptide complex of any one of embodiments 1 to 154, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the C terminus of one of the two Fab or Fab′ that are connected by L₁.

Embodiment 184 comprises the polypeptide or polypeptide complex of any one of embodiments 1 to 154, wherein L₁ connects an immunoglobulin heavy chain of the Fab or Fab′ that binds to STEAP-1 with an immunoglobulin light chain of the second antibody or antigen binding fragment that binds to CD3, and L₂ connects an immunoglobulin heavy chain of the second antibody or antigen binding fragment that binds to CD3 with another immunoglobulin heavy chain of the second Fab or Fab′ that binds to STEAP-1.

Embodiment 185 comprises the polypeptide or polypeptide complex of any one of embodiments 83 to 184, wherein L₁ or L₂ comprises an amino acid sequence according to any one of SEQ ID NOs: 222-224, 270-272, and 371.

Embodiment 186 comprises the polypeptide or polypeptide complex of embodiments 1 or 31, wherein the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 128-137, 139-149, 283-284, 287-288, and 291-292, and an amino acid sequence, that comprises the immunoglobulin heavy chain that binds to STEAP-1, with at least 95%, 98%, 99%, or 100% sequence identity to any one of SEQ ID NOs: 176-185, 187-197, 308-309, 312-313, and 316-317.

Embodiment 187 comprises the polypeptide or polypeptide complex of embodiments 1 or 31, wherein the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 126-127, 138, 273-282, 285-286, and 289-290, and an amino acid sequence, that comprises the immunoglobulin heavy chain that binds to STEAP-1, with at least 95%, 98%, or 99% sequence identity to any one of SEQ ID NOs: 174-175, 186, 299-307, 310-311, and 314-315.

Embodiment 188 comprises the polypeptide or polypeptide complex of embodiments 1 or 31, wherein the polypeptide or polypeptide complex comprises an amino acid sequence, that comprises the immunoglobulin light chain that binds to STEAP-1, according to SEQ ID NO: 283 and an amino acid sequence, that comprises the immunoglobulin heavy chain that binds to STEAP-1, according to SEQ ID NO: 308.

Embodiment 189 comprises the polypeptide or polypeptide complex of embodiments 1 or 31, wherein the polypeptide or polypeptide complex comprises an amino acid sequence that comprises the immunoglobulin light chain that binds to STEAP-1 according to SEQ ID NO: 284 and an amino acid sequence that comprises the immunoglobulin heavy chain that binds to STEAP-1 according to SEQ ID NO: 309.

Embodiment 190 comprises the polypeptide or polypeptide complex of embodiments 1 or 31, wherein the polypeptide or polypeptide complex comprises an amino acid sequence that comprises the immunoglobulin light chain that binds to STEAP-1 according to SEQ ID NO: 287 and an amino acid sequence that comprises the immunoglobulin heavy chain that binds to STEAP-1 according to SEQ ID NO: 312.

Embodiment 191 comprises the polypeptide or polypeptide complex of embodiments 1 or 31, wherein the polypeptide or polypeptide complex comprises an amino acid sequence that comprises the immunoglobulin light chain that binds to STEAP-1 according to SEQ ID NO: 288 and an amino acid sequence that comprises the immunoglobulin heavy chain that binds to STEAP-1 according to SEQ ID NO: 313.

Embodiment 192 comprises the polypeptide or polypeptide complex of embodiments 1 or 31, wherein the polypeptide or polypeptide complex comprises an amino acid sequence that comprises the immunoglobulin light chain that binds to STEAP-1 according to SEQ ID NO: 291 and an amino acid sequence that comprises the immunoglobulin heavy chain that binds to STEAP-1 according to SEQ ID NO: 316.

Embodiment 193 comprises the polypeptide or polypeptide complex of embodiments 1 or 31, wherein the polypeptide or polypeptide complex comprises an amino acid sequence that comprises the immunoglobulin light chain that binds to STEAP-1 according to SEQ ID NO: 292 and an amino acid sequence that comprises the immunoglobulin heavy chain that binds to STEAP-1 according to SEQ ID NO: 317.

Embodiment 194 comprises the polypeptide or polypeptide complex of embodiments 1 or 31, wherein the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 152-161 and 164-173, and an amino acid sequence, that comprises the immunoglobulin heavy chain that binds to STEAP-1, with at least 95%, 98%, 99%, or 100% sequence identity to any one of SEQ ID NOs: 200-209 and 212-221.

Embodiment 195 comprises the polypeptide or polypeptide complex of embodiments 1 or 31, wherein the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 150-151, 162-163, and 330-342 and an amino acid sequence, that comprises the immunoglobulin heavy chain that binds to STEAP-1, with at least 95%, 98%, 99%, or 100% sequence identity to any one of SEQ ID NOs: 198-199, 210-211, and 343-355.

Embodiment 196 comprises the polypeptide or polypeptide complex of embodiments 1 or 31, wherein the polypeptide or polypeptide complex comprises amino acid sequences with at least 95%, 98%, or 99% sequence identity to SEQ ID NOs: 324, 325, and 326.

Embodiment 197 comprises the polypeptide or polypeptide complex of embodiments 1 or 31, wherein the polypeptide or polypeptide complex comprises amino acid sequences with at least 95%, 98%, or 99% sequence identity to SEQ ID NOs: 327, 328, and 329.

Embodiment 198 comprises the polypeptide or polypeptide complex of embodiments 1 or 31, wherein the polypeptide or polypeptide complex comprises amino acid sequences with at least 95%, 98%, or 99% sequence identity to SEQ ID NOs: 356, 357, 358, and 359.

Embodiment 199 comprises the polypeptide or polypeptide complex of embodiments 1 or 31, wherein the polypeptide or polypeptide complex comprises amino acid sequences with at least 95%, 98%, or 99% sequence identity to SEQ ID NOs: 360, 361, 362, and 363.

Embodiment 200 comprises the polypeptide or polypeptide complex of embodiments 1 or 31, wherein the polypeptide or polypeptide complex comprises amino acid sequences with at least 95%, 98%, or 99% sequence identity to SEQ ID NOs: 364, 365, 366, and 367.

Embodiment 201 comprises a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, CDR3-L: SEQ ID NO: 33; and wherein the polypeptide or polypeptide complex further comprises a second antibody or antigen binding fragment thereof that binds to CD3 wherein the second antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H, wherein CDR1-L comprises the amino acid sequence of SEQ ID NO: 80 or SEQ ID NO: 83; wherein CDR2-L comprises the amino acid sequence of GTK; wherein CDR3-L comprises the amino acid sequence of Z₁-Z₂-W-Z₃-Z₄-Z₅-Z₆-W-Z₇-Z₈ or SEQ ID NO: 82; wherein Z₁ is V, G, P, L, I, M, S, T, or A; Z₂ is L, G, P, V, I, M, S, T, or A; Z₃ is Y, F, W, V, L, I, G, or A; Z₄ is S, G, T, M, N, Q, H, or A; Z₅ is N, Q, S, T, D, E, H, K, R, or A; Z₆ is R, S, T, Q, D, E, H, K, N, or A; Z₇ is V, G, P, L, I, M, S, T, or A; and Z₈ is F, Y, W, V, L, I, G, or A; wherein CDR1-H comprises the amino acid sequence of SEQ ID NO: 61 or SEQ ID NO: 64; wherein CDR2-H comprises the amino acid sequence of SEQ ID NO: 62; wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63; and wherein Z₉ is V, G, P, L, I, M, S, T, or A; Z₁₀ is R, S, T, Q, D, E, H, K, N, or A; Z₁₁ is H, R, K, G, T, S, N, Q, or A; Z₁₂ is G, P, V, L, I, M, S, T, or A; Z₁₃ is F, Y, W, V, L, I, G, or A; Z₁₄ is G, P, V, L, I, M, S, T, or A; Z₁₅ is N, Q, S, T, D, E, H, K, R, or A; Z₁₆ is S, G, T, M, N, Q, H, or A; Z₁₇ is Y, F, W, V, L, I, G, or A; Z₁₈ is I, G, P, V, L, M, S, T, or A; Z₁₉ is S, G, T, M, N, Q, H, or A; Z₂₀ is W, F, Y, V, L, I, G, or A; and Z₂₁ is Y, F, W, V, L, I, G, or A.

Embodiment 202 comprises the polypeptide or polypeptide complex of embodiment 201, wherein the antibody or antigen binding fragment thereof that binds to STEAP-1 comprises an amino acid sequence of at least 95% sequence identity to SEQ ID NO: 225 and an amino acid sequence of at least 95% sequence identity to SEQ ID NO: 226.

Embodiment 203 comprises the polypeptide or polypeptide complex of embodiment 201, wherein the antibody or antigen binding fragment thereof that binds to STEAP-1 comprises an amino acid sequence of at least 99% sequence identity to SEQ ID NO: 225 and an amino acid sequence of at least 99% sequence identity to SEQ ID NO: 226.

Embodiment 204 comprises the polypeptide or polypeptide complex of embodiment 201, wherein the antibody or antigen binding fragment thereof that binds to STEAP-1 comprises the amino acid sequence of SEQ ID NO: 225 and the amino acid sequence of SEQ ID NO: 226.

Embodiment 205 comprises the polypeptide or polypeptide complex of embodiment 201, wherein the antibody or antigen binding fragment thereof that binds to STEAP-1 comprises an amino acid sequence of at least 95% sequence identity to SEQ ID NO: 1 and an amino acid sequence of at least 95% sequence identity to SEQ ID NO: 2.

Embodiment 206 comprises the polypeptide or polypeptide complex of embodiment 201, wherein the antibody or antigen binding fragment thereof that binds to STEAP-1 comprises an amino acid sequence of at least 99% sequence identity to SEQ ID NO: 1 and an amino acid sequence of at least 99% sequence identity to SEQ ID NO: 2.

Embodiment 207 comprises the polypeptide or polypeptide complex of embodiment 201, wherein the antibody or antigen binding fragment thereof that binds to STEAP-1 comprises the amino acid sequence of SEQ ID NO: 1 and the amino acid sequence of SEQ ID NO: 2.

Embodiment 208 comprises the polypeptide or polypeptide complex of any one of embodiments 201 to 207, wherein the antibody or antigen binding fragment thereof that binds to STEAP-1 comprises a Fab, Fab′, (Fab′)₂ or a single chain variable fragment (scFv).

Embodiment 209 comprises he polypeptide or polypeptide complex of embodiment any one of embodiments 201 to 208, wherein the polypeptide or polypeptide complex comprises more than one antibody or antigen binding fragment thereof that binds to STEAP-1.

Embodiment 210 comprises the polypeptide or polypeptide complex of any one of embodiments 201 to 209, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁).

Embodiment 211 comprises the polypeptide or polypeptide complex of embodiment 210, wherein L₁ connects an immunoglobulin heavy chain of the Fab or Fab′ that binds STEAP-1 with another immunoglobulin heavy chain of the second Fab or Fab′ that binds STEAP-1.

Embodiment 212 comprises the polypeptide or polypeptide complex of embodiment 210, wherein L₁ connects an immunoglobulin light chain of the Fab or Fab′ that binds STEAP-1 with another immunoglobulin light chain of the second Fab or Fab′ that binds STEAP-1.

Embodiment 213 comprises the polypeptide or polypeptide complex of embodiment 210, wherein L₁ connects an immunoglobulin light chain of the Fab or Fab′ that binds STEAP-1 with an immunoglobulin heavy chain of the second Fab or Fab′ that binds STEAP-1.

Embodiment 214 comprises the polypeptide or polypeptide complex of any one of embodiments 201 to 213, wherein CDR1-H of the second antibody or antigen binding fragment thereof that binds to CD3 comprises the amino acid sequence of SEQ ID NO: 61 or SEQ ID NO: 64; wherein CDR2-H of the second antibody or antigen binding fragment thereof that binds to CD3 comprises the amino acid of SEQ ID NO: 62; wherein CDR3-H of the second antibody or antigen binding fragment thereof that binds to CD3 comprises the amino acid sequence selected from SEQ ID NOs: 63, 65-79, and 124; wherein CDR1-L of the second antibody or antigen binding fragment thereof that binds to CD3 comprises the amino acid sequence of SEQ ID NO: 80 or SEQ ID NO: 83; wherein CDR2-L of the second antibody or antigen binding fragment thereof that binds to CD3 comprises the amino acid sequence of GTK; and wherein CDR3-L of the second antibody or antigen binding fragment thereof that binds to CD3 comprises the amino acid sequence selected from SEQ ID NOs: 82 and 84-93.

Embodiment 215 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 82.

Embodiment 216 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 65, CDR1-L: SEQ ID NO: 80, CDR2-L: GTK, CDR3-L: SEQ ID NO: 82.

Embodiment 217 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 66, CDR1-L: SEQ ID NO: 80, CDR2-L: GTK, CDR3-L: SEQ ID NO: 82.

Embodiment 218 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 67, CDR1-L: SEQ ID NO: 80, CDR2-L: GTK, CDR3-L: SEQ ID NO: 82.

Embodiment 219 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 68, CDR1-L: SEQ ID NO: 80, CDR2-L: GTK, CDR3-L: SEQ ID NO: 82.

Embodiment 220 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 69, CDR1-L: SEQ ID NO: 80, CDR2-L: GTK, CDR3-L: SEQ ID NO: 82.

Embodiment 221 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 70, CDR1-L: SEQ ID NO: 80, CDR2-L: GTK, CDR3-L: SEQ ID NO: 82.

Embodiment 222 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 71, CDR1-L: SEQ ID NO: 80, CDR2-L: GTK, CDR3-L: SEQ ID NO: 82.

Embodiment 223 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 72, CDR1-L: SEQ ID NO: 80, CDR2-L: GTK, CDR3-L: SEQ ID NO: 82.

Embodiment 224 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 73, CDR1-L: SEQ ID NO: 80, CDR2-L: GTK, CDR3-L: SEQ ID NO: 82.

Embodiment 225 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 74, CDR1-L: SEQ ID NO: 80, CDR2-L: GTK, CDR3-L: SEQ ID NO: 82.

Embodiment 226 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 75, CDR1-L: SEQ ID NO: 80, CDR2-L: GTK, CDR3-L: SEQ ID NO: 82.

Embodiment 227 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 76, CDR1-L: SEQ ID NO: 80, CDR2-L: GTK, CDR3-L: SEQ ID NO: 82.

Embodiment 228 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 77, CDR1-L: SEQ ID NO: 80, CDR2-L: GTK, CDR3-L: SEQ ID NO: 82.

Embodiment 229 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 78, CDR1-L: SEQ ID NO: 80, CDR2-L: GTK, CDR3-L: SEQ ID NO: 82.

Embodiment 230 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 79, CDR1-L: SEQ ID NO: 80, CDR2-L: GTK, CDR3-L: SEQ ID NO: 82.

Embodiment 231 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 84.

Embodiment 232 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 85.

Embodiment 233 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 86.

Embodiment 234 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 87.

Embodiment 235 comprises the polypeptide or polypeptide complex of any one of embodiments 201 to 214, wherein the antibody or antigen binding fragment thereof that binds to CD3 comprises the 63, CDR1-L: SEQ ID NO: 80, CDR2-L: GTK, CDR3-L: SEQ ID NO: 88.

Embodiment 236 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 89.

Embodiment 237 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 90.

Embodiment 238 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 91.

Embodiment 239 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 92.

Embodiment 240 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 61, CDR2-H: SEQ ID NO: 62, CDR3-H: SEQ ID NO: 63, CDR1-L: SEQ ID NO: 80, CDR2-L: GTK, CDR3-L: SEQ ID NO: 93.

Embodiment 241 comprises the polypeptide or polypeptide complex of any one of embodiments 201 to 214, wherein the second antibody or antigen binding fragment thereof that binds to CD3 comprises the following set of six CDRs: CDR1-H: SEQ ID NO: 64, CDR2-H: SEQ ID NO: 62, CDR3-H: SEQ ID NO: 124, CDR1-L: SEQ ID NO: 83, CDR2-L: GTK, CDR3-L: SEQ ID NO: 84.

Embodiment 242 comprises the polypeptide or polypeptide complex of any one of embodiments 201 to 241, wherein the second antibody or antigen binding fragment thereof that binds to CD3 comprises a variable domain of an immunoglobulin kappa (IgK) or immunoglobulin lambda (IgL) light chain.

Embodiment 243 comprises the polypeptide or polypeptide complex of any one of embodiments 201 to 242, wherein the second antibody or antigen binding fragment thereof that binds to CD3 comprises a constant domain of an IgG1, IgG2, IgG3, or IgG4 heavy chain.

Embodiment 244 comprises the polypeptide or polypeptide complex of any one of embodiments 201 to 214, wherein the second antibody or antigen binding fragment thereof that binds to CD3 comprises amino acid sequences with at least 90%, 95%, 96%, 97%, 98%, 99%, or 100% sequence identity to SEQ ID NO: 94 and SEQ ID NO: 95.

Embodiment 245 comprises the polypeptide or polypeptide complex of any one of embodiments 201 to 214, wherein the second antibody or antigen binding fragment thereof that binds to CD3 comprises amino acid sequences with at least 90%, 95%, 96%, 97%, 98%, 99%, or 100% sequence identity to SEQ ID NO: 266 and SEQ ID NO: 267.

Embodiment 246 comprises the polypeptide or polypeptide complex of any one of embodiments 201 to 214, wherein the second antibody or antigen binding fragment thereof that binds to CD3 comprises amino acid sequences with at least 90%, 95%, 96%, 97%, 98%, or 100% sequence identity to SEQ ID NO: 268 and SEQ ID NO: 269.

Embodiment 247 comprises the polypeptide or polypeptide complex of any one of embodiments 201 to 246, wherein the second antibody or antigen binding fragment thereof that binds to CD3 comprises a Fab, Fab′, (Fab′)₂ or a single chain variable fragment (scFv).

Embodiment 248 comprises the polypeptide or polypeptide complex of embodiment 247, wherein the second antibody or antigen binding fragment thereof that binds to CD3 comprises the scFv.

Embodiment 249 comprises the polypeptide or polypeptide complex of embodiment 247, wherein the second antibody or antigen binding fragment thereof that binds to CD3 comprises the Fab.

Embodiment 250 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 96.

Embodiment 251 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 97.

Embodiment 252 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 98.

Embodiment 253 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 99.

Embodiment 254 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 100.

Embodiment 255 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 101.

Embodiment 256 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 102.

Embodiment 257 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 103.

Embodiment 258 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 104.

Embodiment 259 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 105.

Embodiment 260 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 106.

Embodiment 261 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 107.

Embodiment 262 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 108.

Embodiment 263 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 109.

Embodiment 264 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 110.

Embodiment 265 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 111.

Embodiment 266 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 112.

Embodiment 267 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 113.

Embodiment 268 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 114.

Embodiment 269 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 115.

Embodiment 270 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 116.

Embodiment 271 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 117.

Embodiment 272 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 118.

Embodiment 273 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 119.

Embodiment 274 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 120.

Embodiment 275 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 121.

Embodiment 276 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 122.

Embodiment 277 comprises the polypeptide or polypeptide complex of any one of embodiments 201 to 214, wherein the second antibody or antigen binding fragment thereof that binds to CD3 comprises an amino acid sequence with at least 90%, 95%, 96%, 97%, 98%, 99%, or 100% sequence identity to SEQ ID NO: 123.

Embodiment 278 comprises the polypeptide or polypeptide complex of any one of embodiments 201 to 214, wherein the antibody or antigen binding fragment thereof that binds to STEAP-1 is a Fab or Fab′, and the second antibody or antigen binding fragment thereof that binds to CD3 is an scFv and the Fab or Fab′ is connected to the scFv by a linker (L₂).

Embodiment 279 comprises the polypeptide or polypeptide complex of any one of embodiments 201 to 214 and 278, wherein the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′.

Embodiment 280 comprises the polypeptide or polypeptide complex of any one of embodiments 201 to 214 and 278, wherein the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′.

Embodiment 281 comprises the polypeptide or polypeptide complex of any one of embodiments 201 to 214 and 278, wherein the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin heavy chain.

Embodiment 282 comprises the polypeptide or polypeptide complex of any one of embodiments 201 to 214 and 278, wherein the antibody or antigen binding fragment thereof that binds to STEAP-1 is a Fab or Fab′, and the second antibody or antigen binding fragment thereof that binds to CD3 is an scFv and the scFv is connected to a C terminus of the Fab or Fab′ immunoglobulin heavy chain.

Embodiment 283 comprises the polypeptide or polypeptide complex of any one of embodiments 201 to 214 and 278, wherein the antibody or antigen binding fragment thereof that binds to STEAP-1 is a Fab or Fab′, and the second antibody or antigen binding fragment thereof that binds to CD3 is an scFv and the scFv is connected to an N terminus of the Fab or Fab′ immunoglobulin light chain.

Embodiment 284 comprises the polypeptide or polypeptide complex of any one of embodiments 201 to 214 and 278, wherein the antibody or antigen binding fragment thereof that binds to STEAP-1 is a

Fab or Fab′, and the second antibody or antigen binding fragment thereof that binds to CD3 is an scFv and the scFv is connected to a C terminus of the Fab or Fab′ immunoglobulin light chain.

Embodiment 285 comprises the polypeptide or polypeptide complex of any one of embodiments 201 to 214, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to STEAP-1 is connected to the Fab or Fab′ that binds to CD3 by a linker (L₂).

Embodiment 286 comprises the polypeptide or polypeptide complex of any one of embodiments 201 to 214 and 285, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the Fab or Fab′ that binds to STEAP-1.

Embodiment 287 comprises the polypeptide or polypeptide complex of any one of embodiments 201 to 214 and 285, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the Fab or Fab′ that binds to STEAP-1.

Embodiment 288 comprises the polypeptide or polypeptide complex of any one of embodiments 201 to 214 and 285, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin heavy chain of the Fab or Fab′ that binds to STEAP-1.

Embodiment 289 comprises the polypeptide or polypeptide complex of any one of embodiments 201 to 214 and 285, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin heavy chain of the Fab or Fab′ that binds to STEAP-1.

Embodiment 290 comprises the polypeptide or polypeptide complex of any one of embodiments 201 to 214 and 285, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to an N terminus of the immunoglobulin light chain of the Fab or Fab′ that binds to STEAP-1.

Embodiment 291 comprises the polypeptide or polypeptide complex of any one of embodiments 201 to 214 and 285, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to CD3 is connected to a C terminus of the immunoglobulin light chain of the Fab or Fab′ that binds to STEAP-1.

Embodiment 292 comprises the polypeptide or polypeptide complex of any one of embodiments 201 to 214, wherein the antibody or antigen binding fragment thereof that binds to STEAP-1 is an scFv, and the second antibody or antigen binding fragment thereof that binds to CD3 is a Fab or Fab′ and the scFv that binds to STEAP-1 is connected to the Fab or Fab′ that binds to CD3 by a linker (L₂).

Embodiment 293 comprises the polypeptide or polypeptide complex of any one of embodiments 201 to 214 and 292, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the scFv that binds to STEAP-1.

Embodiment 294 comprises the polypeptide or polypeptide complex of any one of embodiments 201 to 214 and 292, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the scFv that binds to STEAP-1.

Embodiment 295 comprises the polypeptide or polypeptide complex of any one of embodiments 201 to 214 and 292, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin heavy chain of the scFv that binds to STEAP-1.

Embodiment 296 comprises the polypeptide or polypeptide complex of any one of embodiments 201 to 214 and 292, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin heavy chain of the scFv that binds to STEAP-1.

Embodiment 297 comprises the polypeptide or polypeptide complex of any one of embodiments 201 to 214 and 292, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to an N terminus of the immunoglobulin light chain of the scFv that binds to STEAP-1.

Embodiment 298 comprises the polypeptide or polypeptide complex of any one of embodiments 201 to 214 and 292, wherein the antibody or antigen binding fragment thereof that binds to STEAP-1 is an scFv, and the second antibody or antigen binding fragment thereof that binds to CD3 is a Fab or Fab′ and the Fab or Fab′ that binds to CD3 is connected to a C terminus of the immunoglobulin light chain of the scFv that binds to STEAP-1.

Embodiment 299 comprises the polypeptide or polypeptide complex of any one of embodiments 201 to 214, wherein the antibody or antigen binding fragment thereof that binds to STEAP-1 is an scFv, and wherein a C-terminus of the scFv that binds to STEAP-1 is connected to an N-terminus of the second antibody or antigen binding fragment thereof that binds to CD3 via a linker (L₂).

Embodiment 300 comprises the polypeptide or polypeptide complex of any one of embodiments 201 to 214, wherein the antibody or antigen binding fragment thereof that binds to STEAP-1 is an scFv, and wherein an N-terminus of the scFv that binds to STEAP-1 is connected to a C-terminus of the second antibody or antigen binding fragment thereof that binds to CD3 via a linker (L₂).

Embodiment 301 comprises the polypeptide or polypeptide complex of embodiments 299 or 300, wherein a C-terminus of the immunoglobulin light chain of the scFv that binds to STEAP-1 is connected to an N-terminus of the immunoglobulin heavy chain of the scFv that binds to STEAP-1 via a linker.

Embodiment 302 comprises the polypeptide or polypeptide complex of embodiments 299 or 300, wherein a C-terminus of the immunoglobulin heavy chain of the scFv that binds to STEAP-1 is connected to an N-terminus of the immunoglobulin light chain that binds to STEAP-1 via a linker.

Embodiment 303 comprises the polypeptide or polypeptide complex of any one of embodiments 201 to 214, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to an N terminus of one of the two connected Fab or Fab′ that are connected by L₁.

Embodiment 304 comprises the polypeptide or polypeptide complex of any one of embodiments 201 to 214, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin heavy chain of the Fab or Fab′ that binds STEAP-1 with another immunoglobulin heavy chain of the Fab or Fab′ that binds STEAP-1 and the second antibody or antigen binding fragment thereof that binds to CD3 is an scFv and the scFv is connected by L₂ to the C terminus of one of the two connected Fab or Fab′ that are connected by L₁.

Embodiment 305 comprises the polypeptide or polypeptide complex of any one of embodiments 201 to 214, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the N terminus of one of the two connected Fab or Fab′ that are connected by L₁.

Embodiment 306 comprises the polypeptide or polypeptide complex of any one of embodiments 201 to 214, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁) and L₁ connects an immunoglobulin light chain of the Fab or Fab′ that binds STEAP-1 with another immunoglobulin light chain of the Fab or Fab′ that binds STEAP-1 and the second antibody or antigen binding fragment thereof that binds to CD3 is an scFv and the scFv is connected by L₂ to the C terminus of one of the two Fab or Fab′ that are connected by L₁.

Embodiment 307 comprises the polypeptide or polypeptide complex of any one of embodiments 201 to 214, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by the second antibody or antigen binding fragment thereof that binds to CD3, wherein the second antibody or antigen binding fragment thereof that binds to CD3 comprises a CrossFab configuration, and wherein the Fab or Fab′ that binds to STEAP-1 is connected to the second antibody or antigen binding fragment thereof that binds to CD3 via a linker (L₁) and the second Fab or Fab′ that binds to STEAP-1 is connected to the second antibody or antigen binding fragment thereof that binds to CD3 via a linker (L₂).

Embodiment 308 comprises the polypeptide or polypeptide complex of embodiment 307, wherein L₁ connects an immunoglobulin heavy chain of the Fab or Fab′ that binds to STEAP-1 with an immunoglobulin light chain of the second antibody or antigen binding fragment that binds to CD3, and L₂ connects an immunoglobulin heavy chain of the second antibody or antigen binding fragment that binds to CD3 with another immunoglobulin heavy chain of the second Fab or Fab′ that binds to STEAP-1.

Embodiment 309 comprises the polypeptide or polypeptide complex of any one of embodiments 210 to 308, wherein L₁ or L₂ comprises an amino acid sequence according to any one of SEQ ID NOs: 222-224, 270-272, and 371.

Embodiment 310 comprises the polypeptide or polypeptide complex of embodiment 201, wherein the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 126-127, 138-139, 273-282, 285-286, and 289-290, and an amino acid sequence, that comprises the immunoglobulin heavy chain that binds to STEAP-1, with at least 95%, 98%, 99%, or 100% sequence identity to any one of SEQ ID NOs: 174-175, 186-187, 299-302, 372, 303-307, 310-311, and 314-315.

Embodiment 311 comprises the polypeptide or polypeptide complex of embodiment 201, wherein the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 150-151, 162-163, and 330-342, and an amino acid sequence, that comprises the immunoglobulin heavy chain that binds to STEAP-1, with at least 95%, 98%, 99%, or 100% sequence identity to any one of SEQ ID NOs: 198-199, 210-211, and 343-355.

Embodiment 312 comprises the polypeptide or polypeptide complex of embodiment 201, wherein the polypeptide or polypeptide complex comprises amino acid sequences with at least 95%, 98%, 99%, or 100% sequence identity to SEQ ID NOs: 327, 328, and 329.

Embodiment 313 comprises the polypeptide or polypeptide complex of embodiment 201, wherein the polypeptide or polypeptide complex comprises amino acid sequences with at least 95%, 98%, 99%, or 100% sequence identity to SEQ ID NOs: 366, 357, 358, and 359.

Embodiment 314 comprises the polypeptide or polypeptide complex of embodiment 201, wherein the polypeptide or polypeptide complex comprises amino acid sequences with at least 95%, 98%, 99%, or 100% sequence identity to SEQ ID NOs: 360, 361, 362, and 363.

Embodiment 315 comprises a pharmaceutical composition comprising: (i) the polypeptide or polypeptide complex of any one of embodiments 1 to 314; and (ii) a pharmaceutically acceptable excipient.

Embodiment 316 comprises an isolated recombinant nucleic acid molecule encoding the polypeptide or polypeptide complex of any one of embodiments 1 to 314.

Embodiment 317 comprises an expression vector comprising a nucleic acid encoding the polypeptide or polypeptide complex of any one of embodiments 1 to 314.

Embodiment 318 comprises a host cell comprising the polypeptide or polypeptide complex of any one of embodiments 1 to 314.

Embodiment 319 comprises a method of treating a cancer in a subject in need thereof comprising administering to the subject the polypeptide or polypeptide complex any one of embodiments 1 to 314.

Embodiment 320 comprises the method of embodiment 319, wherein the cancer is sarcoma or a cancer of the prostate, breast, pancreas, bladder, gastrointestinal tract, testis, ovary, or cervix.

Embodiment 321 comprises the method of embodiment 319, wherein the cancer is prostate cancer or Ewing sarcoma.

Embodiment 322 comprises the method of embodiment 319, further comprising administering to the subject an immune checkpoint inhibitor.

Embodiment 323 comprises the method of embodiment 322, wherein the immune checkpoint inhibitor comprises an antibody or antigen binding fragment thereof that binds to PD-1.

### EXAMPLES

### Example 1: STEAP-1 TCE Activity Against Tumor Cell Lines

STEAP-1 TCEs were evaluated in a functional in vitro tumor cell killing assay using the STEAP-1 positive prostate cancer cell lines Lymph Node Carcinoma of the Prostate (LNCaP), Vertebral Cancer of the Prostate (VCaP), 22Rv1, C4-2B, A673, IM-95, or OE33. Tumor cell line STEAP-1 expression was quantified via flow cytometry using a STEAP-1 antibody labeled with phycoerythrin (PE) in a 1:1 antibody: PE molar ratio and compared to PE Quantibrite beads according to manufacturer's instructions. Tumor cell killing was measured using an xCelligence real time analyzer from Agilent that relies on sensor impedance measurements (cell index) that increased as tumor cells adhere, spread, and expand on the surface of the sensor. Likewise, as the tumor cells were killed, the impedance decreased. 10,000 tumor cells were added per well and allowed to adhere overnight on a 96 well plate. The following day, TCEs titrated in human serum supplemented medium along with 30,000 CD8+ T cells were added to the wells. Cell index measurements were taken every 10 minutes for an additional 120 hours. The cell index times number of hours (tumor cell growth kinetics) was then plotted versus concentration of TCE, where the concentration of TCE required to reduce the tumor growth by 50% (IC₅₀) was calculated using Graphpad Prism software.

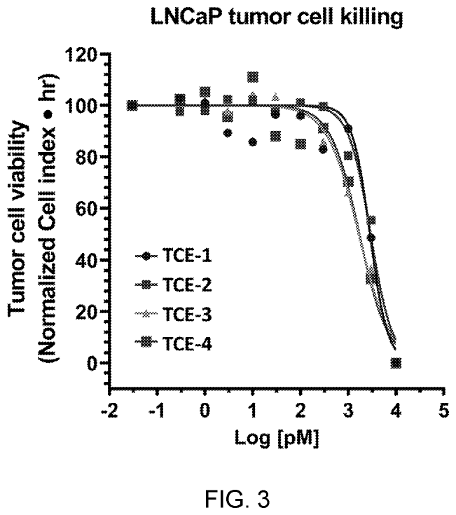

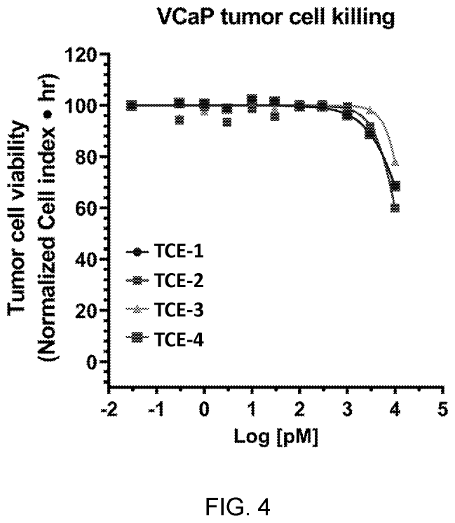

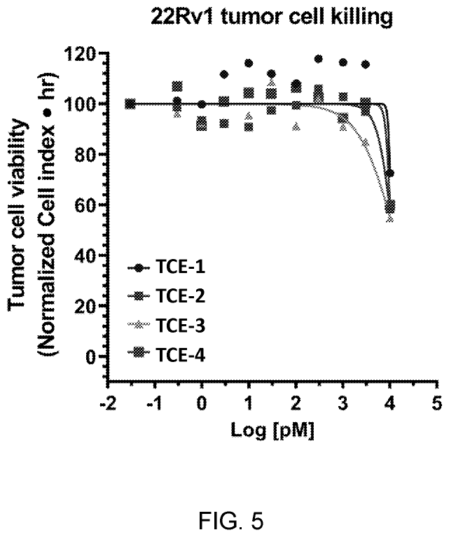

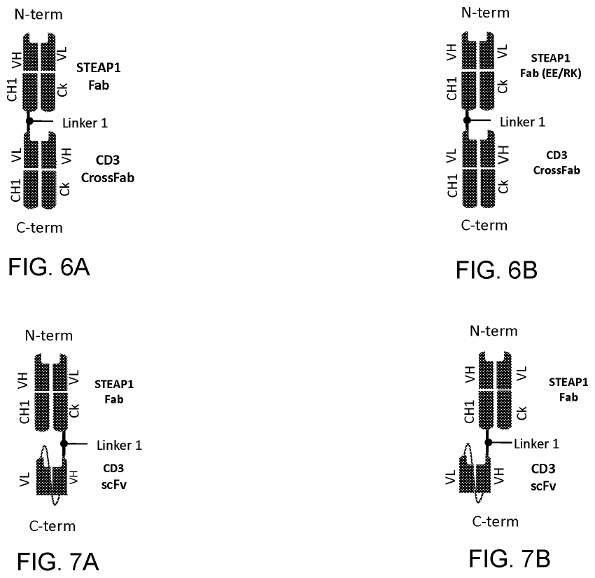

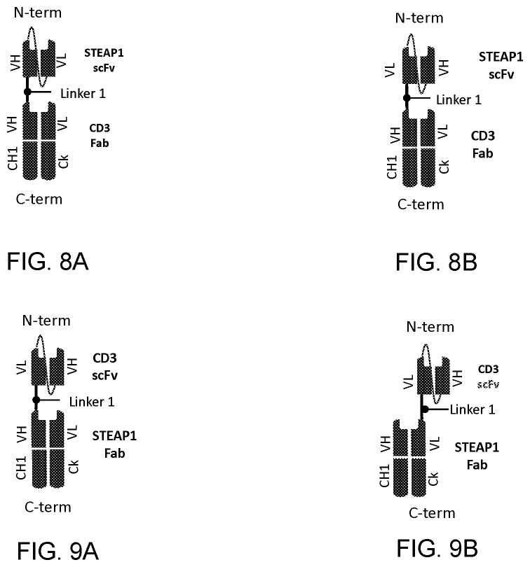

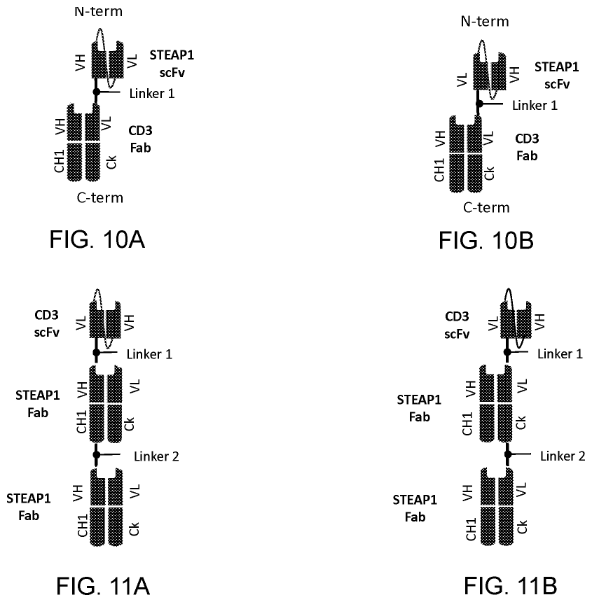

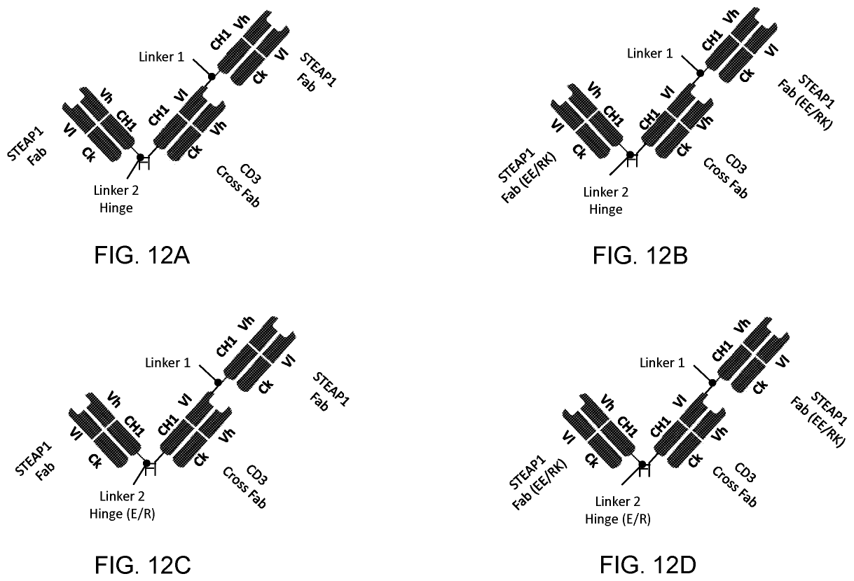

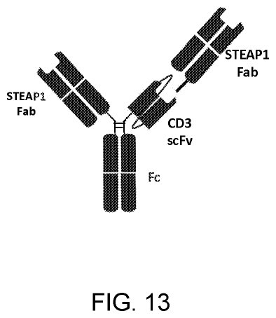

FIGS. 3-5 show representative tumor cell killing data for LNCaP cells (FIG. 3), VCaP cells (FIG. 4), and 22Rv1 cells (FIG. 5) treated with TCE-1, TCE-2, TCE-3, and TCE-4 in the presence of CD8+ T cells. This data is summarized in Tables 17-19. As can be seen, the STEAP-1 TCEs showed higher tumor cell killing activity against the higher STEAP-1 expressing LNCaP tumor cell line (225,000 STEAP-1 antibody binding sites per cell) compared to the lower STEAP-1 expressing VCaP (12,000 STEAP-1 antibody binding sites per cell) and 22Rv1 (8,000 STEAP-1 antibody binding sites per cell) tumor cell lines.

TABLE 17
IC₅₀s for LNCaP Tumor Cell Killing by TCEs

| TCE | IC₅₀ (pM) |
|---|---|
| TCE-1 | 2,856 |
| TCE-2 | 2,943 |
| TCE-3 | 1,670 |
| TCE-4 | 1,711 |

TABLE 18
IC₅₀s for VCaP Tumor Cell Killing by TCEs

| TCE | IC₅₀ (pM) |
|---|---|
| TCE-1 | >10,000 |
| TCE-2 | >10,000 |
| TCE-3 | >10,000 |
| TCE-4 | >10,000 |

TABLE 19
IC₅₀s for 22Rv1 Tumor Cell Killing by TCEs

| TCE | IC₅₀ (pM) |
|---|---|
| TCE-1 | >10,000 |
| TCE-2 | >10,000 |
| TCE-3 | >10,000 |
| TCE-4 | >10,000 |

### Influence of TCE Geometry and Linker Lengths on Cytolytic Potency

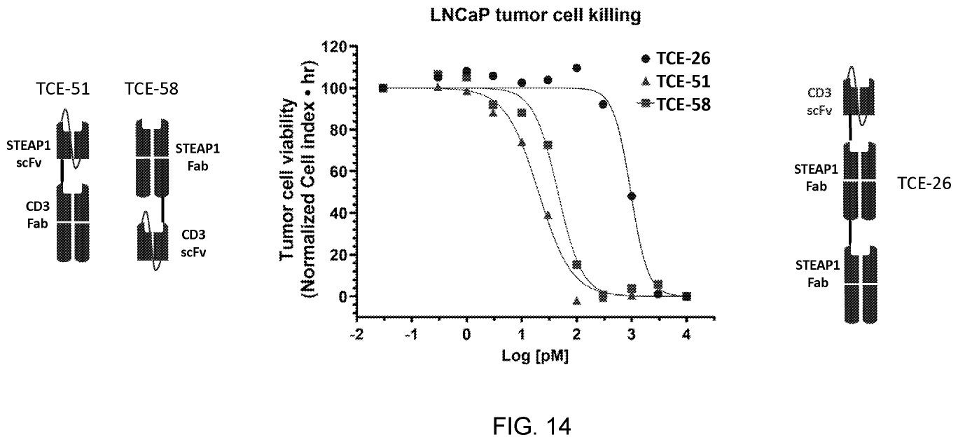

FIG. 14 shows LNCaP tumor cell killing with TCE-51 (N-terminal STEAP-1 scFv, C-terminal CD3 Fab), TCE-58 (N-terminal STEAP-1 Fab, C-terminal CD3 scFv), and TCE-26 (N-terminal CD3 scFv, two C-terminal STEAP-1 Fabs). The constructs with an N-terminal STEAP-1 binding domain (TCE-51 and TCE-58) showed stronger tumor cell killing potency compared to the construct with an N-terminal CD3 binding domain (TCE-26). The data suggests that TCE constructs with an N-terminal STEAP-1 binding domain and a C-terminal CD3 binding domain have stronger cytolytic potencies.

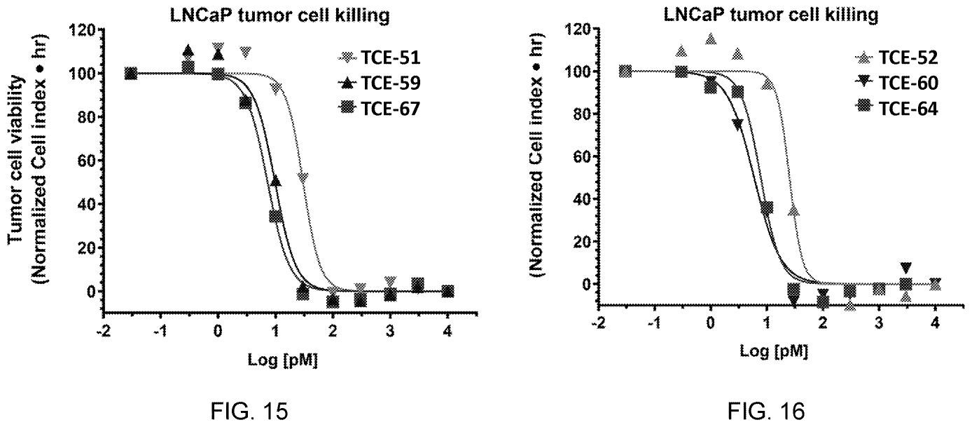

FIGS. 15-16 show LNCaP tumor cell killing with STEAP-1 TCEs having an N-terminal STEAP-1 scFv and a C-terminal CD3 Fab.

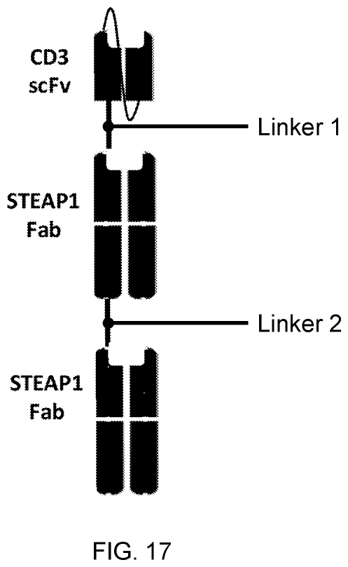

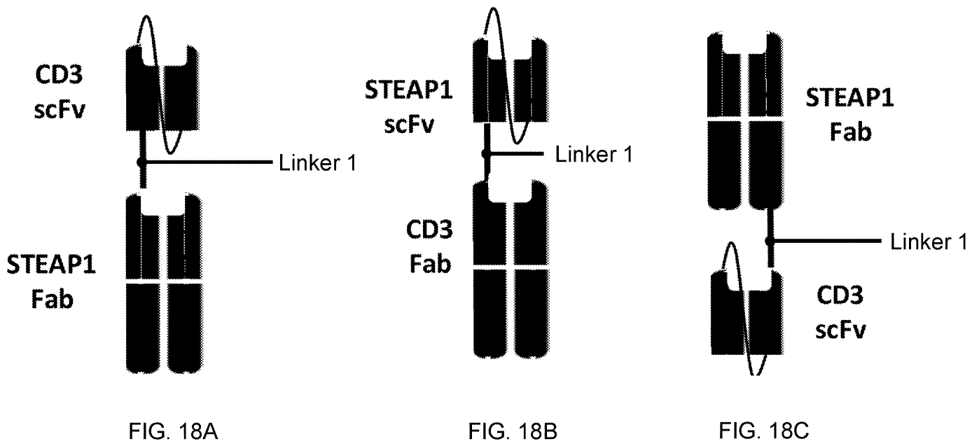

Table 20 below provides EC₅₀ values for LNCaP tumor cell killing with various 1+1 and 2+1 STEAP-1 TCEs of varying linker lengths and binding domain geometries. The corresponding binding domain geometries are referred to in the table and shown in FIG. 17 or FIG. 18. As shown in the table, increasing linker length in the 2+1 TCEs tended to improve tumor cell killing potencies. In the 1+1 TCE data set, higher tumor cell killing potencies were observed when the STEAP-1 binding domain is positioned N-terminal of the CD3 binding domain.

TABLE 20
Functional Assessment of STEAP-1 TCE Valency, Linker Length, and Binding Domain
Geometries (EC₅₀s based on LNCaP target cell lines)

| | | | CD3 | | STEAP1 | | | | | |
|---|---|---|---|---|---|---|---|---|---|---|
| | | | | Orient- | | Orient- | Linker | Linker | EC₅₀/ | Geo- |
| TCE | Valency | Format | Format | ation | Format | ation | 1 | 2 | pM | metry |
| TCE- | 2 + 1 | TCE VI | scFv | N-term | Fab | C-term | GGGG | GGGG | 585 | FIG. |
| 26 | | | | | | | S (SEQ | SGGG | | 17 |
| | | | | | | | ID NO: | GS | | |
| | | | | | | | 223) | (SEQ | | |
| | | | | | | | | ID NO: | | |
| | | | | | | | | 270) | | |
| TCE- | 2 + 1 | TCE VI | scFv | N-term | Fab | C-term | GGGG | GGGG | 163 | FIG. |
| 79 | | | | | | | S (SEQ | SGGG | | 17 |
| | | | | | | | ID NO: | GSGG | | |
| | | | | | | | 223) | GGS | | |
| | | | | | | | | (SEQ | | |
| | | | | | | | | ID NO: | | |
| | | | | | | | | 222) | | |
| TCE- | 2 + 1 | TCE VI | scFv | N-term | Fab | C-term | GGGG | GGGG | 260 | FIG. |
| 80 | | | | | | | S (SEQ | SGGG | | 17 |
| | | | | | | | ID NO: | GSGG | | |
| | | | | | | | 223) | GGSG | | |
| | | | | | | | | GGGS | | |
| | | | | | | | | (SEQ | | |
| | | | | | | | | ID NO: | | |
| | | | | | | | | 271) | | |
| TCE- | 2 + 1 | TCE VI | scFv | N-term | Fab | C-term | GGGG | GGGG | 239 | FIG. |
| 81 | | | | | | | S (SEQ | SGGG | | 17 |
| | | | | | | | ID NO: | GSGG | | |
| | | | | | | | 223) | GGSG | | |
| | | | | | | | | GGGS | | |
| | | | | | | | | GGGG | | |
| | | | | | | | | S (SEQ | | |
| | | | | | | | | ID NO: | | |
| | | | | | | | | 272) | | |
| TCE- | 2 + 1 | TCE VI | scFv | N-term | Fab | C-term | GGGG | GGGG | 214 | FIG. |
| 82 | | | | | | | SGGG | SGGG | | 17 |
| | | | | | | | GS | GS | | |
| | | | | | | | (SEQ | (SEQ | | |
| | | | | | | | ID NO: | ID NO: | | |
| | | | | | | | 270) | 270) | | |
| TCE- | 2 + 1 | TCE VI | scFv | N-term | Fab | C-term | GGGG | GGGG | 209 | FIG. |
| 83 | | | | | | | SGGG | SGGG | | 17 |
| | | | | | | | GS | GSGG | | |
| | | | | | | | (SEQ | GGS | | |
| | | | | | | | ID NO: | (SEQ | | |
| | | | | | | | 270) | ID NO: | | |
| | | | | | | | | 222) | | |
| TCE- | 2 + 1 | TCE VI | scFv | N-term | Fab | C-term | GGGG | GGGG | 192 | FIG. |
| 84 | | | | | | | SGGG | SGGG | | 17 |
| | | | | | | | GS | GSGG | | |
| | | | | | | | (SEQ | GGSG | | |
| | | | | | | | ID NO: | GGGS | | |
| | | | | | | | 270) | (SEQ | | |
| | | | | | | | | ID NO: | | |
| | | | | | | | | 271) | | |
| TCE- | 2 + 1 | TCE VI | scFv | N-term | Fab | C-term | GGGG | GGGG | 361 | FIG. |
| 85 | | | | | | | SGGG | SGGG | | 17 |
| | | | | | | | GS | GSGG | | |
| | | | | | | | (SEQ | GGSG | | |
| | | | | | | | ID NO: | GGGS | | |
| | | | | | | | 270) | GGGG | | |
| | | | | | | | | S (SEQ | | |
| | | | | | | | | ID NO: | | |
| | | | | | | | | 272) | | |
| TCE- | 2 + 1 | TCE VI | scFv | N-term | Fab | C-term | GGGG | GGGG | 233 | FIG. |
| 86 | | | | | | | SGGG | SGGG | | 17 |
| | | | | | | | GSGG | GS | | |
| | | | | | | | GGS | (SEQ | | |
| | | | | | | | (SEQ | ID NO: | | |
| | | | | | | | ID NO: | 270) | | |
| | | | | | | | 222) | | | |
| TCE- | 2 + 1 | TCE VI | scFv | N-term | Fab | C-term | GGGG | GGGG | 235 | FIG. |
| 87 | | | | | | | SGGG | SGGG | | 17 |
| | | | | | | | GSGG | GSGG | | |
| | | | | | | | GGS | GGS | | |
| | | | | | | | (SEQ | (SEQ | | |
| | | | | | | | ID NO: | ID NO: | | |
| | | | | | | | 222) | 222) | | |
| TCE- | 2 + 1 | TCE VI | scFv | N-term | Fab | C-term | GGGG | GGGG | 183 | FIG. |
| 88 | | | | | | | SGGG | SGGG | | 17 |
| | | | | | | | GSGG | GSGG | | |
| | | | | | | | GGS | GGSG | | |
| | | | | | | | (SEQ | GGGS | | |
| | | | | | | | ID NO: | (SEQ | | |
| | | | | | | | 222) | ID NO: | | |
| | | | | | | | | 271) | | |
| TCE- | 2 + 1 | TCE VI | scFv | N-term | Fab | C-term | GGGG | GGGG | 123 | FIG. |
| 89 | | | | | | | SGGG | SGGG | | 17 |
| | | | | | | | GSGG | GSGG | | |
| | | | | | | | GGS | GGSG | | |
| | | | | | | | (SEQ | GGGS | | |
| | | | | | | | ID NO: | GGGG | | |
| | | | | | | | 222) | S (SEQ | | |
| | | | | | | | | ID NO: | | |
| | | | | | | | | 272) | | |
| TCE- | 1 + 1 | TCE | scFv | N-term | Fab | C-term | GGGG | — | 3508 | FIG. |
| 1 | | VH | | | | | S (SEQ | | | 18A |
| | | | | | | | ID NO: | | | |
| | | | | | | | 223) | | | |
| TCE- | 1 + 1 | TCE VL | scFv | N-term | Fab | C-term | GGGG | — | 1564 | FIG. |
| 2 | | | | | | | S (SEQ | | | 18A |
| | | | | | | | ID NO: | | | |
| | | | | | | | 223) | | | |
| TCE- | 1 + 1 | TCE | scFv | N-term | Fab | C-term | GGGG | — | 1014 | FIG. |
| 53 | | VH | | | | | SGGG | | | 18A |
| | | | | | | | GS | | | |
| | | | | | | | (SEQ | | | |
| | | | | | | | ID NO: | | | |
| | | | | | | | 270) | | | |
| TCE- | 1 + 1 | TCE | scFv | N-term | Fab | C-term | GGGG | — | 792 | FIG. |
| 54 | | VH | | | | | SGGG | | | 18A |
| | | | | | | | GSGG | | | |
| | | | | | | | GGS | | | |
| | | | | | | | (SEQ | | | |
| | | | | | | | ID NO: | | | |
| | | | | | | | 222) | | | |
| TCE- | 1 + 1 | TCE VI | scFv | N-term | Fab | C-term | GGGG | — | 1313 | FIG. |
| 55 | | | | | | | SGGG | | | 18A |
| | | | | | | | GS | | | |
| | | | | | | | (SEQ | | | |
| | | | | | | | ID NO: | | | |
| | | | | | | | 270) | | | |
| TCE- | 1 + 1 | TCE VI | scFv | N-term | Fab | C-term | GGGG | — | 1050 | FIG. |
| 56 | | | | | | | SGGG | | | 18A |
| | | | | | | | GSGG | | | |
| | | | | | | | GGS | | | |
| | | | | | | | (SEQ | | | |
| | | | | | | | ID NO: | | | |
| | | | | | | | 222) | | | |
| TCE- | 1 + 1 | Vh-TCE | Fab | C-term | scFv | N-term | GGGG | | 21 | FIG. |
| 51 | | | | | | | SGGG | | | 18B |
| | | | | | | | GS | | | |
| | | | | | | | (SEQ | | | |
| | | | | | | | ID NO: | | | |
| | | | | | | | 270) | | | |
| TCE- | 1 + 1 | V1-TCE | Fab | C-term | scFv | N-term | GGGG | | 25 | FIG. |
| 52 | | | | | | | SGGG | | | 18B |
| | | | | | | | GS | | | |
| | | | | | | | (SEQ | | | |
| | | | | | | | ID NO: | | | |
| | | | | | | | 270) | | | |
| TCE- | 1 + 1 | Ch-TCE | scFv | C-term | Fab | N-term | GGGG | | 45 | FIG. |
| 57 | | | (VH- | | | | SGGG | | | 18C |
| | | | VL) | | | | GS | | | |
| | | | | | | | (SEQ | | | |
| | | | | | | | ID NO: | | | |
| | | | | | | | 270) | | | |
| TCE- | 1 + 1 | CI-TCE | scFv | C-term | Fab | N-term | GGGG | | 45 | FIG. |
| 58 | | | (VH- | | | | SGGG | | | 18C |
| | | | VL) | | | | GS | | | |
| | | | | | | | (SEQ | | | |
| | | | | | | | ID NO: | | | |
| | | | | | | | 270) | | | |

Table 21 provides EC₅₀s for tumor cell killing by different 1+1 TCE constructs of the present disclosure. Table 22 provides EC₅₀s for tumor cell killing by different 2+1 TCE constructs of the present disclosure.

TABLE 21
EC₅₀s for Tumor Cell Killing by 1 + 1 TCEs

| | Cell line cytotoxicity EC₅₀ (pM) | | | | | | |
|---|---|---|---|---|---|---|---|
| TCE | LNCaP | C4-2B | A673 | IM-95 | VCaP | 22RV1 | OE33 |
| TCE-1 | 3507.9 | 23609.0 | 1594.0 | 602.8 | >10,000 | >10,000 | >10,000 |
| TCE-2 | 1564.3 | 1900.0 | 247.7 | 273.8 | 792.7 | >10,000 | 1332.0 |
| TCE-3 | 1336.0 | | | 639.1 | >10,000 | >10,000 | |
| TCE-4 | 2220.0 | | | 647.7 | >10,000 | >10,000 | |
| TCE-5 | | | | | | | |
| TCE-6 | | | | | | | |
| TCE-7 | | | | | | | |
| TCE-8 | | | | | | | |
| TCE-9 | | | | | | | |
| TCE-10 | | | | | | | |
| TCE-11 | | | | | | | |
| TCE-12 | | | | | | | |
| TCE-13 | | | | | | | |
| TCE-14 | | | | | | | |
| TCE-15 | | | | | | | |
| TCE-16 | | | | | | | |
| TCE-17 | | | | | | | |
| TCE-18 | | | | | | | |
| TCE-19 | | | | | | | |
| TCE-20 | | | | | | | |
| TCE-21 | | | | | | | |
| TCE-22 | | | | | | | |
| TCE-23 | | | | | | | |
| TCE-24 | | | | | | | |
| TCE-49 | 1521.0 | | | | | | |
| TCE-50 | 262.4 | | | | | | |
| TCE-51 | 25.3 | 76.1 | 31.7 | 34.7 | | 484.3 | |
| TCE-52 | 23.8 | | | 9.6 | | 316.8 | |
| TCE-53 | 1014.0 | | | | | | |
| TCE-54 | 791.8 | | | | | | |
| TCE-55 | 1313.0 | | | | | | |
| TCE-56 | 1050.0 | | | | | | |
| TCE-57 | 45.2 | | | | | 294.8 | |
| TCE-58 | 44.5 | | | | | 331.8 | |
| TCE-59 | 9.4 | | | | | | |
| TCE-60 | 6.3 | | | | | | |
| TCE-61 | 22.1 | | | | | | |
| TCE-62 | 10.3 | | | | | | |
| TCE-63 | 16.5 | | | | | | |
| TCE-64 | 8.4 | | | | | | |
| TCE-65 | 10.6 | | | | | | |
| TCE-66 | 15.3 | | | | | | |
| TCE-67 | 7.3 | | | | | | |
| TCE-68 | 13.3 | | | | | | |
| TCE-69 | 89.1 | | | 11.2 | | 359.4 | |
| TCE-70 | 230.8 | | | 31.8 | | 916.4 | |
| TCE-71 | 7.9 | | | | | | |
| TCE-72 | 5.4 | | | | | | |
| TCE-73 | 367.1 | | | | | | |
| TCE-74 | 119.4 | | | | | | |
| TCE-75 | 5.2 | | | | | | |
| TCE-76 | 7.9 | | | | | | |

TABLE 22
EC₅₀s for Tumor Cell Killing by 2 + 1 TCEs

| | Cell line cytotoxicity EC₅₀ (pM) | | | | | | |
|---|---|---|---|---|---|---|---|
| TCE | LNCaP | C4-2B | A673 | IM-95 | VCaP | 22RV1 | OE33 |
| TCE-25 | 320.5 | 1204.0 | 115.9 | 49.5 | 2281 | 1986 | 654.9 |
| TCE-26 | 585.0 | 1151.0 | 45.8 | 146.7 | | 2099 | |
| TCE-27 | 751.5 | | | 82.71 | | | |
| TCE-28 | | | | | | | |
| TCE-29 | | | | | | | |
| TCE-30 | | | | | | | |
| TCE-31 | | | | | | | |
| TCE-32 | | | | | | | |
| TCE-33 | | | | | | | |
| TCE-34 | | | | | | | |
| TCE-35 | | | | | | | |
| TCE-36 | | | | | | | |
| TCE-37 | | | | | | | |
| TCE-38 | | | | | | | |
| TCE-39 | | | | | | | |
| TCE-40 | | | | | | | |
| TCE-41 | | | | | | | |
| TCE-42 | | | | | | | |
| TCE-43 | | | | | | | |
| TCE-44 | | | | | | | |
| TCE-45 | | | | | | | |
| TCE-46 | | | | | | | |
| TCE-47 | | | | | | | |
| TCE-48 | | | | | | | |
| TCE-77 | | | | | | | |
| TCE-78 | | | | | | | |
| TCE-79 | 162.9 | | | | | | |
| TCE-80 | 260.3 | | | | | | |
| TCE-81 | 239.1 | | | | | | |
| TCE-82 | 214.2 | | | | | | |
| TCE-83 | 209.1 | | | | | | |
| TCE-84 | 191.7 | | | | | | |
| TCE-85 | 360.5 | | | | | | |
| TCE-86 | 232.5 | | | | | | |
| TCE-87 | 234.9 | | | | | | |
| TCE-88 | 183.2 | | | | | | |
| TCE-89 | 122.8 | | | | | | |
| TCE-90 | 68.5 | | 17.3 | 11.6 | | 170.7 | |
| TCE-91 | | | | | | | |
| TCE-92 | | | | | | | |
| TCE-93 | 0.2 | 0.4 | 0.9 | 3.6 | 1.2 | 23.2 | |

### Example 2: STEAP-1 TCE Binding to LNCaP Cells by Flow Cytometry

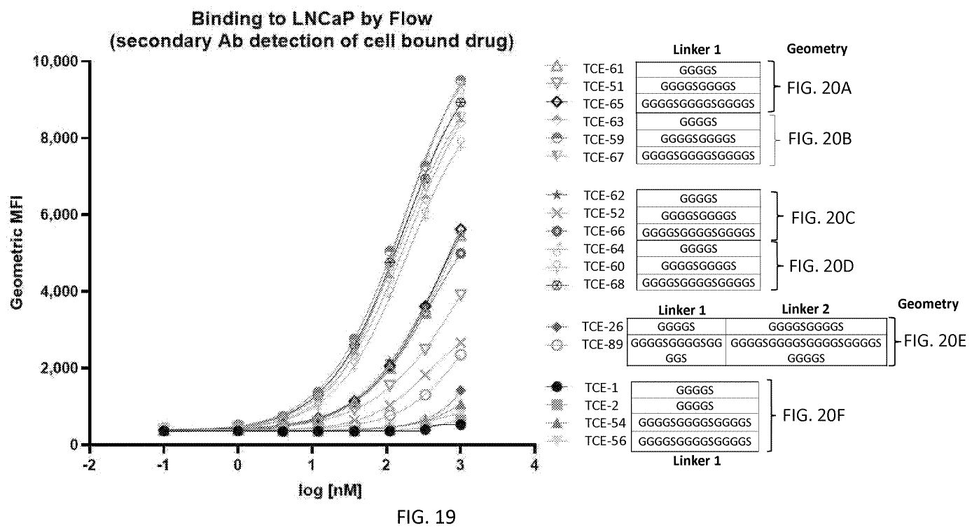

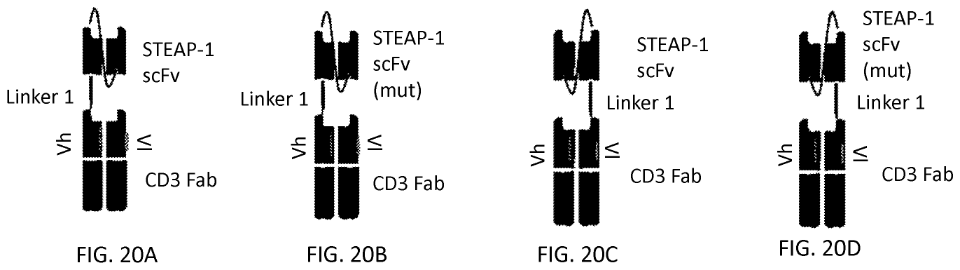

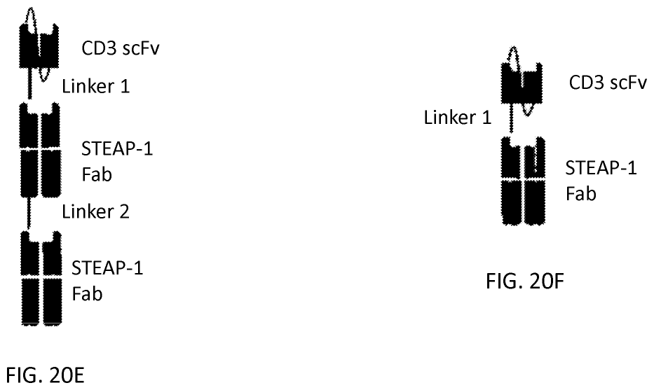

LNCaP cells were stained with polypeptide complexes and bound drug was measured using standard flow cytometry procedures. Briefly, 50,000 LNCaP cells were seeded in a 96 well V-bottom plate and titrated polypeptide complexes were added to the cells. After a brief incubation period with polypeptide complexes, cells were washed, and stained with Live/dead dye and a fluorescently labeled secondary antibody that recognizes the polypeptide complex. After another short incubation period, stained cells were washed, fixed, and washed again. Stained cells were then analyzed in a BD FACs analyzer. Live cells were gated and the corresponding bound polypeptide geometric mean fluorescent intensity (GMFI) signal was measured. GMFI was then plotted against logarithmic polypeptide concentration. Plots of GMFI versus TCE concentration are shown in FIG. 19. The binding domain geometries of the tested TCE constructs are referred to in FIG. 19 and shown in FIGS. 20A-20F. Table 23 provides the highest GMFI levels for each of the tested TCE constructs wherein a higher GMFI signal correlates with stronger binding to the LNCaP cells.

TABLE 23

| | LNCaP binding signal |
|---|---|
| TCE | (maximum GMFI observed) |
| TCE-1 | 532 |
| TCE-2 | 656 |
| TCE-54 | 1068 |
| TCE-56 | 824 |
| TCE-26 | 1426 |
| TCE-89 | 2356 |
| TCE-61 | 5474 |
| TCE-51 | 3908 |
| TCE-65 | 5628 |
| TCE-63 | 8407 |
| TCE-59 | 9509 |
| TCE-67 | 8543 |
| TCE-62 | 5500 |
| TCE-52 | 2673 |
| TCE-66 | 4997 |
| TCE-64 | 9325 |
| TCE-60 | 7852 |
| TCE-68 | 8932 |

### Example 3: Manufacturability of STEAP-1 Binders Containing Mutations and an Engineered Disulfide Bond

Additional experiments are performed to evaluate the manufacturability of STEAP-1 binders including bi-specific (TCEs) or monospecific antibodies containing STEAP-1 binding domains after the introduction of mutations or an engineered disulfide bond into the STEAP-1 binding domain. The manufacturing process utilizes cation exchange chromatography for polishing post affinity chromatography steps. STEAP-1 binders are produced by transient expression in Chinese hamster ovary (CHO) cells. Conditioned medium from CHO cells expressing antibodies of interest are first purified using a AKTA Pure FPLC by affinity chromatography (AC) using CH1-XL resin with a pH 4 acidic elution step. Material eluted from the AC step is neutralized in 1M Tris followed by dilution in salt-free buffer at pH 6.0. Diluted material is then passed onto a cation exchange column followed by elution using a salt gradient (0-500 mM NaCl over 20 column volumes). Protein elution is monitored using UV absorbance and area under the curve (AUC) of distinct peaks is calculated using Unicorn software.

### Example 4: Material Properties Post-Processing of STEAP-1 Binders Containing an Engineered Disulfide Bond

Several experiments are conducted to assess various properties of the polished materials post-cation exchange chromatography polishing. Material properties are assessed at the following conditions:
1. Non-stressed (TO),
2. After 12 days at 25° C. (T12),
3. After 5 days at 40° C. (T5),
4. After 12 days at 40° C. (T12),
5. After 19 days at 40° C. (T19), and
6. After 5× freeze-thaw cycles (FT) to −70° C.

To induce temperature stress, samples are stored upright in tightly sealed glass vials (Agilent Technologies) sealed with a PFTE/red silicone septa (Agilent Technologies) in a temperature-controlled chamber operated at 25±5° C. or 40° C.±5° C., respectively.

To induce free-thaw stress, samples are subjected to 5 freeze-thaw cycles as follows: freezing consisting of storing samples at least 2 h at −70±10° C. Frozen samples are then thawed at room temperature for at least 2 h.

Specifically, studies are conducted to assess the hydrophobicity, charge variants, melting temperature, aggregation temperature, melting onset temperature, diameter, and uniformity of the STEAP-1 binders.

### Hydrophobicity

First, a study is conducted using analytical hydrophobic interaction chromatography (HIC) to measure the hydrophobicity of the exemplary STEAP-1 binders. Hydrophobicity is an important property for reducing aggregation over time during manufacturing.

Analytical HIC is performed on an Agilent 1290 II system (Agilent Technologies, Santa Clara, CA) using a MABPac HIC-10 column (ThermoFisher Scientific, Waltham, MA) 4.6 mm×100 mm, 5 μm particle size. The column is operated at 30° C. with a flow rate of 1.0 mL/min. Samples are diluted 1:1 by volume with mobile phase A prior to injection, and the column load is 10 μg. The mobile phase consists of (A) 50 mM sodium phosphate, 2M ammonium sulfate, pH-6.5 (B) 50 mM sodium phosphate, pH=6.5. The linear gradient is set as follows: 0-2 min, 0% B; 2.0-2.5 min, 0-40% B; 2.5-20.5 min, 40-100% B, 20.5-24.0 min, 100% B; 24.0-24.1 min, 100-0% B and 24.1-31 min, 0% B. Eluting proteins are detected by UV spectroscopy at a wavelength of 214 nm. Data are processed using the Agilent OpenLab CDS software version 3.5.0 (Agilent Technologies).

### Charge Variants

Another study utilizes imaged capillary isoelectric focusing (icIEF) to analyze the charge variants of the exemplary STEAP-1 binders. iCIEF analysis is performed on a Maurice instrument equipped with an iCIEF cartridge (ProteinSimple, #PS-MC02-C). All samples are buffer exchanged into water using 10 kDa Amicon Ultra MWCO devices (Millipore), and the protein concentration is adjusted to 0.5 mg/mL. Twenty microliters of sample is mixed with 80 μl of iCIEF separation mix before sample analysis. The iCIEF separation mix is prepared fresh by mixing the following in a separate tube. Amounts provided are sufficient for 1 sample: 1% methyl cellulose (ProteinSimple #PS-MDK01-C), 35 μl; SimpleSol (ProteinSimple #046-575), 20 μl; pI Marker 7.05 (ProteinSimple #046-032), 0.5 μl; pI Marker 10.17 (ProteinSimple #046-35), 0.5 μl; Pharmalyte 5-8 (Cytiva, PN: 17-0453-01), 1 μl; Pharmalyte 8-10.5 (Cytiva, PN: 17-0455-01), 1 μl; 500 mM arginine (ProteinSimple, #PS-MDK01-C) 0.4 μl and ultrapure water, 19.6 μl. Sample analysis on the Maurice proceeds with the following method: Sample load by vacuum aspiration 90s, focusing for protein separation 1 min at 1500 V followed by 8 min at 3000 V. Focused proteins are detected by native fluorescence detection. Data are processed and analyzed using Compass software (Version 3.0.0, ProteinSimple).

### Purity

Another study utilizes non-reduced capillary gel electrophoresis sodium dodecyl sulfate (CE-SDS) analysis to measure formation of impurities of exemplary STEAP-1 binders under temperature stressed conditions. CE-SDS is performed on a Maurice instrument (ProteinSimple, San Jose, CA) equipped with a CE-SDS plus cartridge (Bio-Techne #PS-MC02-SP). CE-SDS Plus reagent kit (Bio-Techne #PS-MRK01-S), CE-SDS top running buffer (Bio-Techne #046-384), Maurice CE-SDS 25X Internal Standard (Bio-Techne #046-144) and CE-SDS Molecular Weight Markers (Bio-Techne #046-432) follow the instructions provided with the cartridge. All samples are diluted with 1× sample buffer to 0.5 mg/mL. Fifty microliter of diluted sample is mixed with 2 μl internal standard and 2.5 μl of freshly prepared 250 mM iodoacetamide. Samples are incubated for 10 min at 70° C. and 50 μl of the resulting supernatant was transferred to a 96-multiwell plate for analysis. Electrophoresis on the Maurice proceeds with the following method: Samples are loaded electrokinetically for 20s at 4600 V, and separation proceeds for 35 min at 5750 V. Proteins are detected by UV spectroscopy at 220 nm. Electropherograms are processed with Compass software (Version 3.0.0, ProteinSimple, San Jose, CA).

### Thermostability

Another study is conducted to assess the thermostability (e.g., melting temperature, aggregation temperature, and melting onset temperature) of the exemplary STEAP-1 binders, using differential scanning fluorometry (DSF) performed on an UNcle instrument (Unchained Labs).

To conduct this study, nine microliters of sample in its corresponding formulation buffer are loaded in triplicate in single-use Uni devices (Unchained labs, 201-1009) with sample concentrations ranging from 1-3 mg/mL. The excitation wavelength is 266 nm, and full-spectrum intrinsic fluorescence spectra are collected from 250-720 nm. The temperature is ramped from 15 to 95° C. with a thermal ramp of 0.5° C./minute. Data are processed with UNcle software v. 6.01 with the barycentric mean (BCM) method considering the wavelength range from 300-410 nm. Melting temperature (Tm) is defined as the maximum value of the first derivative of the BCM trace, while the melting onset temperature (Tonset) is defined at a 10% threshold from the baseline for the first derivative of the BCM trace. The aggregation temperature (Tagg) is determined from the static light scattering (SLS) trace at a 10% from baseline threshold.

### Diameter

Another study is conducted to assess the average diameter of the exemplary STEAP-1 binders via the UNcle instrument using dynamic light scattering (DLS). Proteins are sized with DLS at 15° C. before initiating the thermal ramp for DSF. The acquisition time for DLS is 5s and an average of 4 measurements is reported. DLS data are processed with UNcle software v. 6.01.

### Uniformity

Appearance of the exemplary STEAP-1 binders is observed. Appearance testing is performed following the procedures outlined in USP <790> using a visual inspection station (Angstrom Supply) providing a black and white background with controlled lighting at 2000-3750 lux. Color and clarity observations are also recorded during appearance testing.

The concentration of the exemplary STEAP-1 binders is measured. Protein concentration is determined by UV absorption spectroscopy at 280 nm on a NanoDrop One (ThermoFisher Scientific) using the pedestal method (1 mm path length). The instrument is blanked with formulation buffer prior to sample analysis. All protein determinations are performed in triplicate with 2 μl sample. Extinction coefficients are calculated with Expasy ProtParam tool (Gasteiger et al., Springer, 2005) using the primary sequence and assuming all disulfide bonds are formed.

The protein size of the exemplary STEAP-1 binders are further analyzed using size exclusion chromatography high performance liquid chromatography (SEC-HPLC). SEC-HPLC is performed on an Agilent 1290 II instrument (Agilent Technologies) fitted with an advanced Bio SEC (Agilent Technologies) pre-column and an analytical SEC column, respectively. The precolumn is a 4.6×50 mm, 300 Å, #PL1580-1301 and the SEC column is 4.6×300 mm, 300 Å, #PL1580-5301. SEC analysis is performed under isocratic conditions at a column temperature of 28° C. The mobile phase, composed of 30 mM sodium acetate and 500 mM sodium chloride (pH 5.5), is delivered at a flow rate of 0.35 mL/min. The column load is 8 μg and chromatographic separation is monitored at 214 nm via UV detection. The total run time for the method is 19.5 minutes. Data are processed using the Agilent OpenLab CDS software version 3.5.0 (Agilent Technologies).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 225 and Q3, S9, S12, A13, V15, D17, V19, T22, Q35, K48, A49, S66, P86, F89, T91, and Q106 of SEQ ID NO: 226, and wherein the polypeptide or polypeptide complex comprises a second antibody or antigen binding thereof that binds to CD3.

2. The polypeptide or polypeptide complex of claim 1, wherein the at least one amino acid mutation is selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226.

3. The polypeptide or polypeptide complex of claim 1, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226.

4. The polypeptide or polypeptide complex of claim 1, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 225, and the immunoglobulin heavy chain comprises an amino acid sequence with at least 90% sequence identity to SEQ ID NO: 225, and the immunoglobulin light chain comprises an amino acid sequence with at least 90% sequence identity to SEQ ID NO: 226.

5. The polypeptide or polypeptide complex of claim 1, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 225 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 226, and the immunoglobulin heavy chain comprises an amino acid sequence with at least 95% sequence identity to SEQ ID NO: 225, and the immunoglobulin light chain comprises an amino acid sequence with at least 95% sequence identity to SEQ ID NO: 226.

6. The polypeptide or polypeptide complex of claim 1, wherein the immunoglobulin heavy chain comprises mutations V24A, Y27F, T58I, and T74N, and at least 90% sequence identity to SEQ ID NO: 225.

7. The polypeptide or polypeptide complex of claim 1, wherein the immunoglobulin heavy chain comprises mutations W35M and L64V and at least 90% sequence identity to SEQ ID NO: 225.

8. The polypeptide or polypeptide complex of claim 1, wherein the immunoglobulin heavy chain comprises mutations V24A, Y27F, W35M, T58I, L64V, S66G, and T74N and at least 90% sequence identity to SEQ ID NO: 225.

9. The polypeptide or polypeptide complex of claim 1, wherein the immunoglobulin heavy chain comprises mutations W35M, L64V, E99D, and M111F and at least 90% sequence identity to SEQ ID NO: 225.

10. The polypeptide or polypeptide complex of claim 1, wherein the immunoglobulin heavy chain comprises mutations V24A, Y27F, W35M, L64V, T74N, E99D, and M111F and at least 90% sequence identity to SEQ ID NO: 225.

11. The polypeptide or polypeptide complex of claim 1, wherein the immunoglobulin heavy chain comprises a G45C mutation and at least 90% sequence identity to SEQ ID NO: 225.

12. The polypeptide or polypeptide complex of claim 1, wherein the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226.

13. The polypeptide or polypeptide complex of claim 1, wherein the immunoglobulin light chain comprises a Q35N mutation and at least 90% sequence identity to SEQ ID NO: 226.

14. The polypeptide or polypeptide complex of claim 1, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

15. The polypeptide or polypeptide complex of claim 1, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

16. The polypeptide or polypeptide complex of claim 1, wherein the immunoglobulin light chain comprises a Q106C mutation and at least 90% sequence identity to SEQ ID NO: 226.

17. The polypeptide or polypeptide complex of claim 1, wherein the immunoglobulin heavy chain comprises mutations V24A, Y27F, T58I, and T74N, and at least 90% sequence identity to SEQ ID NO: 225 and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 226.

18. The polypeptide or polypeptide complex of claim 1, wherein the immunoglobulin heavy chain comprises mutations W35M and L64V and at least 90% sequence identity to SEQ ID NO: 225 and the immunoglobulin light chain comprises the Q35N mutation and at least 90% sequence identity to SEQ ID NO: 226.

19. The polypeptide or polypeptide complex of claim 1, wherein the immunoglobulin heavy chain comprises mutations V24A, Y27F, W35M, T58I, L64V, S66G, and T74N and at least 90% sequence identity to SEQ ID NO: 225 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

20. The polypeptide or polypeptide complex of claim 1, wherein the immunoglobulin heavy chain comprises mutations W35M, L64V, E99D, and M111F and at least 90% sequence identity to SEQ ID NO: 225 and the immunoglobulin light chain comprises a Q35N mutation and at least 90% sequence identity to SEQ ID NO: 226.

21. The polypeptide or polypeptide complex of claim 1, wherein the immunoglobulin heavy chain comprises mutations V24A, Y27F, W35M, L64V, T74N, E99D, and M111F and at least 90% sequence identity to SEQ ID NO: 225 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 226.

22. The polypeptide or polypeptide complex of claim 1, wherein the immunoglobulin heavy chain comprises a G45C mutation and at least 90% sequence identity to SEQ ID NO: 225 and the immunoglobulin light chain comprises a Q106C mutation and at least 90% sequence identity to SEQ ID NO: 226.

23. The polypeptide or polypeptide complex of claim 1, wherein the antibody or antigen binding fragment thereof comprises an engineered disulfide bond between the immunoglobulin heavy chain and the immunoglobulin light chain.

24. The polypeptide or polypeptide complex of claim 22, wherein the antibody or antigen binding fragment thereof comprises an engineered disulfide bond between the cysteine at the position of the G45C mutation and the cysteine at the position of the Q106C mutation.

25. The polypeptide or polypeptide complex of claim 1, wherein the immunoglobulin heavy chain comprises a G45C mutation and the immunoglobulin light chain comprises a Q106C mutation, and wherein the polypeptide or polypeptide complex comprises an amino acid sequence having at least 95%, 98%, 99%, or 100% sequence identity to SEQ ID NO: 261.

26. The polypeptide or polypeptide complex of claim 1, wherein the immunoglobulin heavy chain comprises a G45C mutation and the immunoglobulin light chain comprises a Q106C mutation, and wherein the polypeptide or polypeptide complex comprises an amino acid sequence having at least 95%, 98%, 99%, or 100% sequence identity to SEQ ID NO: 262.

27. A polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of:
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37; and
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38; and wherein the polypeptide or polypeptide complex comprises a second antibody or antigen binding fragment thereof that binds to CD3.

28. The polypeptide or polypeptide complex of claim 1, wherein the immunoglobulin heavy chain and the immunoglobulin light chain comprise a set of six CDR sequences selected from the group consisting of:
CDR1-H: SEQ ID NO: 3, CDR2-H: SEQ ID NO: 9, CDR3-H: SEQ ID NO: 15,
CDR1-L: SEQ ID NO: 21, CDR2-L: SEQ ID NO: 27, CDR3-L: SEQ ID NO: 33;
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37; and
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38.

29. The polypeptide or polypeptide complex of claim 27, wherein the immunoglobulin light chain and the immunoglobulin heavy chain comprise a set of six CDR sequences of
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
and wherein the immunoglobulin heavy chain comprises an amino acid sequence of at least 95% sequence identity to SEQ ID NO: 233 and the immunoglobulin light chain comprises an amino acid sequence of at least 95% sequence identity to SEQ ID NO: 234.

30. The polypeptide or polypeptide complex of claim 29, wherein the immunoglobulin heavy chain comprises an amino acid sequence of at least 99% sequence identity to SEQ ID NO: 233 and the immunoglobulin light chain comprises an amino acid sequence of at least 99% sequence identity to SEQ ID NO: 234.

31. The polypeptide or polypeptide complex of claim 27, wherein the immunoglobulin light chain and the immunoglobulin heavy chain comprise a set of six CDR sequences of
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
and wherein the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40.

32. The polypeptide or polypeptide complex of claim 31, wherein the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40.

33. The polypeptide or polypeptide complex of claim 27, wherein the immunoglobulin light chain and the immunoglobulin heavy chain comprise a set of six CDR sequences of
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
and wherein the immunoglobulin heavy chain comprises an amino acid sequence of at least 95% sequence identity to SEQ ID NO: 235 and the immunoglobulin light chain comprises an amino acid sequence of at least 95% sequence identity to SEQ ID NO: 236.

34. The polypeptide or polypeptide complex of claim 33, wherein the immunoglobulin heavy chain comprises an amino acid sequence of at least 99% sequence identity to SEQ ID NO: 235 and the immunoglobulin light chain comprises an amino acid sequence of at least 99% sequence identity to SEQ ID NO: 236.

35. The polypeptide or polypeptide complex of claim 27, wherein the immunoglobulin light chain and the immunoglobulin heavy chain comprise a set of six CDR sequences of
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
and wherein the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42.

36. The polypeptide or polypeptide complex of claim 35, wherein the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42.

37. The polypeptide or polypeptide complex of claim 27, wherein the immunoglobulin light chain and the immunoglobulin heavy chain comprise a set of six CDR sequences of
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
and wherein the immunoglobulin heavy chain comprises an amino acid sequence of at least 95% sequence identity to SEQ ID NO: 237 and the immunoglobulin light chain comprises an amino acid sequence of at least 95% sequence identity to SEQ ID NO: 238.

38. The polypeptide or polypeptide complex of claim 37, wherein the immunoglobulin heavy chain comprises an amino acid sequence of at least 99% sequence identity to SEQ ID NO: 237 and the immunoglobulin light chain comprises an amino acid sequence of at least 99% sequence identity to SEQ ID NO: 238.

39. The polypeptide or polypeptide complex of claim 27, wherein the immunoglobulin light chain and the immunoglobulin heavy chain comprise a set of six CDR sequences of
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
and wherein the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44.

40. The polypeptide or polypeptide complex of claim 39, wherein the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44.

41. The polypeptide or polypeptide complex of claim 27, wherein the immunoglobulin light chain and the immunoglobulin heavy chain comprise a set of six CDR sequences of
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37;
and wherein the immunoglobulin heavy chain comprises an amino acid sequence of at least 95% sequence identity to SEQ ID NO: 239 and the immunoglobulin light chain comprises an amino acid sequence of at least 95% sequence identity to SEQ ID NO: 240.

42. The polypeptide or polypeptide complex of claim 41, wherein the immunoglobulin heavy chain comprises an amino acid sequence of at least 99% sequence identity to SEQ ID NO: 239 and the immunoglobulin light chain comprises an amino acid sequence of at least 99% sequence identity to SEQ ID NO: 240.

43. The polypeptide or polypeptide complex of claim 27, wherein the immunoglobulin light chain and the immunoglobulin heavy chain comprise a set of six CDR sequences of
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37;
and wherein the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46.

44. The polypeptide or polypeptide complex of claim 43, wherein the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46.

45. The polypeptide or polypeptide complex of claim 27, wherein the immunoglobulin light chain and the immunoglobulin heavy chain comprise a set of six CDR sequences of
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38;
and wherein the immunoglobulin heavy chain comprises an amino acid sequence of at least 95% sequence identity to SEQ ID NO: 241 and the immunoglobulin light chain comprises an amino acid sequence of at least 95% sequence identity to SEQ ID NO: 242.

46. The polypeptide or polypeptide complex of claim 45, wherein the immunoglobulin heavy chain comprises an amino acid sequence of at least 99% sequence identity to SEQ ID NO: 241 and the immunoglobulin light chain comprises an amino acid sequence of at least 99% sequence identity to SEQ ID NO: 242.

47. The polypeptide or polypeptide complex of claim 27, wherein the immunoglobulin light chain and the immunoglobulin heavy chain comprise a set of six CDR sequences of
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38;
and wherein the immunoglobulin heavy chain comprises an amino acid sequence of at least 95% sequence identity to SEQ ID NO: 47 and the immunoglobulin light chain comprises an amino acid sequence of at least 95% sequence identity to SEQ ID NO: 48.

48. The polypeptide or polypeptide complex of claim 47, wherein the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48.

49. The polypeptide or polypeptide complex of claim 1, wherein the immunoglobulin heavy chain comprises an amino acid sequence of at least 95%, 98%, 99%, or 100% sequence identity to SEQ ID NO: 251 and the immunoglobulin light chain comprises an amino acid sequence of at least 95%, 98%, 99%, or 100% sequence identity to SEQ ID NO: 252.

50. The polypeptide or polypeptide complex of claim 1, wherein the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 255 and the immunoglobulin light chain comprises an amino acid sequence of at least 90% sequence identity to SEQ ID NO: 256.

51. The polypeptide or polypeptide complex of claim 1, wherein the polypeptide or polypeptide complex that comprises the antibody or antigen binding fragment thereof that binds to STEAP-1 is a monospecific antibody, bispecific antibody, or trispecific antibody.

52. The polypeptide or polypeptide complex of claim 1, wherein the antibody or antigen binding fragment thereof that binds to STEAP-1 comprises a Fab, Fab′, (Fab′)₂ or a single chain variable fragment (scFv).

53. The polypeptide or polypeptide complex of claim 1, wherein the polypeptide or polypeptide complex comprises more than one antibody or antigen binding fragment thereof that binds to STEAP-1.

54. The polypeptide or polypeptide complex of claim 1, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L₁).

55. The polypeptide or polypeptide complex of claim 1, wherein the second antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H,
wherein CDR1-L comprises the amino acid sequence of SEQ ID NO: 80 or SEQ ID NO: 83;
wherein CDR2-L comprises the amino acid sequence of GTK;
wherein CDR3-L comprises the amino acid sequence of Z₁-Z₂-W-Z₃-Z₄-Z₅-Z₆-W-Z₇-Z₈ or SEQ ID NO: 82;
wherein Z₁ is V, G, P, L, I, M, S, T, or A;
Z₂ is L, G, P, V, I, M, S, T, or A;
Z₃ is Y, F, W, V, L, I, G, or A;
Z₄ is S, G, T, M, N, Q, H, or A;
Z₅ is N, Q, S, T, D, E, H, K, R, or A;
Z₆ is R, S, T, Q, D, E, H, K, N, or A;
Z₇ is V, G, P, L, I, M, S, T, or A; and
Z₈ is F, Y, W, V, L, I, G, or A;
wherein CDR1-H comprises the amino acid sequence of SEQ ID NO: 61 or SEQ ID NO: 64;
wherein CDR2-H comprises the amino acid sequence of SEQ ID NO: 62;
wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63; and
wherein Z₉ is V, G, P, L, I, M, S, T, or A;
Z₁₀ is R, S, T, Q, D, E, H, K, N, or A;
Z₁₁ is H, R, K, G, T, S, N, Q, or A;
Z₁₂ is G, P, V, L, I, M, S, T, or A;
Z₁₃ is F, Y, W, V, L, I, G, or A;
Z₁₄ is G, P, V, L, I, M, S, T, or A;
Z₁₅ is N, Q, S, T, D, E, H, K, R, or A;
Z₁₆ is S, G, T, M, N, Q, H, or A;
Z₁₇ is Y, F, W, V, L, I, G, or A;
Z₁₈ is I, G, P, V, L, M, S, T, or A;
Z₁₉ is S, G, T, M, N, Q, H, or A;
Z₂₀ is W, F, Y, V, L, I, G, or A; and
Z₂₁ is Y, F, W, V, L, I, G, or A.

56. The polypeptide or polypeptide complex of claim 55, wherein:
Z₁ is V, G, L, I, or A;
Z₂ is L, V, I, or A;
Z₃ is Y, W, F, or A;
Z₄ is S, G, T, or A;
Z₅ is N, Q, D, E, or A;
Z₆ is R, K, or A;
Z₇ is V, G, L, I, or A;
Z₈ is F, Y, W, or A;
Z₉ is V, G, L, I, or A;
Z₁₀ is R, K, or A;
Z₁₂ is G, S, T, or A;
Z₁₃ is F, Y, W, or A;
Z₁₄ is G, S, T, or A;
Z₁₅ is N, Q, D, E, or A;
Z₁₆ is S, G, T, or A;
Z₁₇ is Y, W, F, or A;
Z₁₈ is I, V, L, or A;
Z₁₉ is S, G, T, or A;
Z₂₀ is W, Y, F, or A; and
Z₂₁ is Y, W, F, or A.

57. The polypeptide or polypeptide complex of claim 55,
wherein CDR1-H of the second antibody or antigen binding fragment thereof that binds to CD3 comprises the amino acid sequence of SEQ ID NO: 61 or SEQ ID NO: 64;
wherein CDR2-H of the second antibody or antigen binding fragment thereof that binds to CD3 comprises the amino acid sequence of SEQ ID NO: 62;
wherein CDR3-H of the second antibody or antigen binding fragment thereof that binds to CD3 comprises the amino acid sequence selected from SEQ ID NOs: 63, 65-79, and 124;
wherein CDR1-L of the second antibody or antigen binding fragment thereof that binds to CD3 comprises the amino acid sequence of SEQ ID NO: 80 or SEQ ID NO: 83;
wherein CDR2-L of the second antibody or antigen binding fragment thereof that binds to CD3 comprises the amino acid sequence of GTK; and
wherein CDR3-L of the second antibody or antigen binding fragment thereof that binds to CD3 comprises the amino acid sequence selected from SEQ ID NOs: 82 and 84-93.

58. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 82.

59. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 65,
CDR1-L: SEQ ID NO: 80, CDR2-L: GTK, CDR3-L: SEQ ID NO: 82.

60. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 66,
CDR1-L: SEQ ID NO: 80, CDR2-L: GTK, CDR3-L: SEQ ID NO: 82.

61. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 67,
CDR1-L: SEQ ID NO: 80, CDR2-L: GTK, CDR3-L: SEQ ID NO: 82.

62. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 68,
CDR1-L: SEQ ID NO: 80, CDR2-L: GTK, CDR3-L: SEQ ID NO: 82.

63. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 69,
CDR1-L: SEQ ID NO: 80, CDR2-L: GTK, CDR3-L: SEQ ID NO: 82.

64. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 70,
CDR1-L: SEQ ID NO: 80, CDR2-L: GTK, CDR3-L: SEQ ID NO: 82.

65. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 71,
CDR1-L: SEQ ID NO: 80, CDR2-L: GTK, CDR3-L: SEQ ID NO: 82.

66. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 72,
CDR1-L: SEQ ID NO: 80, CDR2-L: GTK, CDR3-L: SEQ ID NO: 82.

67. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 73,
CDR1-L: SEQ ID NO: 80, CDR2-L: GTK, CDR3-L: SEQ ID NO: 82.

68. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 74,
CDR1-L: SEQ ID NO: 80, CDR2-L: GTK, CDR3-L: SEQ ID NO: 82.

69. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 75,
CDR1-L: SEQ ID NO: 80, CDR2-L: GTK, CDR3-L: SEQ ID NO: 82.

70. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 76,
CDR1-L: SEQ ID NO: 80, CDR2-L: GTK, CDR3-L: SEQ ID NO: 82.

71. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 77,
CDR1-L: SEQ ID NO: 80, CDR2-L: GTK, CDR3-L: SEQ ID NO: 82.

72. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 78,
CDR1-L: SEQ ID NO: 80, CDR2-L: GTK, CDR3-L: SEQ ID NO: 82.

73. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 79,
CDR1-L: SEQ ID NO: 80, CDR2-L: GTK, CDR3-L: SEQ ID NO: 82.

74. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 84.

75. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 85.

76. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 86.

77. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 87.

78. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 88.

79. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 89.

80. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 90.

81. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 91.

82. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 92.

83. The polypeptide or polypeptide complex of claim 55, wherein the second antibody or antigen binding fragment thereof that binds to CD3 comprises the following set of six CDRs:
CDR1-H: SEQ ID NO: 61, CDR2-H: SEQ ID NO: 62, CDR3-H: SEQ ID NO: 63,
CDR1-L: SEQ ID NO: 80, CDR2-L: GTK, CDR3-L: SEQ ID NO: 93.

84. The polypeptide or polypeptide complex of claim 55, wherein the second antibody or antigen binding fragment thereof that binds to CD3 comprises the following set of six CDRs:
CDR1-H: SEQ ID NO: 64, CDR2-H: SEQ ID NO: 62, CDR3-H: SEQ ID NO: 124,
CDR1-L: SEQ ID NO: 83, CDR2-L: GTK, CDR3-L: SEQ ID NO: 84.

85. The polypeptide or polypeptide complex of claim 1, wherein the second antibody or antigen binding fragment thereof that binds to CD3 comprises amino acid sequences with at least 90%, 95%, 96%, 97%, 98%, 99%, or 100% sequence identity to SEQ ID NO: 266 and SEQ ID NO: 267.

86. The polypeptide or polypeptide complex of claim 1, wherein the second antibody or antigen binding fragment thereof that binds to CD3 comprises amino acid sequences with at least 90%, 95%, 96%, 97%, 98%, or 100% sequence identity to SEQ ID NO: 268 and SEQ ID NO: 269.

87. The polypeptide or polypeptide complex of claim 1, wherein the second antibody or antigen binding fragment thereof that binds to CD3 comprises a Fab, Fab′, (Fab′)₂ or a single chain variable fragment (scFv).

88. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 96.

89. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 97.

90. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 98.

91. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 99.

92. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 100.

93. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 101.

94. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 102.

95. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 103.

96. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 104.

97. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 105.

98. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 106.

99. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 107.

100. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 108.

101. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 109.

102. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 110.

103. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 111.

104. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 112.

105. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 113.

106. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 114.

107. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 115.

108. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 116.

109. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 117.

110. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 118.

111. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 119.

112. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 120.

113. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 121.

114. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 122.

115. The polypeptide or polypeptide complex of claim 1, wherein the second antibody or antigen binding fragment thereof that binds to CD3 comprises an amino acid sequence with at least 90%, 95%, 96%, 97%, 98%, 99%, or 100% sequence identity to SEQ ID NO: 123.

116. The polypeptide or polypeptide complex of claim 1, wherein the antibody or antigen binding fragment thereof that binds to STEAP-1 is a Fab or Fab′, and the second antibody or antigen binding fragment thereof that binds to CD3 is an scFv and the Fab or Fab′ is connected to the scFv by a linker (L₂).

117. The polypeptide or polypeptide complex of claim 1, wherein the antibody or antigen binding fragment thereof that binds to STEAP-1 is a Fab or Fab′, and the second antibody or antigen binding fragment thereof that binds to CD3 is a Fab or Fab′ and the Fab or Fab′ that binds to STEAP-1 is connected to the Fab or Fab′ that binds to CD3 by a linker (L₂).

118. The polypeptide or polypeptide complex of claim 1, wherein the antibody or antigen binding fragment thereof that binds to STEAP-1 is an scFv, and the second antibody or antigen binding fragment thereof that binds to CD3 is a Fab or Fab′ and the scFv that binds to STEAP-1 is connected to the Fab or Fab′ that binds to CD3 by a linker (L₂).

119. The polypeptide or polypeptide complex of claim 1, wherein the antibody or antigen binding fragment thereof that binds to STEAP-1 is an scFv, and wherein a C-terminus of the scFv that binds to STEAP-1 is connected to an N-terminus of the second antibody or antigen binding fragment thereof that binds to CD3 via a linker (L₂).

120. The polypeptide or polypeptide complex of claim 1, wherein the antibody or antigen binding fragment thereof that binds to STEAP-1 is an scFv, and wherein an N-terminus of the scFv that binds to STEAP-1 is connected to a C-terminus of the second antibody or antigen binding fragment thereof that binds to CD3 via a linker (L₂).

121. The polypeptide or polypeptide complex of claim 54, wherein L₁ comprises an amino acid sequence according to any one of SEQ ID NOs: 222-224, 270-272, and 371.

122. The polypeptide or polypeptide complex of claim 118, wherein L₂ comprises an amino acid sequence according to any one of SEQ ID NOs: 222-224, 270-272, and 371.

123. The polypeptide or polypeptide complex of claim 1, wherein the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 128-137, 139-149, 283-284, 287-288, and 291-292, and an amino acid sequence, that comprises the immunoglobulin heavy chain that binds to STEAP-1, with at least 95%, 98%, 99%, or 100% sequence identity to any one of SEQ ID NOs: 176-185, 187-197, 308-309, 312-313, and 316-317.

124. The polypeptide or polypeptide complex of claim 1, wherein the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 126-127, 138, 273-282, 285-286, and 289-290, and an amino acid sequence, that comprises the immunoglobulin heavy chain that binds to STEAP-1, with at least 95%, 98%, or 99% sequence identity to any one of SEQ ID NOs: 174-175, 186, 299-307, 310-311, and 314-315.

125. The polypeptide or polypeptide complex of claim 1, wherein the polypeptide or polypeptide complex comprises an amino acid sequence, that comprises the immunoglobulin light chain that binds to STEAP-1, according to SEQ ID NO: 283 and an amino acid sequence, that comprises the immunoglobulin heavy chain that binds to STEAP-1, according to SEQ ID NO: 308.

126. The polypeptide or polypeptide complex of claim 1, wherein the polypeptide or polypeptide complex comprises an amino acid sequence that comprises the immunoglobulin light chain that binds to STEAP-1 according to SEQ ID NO: 284 and an amino acid sequence that comprises the immunoglobulin heavy chain that binds to STEAP-1 according to SEQ ID NO: 309.

127. The polypeptide or polypeptide complex of claim 1, wherein the polypeptide or polypeptide complex comprises an amino acid sequence that comprises the immunoglobulin light chain that binds to STEAP-1 according to SEQ ID NO: 287 and an amino acid sequence that comprises the immunoglobulin heavy chain that binds to STEAP-1 according to SEQ ID NO: 312.

128. The polypeptide or polypeptide complex of claim 1, wherein the polypeptide or polypeptide complex comprises an amino acid sequence that comprises the immunoglobulin light chain that binds to STEAP-1 according to SEQ ID NO: 288 and an amino acid sequence that comprises the immunoglobulin heavy chain that binds to STEAP-1 according to SEQ ID NO: 313.

129. The polypeptide or polypeptide complex of claim 1, wherein the polypeptide or polypeptide complex comprises an amino acid sequence that comprises the immunoglobulin light chain that binds to STEAP-1 according to SEQ ID NO: 291 and an amino acid sequence that comprises the immunoglobulin heavy chain that binds to STEAP-1 according to SEQ ID NO: 316.

130. The polypeptide or polypeptide complex of claim 1, wherein the polypeptide or polypeptide complex comprises an amino acid sequence that comprises the immunoglobulin light chain that binds to STEAP-1 according to SEQ ID NO: 292 and an amino acid sequence that comprises the immunoglobulin heavy chain that binds to STEAP-1 according to SEQ ID NO: 317.

131. The polypeptide or polypeptide complex of claim 1, wherein the polypeptide or polypeptide complex comprises an amino acid sequence, that comprises the immunoglobulin light chain that binds to STEAP-1, with at least 90%, 95%, 98%, 99%, or 100% sequence identity to any one of SEQ ID NOs: 152-161 and 164-173, and an amino acid sequence, that comprises the immunoglobulin heavy chain that binds to STEAP-1, with at least 95%, 98%, 99%, or 100% sequence identity to any one of SEQ ID NOs: 200-209 and 212-221.

132. The polypeptide or polypeptide complex of claim 1, wherein the polypeptide or polypeptide complex comprises an amino acid sequence, that comprises the immunoglobulin light chain that binds to STEAP-1, with at least 90%, 95%, 98%, or 99% sequence identity to any one of SEQ ID NOs: 150-151, 162-163, and 330-342 and an amino acid sequence, that comprises the immunoglobulin heavy chain that binds to STEAP-1, with at least 95%, 98%, 99%, or 100% sequence identity to any one of SEQ ID NOs: 198-199, 210-211, and 343-355.

133. The polypeptide or polypeptide complex of claim 1, wherein the polypeptide or polypeptide complex comprises amino acid sequences with at least 95%, 98%, or 99% sequence identity to SEQ ID NOs: 324, 325, and 326.

134. The polypeptide or polypeptide complex of claim 1, wherein the polypeptide or polypeptide complex comprises amino acid sequences with at least 95%, 98%, or 99% sequence identity to SEQ ID NOs: 327, 328, and 329.

135. The polypeptide or polypeptide complex of claim 1, wherein the polypeptide or polypeptide complex comprises amino acid sequences with at least 95%, 98%, or 99% sequence identity to SEQ ID NOs: 356, 357, 358, and 359.

136. The polypeptide or polypeptide complex of claim 1, wherein the polypeptide or polypeptide complex comprises amino acid sequences with at least 95%, 98%, or 99% sequence identity to SEQ ID NOs: 360, 361, 362, and 363.

137. The polypeptide or polypeptide complex of claim 1, wherein the polypeptide or polypeptide complex comprises amino acid sequences with at least 95%, 98%, or 99% sequence identity to SEQ ID NOs: 364, 365, 366, and 367.

138. A polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences according to
CDR1-H: SEQ ID NO: 3, CDR2-H: SEQ ID NO: 9, CDR3-H: SEQ ID NO: 15,
CDR1-L: SEQ ID NO: 21, CDR2-L: SEQ ID NO: 27, CDR3-L: SEQ ID NO: 33;
and wherein the polypeptide or polypeptide complex further comprises a second antibody or antigen binding fragment thereof that binds to CD3 wherein the second antibody or antigen binding fragment thereof that binds to CD3 comprises an immunoglobulin light chain comprising complementarity determining regions (CDRs): CDR1-L, CDR2-L, and CDR3-L, and an immunoglobulin heavy chain comprising CDRs: CDR1-H, CDR2-H, and CDR3-H,
wherein CDR1-L comprises the amino acid sequence of SEQ ID NO: 80 or SEQ ID NO: 83;
wherein CDR2-L comprises the amino acid sequence of GTK;
wherein CDR3-L comprises the amino acid sequence of Z₁-Z₂-W-Z₃-Z₄-Z₅-Z₆-W-Z₇-Z₈ or SEQ ID NO: 82;
wherein Z₁ is V, G, P, L, I, M, S, T, or A;
Z₂ is L, G, P, V, I, M, S, T, or A;
Z₃ is Y, F, W, V, L, I, G, or A;
Z₄ is S, G, T, M, N, Q, H, or A;
Z₅ is N, Q, S, T, D, E, H, K, R, or A;
Z₆ is R, S, T, Q, D, E, H, K, N, or A;
Z₇ is V, G, P, L, I, M, S, T, or A; and
Z₈ is F, Y, W, V, L, I, G, or A;
wherein CDR1-H comprises the amino acid sequence of SEQ ID NO: 61 or SEQ ID NO: 64;
wherein CDR2-H comprises the amino acid sequence of SEQ ID NO: 62;
wherein CDR3-H comprises the amino acid sequence of Z₉-Z₁₀-Z₁₁-Z₁₂-N-Z₁₃-Z₁₄-Z₁₅-Z₁₆-Z₁₇-Z₁₈-Z₁₉-Y-Z₂₀-A-Z₂₁ or SEQ ID NO: 63; and
wherein Z₉ is V, G, P, L, I, M, S, T, or A;
Z₁₀ is R, S, T, Q, D, E, H, K, N, or A;
Z₁₁ is H, R, K, G, T, S, N, Q, or A;
Z₁₂ is G, P, V, L, I, M, S, T, or A;
Z₁₃ is F, Y, W, V, L, I, G, or A;
Z₁₄ is G, P, V, L, I, M, S, T, or A;
Z₁₅ is N, Q, S, T, D, E, H, K, R, or A;
Z₁₆ is S, G, T, M, N, Q, H, or A;
Z₁₇ is Y, F, W, V, L, I, G, or A;
Z₁₈ is I, G, P, V, L, M, S, T, or A;
Z₁₉ is S, G, T, M, N, Q, H, or A;
Z₂₀ is W, F, Y, V, L, I, G, or A; and
Z₂₁ is Y, F, W, V, L, I, G, or A.

139. A pharmaceutical composition comprising:
(i) the polypeptide or polypeptide complex of claim 1; and
(ii) a pharmaceutically acceptable excipient.

140. An isolated recombinant nucleic acid molecule encoding the polypeptide or polypeptide complex of claim 1.

141. An expression vector comprising a nucleic acid encoding the polypeptide or polypeptide complex of claim 1.

142. A host cell comprising the polypeptide or polypeptide complex of claim 1.

143. A method of treating a cancer in a subject in need thereof comprising administering to the subject the polypeptide or polypeptide complex of claim 1.

144. The method of claim 143, wherein the cancer is sarcoma or a cancer of the prostate, breast, pancreas, bladder, gastrointestinal tract, testis, ovary, or cervix.

145. The method of claim 143, wherein the cancer is prostate cancer or Ewing sarcoma.

146. The method of claim 143, further comprising administering to the subject an immune checkpoint inhibitor.

147. The method of claim 146, wherein the immune checkpoint inhibitor comprises an antibody or antigen binding fragment thereof that binds to PD-1.
